SEQUENCE LISTING (83 sequences)

SEQ ID NO: 1 - DNA, 4109 bp, Homo sapiens
  exon (139)..(2055)
cagactttccgggcgcccaccaggctcccgggctctcaccggagaccacagggaggagcttcaaggtaccccaagtccctgcggcctccagcagtgaagagttccacaagggagtcttcctacgtcactgaataatgaatgaagatgagaggggaaaagagaagagacaaagtcaatccaaaaagctctcaaaggaaattaaactggattccatcatttcctacctatgattacttcaaccaagtgacgctacagttattagatggctttatgattacactgagcacagatggagtgatcatttgtgtggctgaaaacatctcttctcttcttggacatttaccagctgagattgtgggcaaaaaattattaagccttctgcctgatgaagagaaagatgaagtctaccaaaagattattctcaaatttcctttactaaactcagaaacacatattgaattttgctgtcatttaaaaagaggaaatgtcgaacatggtgatagttctgcttacgaaaacgtgaaatttattgtgaatgtaagagatatttgtaatgagtttcctgtggtctttagtggcttgttttccagccacctctgtgctgactttgctgcatgtgttcctcaggaggatcggctttatcttgtgggaaatgtttgcattctcaggactcagctcctgcagcaactttacacttcaaaggcagtcagtgatgaagctgtacttacacaagattcagatgaggaaccttttgtgggagagctcagtagctctcaaggtcaaagaggacacactagcatgaaagccgtgtacgttgaacccgctgctgctgctgctgctgctgctatctcagacgaccaaattgatattgcagaggttgagcagtatggaccacaagaaaacgttcacatgtttgtagattctgattcaacttattgctccagtacagttttcctggatactatgcctgaatctccagccttatccttgcaagactttcgaggtgagcctgaggtgaatccattgtacagggcagacccagtggacctggagttctcggtggatcaggtggactcagtggaccaggagggcccaatggaccagcaggacccagagaacccagttgccccgttggaccaggcaggcctgatggatccagtggatccagaggactcagtggacctgggggctgctggcgcaagtgctcagccattacagccatcatcaccagttgcatatgacatcattagccaggaactggaactgatgaagaagttgaaggagcagctagaagagaggacttggttgctgcatgatgccatccaaaaccagcagaatgcattggaattgatgatggatcaccttcagaagcagccaaacacattacgccacgttgtcattcctgatctccaatcttcggaggcagtgcccaagaaacaacagaaacaacacgctgggcaagtgaagcggcctctcccacatcccaaggacgtcaagtgtttctgtggcttatctttatccaactctctcaaaaacactggggagcttcaggagccttgtgttgccttcaaccagcagcaactggtgcagcaagaacaacacctgaaggagcagcagcggcagctgcgggagcagctgcaacagctgagagagcaaaggaaggtgcagaagcagaagaagatgcaggagaagaagaagctgcaggagcagaaaatgcaggagaagaagaagctgcaggagcagaggcggcaaaagaagaagaagctacaggagcggaagaagtggcaggggcagatgctacagaaagagccagaggaggagcagcagaagcagcagctgcaagagcagccactgaagcataatgtcatcgtggggaatgagagggtgcagatatgcctgcaaaacccacgtgacgtatctgtgcccctctgcaatcaccctgttagatttttacaggcccaacccattgttcctgtccagagagcagctgaacaacagccctctggcttctatcaagatgaaaactgtgggcaacaggaagatgagagtcaaaggtaagacatgcatggaatggtgatagtggctatgattattgcttttccctcatggctggatcccatgcctgagatcacagacagacgacctatccatgtggcaccagcgtccctctcatcaatagcaacttgctctcaacttgccttctctgtggccacggccatcatccttctgcatgtgtggtgcatggatgagcatccattgatggaacaaaggggtacccatgaagaaaggactagataaggaagcctcaatgctagggaattagcccacaaaccttgaaatcacttggctggattccccaagcccagtctcttgcacagggtgtctatggacaagtcttgtgcccatagctgaaagacttctgggcttccagtttcattccccaactttcctggaatctggtgatggcagtaggccaagtgcaggtatctccctggcatttatgaacagtaagagggaagtgagtgtgaaacaattgctagattgctaagatcagccaagaacacatgaaggctaagtcctgagtggcaatatagggacatgaacacatttctgctcaataataaggtcttcagttccaaatgctagaaagataaggttcttctcagcaatgaaaggattgcttagcacatagttggggcccactaaatagttttgaatgaatgttagctttctgagacgggcctcctaaagctctctggacttccttctgggccttgctttcgaatcagtgactttgacatgaagactgctttagggccatattgtccacttgacctgatcatcagaatccacctgaggagggtttggaaagtatagattcagagtctgggatggggttcaaaaatctgtattgatcacaagacttccaggcttagggttgtggagagagcactggatttggagtttggaaatctgccttacagtctctgctcagccattagctccctgagtgctttcaaagtaggtgaaatgactttccaaggccacagagagcatagttgtagcaggaacaagactgtccaagtaatataaataaaaccaggtgagacaagggattgtaatttctctcagtgtctacattcacattactttgacaagttgaatcttagtcaggccaaagtgagaatacaaacaagtgaatcctgtggtgtagccaccaaaaccaaatgtgcctgaagcacctactgatgaccacgtgagtgtctcatcagttatcatgtccagatccacatcactgcaacagctttcttctcttacagtttttatcctgaggcgtatcaagggccccccgtgaaccagctgccattgatagatacctcaaactctgaggcaatttcttcttccagcattcctcagtttcccataacttcagactcaaccataagcaccctggagaccccacaggattacatccggctttggcaagagttgtctgattcactcggtcctgttgtccaagtgaacacttggtcttgcgatgagcagggcaccctgcacggccaacccacctaccatcaggtgcaagtttctgaggtaggagtcgagggacctcctgatccacaggctttccaaggccctgctgcataccagccagaccagatgagatctgcggagcagaccagattgatgcctgcagagcaacgtgactcaaataagccgtgctaacagtactttcatgaccagtgatgaggggaaatggggggagggggcaggccaatgaggtctgcatggccaggggaccttcaaggtgcataaagtcccttggggtagggtttagtgggtagagacttgtttcctgataggttatgtttgtaattgtttgttaagcacagcctgtttcttggaagttatgctgtagaggcagcctgtgatccgtagtatgctagggtgtgacagcagccagccacagctggatctgatgtcttgtctgccccgcccagctttgcatatccatgttctaccacaggaaggtggcctgccaagagtctgctcaaagttttcaacataaagaataaagaaaaaaaaatgccaaaaaaaaaaaaaaaaaa

SEQ ID NO: 2 - protein, 639 aa, Homo sapiens
MKMRGEKRRDKVNPKSSQRKLNWIPSFPTYDYFNQVTLQLLDGFMITLSTDGVIICVAENISSLLGHLPAEIVGKKLLSLLPDEEKDEVYQKIILKFPLLNSETHIEFCCHLKRGNVEHGDSSAYENVKFIVNVRDICNEFPVVFSGLFSSHLCADFAACVPQEDRLYLVGNVCILRTQLLQQLYTSKAVSDEAVLTQDSDEEPFVGELSSSQGQRGHTSMKAVYVEPAAAAAAAAISDDQIDIAEVEQYGPQENVHMFVDSDSTYCSSTVFLDTMPESPALSLQDFRGEPEVNPLYRADPVDLEFSVDQVDSVDQEGPMDQQDPENPVAPLDQAGLMDPVDPEDSVDLGAAGASAQPLQPSSPVAYDIISQELELMKKLKEQLEERTWLLHDAIQNQQNALELMMDHLQKQPNTLRHVVIPDLQSSEAVPKKQQKQHAGQVKRPLPHPKDVKCFCGLSLSNSLKNTGELQEPCVAFNQQQLVQQEQHLKEQQRQLREQLQQLREQRKVQKQKKMQEKKKLQEQKMQEKKKLQEQRRQKKKKLQERKKWQGQMLQKEPEEEQQKQQLQEQPLKHNVIVGNERVQICLQNPRDVSVPLCNHPVRFLQAQPIVPVQRAAEQQPSGFYQDENCGQQEDESQR

SEQ ID NO: 3 - DNA, 2878 bp, Homo sapiens
  exon (151)..(2470)
atttccagactttccgggcgcccaccaggctcccgggctctcaccggagaccaccggagaccacagggaggagcttcaaggtaccccaagtccctgcggcctccagcagtaagagttccacaagggagtcttcctacgtcactgaataatgaatgaagatgagaggggaaaagagaagagacaaggtcaatccaaaaagctctcaaaggaaattaaactggattccatcatttcctacctatgattacttcaaccaagtgacgctacagttattagatggctttatgattacactgagcacagatggagtgatcatttgtgtggctgaaaacatctcttctcttcttggacatttaccagctgagattgtgggcaaaaaattattaagccttctgcctgatgaagagaaagatgaagtctaccaaaagattattctcaaatttcctttactaaactcagaaacacatattgaattttgctgtcatttaaaaagaggaaatgtcgaacatggtgatagttctgcttacgaaaacgtgaaatttattgtgaatgtaagagatatttgtaatgagtttcctgtggtctttagtggcttgttttccagccacctctgtgctgactttgctgcatgtgttcctcaggaggatcggctttatcttgtgggaaatgtttgcattctcaggactcagctcctgcagcaactttacacttcaaaggcagtcagtgatgaagctgtacttacacaagattcagatgaggaaccttttgtgggagagctcagtagctctcaaggtcaaagaggacacactagcatgaaagccgtgtacgttgaacccgctgctgctgctgctgctgctgctatctcagacgaccaaattgatattgcagaggttgagcagtatggaccacaagaaaacgttcacatgtttgtagattctgattcaacttattgctccagtacagttttcctggatactatgcctgaatctccagccttatccttgcaagactttcgaggtgagcctgaggtgaatccattgtacagggcagacccagtggacctggagttctcggtggatcaggtggactcagtggaccaggagggcccaatggaccagcaggacccagagaacccagttgccccgttggaccaggcaggcctgatggatccagtggatccagaggactcagtggacctgggggctgctggcgcaagtgctcagccattacagccatcatcaccagttgcatatgacatcattagccaggaactggaactgatgaagaagttgaaggagcagctagaagagaggacttggttgctgcatgatgccatccaaaaccagcagaatgcattggaattgatgatggatcaccttcagaagcagccaaacacattacgccacgttgtcattcctgatctccaatcttcggaggcagtgcccaagaaacaacagaaacaacacgctgggcaagtgaagcggcctctcccacatcccaaggacgtcaagtgtttctgtggtttatctttatccaactctctcaaaaacactggggagcttcaggagccttgtgttgccttcaaccagcagcaactggtgcagcaagaacaacacctgaaggagcagcagcggcagctgcgggagcagctgcaacagctgagagagcaaaggaaggtgcagaagcagaagaagatgcaggagaagaagaagctgcaggagcagaaaatgcaggagaagaagaagctgcaggagcagaggcggcaaaagaagaagaagctacaggagcggaagaagtggcaggggcagatgctacagaaagagccagaggaggagcagcagaagcagcagctgcaagagcagccactgaagcataatgtcatcgtggggaatgagagggtgcagatatgcctgcaaaacccacgtgacgtatctgtgcccctctgcaatcaccctgttagatttttacaggcccaacccattgttcctgtccagagagcagctgaacaacagccctctggcttctatcaagatgaaaactgtgggcaacaggaagatgagagtcaaagtttttatcctgaggcgtatcaagggccccccgtgaaccagctgccattgatagatacctcaaactctgaggcaatttcttcttccagcattcctcagtttcccataacttcagactcaaccataagcaccctggagaccccacaggattacatccggctttggcaagagttgtctgattcactcggtcctgttgtccaagtgaacacttggtcttgcgatgagcagggcaccctgcacggccaacccacctaccatcaggtgcaagtttctgaggtaggagtcgagggacctcctgatccacaggctttccaaggccctgctgcataccagccagaccagatgagatctgcggagcagaccagattgatgcctgcagagcaacgtgactcaaataagccgtgctaacagtactttcatgaccagtgatgaggggaaatggggggagggggcaggccaatgaggtctgcatggccaggggaccttcaaggtgcgtaaagtcccttggggtagggtttagtgggtagagacttatttgtttcctgataggttatgtttgtaattgtttgttaagcacagcctgtttcttggaagttatgctgtagaggcagcctgtgatccgtagtatgctagggtgtgacagcagccagccacagctggatctgatgtcttgtctgccccgcccagctttgcatatccatgttctaccacaggaaggtggcctgccaagagtctgctcaaagttttcaacataaagaataaagaaaaaaaaaaatgcaaaaaaaaaaaaaaaaaaaaaaaaaaaaaaaaaa

SEQ ID NO: 4 - DNA, 3577 bp, Homo sapiens
gctaaattgtgtttccccccaccgaaattcatgcgttgaatcctaatccccagtacctcaaaatgtgactgtatttggaggtagaaactttaaaaggtgattaagttaaaatgaggtcattagggtgggccctaatccaatctgactggtgtctttatgagaagaggagattaggccacatggagaaaccagggatgcctgcacacagagaaaagaccttttgaggatgtagtgagaagataccatctgcaaggcaaggaaaagggcctcagaagaaaccaaactcacaagaaagccaacaccttgatcttagacttccagcctccagaattgtgagaaaacacatttctgtttaagccacccaatctgtggtgttttgtggtggcagccctagcaaactaatacaggtagctactgtgaatgggaacttcccgtattatatattacatctctaaccagttatttgttttgtaggaaatatgatgattttgtagtttattttcctggattttctggatggaaaaatgtttttatctgcaaatgatcacagttttgctgcccactttctagtcattttgccttttgtttttttttcttttcttttttttttttttttttgagacaagagtctcgctctgtcacccaggctggagtgcagtggtgtgatctcggctcactgcaacctccatctcctgggttcaagcgattcttctgcctcagcctcctgagtagctgggactacaggcacatgccaccatgcccggctaatttttgtatttttagtagagatggggtttcaccatgttggccaggatggtctcgatctcctgacctcgtgatccacatgcctcggcctcacaaagtgctgggattacaggcatgagccaccactcctggccttttatttctttttaaatccttgctcatatatctttgagtatatctgtgaatatttctatcagacaaattcataaaagttattactagtttgtaatgttcctttttctaaagaatgggcatgcagttttattaaatatcttttagtgtccattgagatgaccatttttttccctttattctgtttttttatatgtattacattagcccatctccaatattaaactagctttgtgtttctgggataaaaactcctttgacatttgacgtgtgctgacactgacattctttgagtaaataaaataaaaaacaggagtataccagtgtacaccaacataccctgctgctttattcgggttactgactatactttctgtctgaaattgtctgctgtgtcctcagtagatgactctatttagtaactacaaaccctgagtcaggctttcagaatgcccttctttttatttattagtagagatgagatcttgctatgttgccagcgctggtctcgaactcctgggctcaagtaatcctctctcctcagcctcccaaagtgctgggattacaggcatgagccactgttcctggccagagtgaccttctaatggtgcttccatggacctctcctttgctcctattttctttctcttgcttttggagctgctcctttagaggtgagaccacccttcaagtgggtgaaatcagctgtgagtctctcattgcttctgtgtacttcccacagcctagtaaattttctctttgctcctcaagccctggaacatttgggcctccacccacctgtcttactctctgcggagagtcttgcttcatatttagagtcagattaagaccagtggaaatgaactgccttacattttctctgtttcagcatatcttcctctgtcacctattttctccaggtttggataaaaggtagtctctcctgataattctcaacacctgatggacacctattccaggagaacactctgcatcctgaagctcattctgtcctaaatggatcccatatctcctctcaacgcagagacaaaaacaaacattccttaggcctaggaccatcacttcccttgtgtctgttcatggaccacaataccttgcctgcttcaatggctgcaagtgtttatcatctctgtcttgtctgggccctgcatacaagtgacctgccctcatatccaacactccagtttagactccagtaacctcttactggtgggttctaataaccttttaataacttccctctatcttgccccttgctttatattgagatcatattcattgtttcccctccatcttgctgcctggatcatgaatgatgtctataccacagggaccaggatgccatctagggacaatggagcaatgagctgggaggaacctggatcctgaacattgtgtggagtacagctgtcataaggctgaggttgcattgtgtgtgtgtgtgtgtgtgtgtgagtgagtgtgtgtgtgtgtgtgttgtttactttaacctgagcttatgagagctctgggtaatgtaaatagaatagctcctttaggaagtgtcttagtcattttgggctgctataacaaaaatgctgtatactggtggcttaaataactatttatcataattttggaggctgggaagtccaagatccagacactggcagatctggcatcaggtgggggcctgcttcctggcttatagacagttgccttcttgctgtatcttcacatagctgatggagagatcattttgctcttatgtcttcttataagggcactcatcccatttgtgaaggctccaccctcatgacctgattacctccccaaagcactacctgctagtaccatcacattggggattaggcttcaacctatacattttatgaggaaatgaacaatcagtccatatcagggagaaataaatatatgtaactataatcacaagttaagtagttaaaataaaaaatatcaatatcttattctgtgtgtggccctgccaatttactaaaagctatcataaaggacactgatcataaaacttagccaatcattttagcctgcacaaattctatacggtatttgaaataatcaaccaaacaataattcttcaaactttgtttcgttttgttttgttttgagacagtttcgctctgtcgcccaggctggagtgcagtggcacaatctcggttcactgcaacctctgcctcccgggttcaagcagttctcttgcctcagcctcccaagtaactggtactacagtttgtcctcggctgttacagagaattgatacaaaaaatgatcatgtgaagaatttgcatcttctgcagaactaccggtatcagtcagcagtaggagattgccagacacggtggctcacgcctataatcctagcactttgggaggctgaggtgtgtggatcacttgagcccaggagttcaagaccagcctgagcaacatggtgaaaccccatctctgcaaaaaatacaaaaattagccaggcgtggtggtgtgtacctgtggtcccagctgctgggaaggctgaggtgggaggaacatttgagcccagaagttgagccgtgatcacgccactgcactccagcctggatgacacagcaagaccttgtctcaaaaaataaaataaaataaattttaaaaaaaaaaaaaaaaaaaa

SEQ ID NO: 5 - protein, 110 aa, Homo sapiens
MVLPWTSPLLLFSFSCFWSCSFRGETTLQVGEISCESLIASVYFPQPSKFSLCSSSPGTFGPPPTCLTLCGESCFIFRVRLRPVEMNCLTFSLFQHIFLCHLFSPGLDKR

SEQ ID NO: 6 - DNA, 726 bp, Homo sapiens
ctcgtgccgaattcggcacgaggaagatggcgcctgttgtgacaggggaagctatgcgaaattatttaaaagagcgaggggatcaaacagtacttattcttcatgcaaaagttgcacagaagtcatatggaaatgaaaaaaggtttttttgcccacctccttgtgtatatcttatgggcagtggatggaagaaaaaaaaagaacaaatggaacgcgatggttgttctgaacaagagtctcaaccgtgtgcatttattgggataggaaatagtgaccaagaaatgcagcagctaaacttggaaggaaagaactattgcacagccaaaacattgtatatatctgactcagacaagcgaaagcacttcatgttgtctgtaaagatgttctatggcaacagtgatgacattggtgtgttcctcagcaagcggataaaagtcatctccaaaccttccaaaaagaagcagtcattgaaaaatgctgacttatgcattgcctcaggaacaaaggtggctctgtttaatcgactacgatcccagacagttagtaccagatacttgcatgtagaaggaggtaattttcatgccagttcacagcagtggggagccttttttattcatctcttggatgatgatgaatcagaaggagaagaattcacagtccgagatggctacatccattatggacaaacagtcaaacttgtgtgctcagttactggcatggcactcc

SEQ ID NO: 7 - protein, 241 aa, Homo sapiens
RAEFGTRKMAPVVTGEAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMAL

SEQ ID NO: 8 - protein, 485 aa, Homo sapiens
MGGCRKFGERPPPKRLTREAMRNYLKERGDQTVLILHAKVAQKSYGNEKRFFCPPPCVYLMGSGWKKKKEQMERDGCSEQESQPCAFIGIGNSDQEMQQLNLEGKNYCTAKTLYISDSDKRKHFMLSVKMFYGNSDDIGVFLSKRIKVISKPSKKKQSLKNADLCIASGTKVALFNRLRSQTVSTRYLHVEGGNFHASSQQWGAFFIHLLDDDESEGEEFTVRDGYIHYGQTVKLVCSVTGMALPRLIIRKVDKQTALLDADDPVSQLHKCAFYLKDTERMYLCLSQERIIQFQATPCPKEPNKEMINDGASWTIISTDKAEYTFYEGMGPVLAPVTPVPVVESLQLNGGGDVAMLELTGQNFTPNLRVWFGDVEAETMYRCGESMLCVVPDISAFREGWRWVRQPVQVPVTLVRNDGIIYSTSLTFTYTPEPGPRPHCSAAGAILRANSSQVPPNESNTNSEGSYTNASTNSTSVTSSTATVVS

SEQ ID NO: 9 - DNA, 489 bp, Homo sapiens
cgaggactggttccagttcactcggcagcggcgccgggcggagggggagagcgcgggccgcgcgggcgggaagcgaagaggcgggcgggccagcgaggagcgcggagagaaaaggcgcgagcggccaggagggctcaggccgagacaccttgcagctgccgccgccgccaccgagccgccgctgtgctcactgatccgcctccagggccaccgccatgtcgagccgcggtgggaagaagaagtccaccaagacgtccaggtctgccaaagcaggagtcatctttcccgtggggcggatgctgcggtacatcaagaaaggccaccccaagtacaggattggagtgggggcacccgtgtacatggccgccgtcctggaatacctgacagcggagattctggagctggctggcaatgcagcgagagacaacaagaagggacgggtcacaccccggcacatcctgctggctgtggccaatgatgaagagctga

SEQ ID NO: 10 - protein, 769 aa, Homo sapiens
FGHVDQENSPSFMFQGSSNTEFKSTKEGFSIAVSADGFKFGISEPGNQEKKSEKPLENDTGFQAQDISGQKNGRGVIFGQTSSTFTFADVAKSTSGEGFQFGKKDPNFKGFSGAGEKLFSSQCGKMANKANTSGDFEKDDDACKTEDSDDIHFEPVVQMPEKVELVTGEEGEKVLYSQGVKLFRFDAEISQWKERGLGNLKILKNEVNGKPRMLMRRDQVLKVCANHWITTTMNLKPLSGSDRAWMWLASDFSDGDAKLERLAAQFKTPELAEEFKQKFEECQRLLLDIPLQTPHKLVDTGRAAKLIQRAEEMKSGLKDFKTFLTNDQTKVTEEENKGSGTGAAGASDTTIKPNPENTGPTLEWDNYDLREDALDDNVSSSSVHDSPLASSPVRKNIFRFDESTTGFNFSFKSALSLSKSPAKLNQSGTSVGTDEESDVTQEEERDGQYFEPVVPLPDLVEVSSGEENEQVVFSHMAELYRYDKDVGQWKERGIGDIKILQNYDNKQVRIVMRRDQVLKLCANHRITPDMSLQNMKGTERVWVWTACDFADGERKVEHLAVRFKLQDVADSFKKIFDEAKTAQEKDSLITPHVSRSSTPRESPCGKIAVAVLEETTRERTDVIQGDDVADAASEVEVSSTSETTTKAVVSPPKFVFGSESVKRIFSSEKSNPFAFGNSSATGSLFGFSFNAPLKSNDSETSSVAQSGSESKVEPKKCELSKNSDIEQSSDSKVKNLSASFPMEESSINYTFKTPEKAKEKKKPEDSPSD

SEQ ID NO: 11 - DNA, 1288 bp, Homo sapiens
  exon (3)..(1288)
cagaggcagcccgggcagtcgccgggtcggtagaggtgctgacagggcggagccgccgcttgcctcagccggcctcacccctcccgcccgggacaggtggctggagcgcgctccgcctccgccagctgcgagtgggagcgagggagggccggtcccggacggtcgccgccagctgcgagtggaagtgagcgagggctagtcccggaccgccgggccaggggtccggcggcagcagacgggtaccgtggcggccaaaaaaatgctcctgtaccgaggggcccccgccggccctggcgcgccgggctgcgggctggcccggcccggcggcggcccgcaggccttcgggatccgcctgagcacgatgagcccccgctacctccagagcaactccagcagccacacgcgacccttcagtgccatcgcggagctgctagataatgctgtagatccagatgtatctgccaggacggtctttatagatgttgaggaggtcaagaataaatcttgtttgacctttaccgatgatggatgtgggatgacacctcataaactacaccgaatgctcagcagatgaggttgattactctccctgtggcttcctgaactagagtgtgtgaagcacgagtaacaatggtggaatgctgtggaggagggaaagggctcccaactataaaaatggaaaacaaattcatgtggttgtagttacccttaaataaaatggaaaatctgagttggactttgatacagatcaatatgacatcctggtatcagactttgacacagaagaaaaaatgactggcggtgttacctctgagctaccagaaacagaatattctttaagggcattttgtggtattctatacatgaagccacgcatgaaaatttttctgcgtcaaaagaaggtgactacccagatgattgccaagagcctggccaatgtagaatatgatacatataaacctaccttcacaaataagcaggtgagaatcacctttgggttctcttgcaagaatagtaaccagtttggaataatgatgtatcataacaaccgactcataaaatcttttgagaaggtggggtgccaggtgaagccaactcgtggagaaggtgtaggagtaattggagtcattgagtgcaatttcctaaaacctgcctacaacaaacaagactttgagtataccaaggagtaccggctaacaataaatgcccttgcccagaagctcaatgcttactggaaggaaaaaacatctcaagataattttgagacctcaactgtagccaggccaat

SEQ ID NO: 12 - DNA, 3037 bp, Homo sapiens
  exon (19)..(2302)
aaccagcaaaacaaaaaaatgattattaccgaggattcattgcccagcctagaagccatcttgaactattccattttcaaccgtgaaaatgacctgctggcccagtttgatgccatcccaggcaaaaaaggcactcgtgttctcatttggaacatccgcagaaataaaaatggaaaatctgagttggactttgatacagatcaatatgacatcctggtttcagactttgacacagaagaaaaaatgactggcggtgttacctctgagctaccagaaacagaatattctttaagggcattttgtggtattctatacatgaagccacgcatgaaaatttttctgcgtcaaaagaaggtgactacccagatgattgccaagagcctggccaatgtagaatatgatacacataaacctaccttcacaaataagcaggtgagaatcacctttgggttctcttgcaagaatagtaaccagtttggaataatgatgtatcataacaaccgactcataaaatcttttgagaaggtggggtgccaggtgaagccaactcgtggagaaggtgtaggagtaattggagtcattgagtgcaatttcctaaaacctgcctacaacaaacaagactttgagtataccaaggagtaccggctaacaataaatgcccttgcccagaagctcaatgcttactggaaggaaaaaacatctcaagataattttgagacctcaactgtagccaggccaataccgaaggttcctgaccagacatgggttcagtgtgatgagtgtcttaaatggagaaagcttcctgggaagattgatccatccatgttacctgcaagatggttttgttattataattcccatccaaagtacaggagatgctctgttccagaggaacaagaactcattgatgaagacctgtgcttgagcaaagctaagaaacaagaacaaactgttgaggagaagaagaagatgcctatggaaaatgagaaccaccaggtattcagtaatccaccaaagatcctcactgttcaagaaatggctggattgaataacaagacaattggatatgagggaattcatagccctagtgtgcttccttctggtggagaagaaagcagatcaccatctcttcaacttaagcctctggattccagtgttttacagttttccagtaagtacaaatggatcctaggtgaagaaccggtggagaaacgaagaaggctccagaatgagatgacaacaccttctctagattattccatgcctgctccttacaggagggtagaagcacctgttgcctacccagaaggggagaacagccatgataaatcgagttctgagagaagtacaccaccataccttttcccagaatacccagaagcaagcaagaatacaggtcagaatagggaggtttcaattctgtatccaggggccaaagaccaacgccaggggtccctgcttcctgaagaattagaagatcagatgccaagattggtggcagaagaatctaacagaggtagcacaaccataaacaaagaagaagtcaacaagggaccttttgtagctgttgtgggtgttgccaaaggtgttagagattcaggagctcccattcagctgatcccttttaacagagaggagcttgctgagagacgaaaagcagttgaatcctggaacccagtgccttattctgtggcctctgctgcaatccctgctgcagccattggggagaaagcaagaggctatgaggagagcgaaggtcataatacaccaaagttgaagaaccagagagagctggaagaattgaagagaaccacagaaaaattggaacgtgttttggctgaaaggaatttgttccagcaaaaggtggaggagctggaacaggagaggaatcactggcagtctgaattcaagaaagtccaacatgaattggtgatctacagtacccaggaggcggaaggcttgtactggagcaagaaacacatgggttatcgccaagctgaattccagattctgaaagctgagctggaaagaaccaaagaggaaaagcaagagttaaaagagaaactgaaggaaacagagacacacctggaaatgctgcagaaggctcaggtctcctaccggaccccagagggagatgacctagaaagggctttggcaaagcttacgcggctacgtatccacgtcagctatctccttacttctgtcctccctcacttggagcttcgtgagatcgggtatgactcagaacaagtggatgggatcctgtacacggtgctggaggcaaatcacatactggattgagcaccagactgtatacccttctcttctcttatcttctgtctgttctcttttctctccctccctcacgtctctctctctctctctctctctctctctctctctcaccctcacctttatgccttatatagagaatctctgtgtaaatcctggctcataatcagtctcctttttatcagttttggtgtggagaaagaggccagtttaaataggctttcaagagtctagggtcagaaaagcaatagtcactaagctaggtgacctgaaagctttaattttcatgacctggatatgtggtctattgtatatctttttctgaaatggtttgtattcatttaggttagatcaatcagcagatattgggtccggtataccaggtattattttggggtaagctaacaagtacaactcatgtttgcagccttcgaagatgtaacaatctcgttggaaacataagacatacatcacattatacacaaaagtgtatgatatgtacaactgagtggtacagatatgacatggaagatctgggggaggaagttcaaggaaggcaccacaccagaaatgggacctaaattaaggcttaaagaatgagcatggccacactgtcagtgagtgttctttaggtggtaggactatggttgatatttttcttcttcctatcttcctgcctttttcagattttcaatattaaacttgttattcttacattggaaaaaaataaattgtttttaaaaag

SEQ ID NO: 13 - DNA, 644 bp, Homo sapiens
  exon (13)..(644)
aattcggcacgagctcgtgccgacggaaatttaagaaaacgacttatggattatgtacttcagatcacagcacaactagaagatgttgatacattacataaaaaatcaggaaacctatgaagaacatcaaaagtggcaagtactttgttttgaaaagttcagatcaactgataatactagtgctgtgatgagactcagcttgtactcaagaacagatttatgactagcttatagttggctgcttcaccaggactctacaaattgctcagaacccaaggagacctagaggatcaagacataatgggagcatttttagacaagccaaagatggaaaagcataatgcccaggggcagggtaatgggttgcgatatgggctaagcagcatgcaaggctggcgtgttgaaatggaggatgcacatacggctgtgatcggtttgccaagtggacttgaatcgtggtcattctttgctgtgtatgatgggcatgctggttctcaggttgccaaatactgctgtgagcatttgttagatcacatcaccaataaccaagattttaaagggtctgcaggagcaccttctgtggaaaatgtaaagaatggaatcagaacaggttttctggagattgatgaacacatgagagttat

SEQ ID NO: 14 - DNA, 20 bp, Artificial
  Primer
tacaggagcggaagaagtgg

SEQ ID NO: 15 - DNA, 22 bp, Artificial
  Primer
tctcatcaatagcaacttgctc

SEQ ID NO: 16 - DNA, 21 bp, Artificial
  Primer
tccagagagcaggctgaacaa

SEQ ID NO: 17 - DNA, 20 bp, Artificial
  Primer
ggtaatgggttgcgatatgg

SEQ ID NO: 18 - DNA, 20 bp, Artificial
  Primer
acaccaccataccttttccc

SEQ ID NO: 19 - DNA, 20 bp, Artificial
  Primer
aggatttcaaaaccgacctg

SEQ ID NO: 20 - DNA, 21 bp, Artificial
  Primer
acataccacttcaaactcccc

SEQ ID NO: 21 - DNA, 21 bp, Artificial
  Primer
accaggaagaggatgaagaag

SEQ ID NO: 22 - DNA, 20 bp, Artificial
  Primer
tcccaatctcgtcctctgtc

SEQ ID NO: 23 - DNA, 21 bp, Artificial
  Primer
aagagcaagaacataacggac

SEQ ID NO: 24 - DNA, 20 bp, Artificial
  Primer
gaataaacgcaaggcaatgg

SEQ ID NO: 25 - DNA, 22 bp, Artificial
  Primer
ggctttggctttgttacttttg

SEQ ID NO: 26 - DNA, 22 bp, Artificial
  Primer
ggaaattcaactactgaggagg

SEQ ID NO: 27 - DNA, 22 bp, Artificial
  Primer
gctggggaagaaaaaacatagg

SEQ ID NO: 28 - DNA, 21 bp, Artificial
  Primer
atggtttgttgcctacctagc

SEQ ID NO: 29 - DNA, 21 bp, Artificial
  Primer
cacaaccccaaaattaacacc

SEQ ID NO: 30 - DNA, 20 bp, Artificial
  Primer
ttagttctgttcccaccacc

SEQ ID NO: 31 - DNA, 22 bp, Artificial
  Primer
aaactacttcgcaactataccc

SEQ ID NO: 32 - DNA, 19 bp, Artificial
  Primer
aaatctgcccaccgacatc

SEQ ID NO: 33 - DNA, 19 bp, Artificial
  Primer
agaaaagcgacccaagcag

SEQ ID NO: 34 - DNA, 19 bp, Artificial
  Primer
acaaagccaagtcccgttc

SEQ ID NO: 35 - DNA, 20 bp, Artificial
  Primer
atcagaccaatccaaggcag

SEQ ID NO: 36 - DNA, 32 bp, Artificial
  Primer
atctggcaccacaccttctacaatgagctgcg

SEQ ID NO: 37 - DNA, 19 bp, Artificial
  Primer
acaggaacaatgggttggg

SEQ ID NO: 38 - DNA, 21 bp, Artificial
  Primer
tcacactcacttccctcttac

SEQ ID NO: 39 - DNA, 20 bp, Artificial
  Primer
aagccggatgtaatcctgtg

SEQ ID NO: 40 - DNA, 20 bp, Artificial
  Primer
gcggattacttggtttgtga

SEQ ID NO: 41 - DNA, 20 bp, Artificial
  Primer
ttctttccagctcagctttc

SEQ ID NO: 42 - DNA, 19 bp, Artificial
  Primer
caactggatgtctttgggc

SEQ ID NO: 43 - DNA, 22 bp, Artificial
  Primer
atctttatatcaccaatgcccc

SEQ ID NO: 44 - DNA, 22 bp, Artificial
  Primer
acgttttaggcagatttcagtc

SEQ ID NO: 45 - DNA, 21 bp, Artificial
  Primer
accctccctaaatcctagctc

SEQ ID NO: 46 - DNA, 21 bp, Artificial
  Primer
cgaccactcatacttctccac

SEQ ID NO: 47 - DNA, 20 bp, Artificial
  Primer
caaggagagaagaggtgagg

SEQ ID NO: 48 - DNA, 20 bp, Artificial
  Primer
tatcctcctcttcctcctcc

SEQ ID NO: 49 - DNA, 18 bp, Artificial
  Primer
aggtcggagatttgaggg

SEQ ID NO: 50 - DNA, 20 bp, Artificial
  Primer
gatcatccatgaggaactgc

SEQ ID NO: 51 - DNA, 21 bp, Artificial
  Primer
attcatgcttgccttgggatg

SEQ ID NO: 52 - DNA, 19 bp, Artificial
  Primer
acagaaatgaccccactcc

SEQ ID NO: 53 - DNA, 22 bp, Artificial
  Primer
gctcatctctttgtctccactc

SEQ ID NO: 54 - DNA, 21 bp, Artificial
  Primer
tcaccacctaacctatgacac

SEQ ID NO: 55 - DNA, 20 bp, Artificial
  Primer
ccccatcaccacatctttcc

SEQ ID NO: 56 - DNA, 20 bp, Artificial
  Primer
tgaatccacagcagtaggag

SEQ ID NO: 57 - DNA, 21 bp, Artificial
  Primer
atcactacaccacacaaaacc

SEQ ID NO: 58 - DNA, 21 bp, Artificial
  Primer
agcatggagataaaggtgcag

SEQ ID NO: 59 - DNA, 2067 bp, Homo sapiens
atttccagactttccgggcgcccaccaggctcccgggctctcaccggagaccaccggagaccacagggaggagcttcaaggtaccccaagtccctgcggcctccagcagtgaagagttccacaagggagtcttcctacgtcactgaataatgaatgaagatgagaggggaaaagagaagagacaaggtcaatccaaaaagctctcaaaggaaattaaactggattccatcatttcctacctatgattacttcaaccaagtgacgctacagttattagatggctttatgattacactgagcacagatggagtgatcatttgtgtggctgaaaacatctcttctcttcttggacatttaccagctgagattgtgggcaaaaaattattaagccttctgcctgatgaagagaaagatgaagtctaccaaaagattattctcaaatttcctttactaaactcagaaacacatattgaattttgctgtcatttaaaaagaggaaatgtcgaacatggtgatagttctgcttacgaaaacgtgaaatttattgtgaatgtaagagatatttgtaatgagtttcctgtggtctttagtggcttgttttccagccacctctgtgctgactttgctgcatgtgttcctcaggaggatcggctttatcttgtgggaaatgtttgcattctcaggactcagctcctgcagcaactttacacttcaaaggcagtcagtgatgaagctgtacttacacaagattcagatgaggaaccttttgtgggagagctcagtagctctcaaggtcaaagaggacacactagcatgaaagccgtgtacgttgaacccgctgctgctgctgctgctgctgctatctcagacgaccaaattgatattgcagaggttgagcagtatggaccacaagaaaacgttcacatgtttgtagattctgattcaacttattgctccagtacagttttcctggatactatgcctgaatctccagccttatccttgcaagactttcgaggtgagcctgaggtgaatccattgtacagggcagacccagtggacctggagttctcggtggatcaggtggactcagtggaccaggagggcccaatggaccagcaggacccagagaacccagttgccccgttggaccaggcaggcctgatggatccagtggatccagaggactcagtggacctgggggctgctggcgcaagtgctcagccattacagccatcatcaccagttgcatatgacatcattagccaggaactggaactgatgaagaagttgaaggagcagctagaagagaggacttggttgctgcatgatgccatccaaaaccagcagaatgcattggaattgatgatggatcaccttcagaagcagccaaacacattacgccacgttgtcattcctgatctccaatcttcggaggcagtgcccaagaaacaacagaaacaacacgctgggcaagtgaagcggcctctcccacatcccaaggacgtcaagtgtttctgtggtttatctttatccaactctctcaaaaacactggggagcttcaggagccttgtgttgccttcaaccagcagcaactggtgcagcaagaacaacacctgaaggagcagcagcggcagctgcgggagcagctgcaacagctgagagagcaaaggaaggtgcagaagcagaagaagatgcaggagaagaagaagctgcaggagcagaaaatgcaggagaagaagaagctgcaggagcagaggcggcaaaagaagaagaagctacaggagcggaagaagtggcaggggcagatgctacagaaagagccagaggaggagcagcagaagcagcagctgcaagagcagccactgaagcataatgtcatcgtggggaatgagagggtgcagatatgcctgcaaaacccacgtgacgtatctgtgcccctctgcaatcaccctgttagatttttacaggcccaacccattgttcctgtccagagagcagctgaacaacagccctctggcttctatcaagatgaaaactgtgggcaacaggaagatgagagtcaa

SEQ ID NO: 60 - DNA, 3861 bp, Homo sapiens
cagactttccgggcgcccaccaggctcccgggctctcaccggagaccaccggagaccacagggaggagcttcaaggtaccccaagtccctgcggcctccagcagtgaagagttccacaagggagtcttcctacgtcactgaataatgaatgaagatgagaggggaaaagagaagagacaaagtcaatccaaaaagctctcaaaggaaattaaactggattccatcatttcctacctatgattacttcaaccaagtgacgctacagttattagatggctttatgattacactgagcacagatggagtgatcatttgtgtggctgaaaacatctcttctcttcttggacatttaccagctgagattgtgggcaaaaaattattaagccttctgcctgatgaagagaaagatgaagtctaccaaaagattattctcaaatttcctttactaaactcagaaacacatattgaattttgctgtcatttaaaaagaggaaatgtcgaacatggtgatagttctgcttacgaaaacgtgaaatttattgtgaatgtaagagatatttgtaatgagtttcctgtggtctttagtggcttgttttccagccacctctgtgctgactttgctgcatgtgttcctcaggaggatcggctttatcttgtgggaaatgtttgcattctcaggactcagctcctgcagcaactttacacttcaaaggcagtcagtgatgaagctgtacttacacaagattcagatgaggaaccttttgtgggagagctcagtagctctcaaggtcaaagaggacacactagcatgaaagccgtgtacgttgaacccgctgctgctgctgctgctgctgctatctcagacgaccaaattgatattgcagaggttgagcagtatggaccacaagaaaacgttcacatgtttgtagattctgattcaacttattgctccagtacagttttcctggatactatgcctgaatctccagccttatccttgcaagactttcgaggtgagcctgaggtgaatccattgtacagggcagacccagtggacctggagttctcggtggatcaggtggactcagtggaccaggagggcccaatggaccagcaggacccagagaacccagttgccccgttggaccaggcaggcctgatggatccagtggatccagaggactcagtggacctgggggctgctggcgcaagtgctcagccattacagccatcatcaccagttgcatatgacatcattagccaggaactggaactgatgaagaagttgaaggagcagctagaagagaggacttggttgctgcatgatgccatccaaaaccagcagaatgcattggaattgatgatggatcaccttcagaagcagccaaacacattacgccacgttgtcattcctgatctccaatcttcggaggcagtgcccaagaaacaacagaaacaacacgctgggcaagtgaagcggcctctcccacatcccaaggacgtcaagtgtttctgtggcttatctttatccaactctctcaaaaacactggggagcttcaggagccttgtgttgccttcaaccagcagcaactggtgcagcaagaacaacacctgaaggagcagcagcggcagctgcgggagcagctgcaacagctgagagagcaaaggaaggtgcagaagcagaagaagatgcaggagaagaagaagctgcaggagcagaaaatgcaggagaagaagaagctgcaggagcagaggcggcaaaagaagaagaagctacaggagcggaagaagtggcaggggcagatgctacagaaagagccagaggaggagcagcagaagcagcagctgcaagagcagccactgaagcataatgtcatcgtggggaatgagagggtgcagatatgcctgcaaaacccacgtgacgtatctgtgcccctctgcaatcaccctgttagatttttacaggcccaacccattgttcctgtccagagagcagctgaacaacagccctctggcttctatcaagatgaaaactgtgggcaacaggaagatgagagtcaaaggtaagacatgcatggaatggtgatagtggctatgattattgcttttccctcatggctggatcccatgcctgagatcacagacagacgacctatccatgtggcaccagcgtccctctcatcaatagcaacttgctctcaacttgccttctctgtggccacggccatcatccttctgcatgtgtggtgcatggatgagcatccattgatggaacaaaggggtacccatgaagaaaggactagataaggaagcctcaatgctagggaattagcccacaaaccttgaaatcacttggctggattccccaagcccagtctcttgcacagggtgtctatggacaagtcttgtgcccatagctgaaagacttctgggcttccagtttcattccccaactttcctggaatctggtgatggcagtaggccaagtgcacgtatctccctggcatttatgaacagtaagagggaagtgagtgtgaaacaattgctagattgctaagatcagccaagaacacatgaaggctaagtcctgagtggcaatatagggacatgaacacatttctgctcaataataaggtcttcagttccaaatgctagaaagataaggttcttctcagcaatgaaaggattgcttagcacatagttggggcccactaaatagttttgaatgaatgttagctttctgagacgggcctcctaaagctctctggacttccttctgggccttgctttcgaatcagtgactttgacatgaagactgctttagggccatattgtccacttgacctgatcatcagaatccacctgaggagggtttggaaagtatagattcagagtctgggatggggttcaaaaatctgtattgatcacaagacttccaggcttagggttgtggagagagcactggatttggagtttggaaatctgccttacagtctctgctcagccattagctccctgagtgctttcaaagtaggtgaaatgactttccaaggccacagagagcatagttgtagcaggaacaagactgtccaagtaatataaataaaaccaggtgagacaagggattgtaatttctctcagtgtctacattcacattactttgacaagttgaatcttagtcaggccaaagtgagaatacaaacaagtgaatcctgtggtgtagccaccaaaaccaaatgtgcctgaagcacctactgatgaccacgtgagtgtctcatcagttatcatgtccagatccacatcactgcaacagctttcttctcttacagtttttatcctgaggcgtatcaagggccccccgtgaaccagctgccattgatagatacctcaaactctgaggcaatttcttcttccagcattcctcagtttcccataacttcagactcaaccataagcaccctggagaccccacaggattacatccggctttggcaagagttgtctgattcactcggtcctgttgtccaagtgaacacttggtcttgcgatgagcagggcaccctgcacggccaacccacctaccatcaggtgcaagtttctgaggtaggagtcgagggacctcctgatccacaggctttccaaggccctgctgcataccagccagaccagatgagatctgcggagcagaccagattgatgcctgcagagcaacgtgactcaaataagccgtgctaacagtactttcatgaccagtgatgaggggaaatggggggagggggcaggccaatgaggtctgcatggccaggggaccttcaaggtgcataaagtcccttggggtagggtttagtgggtacagactt

SEQ ID NO: 61 - protein, 639 aa, Homo sapiens
MKMRGEKRRDKVNPKSSQRKLNWIPSFPTYDYFNQVTLQLLDGFMITLSTDGVIICVAENISSLLGHLPAEIVGKKLLSLLPDEEKDEVYQKIILKFPLLNSETHIEFCCHLKRGNVEHGDSSAYENVKFIVNVRDICNEFPVVFSGLFSSHLCADFAACVPQEDRLYLVGNVCILRTQLLQQLYTSKAVSDEAVLTQDSDEEPFVGELSSSQGQRGHTSMKAVYVEPAAAAAAAAISDDQIDIAEVEQYGPQENVHMFVDSDSTYCSSTVFLDTMPESPALSLQDFRGEPEVNPLYRADPVDLEFSVDQVDSVDQEGPMDQQDPENPVAPLDQAGLMDPVDPEDSVDLGAAGASAQPLQPSSPVAYDIISQELELMKKLKEQLEERTWLLHDAIQNQQNALELMHDHLQKQPNTLRHVVIPDLQSSEAVPKKQQKQHAGQVKRPLPHPKDVKCFCGLSLSNSLKNTGELQEPCVAFNQQQLVQQEQHLKEQQRQLREQLQQLREQRKVQKQKKMQEKKKLQEQKMQEKKKLQEQRRQKKKKLQERKKWQGQMLQKEPEEEQQKQQLQEQPLKHNVIVGNERVQICLQNPRDVSVPLCNHPVRFLQAQPIVPVQRAAEQQPSGFYQDENCGQQEDESQR

SEQ ID NO: 62 - protein, 773 aa, Homo sapiens
MKMRGEKRRDKVNPKSSQRKLNWIPSFPTYDYFNQVTLQLLDGFMITLSTDGVIICVAENISSLLGHLPAEIVGKKLLSLLPDEEKDEVYQKIILKFPLLNSETHIEFCCHLKRGNVEHGDSSAYENVKFIVNVRDICNEFPVVFSGLFSSHLCADFAACVPQEDRLYLVGNVCILRTQLLQQLYTSKAVSDEAVLTQDSDEEPFVGELSSSQGQRGHTSMKAVYVEPAAAAAAAAISDDQIDIAEVEQYGPQENVHMFVDSDSTYCSSTVFLDTMPESPALSLQDFRGEPEVNPLYRADPVDLEFSVDQVDSVDQEGPMDQQDPENPVAPLDQAGLMDPVDPEDSVDLGAAGASAQPLQPSSPVAYDIISQELELMKKLKEQLEERTWLLHDAIQNQQNALELMMDHLQKQPNTLRHVVIPDLQSSEAVPKKQQKQHAGQVKRPLPHPKDVKCFCGLSLSNSLKNTGELQEPCVAFNQQQLVQQEQHLKEQQRQLREQLQQLREQRKVQKQKKMQEKKKLQEQKMQEKKKLQEQRRQKKKKLQERKKWQGQMLQKEPEEEQQKQQLQEQPLKHNVIVGNERVQICLQNPRDVSVPLCNHPVRFLQAQPIVPVQRAAEQQPSGFYQDENCGQQEDESQSFYPEAYQGPPVNQLPLIDTSNSEAISSSSIPQFPITSDSTISTLETPQDYIRLWQELSDSLGPVVQVNTWSCDEQGTLHGQPTYHQVQVSEVGVEGPPDPQAFQGPAAYQPDQMRSAEQTRLMPAEQRDSNKPC

SEQ ID NO: 63 - DNA, 1291 bp, Homo sapiens
aattcggcacgagcagaggcagcccgggcagtcgccgggtcggtagaggtgctgacagggcggagccgccgcttgcctcagccggcctcacccctcccgcccgggacaggtggctggagcgcgctccgcctccgccagctgcgagtgggagcgagggagggccggtcccggacggtcgccgccagctgcgagtggaagtgagcgagggctagtcccggaccgccgggccaggggtccggcggcagcagacgggtaccgtggcggccaaaaaaatgctcctgtaccgaggggcccccgccggccctggcgcgccgggctgcgggctggcccggcccggcggcggcccgcaggccttcgggatccgcctgagcacgatgagcccccgctacctccagagcaactccagcagccacacgcgacccttcagtgccatcgcggagctgctagataatgctgtagatccagatgtatctgccaggacggtctttatagatgttgaggaggtcaagaataaatcttgtttgacctttaccgatgatggatgtgggatgacacctcataaactacaccgaatgctcagcagatgaggttgattactctccctgtggcttcctgaactagagtgtgtgaagcacgagtaacaatggtggaatgctgtggaggagggaaagggctcccaactataaaaatggaaaagaaattcatgtggttgtagttacccttaaataaaatggaaaatctgagttggactttgatacagatcaatatgatcagactttgacacagaagaaaaaatgactggcggtgttacctctgagctaccagaaacagaatattctttaagggcattttgtggtattctatacatgaagccacgcatgaaaatttttctgcgtcaaaagaaggtgactacccagatgattgccaagagcctggccaatgtagaatatgatacatataaacctaccttcacaaataagcaggtgagaatcacctttgggttctcttgcaagaatagtaaccagtttggaataatgatgtatcataacaaccgactcataaaatcttttgagaaggtggggtgccaggtgaagccaactcgtggagaaggtgtaggagtaattggagtcattgagtgcaatttcctaaaacctgcctacaacaaacaagactttgagtataccaaggagtaccggctaacaataaatgcccttgcccagaagctcaatgcttactggaaggaaaaaacatctcaagataattttgagacctcaactgtagccaggccaat

SEQ ID NO: 64 - DNA, 1186 bp, Homo sapiens
aaccagcaaaacaaaaaatgattattaccgaggttcattgcccagcctagaagccatcttgaactattccattttcaaccgtgaaaatgacctgctggcccagtttgatgccatcccaggcaaaaaaggcactcgtgttctcatttggaacatccgcagaaataaaaatggaaaatctgagttggactttgatacagatcaatatgacatcctggtttcagactttgacacagaagaaaaaatgactggcggtgttacctctgagctaccagaaacagaatattctttaagggcattttgtggtattctatacatgaagccacgcatgaaaatttttctgcgtcaaaagaaggtgactacccagatgattgccaagagcctggccaatgtagaatatgatacacataaacctaccttcacaaataagcaggtgagaatcacctttgggttctcttgcaagaatagtaaccagtttggaataatgatgtatcataacaaccgactcataaaatcttttgagaaggtggggtgccaggtgaagccaactcgtggagaaggtgtaggagtaattggagtcattgagtgcaatttcctaaaacctgcctacaacaaacaagactttgagtataccaaggagtaccggctaacaataaatgcccttgcccagaagctcaatgcttactggaaggaaaaaacatctcaagataattttgagacctcaactgtagccaggccaataccgaaggttcctgaccagacatgggttcagtgtgatgagtgtcttaatggagaaagcttcctgggaagattgatccatccatgttacctgcaagatggttttgttattataattcccatccaaagtacaggagatgctctgttccagaggaacaagaactcattgatgaagacctgtgcttgagcaaagctaagaaacaagaacaaactgttgaggagaagaagaagatgcctatggaaaatgagaaccaccaggtattcagtaatccaccaaagatcctcactgttcaagaaatggctggattgaataacaagacaattggatatgagggaattcatagccctagtgtgcttccttctggtggagaagaaagcagatcaccatctcttcaacttaagcctctggattccagtgttttacagttttccagtaagtacaaatggatcc

SEQ ID NO: 65 - protein, 61 aa, Homo sapiens
EAARAVAGSVEVLTGRSRRLPQPASPLPPGTGGWSALRLRCLRVGAREGRSRTVAASCEWK

SEQ ID NO: 66 - protein, 55 aa, Homo sapiens
ARASPGPPGQGSGGSRRVPWRPKKCSCTESPPPALARRAAGWPGPAAARRPSGSA

SEQ ID NO: 67 - protein, 22 aa, Homo sapiens
APATSRATPAATRDPSVPSRSC

SEQ ID NO: 68 - protein, 10 aa, Homo sapiens
IQMYLPGRSL

SEQ ID NO: 69 - protein, 10 aa, Homo sapiens
MLRRSRINLV

SEQ ID NO: 70 - protein, 8 aa, Homo sapiens
PLPMMDVG

SEQ ID NO: 71 - protein, 22 aa, Homo sapiens
HLINYTECSADEVDYSPCGFLN

SEQ ID NO: 72 - protein, 29 aa, Homo sapiens
STSNNGQMLWRRERAPNYKNGKQIHWWTI

SEQ ID NO: 73 - protein, 190 aa, Homo sapiens
NGKSELDFDTDQYDILVSDFDTEEKMTGGVTSELPETEYSLRAFCGILYMKPRMKIFLRQKKVTTQMIAKSLANVEYDTYKPTFTNKQVRITFGFSCKNSNQFGIMMYHNNRLIKSFEKVGCQVKPTRGEGVGVIGVIECNFLKPAYWKQDFEYTKEYRLTINALAQKLNAYWKEKTSQDNFETSTVARP

SEQ ID NO: 74 - protein, 12 aa, Homo sapiens
MIITEDSLPSLE

SEQ ID NO: 75 - protein, 13 aa, Homo sapiens
AILNYSIFNREND

SEQ ID NO: 76 - protein, 10 aa, Homo sapiens
LLAQFDAIPG

SEQ ID NO: 77 - protein, 725 aa, Homo sapiens
KKGTRVLIWNIRRNKNGKSELDFDTDQYDILVSDFDTEEKMTGGVTSELPETEYSLRAFCGILYMKPRMKIFLRQKKVTTQMIAKSLANVEYDTHKPTFTNKQVRITFGFSCKNSNQFGIMMYHNNRLIKSFEKVGCQVKPTRQEGVGVIGVIECNPLKPAYNKQDFEYTKEYRLTINALAQKLNAYWKEKTSQDNFETSTVARPIPKVPDQTWVQCDECLKWRKLPGKIDPSMLPARWFCYYNSHPKYRRCSVPEEQELIDEDLCLSKAKKQEQTVEEKKKMPMENENHQVFSNPPKILTVQEMAGLNNKTIGYEGIHSPSVLPSGGEESRSPSLQLKPLDSSVLQFSSKYKWILGEEPVEKRRRLQNEMTTPSLDYSMPAPYRRVEAPVAYPEGENSHDKSSSERSTPPYLFPEYPEASKNTGQNREVSILYPGAKDQRQGSLLPEELEDQMPRLVAEESNRGSTTINKEEVNKGPFVAVVGVAKGVRDSGAPIQLIPFNREELAERRKAVESWNPVPYSVASAAIPAAAIGEKARGYEESEGHNTPKLKNQRELEELKRTTEKLERVLAERNLFQQKVEELEQERNHWKSEFKKVQHELVIYSTQEAEGLYWSKKHMGYRQAEFQILKAELERTKEEKQELKEKLKETETHLEMLQKAQVSYRTPEGDDLERALAKLTRLRIHVSYLLTSVLPHLELREIGYDSEQVDGILYTVLEANHILD

SEQ ID NO: 78 - DNA, 32 bp, Artificial
  Primer
cgtcatactcctgcttgctgatccacatctgc

SEQ ID NO: 79 - DNA, 798 bp, Homo sapiens
agtttttatcctgaggcgtatcaagggccccccgtgaaccagctgccattgatagatacctcaaactctgaggcaatttcttcttccagcattcctcagtttcccataacttcagactcaaccataagcaccctggagaccccacaggattacatccggctttggcaagagttgtctgattcactcggtcctgttgtccaagtgaacacttggtcttgcgatgagcagggcaccctgcacggccaacccacctaccatcaggtgcaagtttctgaggtaggagtcgagggacctcctgatccacaggctttccaaggccctgctgcataccagccagaccagatgagatctgcggagcagaccagattgatgcctgcagagcaacgtgactcaaataagccgtgctaacagtactttcatgaccagtgatgaggggaaatggggggagggggcaggccaatgaggtctgcatggccaggggaccttcaaggtgcgtaaagtcccttggggtagggtttagtgggtagagacttatttgtttcctgataggttatgtttgtaattgtttgttaagcacagcctgtttcttggaagttatgctgtagaggcagcctgtgatccgtagtatgctagggtgtgacagcagccagccacagctggatctgatgtcttgtctgccccgcccagctttgcatatccatgttctaccacaggaaggtggcctgccaagagtctgctcaaagttttcaacataaagaataaagaaaaaaaaaaatgcaaaaaaaaaaaaaaaaaaaa

SEQ ID NO: 80 - DNA, 261 bp, Homo sapiens
gtttcctgataggttatgtttgtaattgtttgttaagcacagcctgtttcttggaagttatgctgtagaggcagcctgtgatccgtagtatgctagggtgtgacagcagccagccacagctggatctgatgtcttgtctgccccgcccagctttgcatatccatgttctaccacaggaaggtggcctgccaagagtctgctcaaagttttcaacataaagaataaagaaaaaaaaaaatgcaaaaaaaaaaaaaaaaaaaa

SEQ ID NO: 81 - DNA, 4109 bp, Homo sapiens
ttttttttttttttttttggcatttttttttctttattctttatgttgaaaactttgagcagactcttggcaggccaccttcctgtggtagaacatggatatgcaaagctgggcggggcagacaagacatcagatccagctgtggctggctgctgtcacaccctagcatactacggatcacaggctgcctctacagcataacttccaagaaacaggctgtgcttaacaaacaattacaaacataacctatcaggaaacaagtctctacccactaaaccctaccccaagggactttatgcaccttgaaggtcccctggccatgcagacctcattggcctgccccctccccccatttcccctcatcactggtcatgaaagtactgttagcacggcttatttgagtcacgttgctctgcaggcatcaatctggtctgctccgcagatctcatctggtctggctggtatgcagcagggccttggaaagcctgtggatcaggaggtccctcgactcctacctcagaaacttgcacctgatggtaggtgggttggccgtgcagggtgccctgctcatcgcaagaccaagtgttcacttggacaacaggaccgagtgaatcagacaactcttgccaaagccggatgtaatcctgtggggtctccagggtgcttatggttgagtctgaagttatgggaaactgaggaatgctggaagaagaaattgcctcagagtttgaggtatctatcaatggcagctggttcacggggggcccttgatacgcctcaggataaaaactgtaagagaagaaagctgttgcagtgatgtggatctggacatgataactgatgagacactcacgtggtcatcagtaggtgcttcaggcacatttggttttggtggctacaccacaggattcacttgtttgtattctcactttggcctgactaagattcaacttgtcaaagtaatgtgaatgtagacactgagagaaattacaatcccttgtctcacctggttttatttatattacttggacagtcttgttcctgctacaactatgctctctgtggccttggaaagtcatttcacctactttgaaagcactcagggagctaatggctgagcagagactgtaaggcagatttccaaactccaaatccagtgctctctccacaaccctaagcctggaagtcttgtgatcaatacagatttttgaaccccatcccagactctgaatctatactttccaaaccctcctcaggtggattctgatgatcaggtcaagtggacaatatggccctaaagcagtcttcatgtcaaagtcactgattcgaaagcaaggcccagaaggaagtccagagagctttaggaggcccgtctcagaaagctaacattcattcaaaactatttagtgggccccaactatgtgctaagcaatcctttcattgctgagaagaaccttatctttctagcatttggaactgaagaccttattattgagcagaaatgtgttcatgtccctatattgccactcaggacttagccttcatgtgttcttggctgatcttagcaatctagcaattgtttcacactcacttccctcttactgttcataaatgccagggagatacctgcacttggcctactgccatcaccagattccaggaaagttggggaatgaaactggaagcccagaagtctttcagctatgggcacaagacttgtccatagacaccctgtgcaagagactgggcttggggaatccagccaagtgatttcaaggtttgtgggctaattccctagcattgaggcttccttatctagtcctttcttcatgggtacccctttgttccatcaatggatgctcatccatgcaccacacatgcagaaggatgatggccgtggccacagagaaggcaagttgagagcaagttgctattgatgagagggacgctggtgccacatggataggtcgtctgtctgtgatctcaggcatgggatccagccatgagggaaaagcaataatcatagccactatcaccattccatgcatgtcttacctttgactctcatcttcctgttgcccacagttttcatcttgatagaagccagagggctgttgttcagctgctctctggacaggaacaatgggttgggcctgtaaaaatctaacagggtgattgcagaggggcacagatacgtcacgtgggttttgcaggcatatctgcaccctctcattccccacgatgacattatgcttcagtggctgctcttgcagctgctgcttctgctgctcctcctctggctctttctgtagcatctgcccctgccacttcttccgctcctgtagcttcttcttcttttgccgcctctgctcctgcagcttcttcttctcctgcattttctgctcctgcagcttcttcttctcctgcatcttcttctgcttctgcaccttcctttgctctctcagctgttgcagctgctcccgcagctgccgctgctgctccttcaggtgttgttcttgctgcaccagttgctgctggttgaaggcaacacaaggctcctgaagctccccagtgtttttgagagagttggataaagataagccacagaaacacttgacgtccttgggatgtgggagaggccgcttcacttgcccagcgtgttgtttctgttgtttcttgggcactgcctccgaagattggagatcaggaatgacaacgtggcgtaatgtgtttggctgcttctgaaggtgatccatcatcaattccaatgcattctgctggttttggatggcatcatgcagcaaccaagtcctctcttctagctgctccttcaacttcttcatcagttccagttcctggctaatgatgtcatatgcaactggtgatgatggctgtaatggctgagcacttgcgccagcagcccccaggtccactgagtcctctggatccactggatccatcaggcctgcctggtccaacggggcaactgggttctctgggtcctgctggtccattgggccctcctggtccactgagtccacctgatccaccgagaactccaggtccactgggtctgccctgtacaatggattcacctcaggctcacctcgaaagtcttgcaaggataaggctggagattcaggcatagtatccaggaaaactgtactggagcaataagttgaatcagaatctacaaacatgtgaacgttttcttgtggtccatactgctcaacctctgcaatatcaatttggtcgtctgagatagcagcagcagcagcagcagcagcgggttcaacgtacacggctttcatgctagtgtgtcctctttgaccttgagagctactgagctctcccacaaaaggttcctcatctgaatcttgtgtaagtacagcttcatcactgactgcctttgaagtgtaaagttgctgcaggagctgagtcctgagaatgcaaacatttcccacaagataaagccgatcctcctgaggaacacatgcagcaaagtcagcacagaggtggctggaaaacaagccactaaagaccacaggaaactcattacaaatatctcttacattcacaataaatttcacgttttcgtaagcagaactatcaccatgttcgacatttcctctttttaaatgacagcaaaattcaatatgtgtttctgagtttagtaaaggaaatttgagaataatcttttggtagacttcatctttctcttcatcaggcagaaggcttaataattttttgcccacaatctcagctggtaaatgtccaagaagagaagagatgttttcagccacacaaatgatcactccatctgtgctcagtgtaatcataaagccatctaataactgtagcgtcacttggttgaagtaatcataggtaggaaatgatggaatccagtttaatttcctttgagagctttttggattgactttgtctcttctcttttcccctctcatcttcattcattattcagtgacgtaggaagactcccttgtggaactcttcactgctggaggccgcagggacttggggtaccttgaagctcctccctgtggtctccggtgagagcccgggagcctggtgggcgcccggaaagtctg

SEQ ID NO: 82 - DNA, 1288 bp, Homo sapiens
attggcctggctacagttgaggtctcaaaattatcttgagatgttttttccttccagtaagcattgagcttctgggcaagggcatttattgttagccggtactccttggtatactcaaagtcttgtttgttgtaggcaggttttaggaaattgcactcaatgactccaattactcctacaccttctccacgagttggcttcacctggcaccccaccttctcaaaagattttatgagtcggttgttatgatacatcattattccaaactggttactattcttgcaagagaacccaaaggtgattctcacctgcttatttgtgaaggtaggtttatatgtatcatattctacattggccaggctcttggcaatcatctgggtagtcaccttcttttgacgcagaaaaattttcatgcgtggcttcatgtatagaataccacaaaatgcccttaaagaatattctgtttctggtagctcagaggtaacaccgccagtcattttttcttctgtgtcaaagtctgataccaggatgtcatattgatctgtatcaaagtccaactcagattttccattttatttaagggtaactacaaccacatgaatttgttttccatttttatagttgggagccctttccctcctccacagcattccaccattgttactcgtgcttcacacactctagttcaggaagccacagggagagtaatcaacctcatctgctgagcattcggtgtagtttatgaggtgtcatcccacatccatcatcggtaaaggtcaaacaagatttattcttgacctcctcaacatctataaagaccgtcctggcagatacatctggatctacagcattatctagcagctccgcgatggcactgaagggtcgcgtgtggctgctggagttgctctggaggtagcgggggctcatcgtgctcaggcggatcccgaaggcctgcgggccgccgccgggccgggccagcccgcagcccggcgcgccagggccggcgggggcccctcggtacaggagcatttttttggccgccacggtacccgtctgctgccgccggacccctggcccggcggtccgggactagccctcgctcacttccactcgcagctggcggcgaccgtccgggaccggccctccctcgctcccactcgcagctggcggaggcggagcgcgctccagccacctgtcccgggcgggaggggtgaggccggctgaggcaagcggcggctccgccctgtcagcacctctaccgacccggcgactgcccgggctgcctctg

SEQ ID NO: 83 - DNA, 3037 bp, Homo sapiens
ctttttaaaaacaatttatttttttccaatgtaagaataacaagtttaatattgaaaatctgaaaaaggcaggaagataggaagaagaaaaatatcaaccatagtcctaccacctaaagaacactcactgacagtgtggccatgctcattctttaagccttaatttaggtcccatttctggtgtggtgccttccttgaacttcctcccccagatcttccatgtcatatctgtaccactcagttgtacatatcatacacttttgtgtataatgtgatgtatgtcttatgtttccaacgagattgttacatcttcgaaggctgcaaacatgagttgtacttgttagcttaccccaaaataatacctggtataccggacccaatatctgctgattgatctaacctaaatgaatacaaaccatttcagaaaaagatatacaatagaccacatatccaggtcatgaaaattaaagctttcaggtcacctagcttagtgactattgcttttctgaccctagactcttgaaagcctatttaaactggcctctttctccacaccaaaactgataaaaaggagactgattatgagccaggatttacacagagattctctatataaggcataaaggtgagggtgagagagagagagagagagagagagagagagagagacgtgagggagggagagaaaagagaacagacagaagataagagaagagaagggtatacagtctggtgctcaatccagtatgtgatttgcctccagcaccgtgtacaggatcccatccacttgttctgagtcatacccgatctcacgaagctccaagtgagggaggacagaagtaaggagatagctgacgtggatacgtagccgcgtaagctttgccaaagccctttctaggtcatctccctctggggtccggtaggagacctgagccttctgcagcatttccaggtgtgtctctgtttccttcagtttctcttttaactcttgcttttcctctttggttctttccagctcagctttcagaatctggaattcagcttggcgataacccatgtgtttcttgctccagtacaagccttccgcctcctgggtactgtagatcaccaattcatgttggactttcttgaattcagactgccagtgattcctctcctgttccagctcctccaccttttgctggaacaaattcctttcagccaaaacacgttccaatttttctgtggttctcttcaattcttccagctctctctggttcttcaactttggtgtattatgaccttcgctctcctcatagcctcttgctttctccccaatggctgcagcagggattgcagcagaggccacagaataaggcactgggttccaggattcaactgcttttcgtctctcagcaagctcctctctgttaaaagggatcagctgaatgggagctcctgaatctctaacacctttggcaacacccacaacagctacaaaaggtcccttgttgacttcttctttgtttatggttgtgctacctctgttagattcttctgccaccaatcttggcatctgatcttctaattcttcaggaagcagggacccctggcgttggtctttggcccctggatacagaattgaaacctccctattctgacctgtattcttgcttgcttctgggtattctgggaaaaggtatggtggtgtacttctctcagaactcgatttatcatggctgttctccccttctgggtaggcaacaggtgcttctaccctcctgtaaggagcaggcatggaataatctagagaaggtgttgtcatctcattctggagccttcttcgtttctccaccggttcttcacctaggatccatttgtacttactggaaaactgtaaaacactggaatccagaggcttaagttgaagagatggtgatctgctttcttctccaccagaaggaagcacactagggctatgaattccctcatatccaattgtcttgttattcaatccagccatttcttgaacagtgaggatctttggtggattactgaatacctggtggttctcattttccataggcatcttcttcttctcctcaacagtttgttcttgtttcttagctttgctcaagcacaggtcttcatcaatgagttcttgttcctctggaacagagcatctcctgtactttggatgggaattataataacaaaaccatcttgcaggtaacatggatggatcaatcttcccaggaagctttctccatttaagacactcatcacactgaacccatgtctggtcaggaaccttcggtattggcctggctacagttgaggtctcaaaattatcttgagatgttttttccttccagtaagcattgagcttctgggcaagggcatttattgttagccggtactccttggtatactcaaagtcttgtttgttgtaggcaggttttaggaaattgcactcaatgactccaattactcctacaccttctccacgagttggcttcacctggcaccccaccttctcaaaagattttatgagtcggttgttatgatacatcattattccaaactggttactattcttgcaagagaacccaaaggtgattctcacctgcttatttgtgaaggtaggtttatgtgtatcatattctacattggccaggctcttggcaatcatctgggtagtcaccttcttttgacgcagaaaaattttcatgcgtggcttcatgtatagaataccacaaaatgcccttaaagaatattctgtttctggtagctcagaggtaacaccgccagtcattttttcttctgtgtcaaagtctgaaaccaggatgtcatattgatctgtatcaaagtccaactcagattttccatttttatttctgcggatgttccaaatgagaacacgagtgccttttttgcctgggatggcatcaaactgggccagcaggtcattttcacggttgaaaatggaatagttcaagatggcttctaggctgggcaatgaatcctcggtaataatcatttttttgttttgctggtt

(19) United States
(12) Patent Application Publication
(10) Pub. No.: US 2005/0158737 A1
(43) Pub. Date: Jul. 21, 2005

(54) Tumour associated antigens

(76) Inventors: Alison Banham, Oxford (GB); Karen Pulford, Oxford (GB); Amanda Liggins, Oxford (GB); Barbara Guinn, London (GB)

Correspondence Address: FOLEY & LARDNER LLP, 111 HUNTINGTON AVENUE, 26TH FLOOR, BOSTON, MA, 02199-7610, US

(21) Appl. No.: 10/951,163

(22) Filed: Sep. 27, 2004

Related U.S. Application Data
(63) Continuation of application No. PCT/GB03/01378, filed on Mar. 27, 2003.

(30) Foreign Application Priority Data
Mar. 27, 2002 (GB) 0207251.0

(51) Int. Cl. C12Q 1/68; G01N 33/574; C07H 21/04; C12N 9/64

(52) U.S. Cl. 435/6; 435/7.23; 435/69.3; 435/226; 435/320.1; 435/325; 530/388.8; 536/23.2

(57) ABSTRACT

There are disclosed a number of tumour associated antigens that can be utilised in the diagnosis and treatment of tumours in a patient. Methods of treating patients having such tumours are also disclosed.

The present invention is concerned with lymphoma associated antigens and in particular with nucleic molecules encoding such antigens and the polypeptides encoded therefrom. The invention further relates to treating such lymphomas, methods for their diagnosis and using the polypeptides and nucleic acid molecules of the invention.

The tumour or lymphoma-associated antigens of the present invention and the nucleic acid molecules encoding them are believed to be important markers that can be used for prognostic purposes or therapeutic treatment for lymphomas of various types such as diffuse large B cell lymphomas (DLBCL). Phenotypic changes which distinguish a tumour cell from a normal cell are often the result of one or more chromosomal abnormalities, gene mutations or changes in gene regulation. The genes can therefore be expressed in tumour cells but not the normal counterpart, can be over expressed in the tumour cells compared to normal cells, or may be aberrantly expressed at different stages of the cell maturation. These genes can be described as ‘tumour associated’ genes and are therefore useful markers for the tumour phenotype. The tumour markers or ‘tumour antigens’, encoded by these genes can thus be targeted by antigen-specific immune mechanisms which lead to the destruction of the tumour cell. Their expression can also be used to diagnose the occurrence of such tumour cells or as prognostic markers.

Differences between a wild type protein expressed by ‘normal’ cells and a corresponding tumour antigen protein may, in some instances, lead to the tumour antigen being recognised by an individuals immune system as ‘non-self’ and thereby eliciting an immune response in the individual against the tumour antigen. This may be a humoral (i.e. B cell mediated) immune response leading to the production of autoantibodies immunologically specific to the tumour antigen.

Autoantibodies are naturally occurring antibodies directed to an antigen which the individual's immune system recognises as foreign even though that antigen originated in the individual. They may be present in the circulation as circulating free antibodies or in the form of circulating immune complexes consisting of the autoantibodies bound to their target tumour antigen.

Diffuse large B-cell lymphoma accounts for 30-40% of all adult non-Hodgkin's lymphomas and is heterogeneous in terms of its morphology and clinical features (Harris et al., 1994). Approximately 50% of patients relapse after treatment (Project., 1997) and their tumours frequently become resistant to therapy. The genetic abnormalities underlying DLBCL remain poorly understood and, in contrast to other lymphoma types (e.g. follicular lymphoma (FL) or Burkitt's lymphoma (BL)), no single characteristic genetic alteration has been found.

Genomic instability promotes tumourigenesis and can occur through various mechanisms, including defective segregation of chromosomes or inactivation of DNA mismatch repair. Although some B-cell lymphomas are associated with chromosomal translocations that deregulate oncogene expression, a mechanism for genome-wide instability during lymphomagenesis has not until recently been described. During B-cell development, the immunoglobulin variable (V) region genes are subject to somatic hypermutation in germinal-centre B cells. It has been reported recently that an aberrant hypermutation activity targets multiple loci, including the proto-oncogenes PIM1, MYC, RhoH/TTF (ARHH) and PAX5, in more than 50% of diffuse large-cell lymphomas (DLCLs), which are tumours derived from germinal centres. The mutations described were distributed in the 5′ untranslated or coding sequences, were independent of chromosomal translocations, and shared features typical of V-region-associated somatic hypermutation. In contrast to mutations in V regions, however, these mutations were not detectable in normal germinal-centre B cells nor in other germinal-centre-derived lymphomas, suggesting a DLBCL-associated malfunction of somatic hypermutation. Intriguingly, the four hypermutable genes are susceptible to chromosomal translocations in the same region, consistent with a role for hypermutation in generating translocations by DNA double-strand breaks. By mutating multiple genes, and possibly by favouring chromosomal translocations, aberrant hypermutation may represent the major contributing factor to lymphomagenesis (Pasqualucci et al., 2001). It is this phenomenon which may account for the genetic variability in DLBCL.

Microarray based gene expression profiling has been utilised to identify different subtypes of DLBCL with different clinical outcomes. These include a good prognosis germinal centre-like (GC-like) subtype and poor prognosis type 3 and activated B-cell-like (ABC-like) subtypes (Rosenwald et al., 2002). The routine subtyping of DLBCL patients will enable the identification of those that do not respond to current treatment regimens and will improve treatment management.

An important factor concerning the results from gene-expression profiling studies is that the expression of mRNA does not always accurately reflect the levels of protein expression. Many proteins whose expression levels are regulated by mechanisms such as proteolysis would not necessarily show changes in mRNA levels. In addition other proteins are regulated by post translational modification, such as phosphorylation, or by changes in subcellular localisation. Thus the mRNA expression profile will fail to identify many molecules whose protein expression is relevant to the pathogenesis of DLBCL. One approach to the identification of tumour-associated molecules is to exploit circulating antibodies present in patients. Antibodies to tumour-related antigenic molecules have been identified in several non-haematopoietic cancers (Crawford et al., 1982; Lubin et al., 1995; Disis and Cheever, 1996; Jäger et al., 1998; Bei et al., 1999; Mosolits et al., 1999), while antibodies to antigens such as p53, c-myc and viral proteins have also been identified in a number of B cell lymphomas and leukaemias (Caron de Fromental et al., 1987; Kamihira et al., 1989; LaFond et al., 1992; Jarrett and MacKenzie, 1999). In addition, levels of antibodies to tumour idiotypes in B cell lymphomas and myelomas can be increased, in vivo, by immunisation (Kwak et al., 1993; Kwak et al., 1995; Syrengelas et al., 1996; Hsu et al., 1997; King et al., 1998). The prognostic relevance of these antibody responses is still under investigation and may depend on the tumour type under investigation.

A recent study of ALCL by the LRF Immunodiagnostics Unit has, for the first time, demonstrated the presence of circulating antibodies to the tumour-specific protein NPM-ALK in all ALK-positive ALCL patients studied (Pulford et al., 2000). The inventors have also shown, in a more recent investigation, that antibodies to another tumour-associated protein, BCL-2, are present in plasma from patients with follicular Lymphoma (FL) (Pulford et al., 2002). This result is important since it demonstrates that, although BCL-2 is a ‘self’ protein present in normal cells, it can still be recognised as antigenic in patients in whom it appears to be a tumour-associated molecule.

Current therapy does not cure the majority of patients with non-Hodgkins' lymphoma (NHL). The identification of tumour-associated antigens has, however, opened up the possibility of immunotherapy for patients with many different types of tumours who are refractory to conventional treatment (Rosenberg, 1996; Nestle et al., 1998; Marchand et al., 1999; Jäger et al., 2000). CD8-positive cytotoxic T lymphocytes (CTLs) have been shown to play a major role in the cell-mediated recognition of tumour-associated antigens such as NY-ESO-1 (Jäger et al., 1998), tyrosinase, gp100 and MART-1/Melan-A (Boon and Old, 1997). Tetrameric soluble class I MHC/peptide complexes (tetramers) have also been used with great success to identify and monitor the presence of CTLs in the peripheral blood and tumour infiltrated lymph nodes of melanoma patients (Dunbar et al., 1998; Romero et al., 1998; Jäger et al., 2000). In the case of FL, immunisation studies have demonstrated that an anti-tumour B and T cell response of these patients can be induced following the in vivo administration of either DNA vaccines or purified preparations of idiotypic proteins (i.e. patient specific proteins produced by the lymphoma cells) (Hsu et al., 1997; Stevenson, 1999; Stevenson et al., 2001). Other types of immunotherapeutic treatments currently under investigation include a) the administration of autologous antigen loaded dendritic cells (DCs) to patients (Nestle et al., 1998), b) increasing the expression of co-stimulatory molecules, such as CD80 and CD86 and CTLA4, by the autologous tumour cells or DCs increasing their capacity as antigen presenting cells (Tarte et al., 1999; Takayama et al., 2000), and c) the use of toxins linked to monoclonal antibodies specific for tumour-associated antigens (Grillo-Lopez et al., 2001). The discovery of tumour-associated molecules also identified proteins which may be targets for therapies using anti-sense oligonucleotides (Banerjee, 1999). Currently explored and future innovative therapies for B cell lymphomas and other tumours are reviewed by (Schultze, 1997; Jäger et al., 2000; Smith and Cerundolo, 2001; Stevenson et al., 2001).

The SEREX technique (serological analysis of recombinant cDNA expression libraries) was first used by Sahin et al., 1995. This technique has now been used in a number of laboratories to identify a range of tumour associated antigens (Ling et al., 1998; Scanlan et al., 1998; Itoh et al., 1999) reviewed by (Tureci et al., 1999). These have included molecules that were originally identified by cloning cytotoxic T lymphocyte (CTL)-recognised epitopes (van der Bruggen et al., 1991; Brichard et al., 1993), e.g. MAGE-1 and tyrosinase (Sahin et al., 1995). This technique thus enables the detection of tumour antigens that elicit both cellular and humoral immunity. The SEREX approach is characterised by the analysis of antigens that elicit high-titre IgG responses that usually depend on cognate T cell help in the patient in vivo (Tureci et al., 1997). As such, it provides a direct route to the analysis of the CD4 T-cell repertoire against tumour antigens (Old and Chen 1998). There is growing evidence that the antitumour response is a process integrating different effectors of the immune system, and studies have shown the capability of SEREX to retrieve tumour-associated antigens (TAAs) initially defined using CTL epitope approaches (van der Bruggen et al., 1994), and the co-existence of cellular and immune responses has been reported against tumour antigens such as NY-ESO-1 (Jager et al., 1998) and HER-2/neu (Disis et al., 1994).

The original SEREX technique relied on the detection of antigens expressed by a tumour derived cDNA library that was screened with autologous serum (Tureci et al., 1997). However, one variant of this technique aims to identify tumour antigens expressed solely in normal testis and germline cells (Tureci et al., 1998). Since testis is protected from the immune system by the blood-testis barrier, these cancer testis antigens can be considered immunologically tumour-specific (Takahashi et al., 1995; Chen and Old, 1999) and, as such, are not expected to trigger tolerance or induce autoimmunity when used in tumour immunotherapy applications (Gilboa, 2001).

Ideal cancer vaccine candidates should be selectively expressed in tumour tissue and be detectable in most patients. The enormous potential of Cancer-Testis Antigens (CTAs) as vaccine targets is based on their restricted pattern of expression and their high frequency of immunogenicity in cancer patients (Scanlan and Jager 2001). However, the majority of SEREX-defined antigens have a much broader expression profile in healthy tissue (Krackhardt et al., 2002), which may hinder their usefulness as direct vaccines. Recent work has shown that the co-presentation of a plasmid encoding a SEREX defined antigen with a tumour specific protein leads to a profound increase in CD8+ T cells specific for the tumour protein not achieved when immunization is only with the tumour protein (Nishikawa et al., 2001). This co-immunisation of SEREX-defined antigens with tumour proteins presents an attractive alternative vaccine strategy for human cancer immunotherapy and one that will be facilitated by the extensive base of information about SEREX-defined human tumour antigens (Nishikawa et al., 2001).

As explained in more detail in the example provided, the present inventors have, by screening a testis cDNA expresion library with serum from a patient suffering from a lymphoid tumour (DLBCL), identified antigens which are candidates for immunotherapeutic applications. Some of the antigens identified represent novel antigens hitherto unidentified while others represent partially characterised antigens for which no function has yet been ascribed.

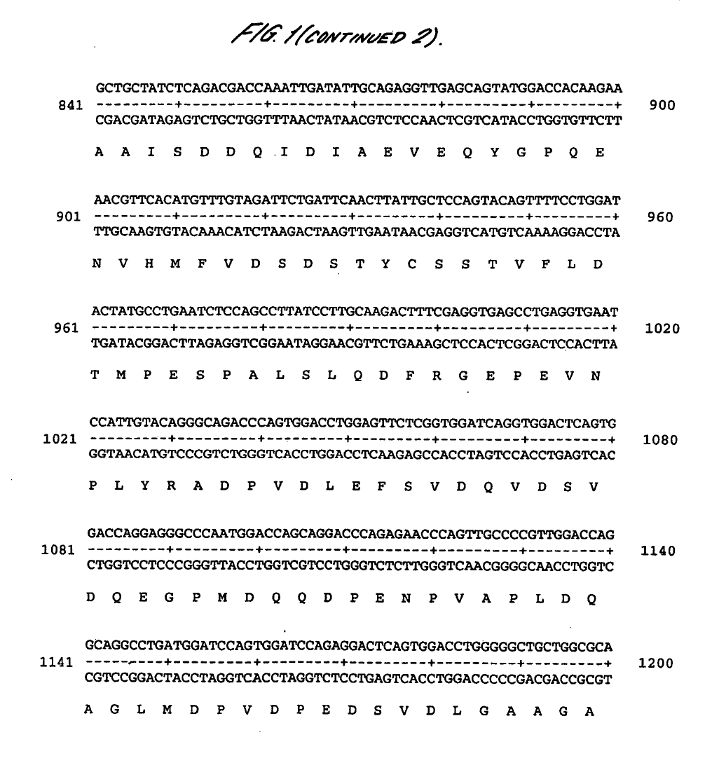

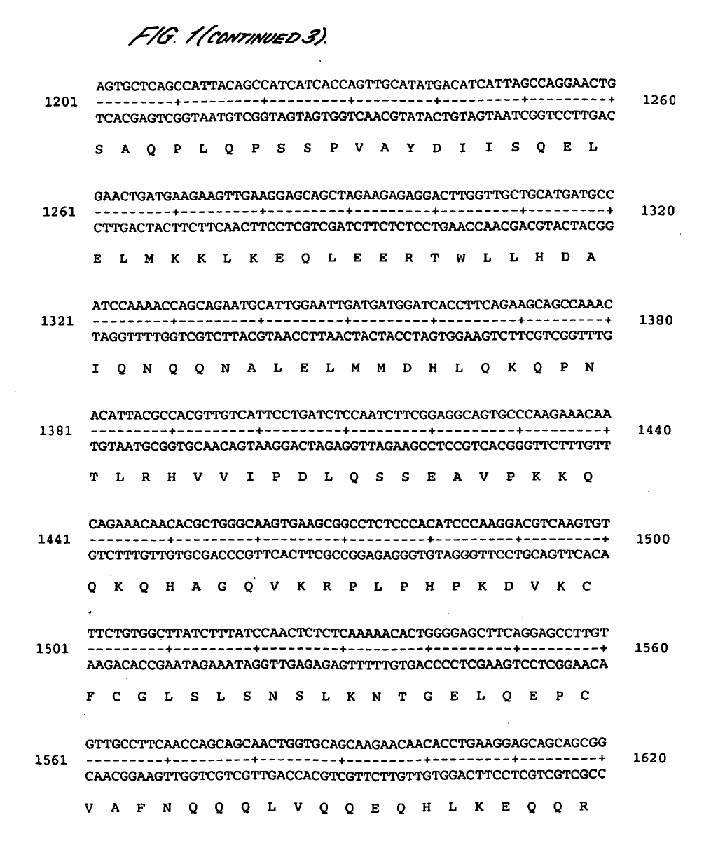

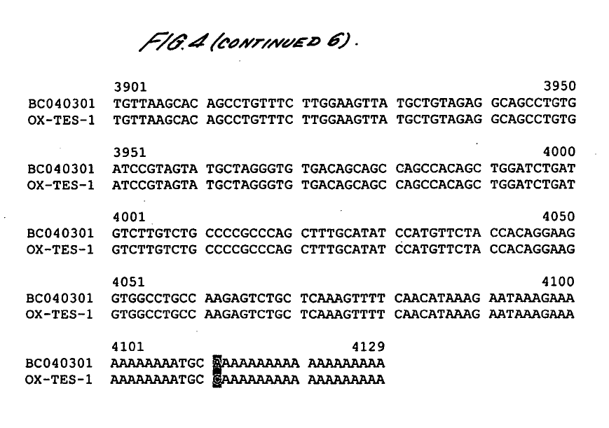

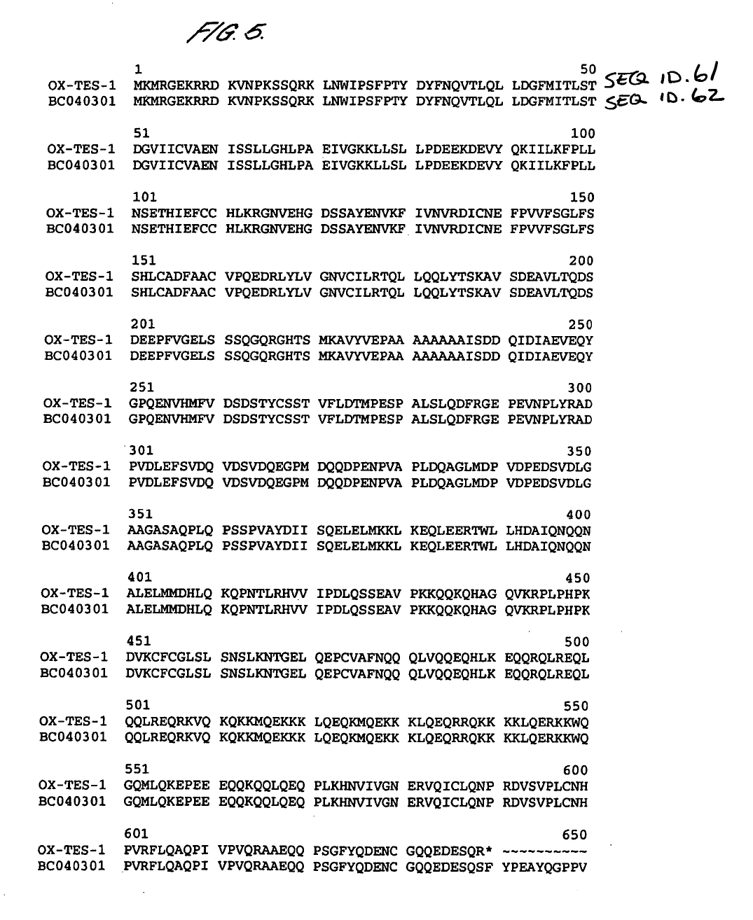

Therefore, according to a first aspect of the invention there is provided a nucleic acid molecule encoding a tumour antigen having the amino acid sequence illustrated in any of FIGS. 2 or 10 or a functional equivalent or fragment thereof. The nucleic acid molecules according to this aspect of the invention encode novel tumour-associated antigens which have not been identified hitherto and which may, advantageously, be utilised to diagnose or treat solid tumours such as kidney, breast, uterus, cervix, colon, lung, stomach, rectum or small intestine. Alternatively, they may be used to diagnose, for example lymphomas, such as DLBCL and therefore represent novel targets for therapy.

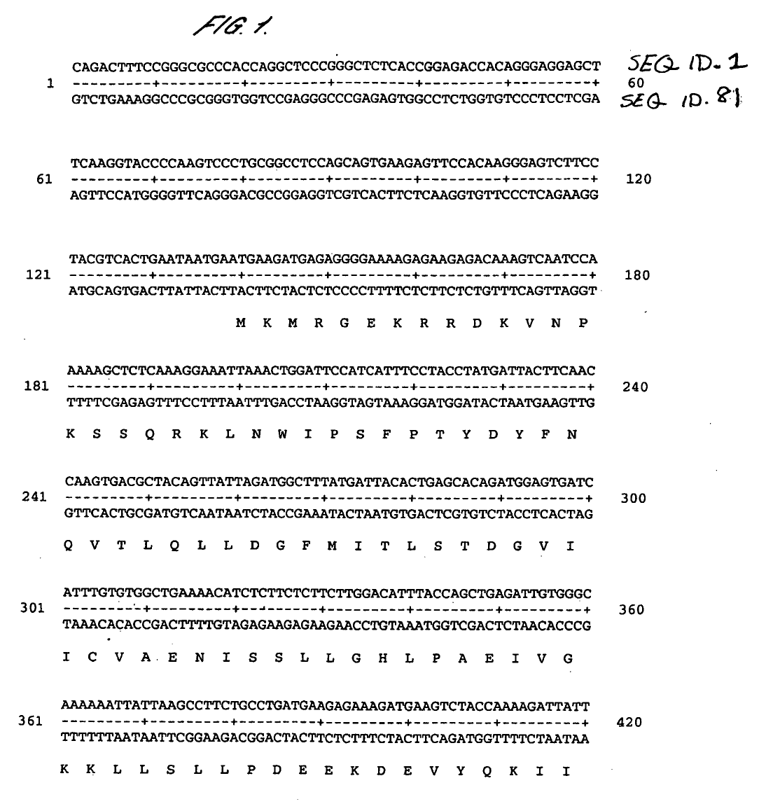

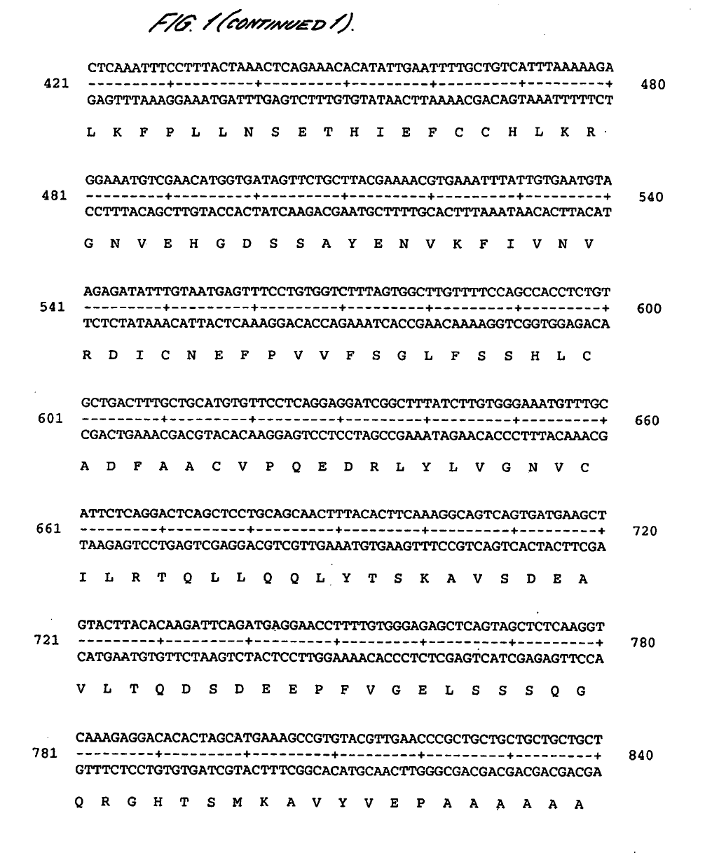

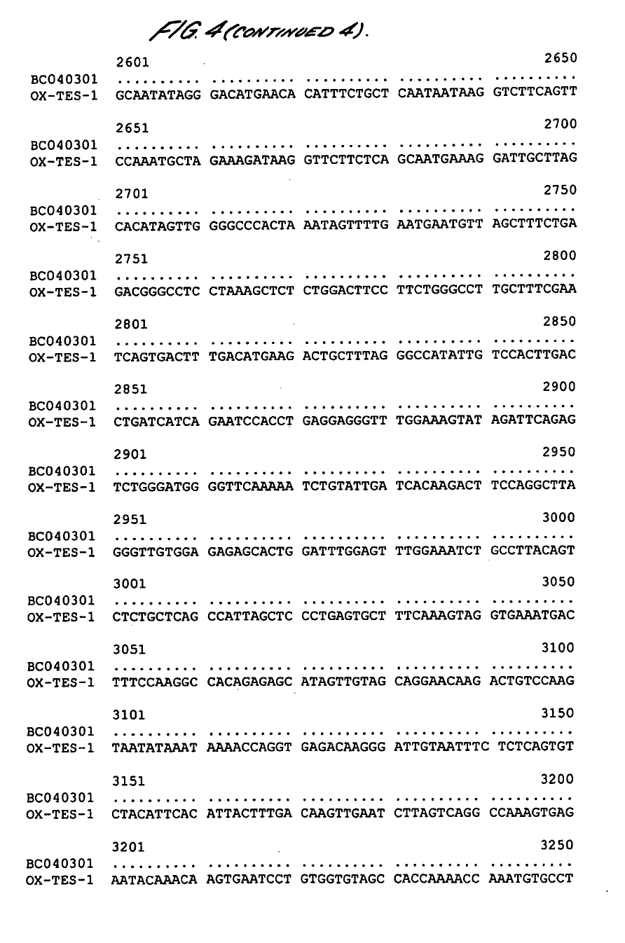

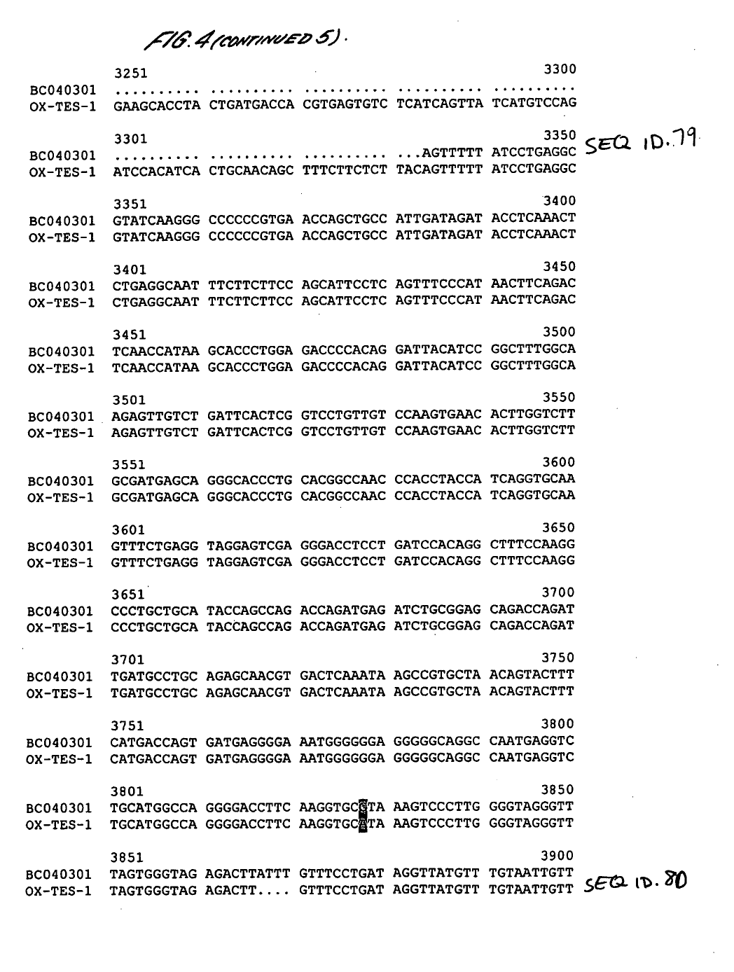

The nucleic acid molecule is preferably DNA and more preferably a cDNA molecule, which is preferably of human origin. In a preferred embodiment, the nucleic acid molecule encoding said antigen comprises the sequence of nucleotides in FIGS. 1 and 9 respectively.

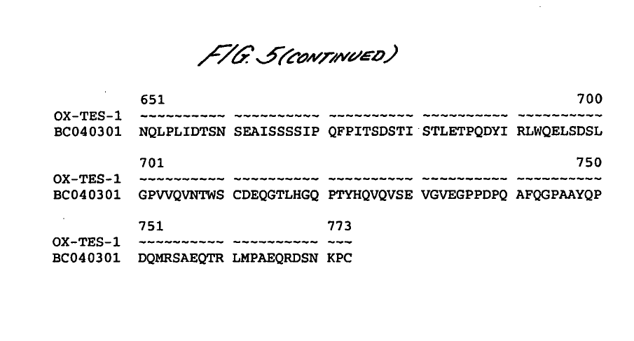

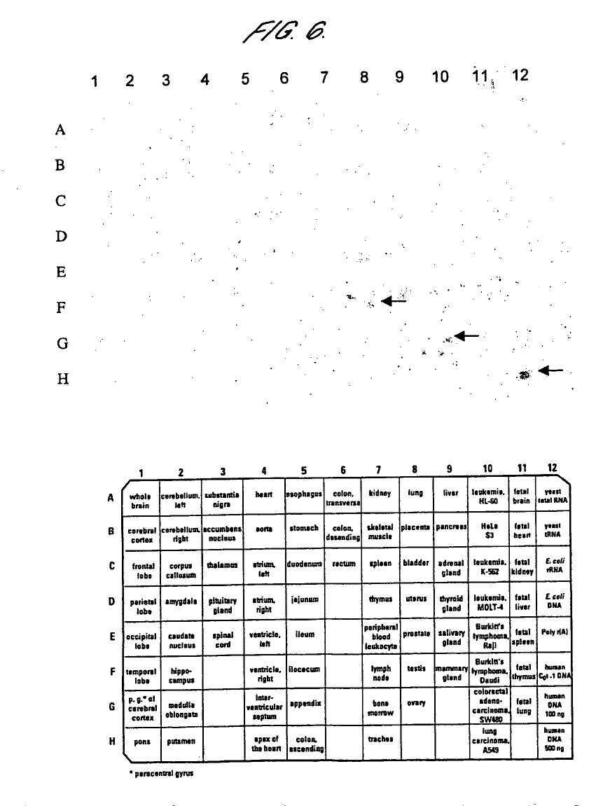

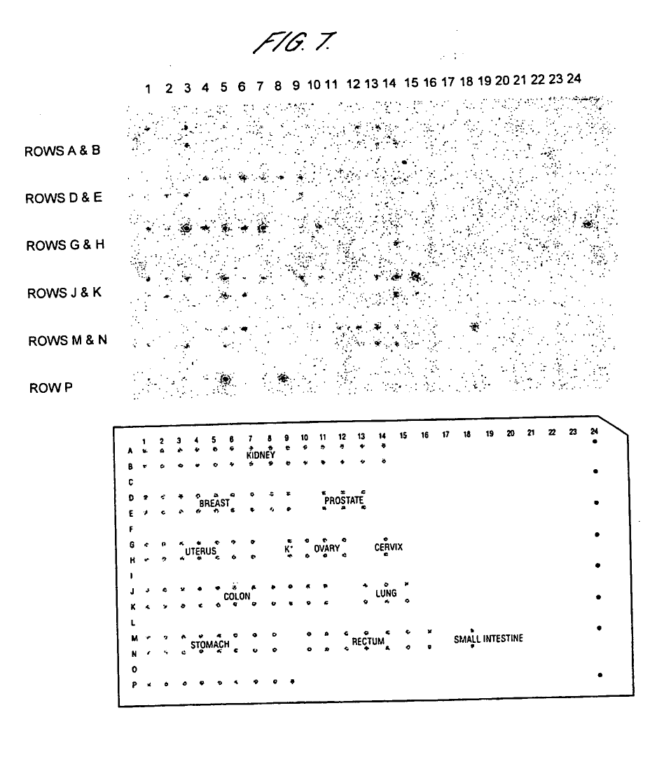

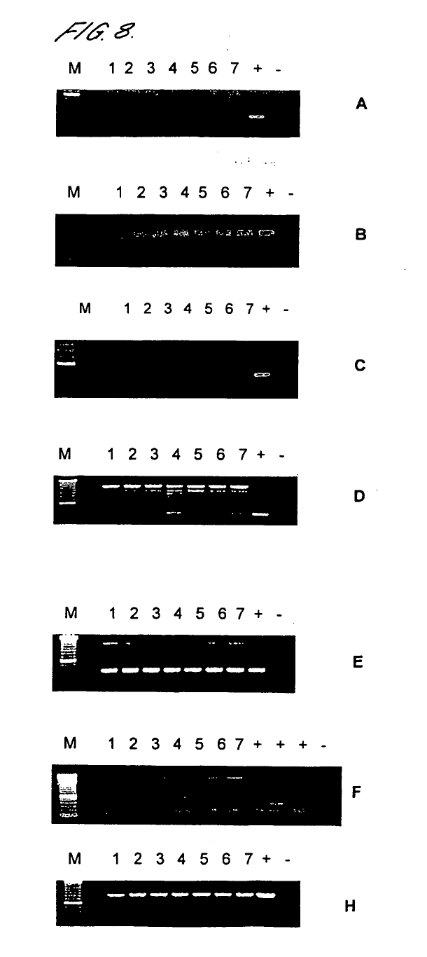

Also provided by the present invention is a nucleic acid molecule encoding a tumour associated antigen having the amino acid sequence illustrated in any of FIGS. 12, 14, 18 or 23. These antigens represent variants of those antigens that have been previously characterised as set out more fully in the examples provided. These antigens are encoded by the the nucleic acid molecules identified in FIGS. 11, 13, 17 or 22.

The antigens themselves encoded by the nucleotides according to the invention also form part of the invention.

A further aspect of the invention comprises nucleic acid molecules capable of hybridising to the nucleic acid molecules of the invention, under conditions of high stringency.

Stringency of hybridisation as used herein refers to conditions under which polynucleic acids are stable. The stability of hybrids is reflected in the melting temperature (Tm) of the hybrids. Tm can be approximated by the formula:
81.5° C.+16.6(log₁₀ [Na⁺]+0.41 (% G&C)-600/1
wherein 1 is the length of the hybrids in nucleotides. Tm decreases approximately by 1-1.5° C. with every 1% decrease in sequence homology.

The term “stringency” refers to the hybridisation conditions wherein a single-stranded nucleic acid joins with a complementary strand when the purine and pyrimidine bases therein pair with their corresponding base by hydrogen bonding. High stringency conditions favour homologous base pairing whereas low stringency conditions favour non-homologous base pairing.

“Low stringency” conditions comprise, for example, a temperature of about 37° C. or less, a formamide concentration of less than about 50%, and a moderate to low salt (SSC) concentration; or, alternatively, a temperature of about 50° C. or less, and a moderate to high salt (SSPE) concentration, for example 1M NaCl.

“High stringency” conditions comprise, for example, a temperature of about 42° C. or less, a formamide concentration of less than about 20%, and a low salt (SSC) concentration; or, alternatively, a temperature of about 65° C., or less, and a low salt (SSPE) concentration. For example, high stringency conditions comprise hybridization in 0.5 M NaHPO₄, 7% sodium dodecyl sulfate (SDS), 1 mM EDTA at 65° C. (Ausubel, F. M. et al. Current Protocols in Molecular Biology, Vol. I, 1989; Green Inc. New York, at 2.10.3).

“SSC” comprises a hybridization and wash solution. A stock 20×SSC solution contains 3M sodium chloride, 0.3M sodium citrate, pH 7.0.

“SSPE” comprises a hybridization and wash solution. A 1×SSPE solution contains 180 mM NaCl, 9mM Na₂HPO₄ and 1 mM EDTA, pH 7.4.

There are other conditions, reagents and so forth which can be used, which result in stringent hybridisation and the skilled practitioner is familiar with such conditions.

The nucleic acid capable of hybridising to nucleic acid molecules according to the invention will generally exhibit at least 70%, preferably at least 80, 85 or 90% and more preferably at least 95% and even more preferably at least 97% similarity or identity to the nucleotide sequences according to the invention.

An antisense molecule capable of hybridising to the nucleic acid according to the invention may be used as a probe or as a medicament or may be included in a pharmaceutical composition with a pharmaceutically acceptable carrier, diluent or excipient therefor to treat the particular lymphoma.

The term “homologous” describes the relationship between different nucleic acid molecules or amino acid sequences wherein said sequences or molecules are related by partial identity or similarity at one or more blocks or regions within said molecules or sequences. Homology may be determined by means of computer programs known in the art.

Substantial homology preferably carries with it that the nucleotide and amino acid sequences of the protein of the invention comprise a nucleotide and amino acid sequence fragment, respectively, corresponding and displaying a certain degree of sequence identity to the amino acid and nucleic acid sequences identified in the figures. Preferably they share an identity of at least 30%, preferably 40%, more preferably 50%, still more preferably 60%, most preferably 70%, and particularly an identity of at least 80%, preferably more than 90% and still more preferably more than 95% is desired with respect to the nucleotide or amino acid sequences according to the invention. A preferred method for determining the best overall match between a query sequence (a sequence of the present invention) and a subject sequence, also referred to as a global sequence alignment, can be determined using, for example, the Blast program described in Altschul, S. T., et al., (1990) Basic Local Alignment Search Tool, J. Mol. Biol., 215, 403-410. In a sequence alignment the query and subject sequences are both DNA sequences. An RNA sequence can be compared by converting U's to T's. The result of said global sequence alignment is in percent identity. Further programs that can be used in order to determine homology/identity are described below and in the examples. The sequences that are homologous to the sequences described above are, for example, variations of said sequences which represent modifications having the same biological function, in particular encoding proteins with the same or substantially the same specificity, e.g. binding specificity. They may be naturally occurring variations, such as sequences from other mammals, or mutations. These mutations may occur naturally or may be obtained by mutagenesis techniques. The allelic variations may be naturally occurring allelic variants as well as synthetically produced or genetically engineered variants. In a preferred embodiment the sequences are derived from human.

The nucleic acid molecules according to the invention may, advantageously, be included in a suitable expression vector to express the proteins encoded therefrom in a suitable host. Incorporation of cloned DNA into a suitable expression vector for subsequent transformation of said cell and subsequent selection of the transformed cells is well known to those skilled in the art as provided in Sambrook et al. (1989), Molecular cloning: A Laboratory Manual, Cold Spring Harbour Laboratory.

An expression vector, according to the invention, includes a vector having a nucleic acid according to the invention operably linked to regulatory sequences, such as promoter regions, that are capable of effecting expression of said DNA fragments. A vector can include a large number of nucleic acids which can have a desired sequence inserted therein by, for example, using an appropriate restriction enzyme and ligating the sequence in the vector, for transport between cells of different genetic composition. The term “operably linked” refers to a juxtaposition wherein the components described are in a relationship permitting them to function in their intended manner. Such vectors may be transformed into a suitable host cell to provide for expression of a protein according to the invention. The vectors are usually capable of replicating within a host environment and they also comprise one of a number of restriction sites for endonucleases which permits them to be cut in a selective manner at a particular location for insertion of a new nucleic acid molecule or sequence therein. Thus, in a further aspect, the invention provides a process for preparing polypeptides according to the invention, which comprises cultivating a host cell, transformed or transfected with an expression vector as described above under conditions to provide for expression by the vector of a coding sequence encoding the protein, and recovering the expressed protein.

In this regard, the nucleic acid molecule may encode a mature protein or a protein having a prosequence, including encoding a leader sequence on the preprotein which is cleaved by the host cell to form a mature protein.

The vectors may be, for example, plasmid, virus or phagemid vectors provided with an origin of replication, and optionally a promoter for the expression of said nucleotide and optionally a regulator of the promoter. The vectors may contain one or more selectable markers, such as, for example, an antibiotic resistance.

Regulatory elements required for expression include promoter sequences to bind RNA polymerase and to direct an appropriate level of transcription initiation and also translation initiation sequences for ribosome binding. For example, a bacterial expression vector may include a promoter such as the lac promoter and for translation initiation the Shine-Dalgarno sequence and the start codon AUG. Similarly, a eukaryotic expression vector may include a heterologous or homologous promoter for RNA polymerase II, a downstream polyadenylation signal, the start codon AUG, and a termination codon for detachment of the ribosome. However, the precise regulatory elements required for expression of a gene of interest may vary between different cell types but generally include 5′ non-transcribing and non-translating regions which are required for initiation of translation and transcription. Such vectors may be obtained commercially or be assembled from the sequences described by methods well known in the art.

Transcription of DNA encoding the polypeptides of the present invention by higher eukaryotes is optimised by including an enhancer sequence in the vector. Enhancers are cis-acting elements of DNA that act on a promoter to increase the level of transcription. Vectors will also generally include origins of replication in addition to the selectable markers.

Nucleic acid molecules according to the invention may be inserted into the vectors described in an antisense orientation in order to provide for the production of antisense RNA. Antisense RNA or other antisense nucleic acids, including antisense peptide nucleic acid (PNA), may be produced by synthetic means.

In accordance with the present invention, a defined nucleic acid includes not only the identical nucleic acid but also any minor base variations including in particular, substitutions in cases which result in a synonymous codon (a different codon specifying the same amino acid residue) due to the degenerate code in conservative amino acid substitutions. The term “nucleic acid sequence” also includes the complementary sequence to any single stranded sequence given regarding base variations.

As used herein with respect to nucleic acids “isolated” means any of a) amplified in vitro by, for example, polymerase chain reaction (PCR), b) recombinantly produced by cloning, c) purified by, for example, gel separation, or d) synthesised, such as by chemical synthesis.

The present invention also advantageously provides oligonucleotides comprising at least 10 consecutive nucleotides of a nucleic acid according to the invention and preferably from 10 to 40 consecutive nucleotides of a nucleic acid according to the invention. As would be appreciated by one of skill in the art, it is also possible to use as primers those untranslated regions (UTR's) of the gene encoding the polypeptides of the invention. For example, 3′ and 5′ UTR's can be used to identify homologues of the polypeptide of the invention. The oligonucleotides of the invention may, advantageously be used as probes or primers to initiate replication, or the like. Oligonucleotides having a defined sequence may be produced according to techniques well known in the art, such as by recombinant or synthetic means. They may also be used in diagnostic kits or the like for detecting the presence of a nucleic acid according to the invention. These tests generally comprise contacting the probe with the sample under hybridising conditions and detecting for the presence of any duplex or triplex formation between the probe and any nucleic acid in the sample.

According to the present invention these probes may be anchored to a solid support. Preferably, they are present on an array so that multiple probes can simultaneously hybridize to a single biological sample. The probes can be spotted onto the array or synthesised in situ on the array. (See Lockhart et al., Nature Biotechnology, vol. 14, December 1996 “Expression monitoring by hybridisation to high density oligonucleotide arrays”.

The nucleic acid sequences according to the invention may be produced using recombinant or synthetic techniques, such as for example using PCR which generally involves making a pair of primers, which may be from approximately 10 to 50 nucleotides to a region of the gene which is desired to be cloned, bringing the primers into contact with cDNA, or genomic DNA from a human cell, performing a polymerase chain reaction under conditions which bring about amplification of the desired region, isolating the amplified region or fragment and recovering the amplified DNA. Generally, such techniques are well known in the art, such as described in Sambrook et al. (Molecular Cloning: a Laboratory Manual, 1989).

The nucleic acids or oligonucleotides according to the invention may carry a revealing label. Suitable labels include radioisotopes such as ³²P or ³⁵S, enzyme labels or other protein labels such as biotin or fluorescent markers. Such labels may be added to the nucleic acids or oligonucleotides of the invention and may be detected using known techniques per se.

Advantageously, human allelic variants or polymorphisms of the nucleic acid according to the invention may be identified by, for example, probing cDNA or genomic libraries from a range of individuals, for example, from different populations. Furthermore, nucleic acids and probes according to the invention may be used to sequence genomic DNA from patients using techniques well known in the art, such as the Sanger Dideoxy chain termination method, which may, advantageously, ascertain any predisposition of a patient to disorders associated with variants of the polypeptides of the invention.

In the very least, the nucleotide sequences can be used as molecular weight markers on Southern gels, as diagnostic probes for the presence of a specific mRNA in a particular cell type, as a probe to “subtract-out” known sequences in the process of discovering novel nucleotide sequences, for selecting and making oligomers for attachment to a “gene chip” or other support, to raise anti-DNA antibodies using DNA immunization techniques, and as an antigen to elicit an immune response.

The nucleotide sequences identified herein according to the invention can be used in numerous ways as a reagent. The following description should be considered exemplary and utilizes known techniques.

There exists an ongoing need to identify new chromosome markers, since few chromosome marking reagents, based on actual sequence data (repeat polymorphisms), are presently available. Other mapping techniques that may be used include in situ hybridization to chromosomal spreads, flow-sorted chromosomal preparations, or artificial chromosome constructions such as yeast artificial chromosomes, bacterial artificial chromosomes, bacterial P1 constructions or single chromosome cDNA libraries as reviewed in Price (Blood Rev. 7 (1993), 127-134) and Trask (Trends Genet. 7 (1991), 149-154). The technique of fluorescent in situ hybridization of chromosome spreads has been described, among other places, in Verma, (1988) Human Chromosomes: A Manual of Basic Techniques, Pergamon Press, New York N.Y. Fluorescent in situ hybridization of chromosomal preparations and other physical chromosome mapping techniques may be correlated with additional genetic map data. Examples of genetic map data can be found in the art. Correlation between the location of the gene encoding a polypeptide of the invention on a physical chromosomal map and a specific feature, e.g., a disease related to the dysfunction of the gene may help to delimit the region of DNA associated with this feature. The nucleotide sequences of the subject invention may be used to detect differences in gene sequences between normal, carrier or affected individuals. Furthermore, the means and methods described herein can be used for marker-assisted animal breeding.

In situ hybridization of chromosomal preparations and physical mapping techniques such as linkage analysis using established chromosomal markers may be used for extending genetic maps. For example a sequence tagged site based map of the human genome was recently published by the Whitehead-MIT Center for Genomic Research (Hudson, Science 270 (1995), 1945-1954) and is also available on the internet. Often the placement of a gene on the chromosome of another species may reveal associated markers even if the number or arm of a particular chromosome is not known. New sequences can be assigned to chromosomal arms, or parts thereof, by physical mapping. This provides valuable information to investigators searching for interacting genes using positional cloning or other gene discovery techniques. Once such gene has been crudely localized by genetic linkage to a particular genomic region, any sequences mapping to that area may represent associated or regulatory genes for further investigation. The nucleotide sequence of the subject invention may also be used to detect differences in the chromosomal location due to translocation, inversion, etc. among normal, carrier or affected individuals.

Briefly, sequences can be mapped to chromosomes by preparing PCR primers (preferably 15-25 bp) from the sequences of the invention. Primers can be selected using computer analysis so that primers do not span more than one predicted exon in the genomic DNA. These primers are then used for PCR screening of somatic cell hybrids containing individual human chromosomes. Only those hybrids containing the human gene of interest corresponding to the above sequences will yield an amplified fragment.

Similarly, somatic hybrids provide a rapid method of PCR mapping the nucleotide sequences to particular chromosomes. Three or more clones can be assigned per day using a single thermal cycler. Moreover, sublocalization of the nucleotide sequences can be achieved with panels of specific chromosome fragments. Other gene mapping strategies that can be used include in situ hybridization, prescreening with labeled flow-sorted chromosomes, and preselection by hybridization to construct chromosome specific cDNA libraries.

Precise chromosomal location of the nucleotide sequences can also be achieved using fluorescence in situ hybridization (FISH) of a metaphase chromosomal spread. This technique uses nucleotide sequences as short as 300 to 600 bases; however, nucleotide sequences 1,000-4,000 bp are preferred. For a review of this technique, see Verma et al., “Human Chromosomes: a Manual of Basic Techniques,” Pergamon Press, New York (1988).

For chromosome mapping, the nucleotide sequences can be used individually (to mark a single chromosome or a single site on that chromosome) or in panels (for marking multiple sites and/or multiple chromosomes).

Once a nucleotide sequence has been mapped to a precise chromosomal location, the physical position of the nucleotide sequence can be used in linkage analysis. Linkage analysis establishes coinheritance between a chromosomal location and presentation of a particular disease. (Disease mapping data are found, for example, in McKusick, Mendelian Inheritance in Man (available on line through Johns Hopkins University Welch Medical Library)). Assuming 1 megabase mapping resolution and one gene per 20 kb, a cDNA precisely localized to a chromosomal region associated with the disease could be one of 50-500 potential causative genes.

Thus, once coinheritance is established, differences in the nucleotide sequences of the invention and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the polypeptide encoded and the corresponding gene from several normal individuals is required to distinguish the mutation from a polymorphism. If a new polymorphism is identified, this polymorphic polypeptide can be used for further linkage analysis.

According to a further aspect of the invention, there is also provided an isolated polypeptide encoded by the nucleic acid molecules of the invention. Preferably, the polypeptide comprises the sequence of amino acids set forth in FIGS. 2 or 10. According to a further aspect there is also provided a tumour associated antigen encoded by the nucleic acid molecules identified in FIGS. 1 or 9 or FIGS. 11, 13, 17 or 22.

According to a further aspect, the invention comprises an isolated lymphoid antigen comprising an amino acid sequence exhibiting at least 70% sequence similarity/identity to the amino acid sequences illustrated in FIGS. 2 or 10, or 12, 14, 18, or 23 or a functional equivalent or derivative thereof. Preferably, the invention comprises an isolated polypeptide exhibiting at least 75%, preferably 80, more preferably 85, even more preferably 90, 95 or 97% sequence similarity/identity to the amino acid sequences depicted in FIGS. 2, 10, 12, 14, 18 or 23. Functional homologues or equivalents of the polypeptide of the invention can be prepared according to methods known in the art, and which comprise, amongst others, altering the polypeptide sequence as set out in Molecular Cloning, A Laboratory Manual, Sambrook et al. Conservative amino acid substitutions can be performed by altering the nucleic acid encoding the polypeptide, using, for example, PCR or site directed mutagenesis or by chemical synthesis of the nucleic acid molecule. Computer algorithms can also be utilised which predict the amino acid sequences that may be altered or substituted to prepare said functional equivalents.

A polypeptide according to the invention includes all possible amino acid variants encoded by its corresponding nucleic acid molecule, including a polypeptide encoded by said molecule and having conservative amino acid changes. Proteins or polypeptides according to the invention further include variants of such sequences, including naturally occurring allelic variants which are substantially homologous to said proteins or polypeptides. In this context, substantial similarity/identity is regarded as a sequence which has at least 60%, 70%, preferably 80 or 90%, more preferably 95% and even more preferably 97% amino acid homology with the proteins or polypeptides encoded by the nucleic acid molecules according to the invention. The protein according to the invention may be recombinant, synthetic or naturally occurring, but is preferably recombinant.

As used herein with respect to polypeptides, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use.

As aforementioned, the polypeptides according to the invention and those antigens identified in Table 1 can be used to identify tumour antigens, and constitute antigens that are expressed in either solid tumours or lymphomas and which are recognised by the hosts immune response. Therefore, in some instances down-regulation or inhibition of the tumour-associated antigen may, advantageously, be used as a therapeutic treatment for such tumours. Therefore, the present invention is further directed to inhibiting expression or activity of the polypeptides of the invention, including those identified in Table 1 in vivo by, for example, inhibiting transcription by, for example, the use of antisense technology. However, as would be appreciated by the skilled practitioner any other suitable method may be utilised. Other methods of inhibiting protein expression may utilise antibodies or binding polypeptides or other small molecules which, for example, bind or block the binding region of the polypeptides of the invention. As used herein, the term “antisense nucleotide” or “antisense” describes an oligonucleotide that is an oligoribonucleotide, oligodeoxyribonucleotide, modified oligoribonucleotide, or modified oligodeoxyribonucleotide which hybridises under physiological conditions to DNA encoding the polypeptide of the invention or to an mRNA transcript of the gene and, thereby, inhibits the transcription of that gene and/or translation of mRNA. Antisense technology can be used to control gene expression through triple-helix formation of antisense DNA or RNA, both of which methods are based on binding of a polynucleotide to DNA or RNA. For example, the 5′ coding portion or the mature protein sequence, which encodes for the protein of the present invention, is used to design an antisense RNA oligonucleotide of from 10 to 40 base pairs in length. The antisense RNA oligonucleotide hybridises to the mRNA in vivo and blocks translation of an mRNA molecule into the protein (antisense—Okano, J. Neurochem., 56:560 (1991); Oligodeoxynucleotides as Antisense Inhibitors of Gene Expression, CRC Press, Boca Raton, Fla. (1988)). A DNA oligonucleotide is designed to be complementary to a region of the gene involved in transcription (triple-helix—see Lee et al. Nucl. Acids Res., 6:3073 (1979); Cooney et al., Science, 241:456 (1988); and Dervan et al., Science, 251: 1360 (1991), thereby preventing transcription and the production of the polypeptide.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, peptides, and carboxymethyl esters.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus, modified oligonucleotides may include a 2′-O-alkylated ribose group. In addition, modified oligonucleotides may include sugars such as arabinose instead of ribose. Modified oligonucleotides also can include base analogs such as C-5 propyne modified bases (Wagner et al., Nature Biotechnology 14:840-844, 1996). The present invention, thus, contemplates pharmaceutical preparations containing modified antisense molecules that are complementary to and hybridizable with, under physiological conditions, nucleic acids encoding polypeptides of the invention together with pharmaceutically acceptable carriers.

The hybrid and modified forms include, for example, when certain amino acids have been subjected to some modification or replacement, such as for example, by point mutation and yet which results in a polypeptide or protein which possesses the same function as the polypeptides of the invention.

The antisense oligonucleotide described above can be delivered to cells by procedures in the art such that the anti-sense RNA and DNA may be expressed in vivo to inhibit production of the protein in the manner described above.

A further aspect of the invention provides a host cell or organism, transformed or transfected with an expression vector according to the invention. The cell or organism may be transformed or transfected using techniques that are well known in the art, such as, electroporation or by using liposomes. The host cell or organism may advantageously be used in a method of producing polypeptides, which comprises recovering any expressed polypeptide from the host or organism transformed or transfected with the expression vector.

According to a further aspect of the invention there is also provided a transgenic cell, tissue or organism comprising a transgene capable of expressing a polypeptide according to the invention. The term “transgene capable of expressing” as used herein encompasses any suitable nucleic acid sequence which leads to expression of a polypeptide(s) having the same function and/or activity as the polypeptides of the invention. The transgene, may include, for example, genomic nucleic acid isolated from human cells or synthetic nucleic acid, including DNA integrated into the genome or in an extrachromosomal state. Preferably, the transgene comprises the nucleic acid sequence encoding the polypeptide according to the invention as described herein, or a functional fragment of said nucleic acid. A functional fragment of said nucleic acid should be taken to mean a fragment of the gene comprising said nucleic acid coding for the polypeptides according to the invention or a functional equivalent, derivative or a non-functional derivative such as a dominant negative mutant of said polypeptides.

Transgenic non-human organisms are being utilised as model systems for studying both normal and disease cell processes. In general, to create such transgenic animals an exogenous gene with or without a mutation is transferred to the animal host system and the phenotype resulting from the transferred gene is observed. Other genetic manipulations can be undertaken in the vector or host system to improve the gene expression leading to the observed phenotype (phenotypic expression). The gene may be transferred on a vector under the control of different inducible or constitutive promoters, may be overexpressed or the endogenous homologous gene may be rendered unexpressible, and the like (WO 92/11358). The vector may be introduced by transfection or other suitable techniques such as electroporation, for example, in embryonic stem cells. The cells that have the exogenous DNA incorporated into their genome, for example, by homologous recombination, may subsequently be injected into blastocytes for generation of the transgenic animals with the desired phenotype. Successfully transformed cells containing the vector may be identified by well known techniques such as lysing the cells and examining the DNA, by, for example, Southern blotting or using the polymerase chain reaction.

Knock-out organisms may be generated to further investigate the role of the polypeptide of the invention in vivo. By “knock-out” it is meant an animal which has its endogenous gene knocked out or inactivated. Typically, homologous recombination is used to insert a selectable gene into an essential exon of the gene of interest. Furthermore, the gene of interest can be knocked out in favour of a homologous exogenous gene to investigate the role of the exogenous gene (Robbins, J., GENE TARGETING. The Precise Manipulation of the Mammalian Genome Circ. Res. 1993, J.W.; 73; 3-9). Transgenic animals, such as mice or Drosophila or the like, may therefore be used to over or under express the proteins according to the invention to further investigate their role in vivo and in the progression or treatment of lymphoma.

The polypeptide expressed by said transgenic cell, tissue or organism or a functional equivalent thereof also forms part of the present invention. Recombinant proteins or polypeptides may be recovered and purified from host cell cultures by methods known in the art, including ammonium sulfate or ethanol precipitation, acid extraction, anion or cation exchange chromatography, phosphocellulose, chromatography, hydrophobic interaction chromatography, affinity chromatography, hydroxyapatite chromatography and lectin chromatography.

The polypeptide of the present invention may be a naturally purified product, or a product of chemical synthetic procedures, or produced by recombinant techniques from a prokaryotic or eukaryotic host (for example, by bacteria, yeast, higher plant, insect and mammalian cells in culture). Depending upon the host employed in a recombinant production procedure, the expressed polypeptide may lack the initiating methionine residue as a result of post-translational cleavage. Proteins or polypeptides which have been modified in this way are also included within the scope of the invention.

In a still further aspect the invention provides a binding polypeptide which is capable of binding to the polypeptide of the invention or an epitope thereof, including those as identified in Table 1, which form part of this aspect of the present invention.

In one embodiment, the binding polypeptide comprises an antibody, for example, or a polypeptide exhibiting regions of similarity/identity with the polypeptide of the invention and capable of binding thereto. Such an antibody may be polyclonal, for example, and may be raised according to standard techniques well known to those skilled in the art by using the polypeptide of the invention or a fragment or single epitope thereof as the challenging antigen. Alternatively, the antibody may be monoclonal in nature and may be produced according to the techniques described by Kohler & Milstein (Nature (1975) 256, 495-497).

When the binding protein or polypeptide is an antibody the present invention includes not only complete antibody molecules but fragments thereof. Antibody fragments which contain the idiotype of the molecule can be generated by known techniques, for example, such fragments include but are not limited to the F(ab′)₂ fragment which can be produced by pepsin digestion of the antibody molecule; the Fab′ fragments which can be generated by reducing the disulfide bridges of the F(ab′)₂ fragments and the Fab fragments which can be generated by treating the antibody molecule with papain and a reducing agent. Chimeric, humanized and fully humanized monoclonal antibodies can now be made by recombinant engineering. By addition of the human constant chain to F(ab′)₂ fragments it is possible to create a humanized monoclonal antibody which is useful in immunotherapy applications where patients making antibodies against the mouse Ig would otherwise be at a disadvantage. Breedveld F. C. Therapeutic Monoclonal Antibodies. Lancet Feb. 26, 2000; 335, P735-40.

Furthermore, as is well-known in the art, only a small portion of an antibody molecule, the paratope, is involved in the binding of the antibody to its epiptope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Polypeptides that bind to the polypeptide of the invention may be identified by Phage Display. In this technique a phage library is prepared, displaying inserts from between about 4 to 80 amino acid residues using techniques which are well known in the art. It is then possible to select those Phage bearing inserts that bind to the polypeptide of the invention. DNA sequence analysis is then performed to identify the nucleic acid sequences encoding the expressed polypeptides.

Antibody fragments of predetermined binding specificity may also be constructed using Phage Display technology, which obviates the need for hybridoma technology and immunization. These antibody fragments are created from repertoires of antibody V genes which are harvested from populations of lymphocytes, or assembled in vitro, and cloned for display of associated heavy and light chain variable domains on the surface of filamentous bacteriophage. The process mimics immune selection and antibodies with many different binding specificities have been isolated from the same Phage repertoire. (Winter et al., Annu. Rev. Immunol. 1994; 12:433-55). Such antibodies are also embraced within the scope of the binding polypeptides of the present invention.

Other types of binding polypeptides that may be utilised in accordance with the invention are termed immunoadhesins. Immunoadhesins are a class of fusion proteins, which combine the target-binding region of a receptor, an adhesion molecule, a ligand or an enzyme, with the Fc portion or an immunoglobulin. Production of immunoadhesins is described in Byrn et al (1990) Nature 344, pp 667-670.

In a preferred embodiment an antibody according to the invention is an autoantibody isolated from the sera of a patient suffering from a particular tumour type which, for certain of the antigens in Table 1 for which no previous SEREX association has been identified, directed against a solid tumour or lymphoma and particularly DLBCL.

It is also within the knowledge of the skilled artisan to produce immortalised cell lines capable of producing antibodies according to the invention and these are also embraced within the scope of the invention.

The nucleic acid molecules or the polypeptides of the invention may also be included in a pharmaceutical composition together with any suitable pharmaceutically acceptable carrier diluent or excipient therefor. The nucleic acid molecule or polypeptides or antibodies/binding polypeptide may be encapsulated and/or combined with suitable carriers in solid dosage forms for oral administration which would be well known to those of skill in the art or alternatively with suitable carriers for administration in an aerosol spray.

In the pharmaceutical composition of the invention, preferred compositions include pharmaceutically acceptable carriers including, for example, non-toxic salts, sterile water or the like. A suitable buffer may also be present allowing the compositions to be lyophilized and stored in sterile conditions prior to reconstitution by the addition of sterile water for subsequent administration. The carrier can also contain other pharmaceutically acceptable excipients for modifying other conditions such as pH, osmolarity, viscosity, sterility, lipophilicity, somobility or the like. Pharmaceutical compositions which permit sustained or delayed release following administration may also be used.

Furthermore, as would be appreciated by the skilled practitioner, the specific dosage regime may be calculated according to the body surface area of the patient or the volume of body space to be occupied, dependent on the particular route of administration to be used. The amount of the composition actually administered will, however, be determined by a medical practitioner based on the circumstances pertaining to the disorder to be treated, such as the severity of the symptoms, the age, weight and response of the individual.

The invention also contemplates gene therapy. This involves introduction in vitro of a functional copy of a gene into a cell(s) of a subject which contains a defective copy of the gene and returning the genetically engineered cell(s) to the subject. The functional copy of the gene is under operable control of regulatory elements which permit expression of the gene in the genetically engineered cells. Numerous transfection and transduction techniques as well as appropriate expression vectors for carrying out such procedures are well known in the art. In vivo gene therapy using plasmids or viral vectors, such as adenovirus, vaccina virus and the like, is also contemplated according to the invention. Thus, incorporation of a nucleic acid molecule encoding the polypeptide according to the present invention using gene therapy should permit replacement of any defective protein contributing to the lymphoma and which should resume its normal function.

A further aspect of the present invention also provides a method of identifying a polypeptide of the invention in a sample, which method comprises contacting said sample with a binding polypeptide as described herein and monitoring for any specific binding of any polypeptides to said binding polypeptide. A kit for identifying the presence of such polypeptides in a sample is also provided comprising a binding polypeptide as described above and means for contacting said binding polypeptide with said sample.

In a further aspect the invention provides an in vitro method of detecting expression of polypeptides of the invention which method comprises contacting a sample of bodily fluid, particularly blood serum, cells or cell lysates from a subject with a binding protein as previously described and detecting any binding of said binding polypeptide to a protein in the sample.

The invention also comprises a method of modulating activity/function of the tumour antigens of the invention, as set out in Table 1, which method comprises inhibiting or enhancing expression or activity in a cell of a polypeptide according to the invention. Enhancing expression of a particular protein may be beneficial where it is a tumour suppressor. Numerous methods and techniques are available in the art for inhibiting or enhancing expression or function of the polypeptide of the invention which would be known to the skilled practitioner. For example, increased expression of antigens of the invention may be achieved by transformation of a suitable expression vector incorporating the nucleic acid sequence of the invention, whereas inhibiting its function or expression may be accomplished using antisense techniques described herein or by using a blocking or binding protein. Furthermore, as would be well known to the skilled practitioner, other small molecules, such as binding peptides or polypeptides or other compounds may be synthesised or produced which can inhibit function or activity of the polypeptides of the invention. Furthermore, double stranded RNA inhibition or small interfering molecules (siRNA) may also be applied and which offers increased efficiency in inhibiting the target gene of interest.

The polypeptide expressed by expression of the said transgenic cell, tissue or organism or a functional equivalent thereof also forms part of the present invention.

As aforementioned, polypeptides according to the invention as set out heerein are considered to represent important markers that may be utilised for the prophylactic or therapeutic immunisation against certain tumours or lymphomas because they are specifically expressed or over-expressed in such tumours or lymphomas compared to normal cells and as such they may be targeted by antigen-specific immune mechanisms leading to the destruction of the tumour.

The invention therefore also contemplates a vaccine composition including, not only the polypeptides according to the invention as already described, but immunogenic fragments or epitopes thereof, which are capable of initiating an immune response, either by themselves or coupled to a suitable carrier. Also comprised within the scope of the invention are mimotopes which exhibit the same immune response initiating characteristics as the epitopes. The invention also therefore includes polypeptides incorporating the epitopes or mimotopes described. A mimotope is described as an entity which is sufficiently similar to the epitopes of the polypeptides of the invention so as to be capable of being recognised by antibodies which recognise the native molecule. They may be generated by addition, deletion or substitution of elected amino acids which, advantageously, means that the polypeptides or epitopes thereof of the invention may be modified, for example, for ease of delivery on a carrier.

Carriers which may be used with the immunogens of the present invention will be well known to those of skill in the art. The function of the carrier is to provide cytokine help to facilitate the induction of an immune response following administration of the vaccine composition to an individual. Methods for immunisation, including formulating the vaccine composition and selecting appropriate doses are well known to those of skill in the art.

According to another embodiment, the vaccine compositions described herein will comprise one or more immunostimulants in addition to the immunogenic polynucleotide, polypeptide, antibody, T-cell and/or antigen presenting cell (APC) compositions of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as a lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition is preferably one that includes an immune response predominantly of the Th1 type. High levels of Th1 type cytokines (e.g., IFN_{-γ}, TNFα, IL-2 and IL-12) tend to favor the induction of cell mediated immune responses to an administered antigen. In contrast, high levels of Th2-type cytokines (e.g., IL-4, IL-5, IL-6 and IL10) tend to favor the induction of humoral immune responses. Following application of a vaccine as provided herein, a patient will support an immune response that includes both Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

The present invention also provides a polyvalent vaccine composition comprising a vaccine formulation of the invention in combination with other antigens, in particular antigens useful for treating cancers, autoimmune diseases and related conditions. Such a polyvalent vaccine composition may include a Th-1 inducing adjuvant as hereinbefore described.

According to another embodiment of this invention, an immunogenic composition described herein is delivered to a host via antigen presenting cells (APCs), such as dendritic cells, macrophages, B cells, monocytes, and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). Animal models have shown that APCs may generally be isolated from any of a variety of biological fluids and organs, including tumour and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumour immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), they possess a characteristic immunophenotype their ability to take up, process and present antigens with high efficiency and their ability to activate native T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (caled exosomes) may be used within a vaccine (see Zitvogel et al., Nature Med. 4:594-600, 1998).

Dendritic cells and progenitors may be obtained from peripheral blood, bone marrow, tumour-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide of the invention (or portion or other variant thereof) or a nucleic acid sequence encoding any of the antigens identified in Table 1, such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75: 456-460, 1997. Antigen loading of dendritic cells may be achieved by incubating dendritic cells or progenitor cells with the tumour polypeptide, tumour protein DNA (naked or within a plasmid vector) or RNA; or with antigen-expressing recombinant bacterium or viruses (e.g., vaccinia, fowlpox, adenovirus or lentivirus vectors). Prior to loading, the protein or polypeptide may be covalently conjugated to an immunological partner that provides T cell help (e.g., a carrier molecule). Alternatively, a dendritic cell may be pulsed with a non-conjugated immunological partner, separately or in the presence of the antigen.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, destran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO94/20078, WO/94/23701 and WO96/06638). The amount of active compound contained within a sustained release formulation depends upon the site of implantation, the rate and expected duration of release and the nature of the condition to be treated or prevented.

The identification of antigens in certain tumours and lymphomas renders it possible to detect or identify patients suffering from this form of cancer and will help in determining the appropriate course of treatment. Identification of various expression levels of the antigens will be useful in both diagnosis and in prognosis by grading the nature and advancement of the tumour or lymphoma.

Therefore, in one aspect of the invention, there is provided a method of identifying tumours or malignant cells in a mammal which method comprises identifying in a sample of bodily fluid from said mammal antibodies from said mammal capable of forming complexes with an antigen according to the invention. In one embodiment the antibodies are specific for the antigens as set out in FIGS. 2, 10, 12, 14, 18 or 23. Alternatively, the antibodies are directed to the antigens as designated herein as OX-TES-1, 2, 3, 4, 5, 15, 19, 20, 21, 22, 26, 27 or 28 Preferably, the mammal is a human and the antibodies are human autoantibodies. Preferably, the malignant cell or tumour is either a solid tumour or a leukaemia or lymphoma which itself may be a B cell lymphoma such as any of DLBCL, follicular lymphoma, or Burkitt's lymphoma, but preferably DLBCL. In a further aspect of the invention, there is provided a method of detecting lymphoma in a patient which method comprises identifying autoantibodies from a bodily fluid of said patient directed to any of said lymphoid tumour antigens designated OX-TES-1 to 28 illustrated in Table 1.

The invention may be more clearly understood from the following examples with reference to the accompanying Tables and Figures wherein:

FIG. 1 is a nucleic acid molecule encoding a novel, PAS domain containing polypeptide which was identified as a DLBCL-associated antigen (OX-TES-1).

FIG. 2 is an amino acid sequence of the polypeptide encoded by the nucleic acid molecule of FIG. 1 which also identifies the major domains present within the polypeptide.

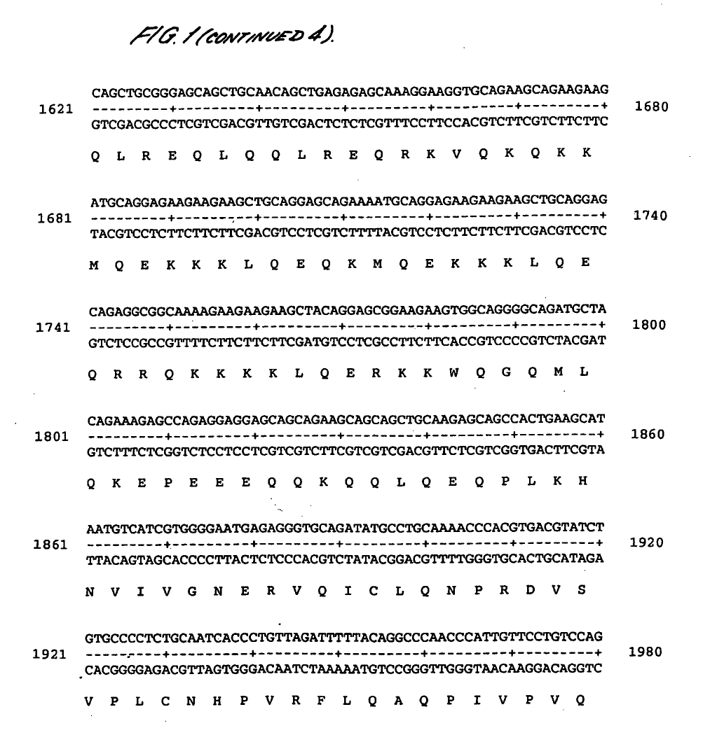

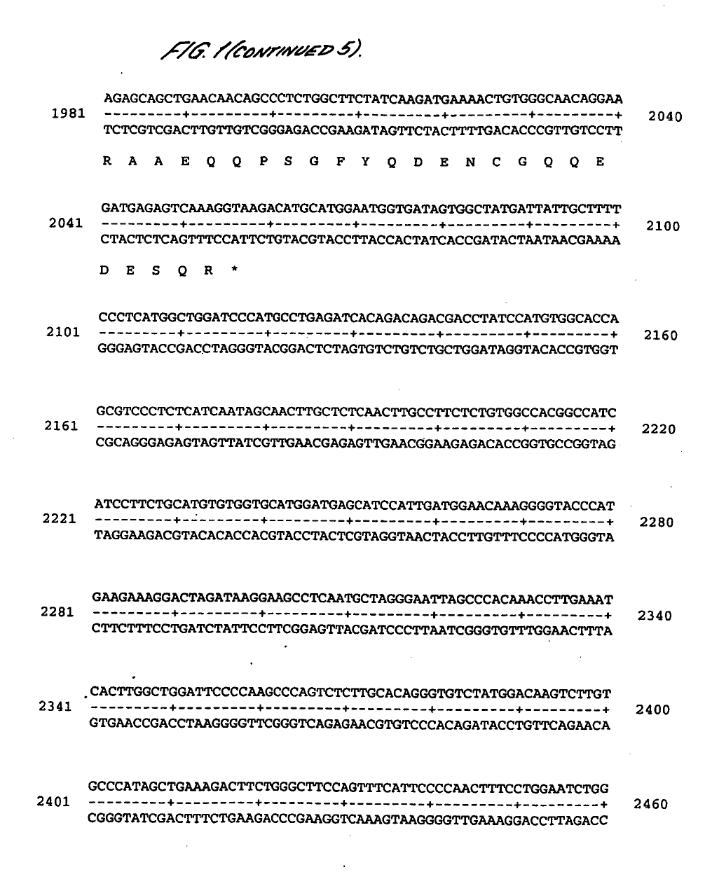

FIG. 3 shows the genomic arrangement of OX-TES-1 (A) and BC040301 (B).

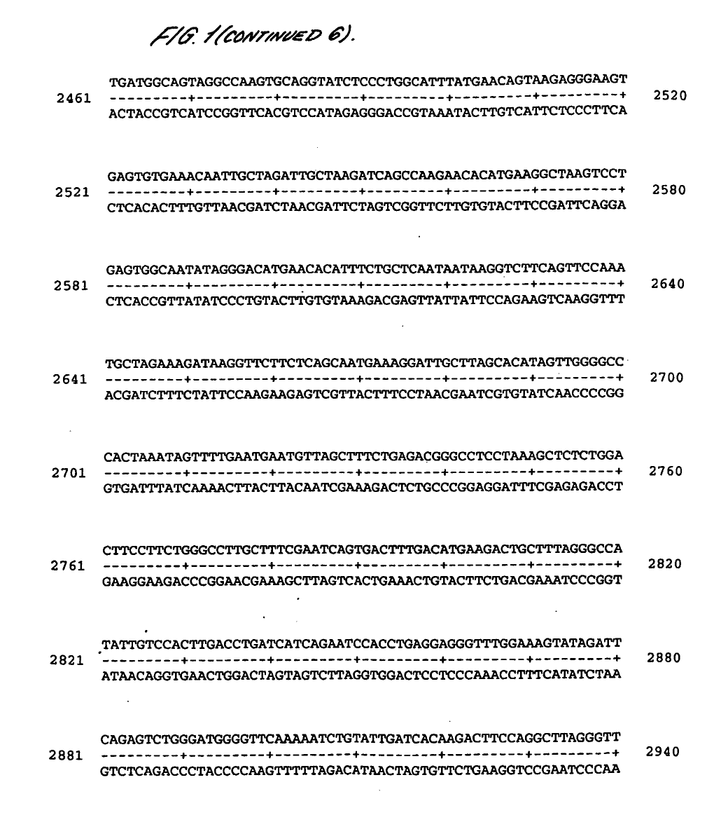

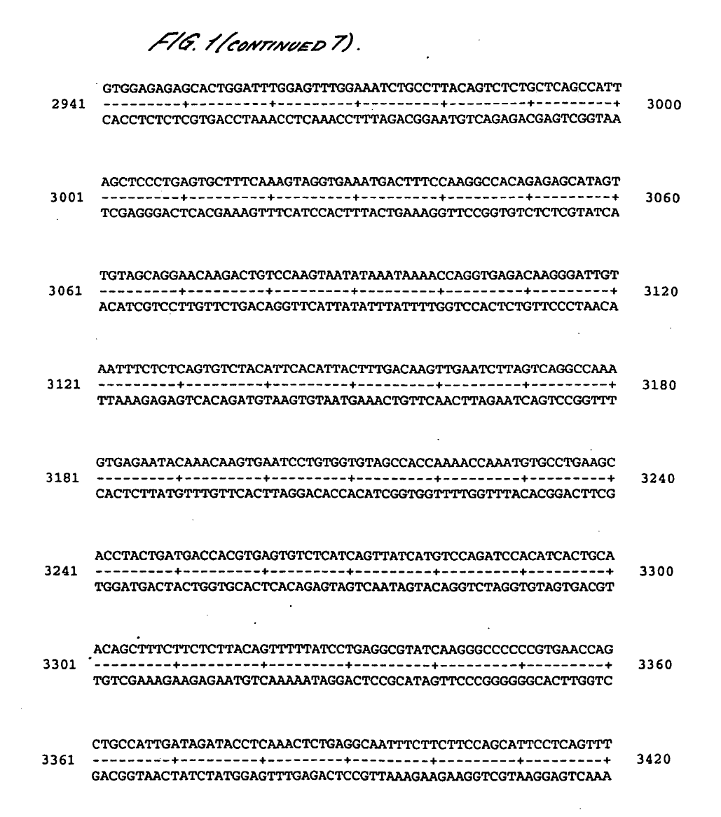

FIG. 4 is an alignment of the cDNA sequences of OX-TES-1 and BC040301, showing clearly the retained region in OX-TES-1. Differences at the nucleotide level are shaded.

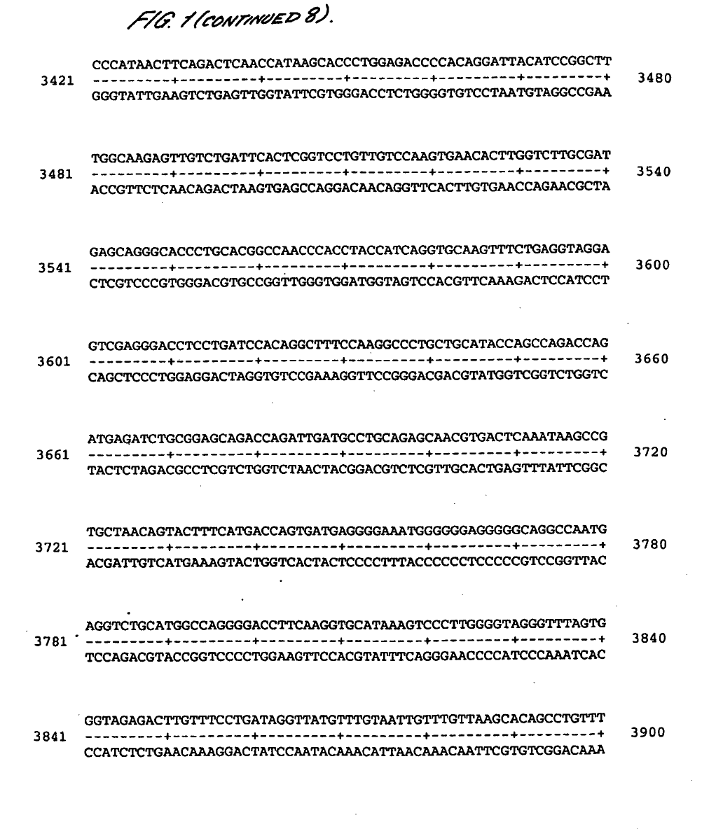

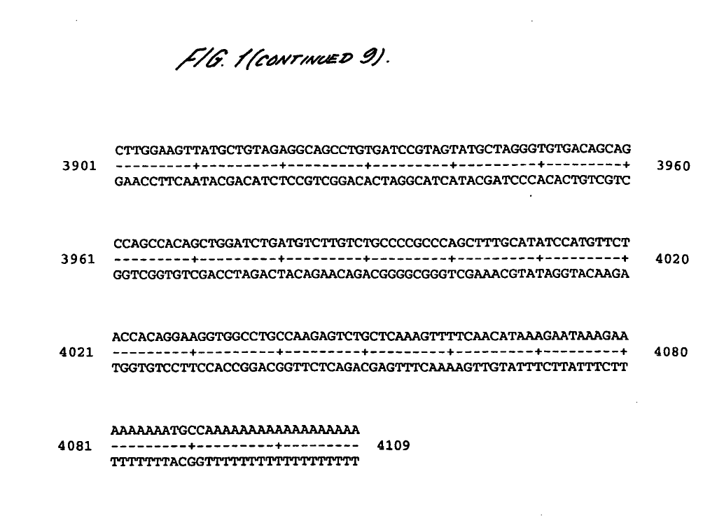

FIG. 5 is an alignment of the predicted protein sequences of OX-TES-1 and BC040301.

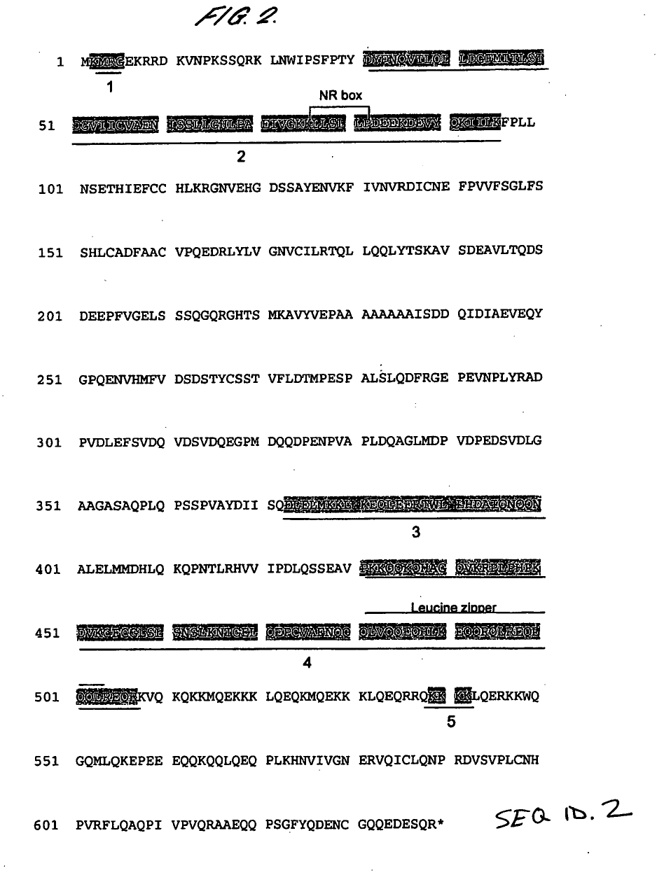

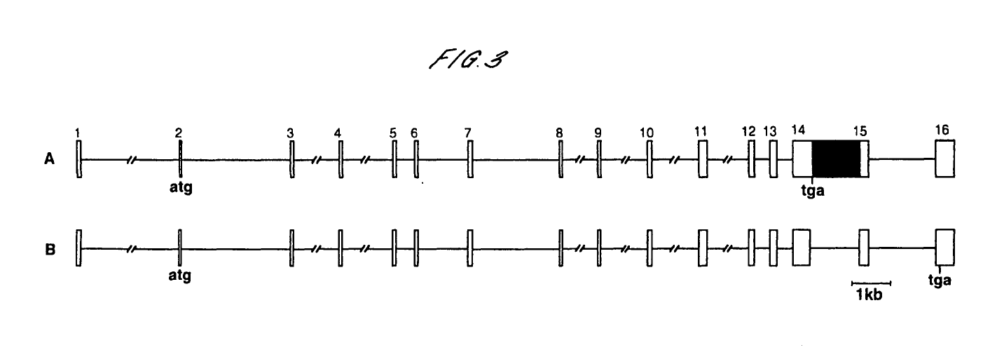

FIG. 6 illustrates the results obtained from hybridising the OX-TES-1 cDNA to BD Biosciences' Multiple Tissue Expression Array. The identity and position of tissues on the array is shown in the lower panel. The positive signals are arrowed.

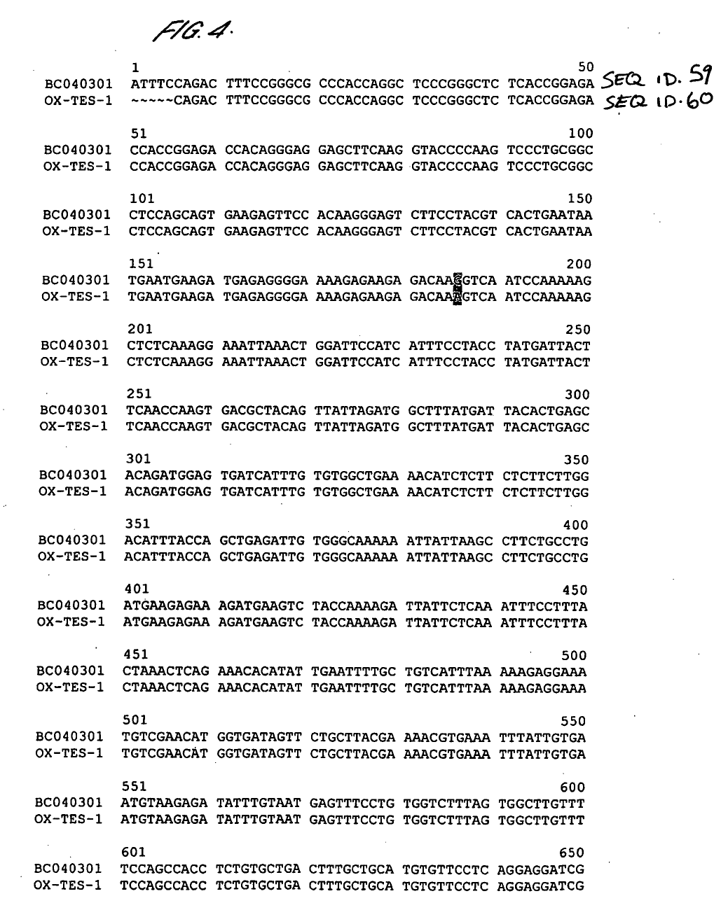

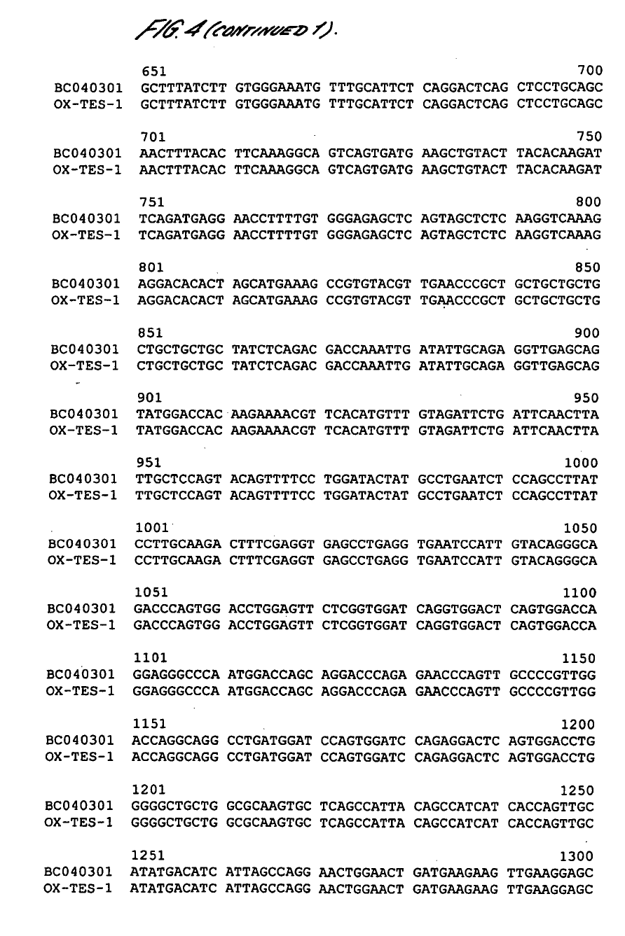

FIG. 7 illustrates the results obtained from hybridising the OX-TES-1 cDNA to BD Biosciences' Matched Tumour/Normal expression array. The identity and position of tissues on the array is shown in the lower panel. K*=kidney. Row P is the human cancer cell lines: 1 HeLa, 2 Daudi (Burkitt's lymphoma), 3 K562 (chromic myeloid leukaemia), 4 HL-60 (promyelocytic leukaemia), 5 G361 (melanoma), 6 A549 (lung carcinoma), 7 MOLT-4 (lymphoblastic leukaemia), 8 SW480 (colorectal adenocarcinoma), and 9 Raji (Burkitt's lymphoma).

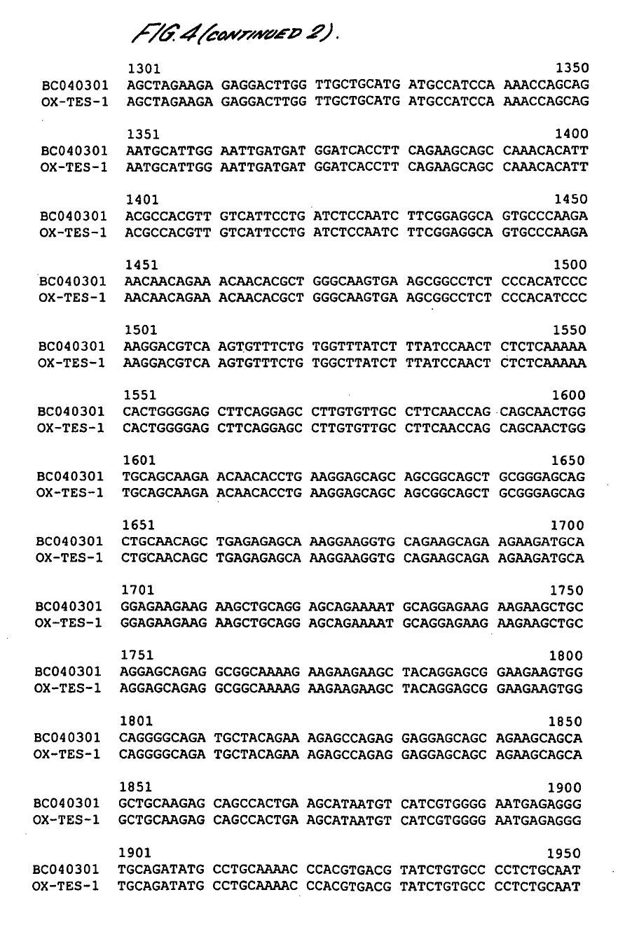

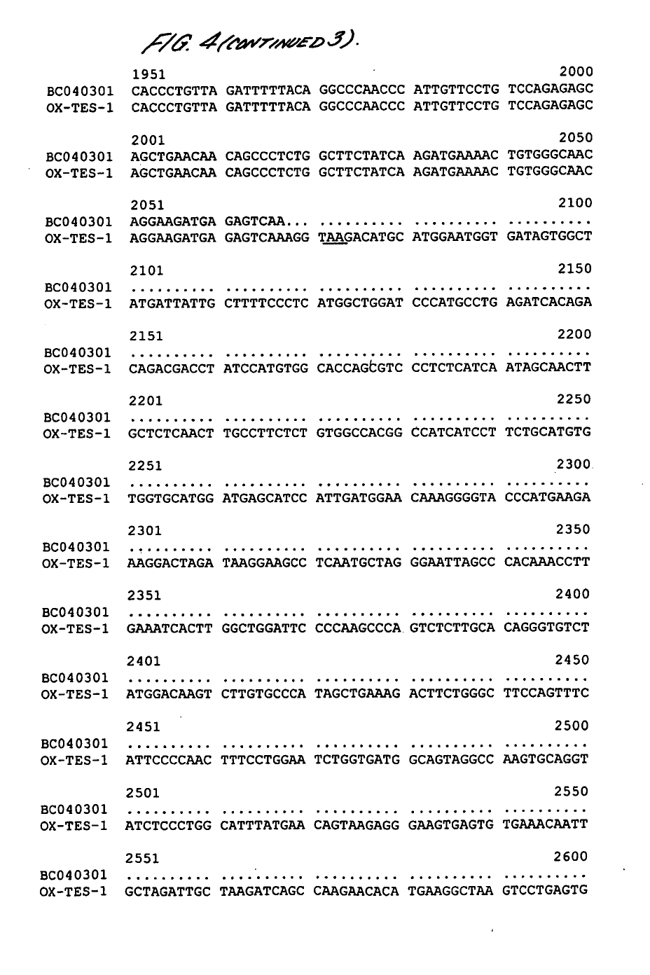

FIG. 8 shows the products amplified by RT-PCR on DLBCL cell lines for OX-TES-1. A: Primary PCR with primer set A (detects both transcripts); B: Secondary PCR with primer set A; C: Primary PCR with primer set B (OX-TES-1 specific); D: Secondary PCR with primer set B-dT primed cDNA; E: Secondary PCR with primer set B-hexamer primed cDNA; F: Primary PCR with primer set C (flanking primers); G: Secondary PCR with primer set C; H: b-actin control.

FIG. 9 is a nucleic acid molecule encoding a novel DLBCL tumour antigen designated herein as BAC1 (OX-TES-2).

FIG. 10 is an amino acid sequence of the longest polypeptide encoded by the nucleic acid molecule of FIG. 9.

FIG. 11 is a nucleic acid molecule encoding a novel DLBCL tumour antigen described as ‘similar to recombining binding protein suppressor of hairless (Drosophila). The cDNA sequence was not a perfect match for anything in the database but may represent a variant of the RBP-J Kappa gene. The closest matches were to BC 020780, an uncharacterised gene and H. sapiens recombination signal binding protein (LO78721). The cDNA sequence in BG 614689 matched some of the 5′ end of the present (TA195) sequence that differed from the BC 020780 sequence suggesting that there are a number of 5′ variants of the RBP-J Kappa DNA. All the genes map to chromosome 4 (close NT_{—}006324.7).

FIG. 12 is an amino acid sequence of the polypeptide encoded by the nucleic acid molecule of FIG. 11. FIG. 6C is an amino acid sequence of the polypeptide encoded by the nucleic acid of an EST sequence with the accession number BC020780.

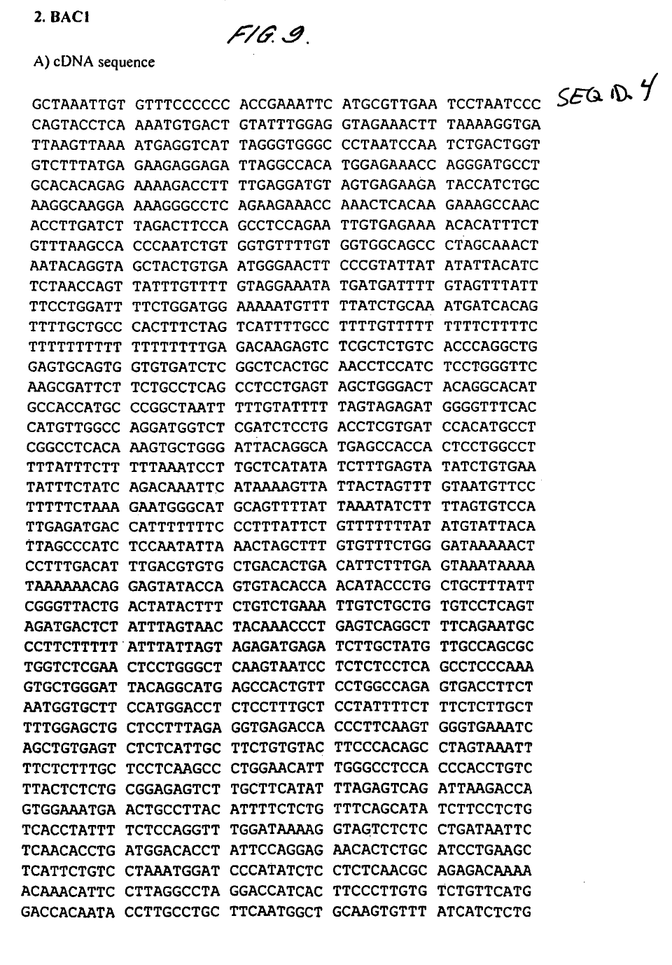

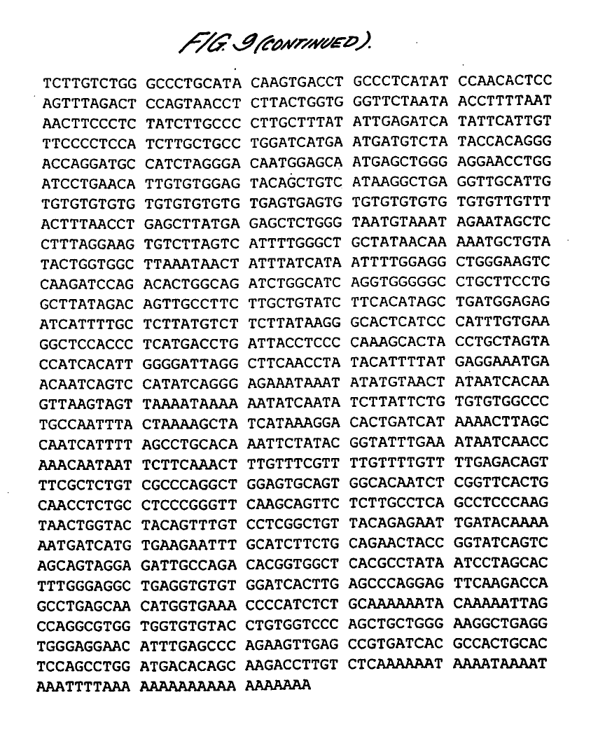

FIG. 13 is a nucleotide sequence encoding a lymphoma-associated antigen encoded by the cDNA sequence identified as Histone Macro H2A1. The first 86 nucleotides in this cDNA do not correspond to that of publicly disclosed sequences and this clone therefore has a longer 5′ untranslated region than other cDNA clones encoding this molecule.

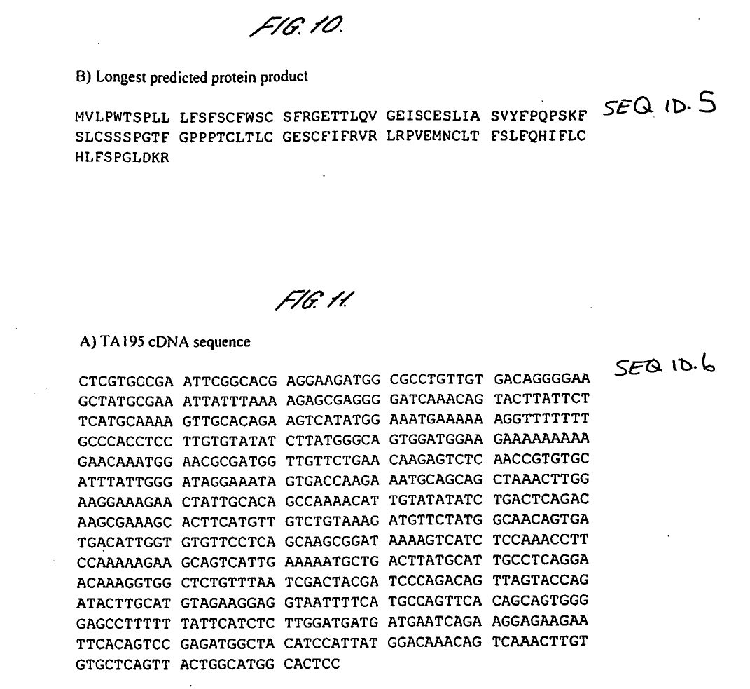

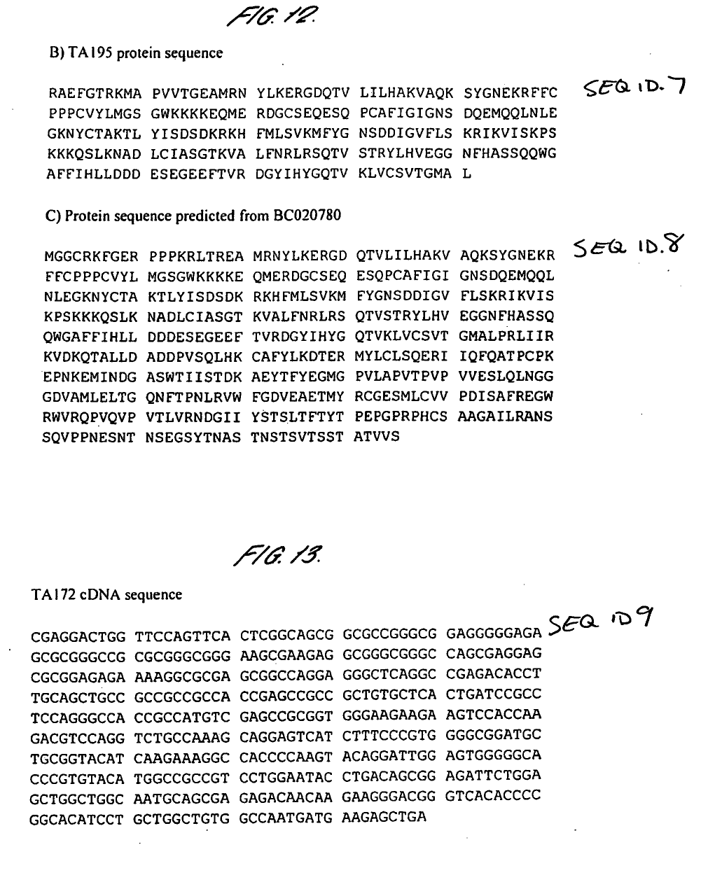

FIG. 14 is an amino acid sequence encoded by a cDNA clone designated TA204 which is most similar to a hypothetical gene (XM_{—}016176) both of which are highly related to RanBP2. Both the present sequence and XM_{—}016176 sequence map (and are identical to) the human genome sequence on chromosome 2 (NT_{—}029235). Human RanBP2 also maps to chromosome 2 but is encoded by the sequences in NT_{—}005224 and NT_{—}029895. RanBP2 and the protein produced from TA204 are somewhat different and represent highly-related proteins raising the probability that this protein and/or RanBP2 may be recognised by the serum from patients with DLBCL.

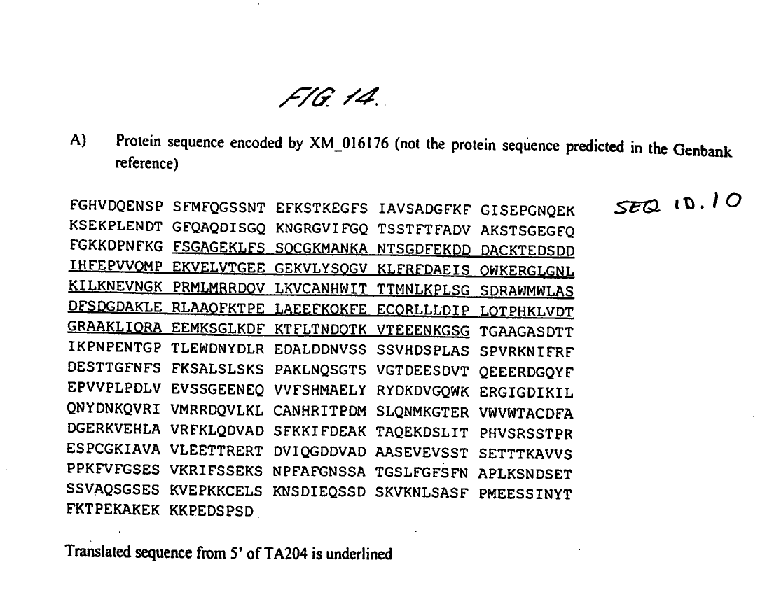

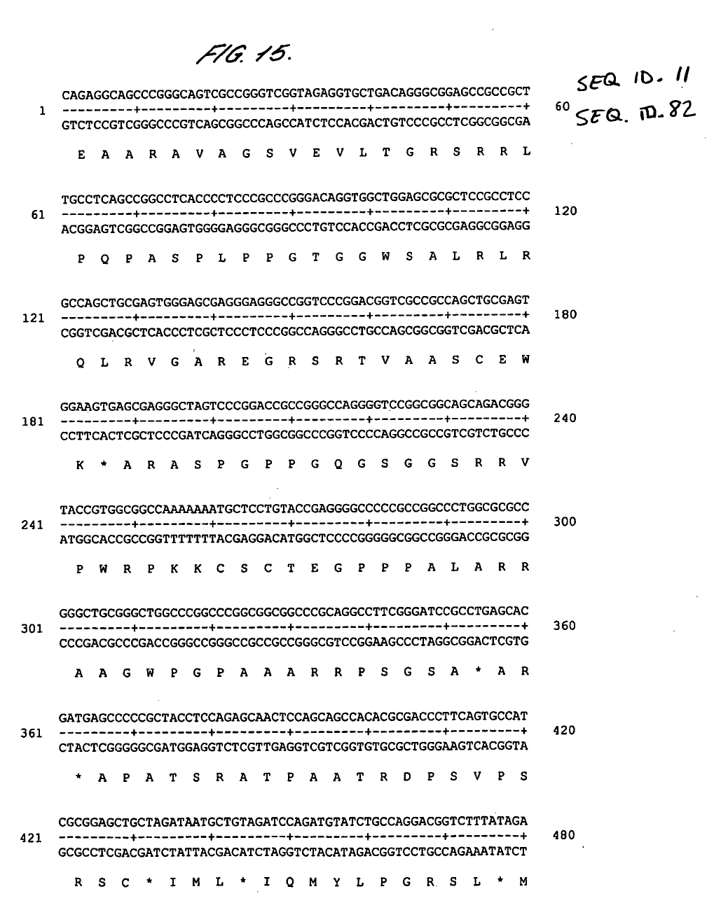

FIG. 15 illustrates the cDNA sequence of OX-TES-4 and the predicted encoded protein.

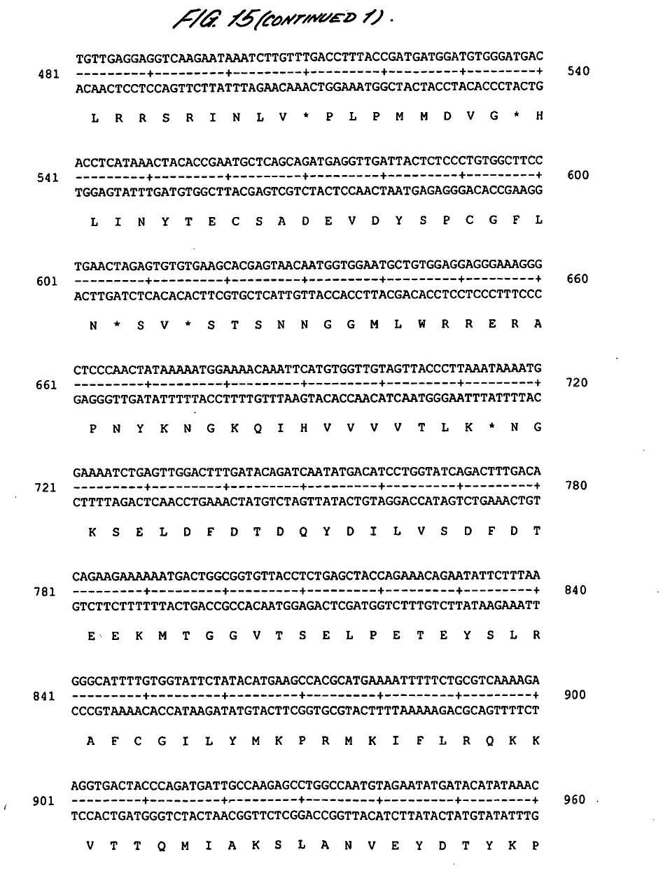

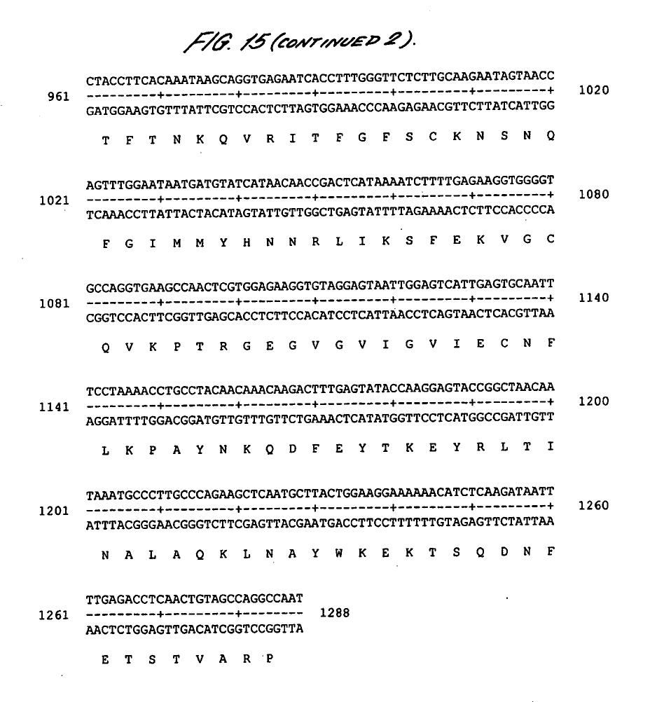

FIG. 16 shows the cDNA sequence of (OX-TES-4)FLJ31673 and the predicted encoded protein. Domains present with the protein sequence are annotated.

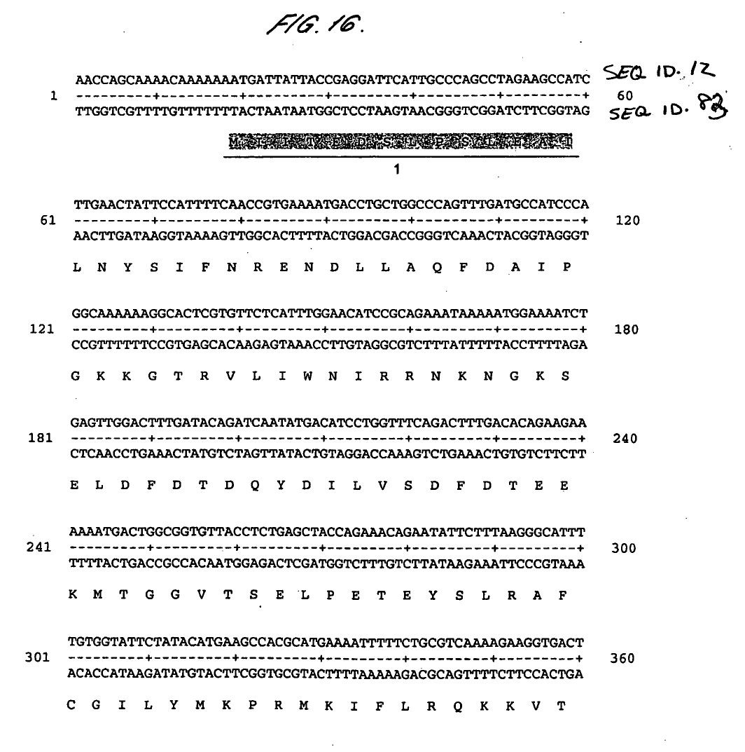

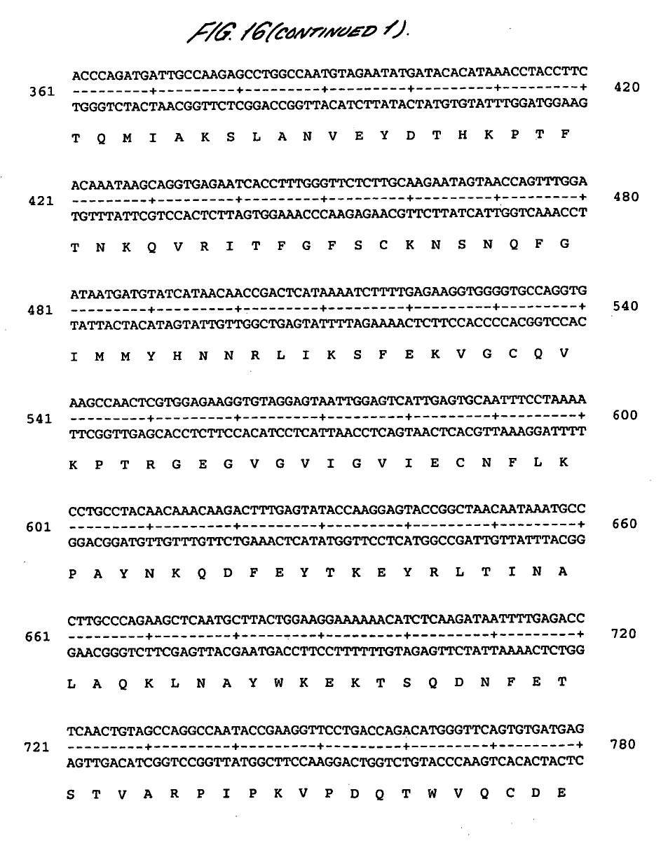

FIG. 17 is an alignment of the cDNA sequences of OX-TES-4 and FLJ31673. Similarities at the nucleotide level are shaded.

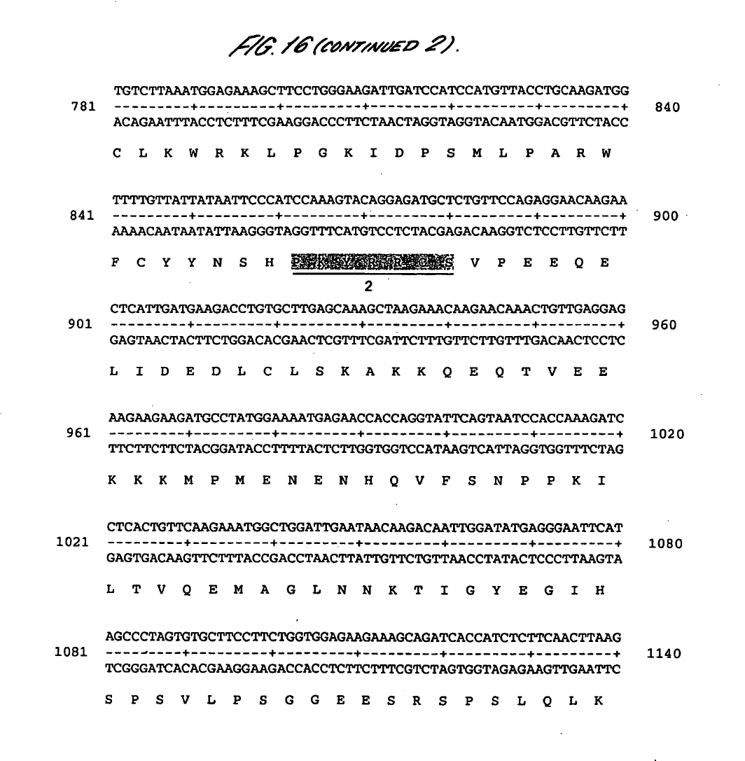

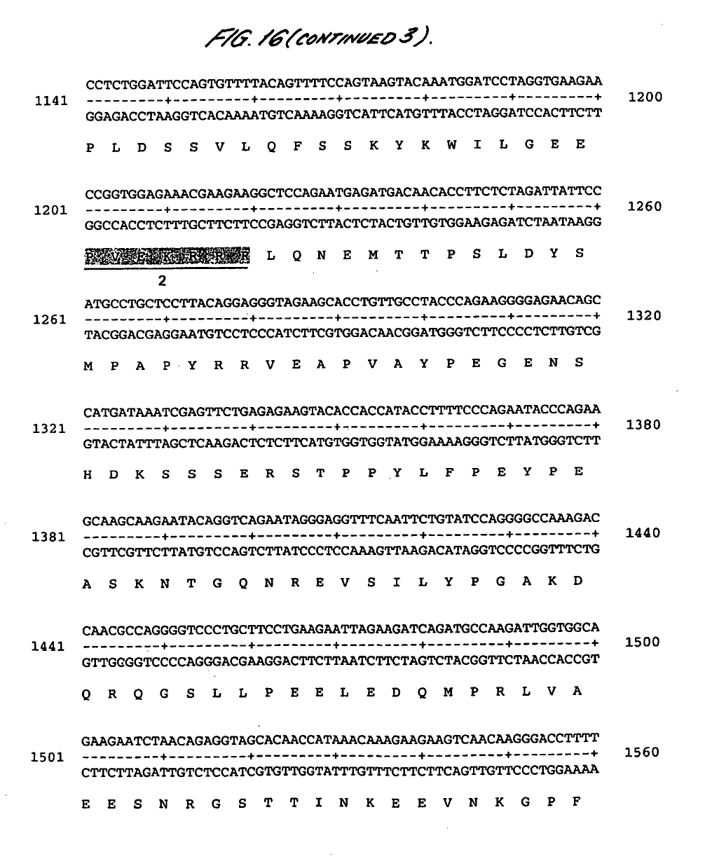

FIG. 18 is an alignment of the predicted protein sequences of OX-TES-4 and FLJ31673.

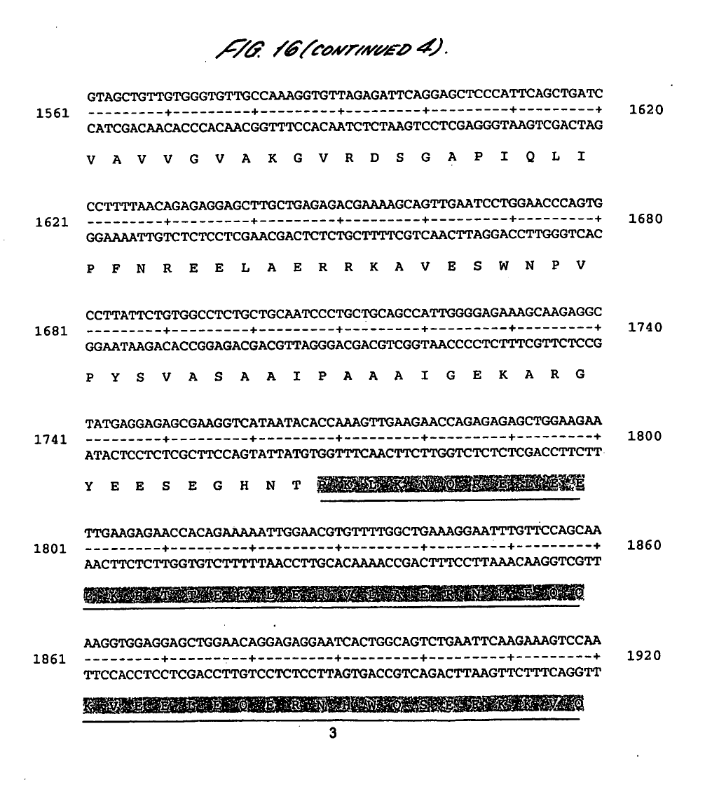

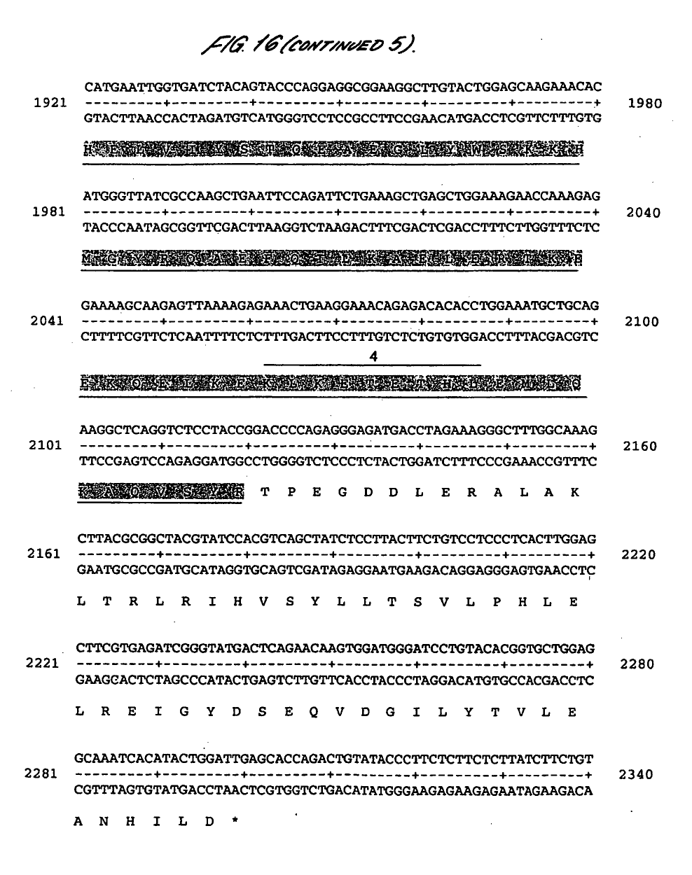

FIG. 19 illustrates the results obtained from hybridising the OX-TES-4 cDNA to BD Biosciences' Multiple Tissue Expression Array. The identities and positions of tissues on the array are shown in the lower panel.

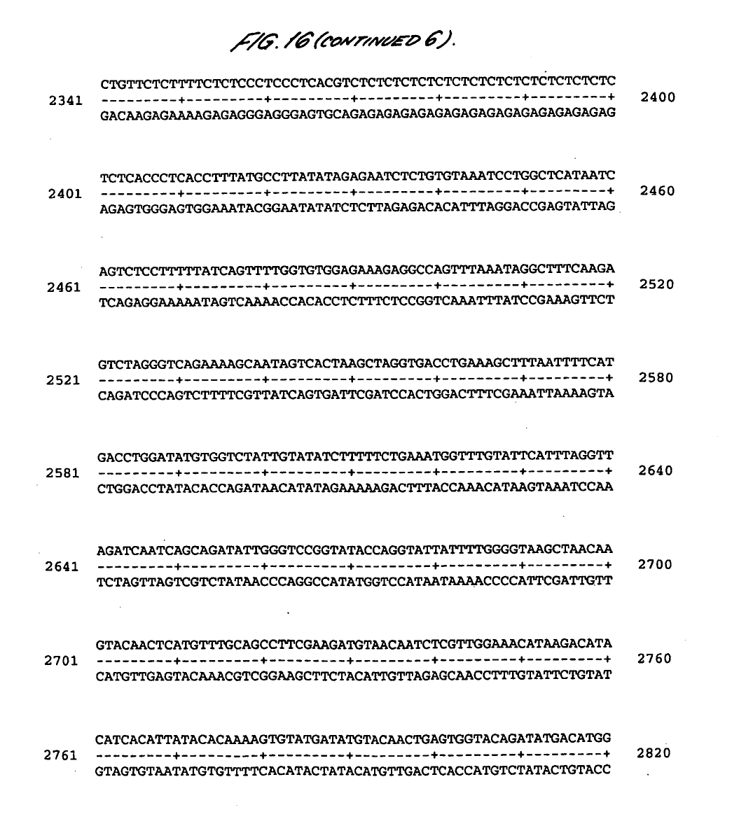

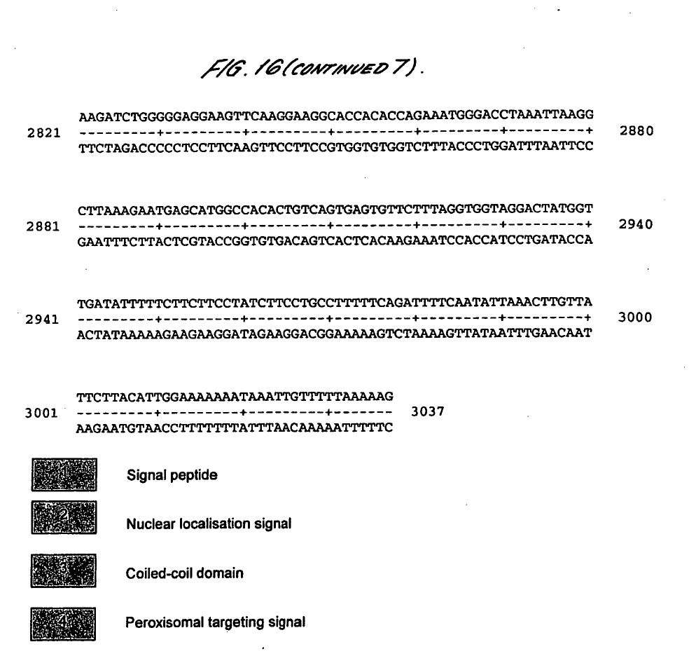

FIG. 20 illustrates the results obtained from hybridising the OX-TES-4 cDNA to BD Biosciences' Matched Tumour/Normal expression array. The identities and positions of tissues on the array are again shown in the lower panel. K*=kidney. Row P is the human cancer cell lines: 1 HeLa, 2 Daudi (Burkitt's lymphoma), 3 K562 (chromic myeloid leukaemia), 4 HL-60 (promyelocytic leukaemia), 5 G361 (melanoma), 6 A549 (lung carcinoma), 7 MOLT-4 (lymphoblastic leukaemia), 8 SW480 (colorectal adenocarcinoma), and 9 Raji (Burkitt's lymphoma).

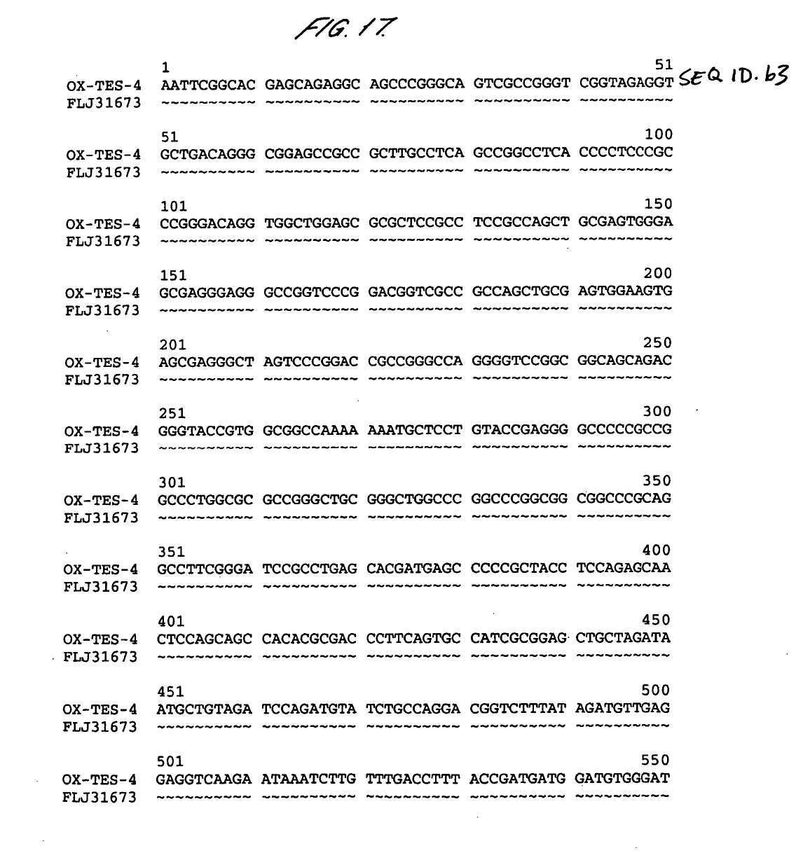

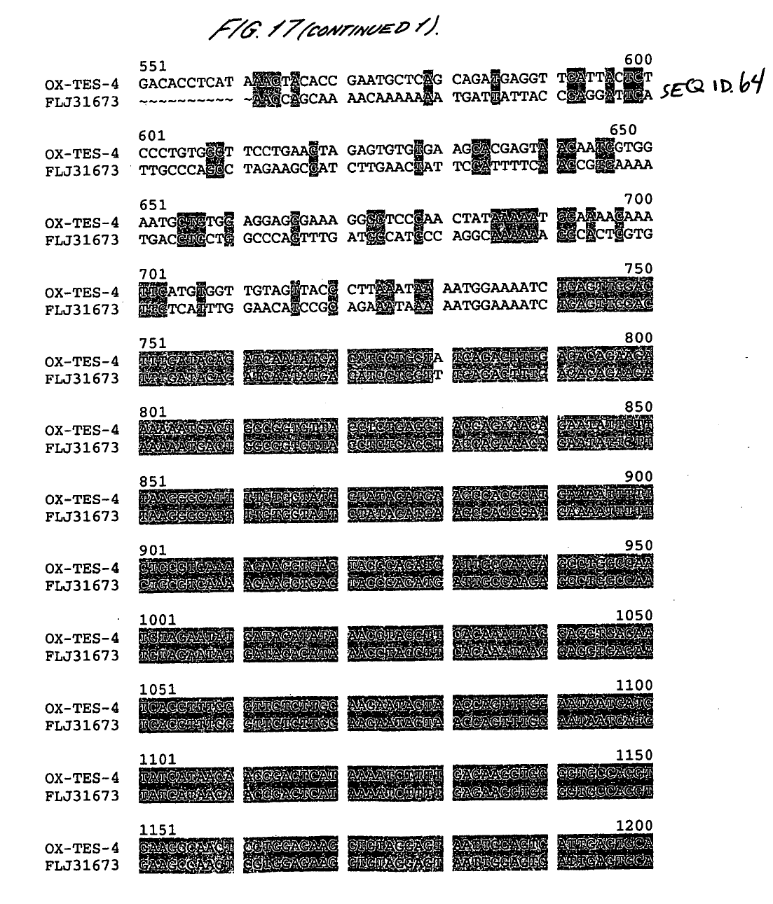

FIG. 21a illustrates the results of RT-PCR on DLBCL cell lines for OX-TES 4. A:Primary PCR with OX-TES-4 primers; B: Secondary PCR with OX-TES-4 primers; C: B actin control.

FIG. 21b illustrates the results of RT-PCR analysis of OX_TE_{—}4 expression in germinal centre, activated and subtype unknown DLBCL cell lines.

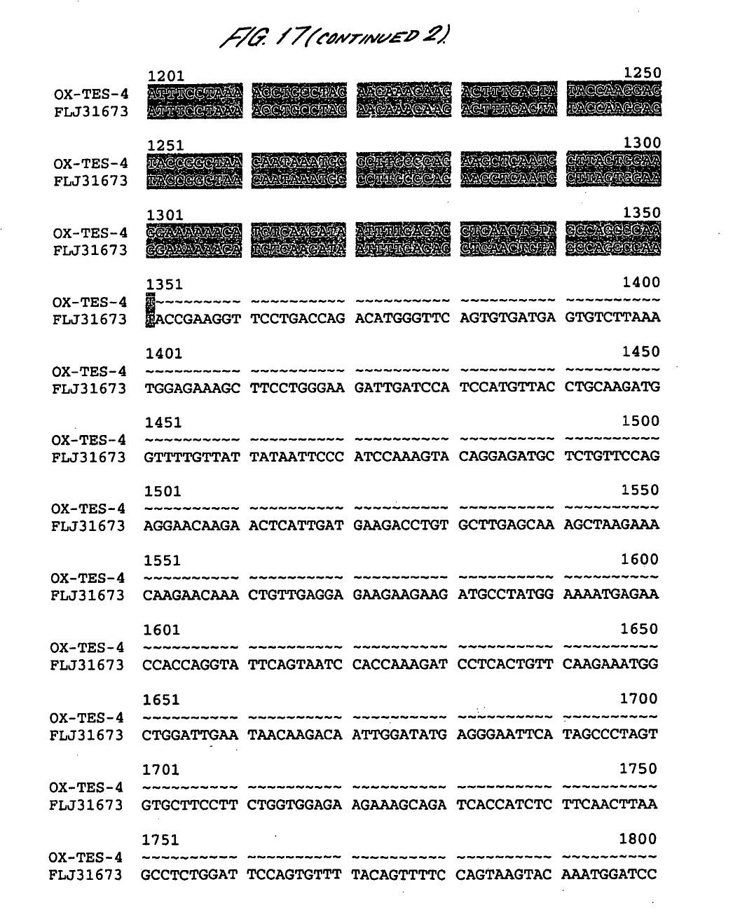

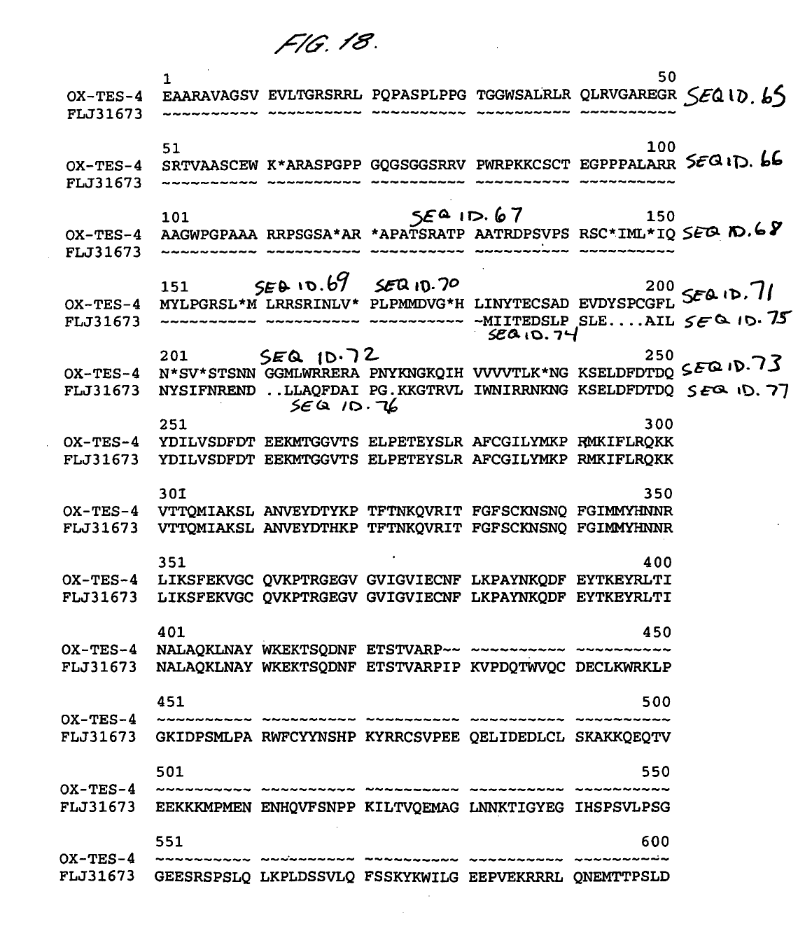

FIG. 22 is a cDNA sequence encoding a protein PPM1A. This cDNA clone is a 5′ splice variant of the other PPM1A cDNA clones in the public databases. The first 278 bp do not match the PPM1A sequences although the PPM1A sequences do have an additional 337 bp before the homologous region. Searches against the human genome sequence confirm that this 5′ sequence comes from an adjacent region on chromosome 14, and that nt 279 is the beginning of the 4^{th} exon. These changes are not predicted to change the protein sequence but may affect gene regulation.

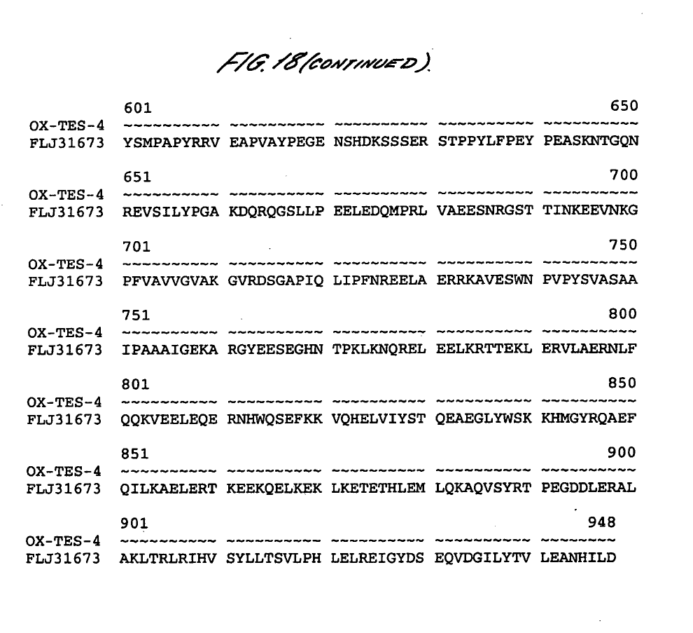

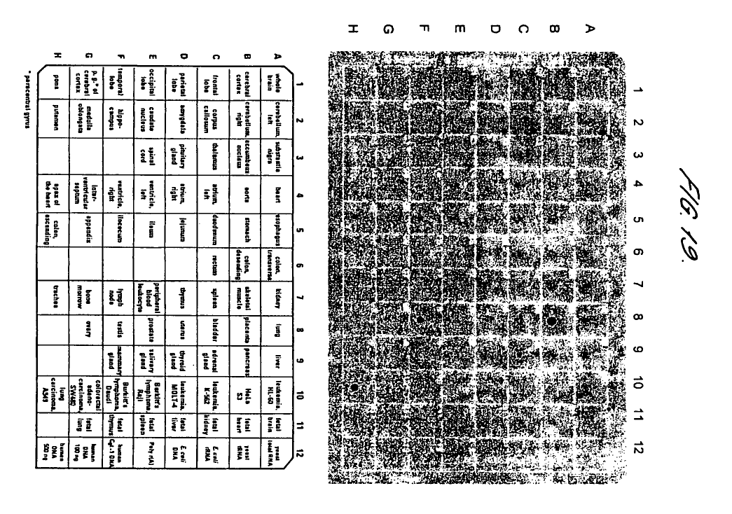

FIG. 23 is an amino acid sequence encoded by the cDNA sequence of FIG. 22.

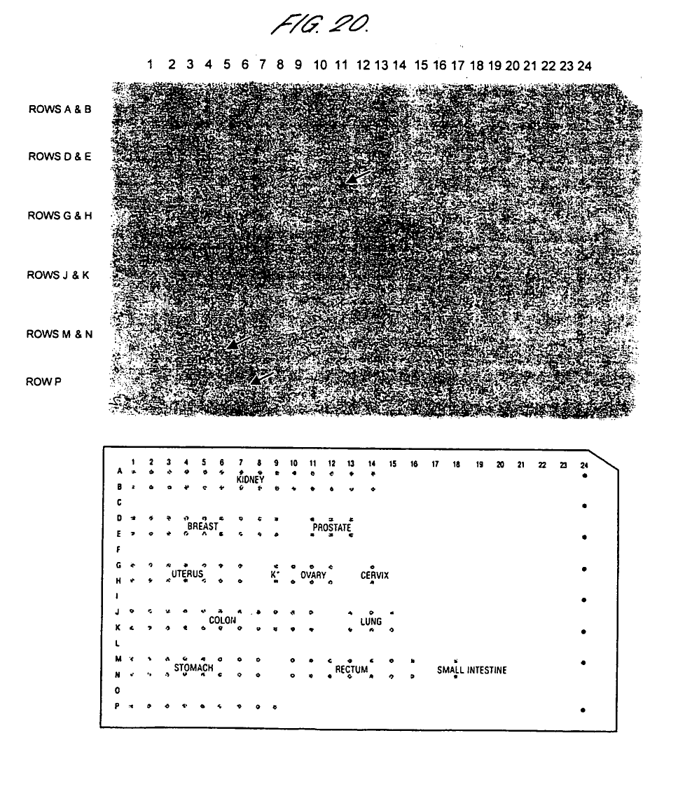

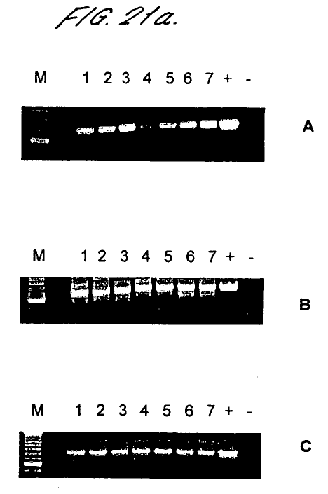

FIG. 24 is an illustration of the results of RT-PCR on DLBCL cell lines for a selection of antigens. Primary PCR results are shown in the left hand column and secondary PCR results (where appropriate) are shown in the right hand column.

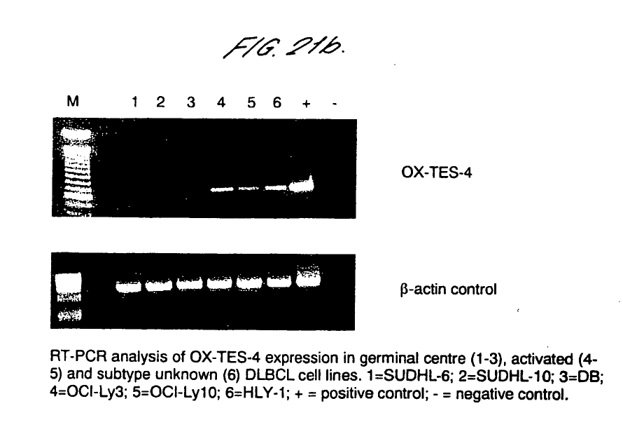

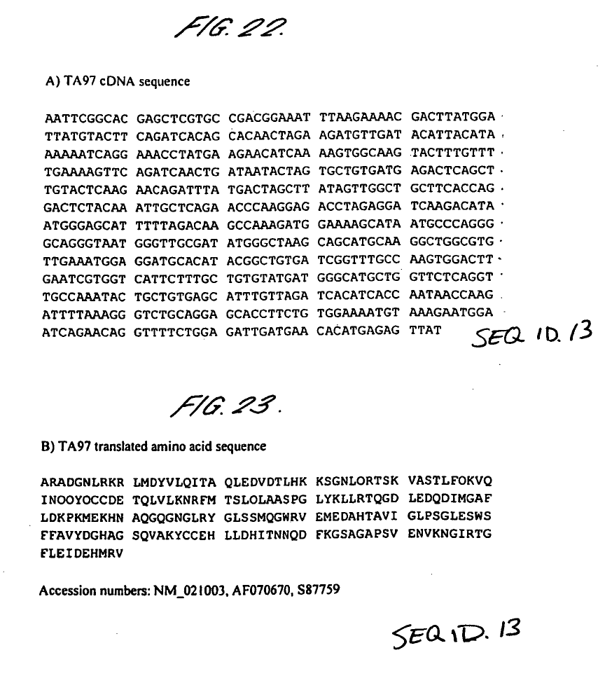

FIG. 25 is an illustration of the results obtained from immunostaining of DLBCL cell lines with the DLBCL patients serum used to screen the testis cDNA library.

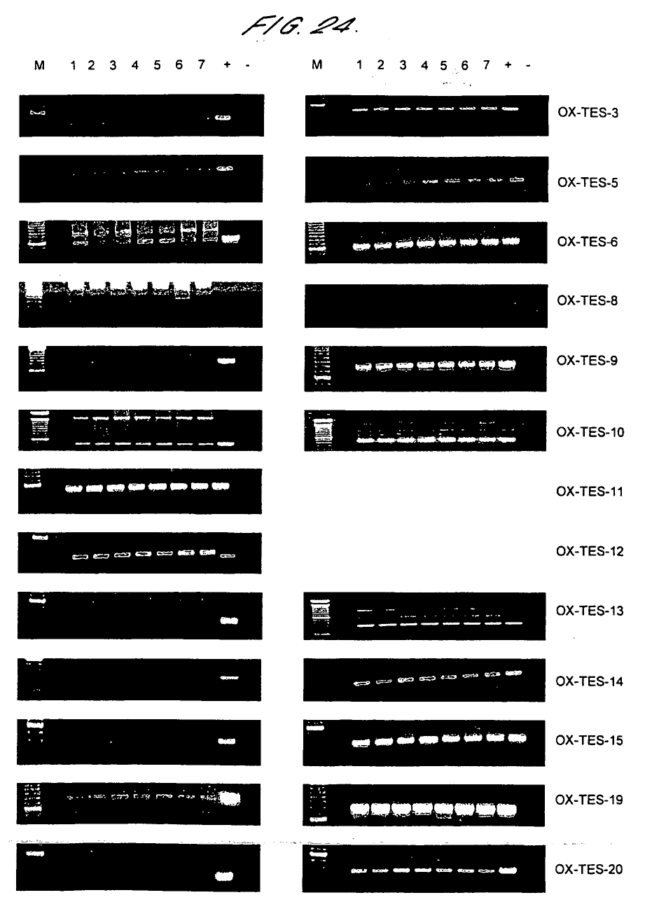

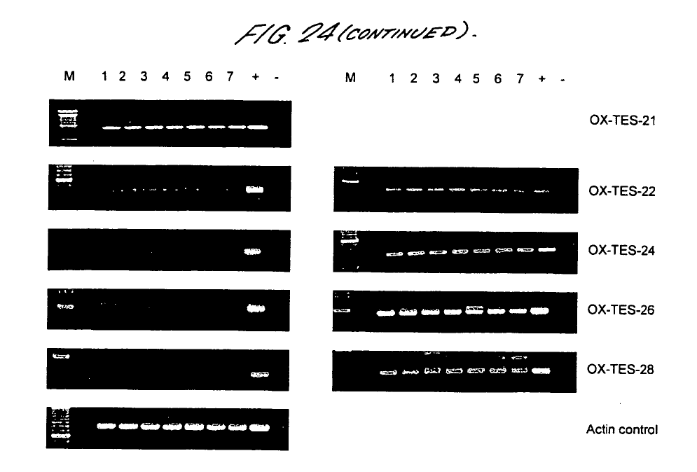

FIG. 26 is an illustration of the results obtained from immunostaining of DLBCL cell lines with serum from a DLBCL patient that was subsequently used in tertiary screening of the DLBCL tests antigens.

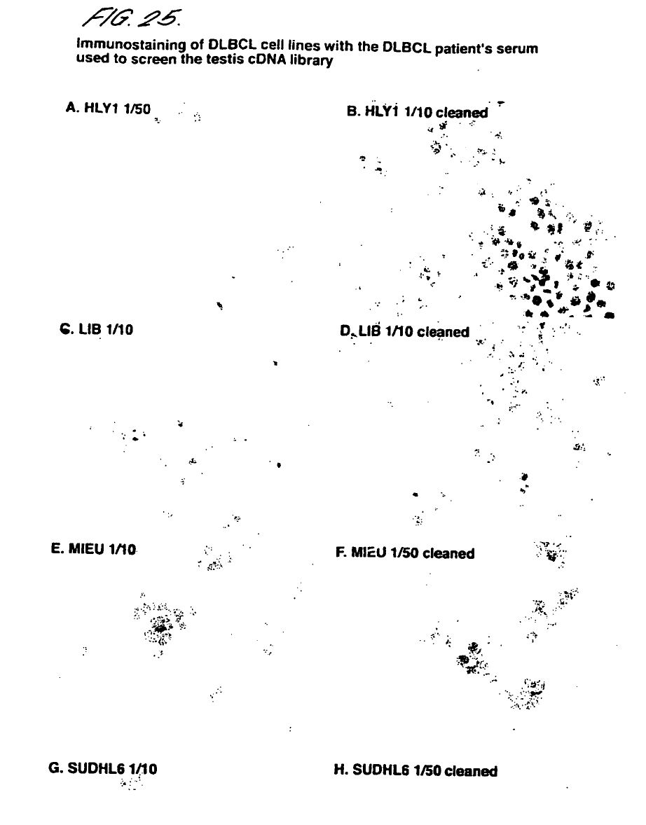

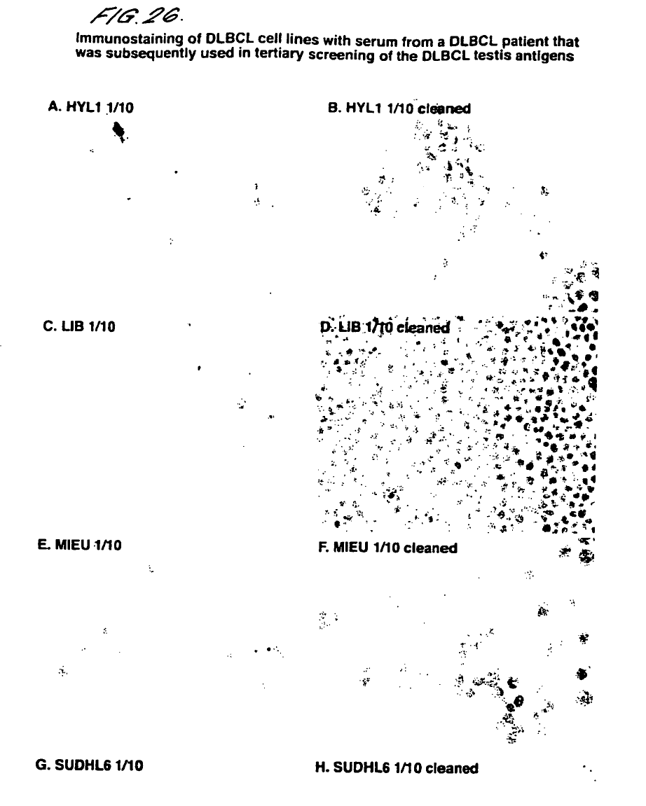

FIG. 27 is an illustration of the results obtained from immunostaining lytic membranes from the secondary screening of cDNA clones to identify DLBCL-associated lymphoma tumour antigens. The strongly stained plagues indicate a positive reaction between the patient's serum and a protein expressed from a testis cDNA clone. This can be easily distinguished from the weak background reactivity seen with the surrounding negative plagues.

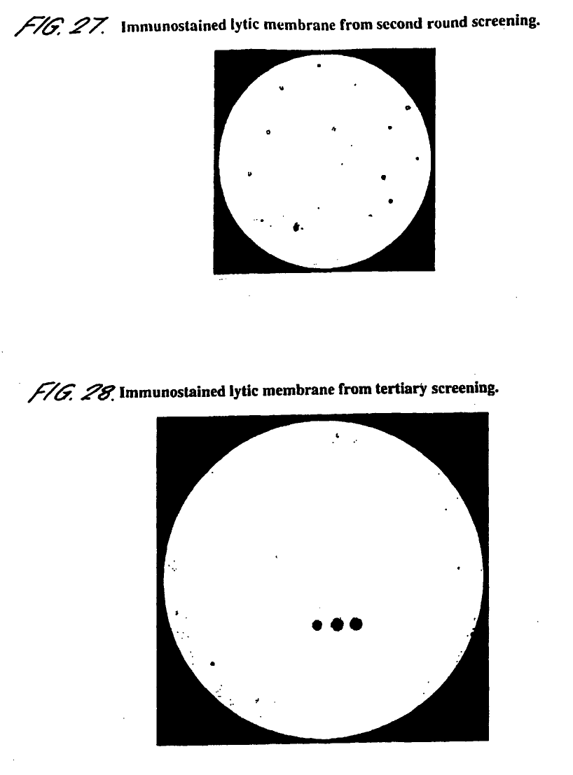

FIG. 28 illustrates an immunostained lytic membrane from the tertiary screening used to assess the reactivity of positive clones with serum from a second patient with DLBCL. Each plaque of interest was spotted three times in a row with two control spots from a phage clone without a cDNA insert spotted both above and below the clone of interst to form a rosette phage. A positive reaction is indicated when the triple spots in the centre have a stronger positivity than the flanking phage clones.

### EXPERIMENTAL APPROACH

The inventors have screened a testis cDNA expression library with serum from a single 40 year old female patient with DLBCL. Immunocytochemical screening of a number of DLBCL patients' sera on DLBCL cell lines was used prior to starting the technique to identify patients' sera that contained high titre IgG antibodies. The reasons for screening a testis library are that testis has a uniquely broad transcriptional repertoire, and there would therefore be the opportunity to identify cancer testis antigens which, as mentioned above, are particularly good candidates for immunotherapy applications. The DLBCL patient was seen at the John Radcliffe Hospital in November 2000 with stage IIA progressive disease, despite PMitcebo chemotherapy. Salvage chemotherapy was used to treat progressive disease and she had palliative chemotherapy until death in November 2001. This patient was young to have this disease and had a very aggressive form of DLBCL that was never in remission. The serum was taken post-treatment and therefore the expression of antigens identified in this study may be related to the failure of this treatment.

SEREX Protocol

Cleaning Human Serum

These steps are required before using patients' sera to screen the expression library as it is essential to remove background serum reactivity with either the E. coli or phage proteins.

A. Lytic Column

1. Grow E. coli XL1 Blue MRF′ cells in 50 ml LB (supplemented with 0.2% maltose and 10 mM MgSO₄) overnight at 37° C. and 225 rpm.
2. Pellet cells in a 50 ml Falcon tube by centrifuging at 3000 rpm for 10 minutes. Resuspend the pellet in 2 ml LB containing 10 mM MgSO_{4.}
3. Introduce 200 μl of cells (store remainder at 4° C.) into a 50 ml Falcon and add 5 ml LB, 50 μl 20% maltose, 50 μl 1M MgSO₄ and 7.5 μl tetracycline (12.5 μg/ml)
4. Inoculate with the volume of one eluted blue phage (spin phage solution for 1 minute at maximum speed and take supernatant, leaving chloroform behind).
5. Incubate culture at 37° C. and 250 rpm for 4 hours.
6. To remainder of culture from step 3, add 5 ml LB, 50 μl 20% maltose, 50 μl M MgSO₄ and 7.5 μl tetracycline (12.5 μg/ml). Mix this with the phage-inoculated culture from step 5.
7. Incubate culture at 37° C. and 250 rpm for 2 hours.
8. Freeze-thaw the culture three times, then sonicate the culture for eight 5-second pulses.
9. Resuspend 1 g CNBr-activated sepharose in 10 ml 1 mM HCl. Wash beads with 200 ml 1 mM HCl over a sintered glass filter to remove any additives.
10. Resuspend the beads in 7 ml (final volume) of 1 mM HCl.
11. Add 5 ml coupling buffer to the lysed bacteria from step 8. Add 4 ml (out of 7 ml final volume) washed sepharose beads to the lysate. Seal neck of falcon with parafilm and rotate mixture overnight at 4° C. to couple bacterial/phage proteins to the sepharose beads.
12. Pellet the matrix (sepharose beads+proteins) by centrifuging at 3000 rpm for 10 minutes and pour the supernatant into Virkon. Save the matrix.
13. Wash the matrix in 30 ml coupling buffer by resuspending matrix in buffer, centrifuging at 3000 rpm for 10 minutes, and discarding supernatant into Virkon.
14. Block any remaining active groups by resuspending matrix in 30 ml 0.1M Tris-HCl pH 8.0 and leaving to stand at room temperature for two hours.
15. Pellet matrix as above and discard supernatant.
16. Wash matrix in 30 ml Wash Buffer 1 followed by 30 ml Wash Buffer 2.
17. Repeat step 16 twice more.
18. Wash the matrix twice in 50 ml 1× TBS/0.1% sodium azide. Matrix can be stored in 1× TBS/0.1% sodium azide at 4° C. until required.
19. If using straight away, resuspend the matrix in a volume of 1× TBS/0.1% sodium azide that will yield a 1:10 dilution of serum (e.g. 18 ml 1× TBS/0.1% sodium azide+2 ml serum).
20. Add the serum to the resuspended matrix.
21. Seal the neck of the tube with parafilm and rotate overnight at 4° C.
22. Pellet the matrix by centrifuging at 3000 rpm for 10 minutes. Save the supernatant=serum. Discard the matrix. If not proceeding directly to mechanical column method (B), store the serum at 4° C. with the neck of the tube sealed with parafilm.
   B. Mechanical Column.
1. Grow E. coli XL1 Blue MRF′ cells in 50 ml LB (supplemented with 0.2% maltose and 10 mM MgSO₄) overnight at 37° C. and 225 rpm.
2. Pellet cells in a 50 ml Falcon tube by centrifuging at 3000 rpm for 10 minutes.
3. Resuspend the pellet in 5 ml PBS.
4. Freeze-thaw the cells three times, then sonicate the culture for eight 5-second pulses.
5. Repeat steps 9-18 for lytic column method.
6. Do not resuspend the matrix in 1× TBS/0.1% sodium azide. Instead, add the serum (already at 1:10 from lytic column method) directly to the matrix.
7. Seal the neck of the tube with parafilm and rotate overnight at 4° C.
8. Pellet the matrix by centrifuging at 3000 rpm for 10 minutes. Save the supernatant=serum. Discard the matrix. If not proceeding directly to lytic membrane method (C), store the serum at 4° C. with the neck of the tube sealed with parafilm.
   C. Lytic Membrane.
1. Prepare plates (1 per serum sample to be cleaned):
   600 μl E. coli XL1Blue MRF′ cells (OD₆₀₀=0.5)
   250 μl eluted blue phage
   Incubate at 37° C. for 15 minutes to allow phage to infect cells
   Add 10 ml NZY top agar
      40 μl 0.5M IPTG
      50 μl 250 mg/ml X-gal (only necessary if you want to see blue color)
2. Plate onto dried NZY agar plates (140 mm) and leave to set.
3. Incubate plate(s), inverted, overnight at 37° C.
4. Next morning, place a nitrocellulose membrane onto the plate and perform a plaque lift at 37° C. for 4 hours.
5. Prepare block solution (see step 8) without tween-20 in a sterile container. Use 20 ml per membrane. Boil ˜50 ml dH2O in a 500 ml bottle to sterilise it, then microwave the appropriate volume of 5% marvel in TBS to ensure sterilisation.
6. Remove membrane carefully, using forceps, and place into TBS. Gently brush off any excess agar that is stuck to the membrane.
7. Wash membrane twice in TBS-T for 5 minutes followed by once in TBS for 5 minutes.
8. Block membrane for 1 hour at room temperature in 20 ml 5% marvel in TBS-T.
9. Wash membrane four times in TBS-T then once in TBS for 5 minutes each wash. Following the third wash, change the petri dish.
10. Pour the total volume of serum from step B8 (Mechanical column) onto the membrane and incubate overnight at room temperature on shaker.
11. Remove serum, using a 10 ml pipette, into a sterile 50 ml Falcon. Store at 4° C. with the neck of the tube sealed with parafilm.
12. Repeat this process twice more. Serum should now be clean. Store in 2 ml aliquots at 4° C. for 6 months or −80° C. long term.
   D. Primary Screening
1. Inoculate 50 ml LB (supplemented with 0.2% maltose and 10 mM MgSO₄) with an E. coli XL1Blue MRF′ colony from a freshly streaked plate and grow overnight at 30° C. and 225 rpm.
2. Pellet cells in 50 ml Falcon tube by centrifuging at 3000 rpm for 10 minutes and resuspend in 25 ml 10 mM MgSO₄. Measure OD₆₀₀ and dilute some of the cells to OD₆₀₀=0.5 in MgSO₄.
3. Dry large NZY agar plates at 37° C. for approximately 1 hour, and prepare NZY top agar.
4. Into a 15 ml Falcon tube introduce 600 μl MRF′ cells (OD₆₀₀=0.5) and the appropriate volume of phage in SM buffer. The usual density for library screening is 50000 pfu/140 mm plate but the inventors tend to use considerably lower densities. Mix phage and cells by inverting 2-3 times then incubate at 37° C. for 15 minutes.
5. Quickly add 10 ml NZY top agar and 40 μl IPTG (0.5M) to the phage/cells and pour onto the NZY agar plate. Leave to set at room temperature for 10 minutes and then incubate plates, inverted, overnight at 37° C.
6. Perform a 4-hour plaque lift onto nitrocellulose membranes at 37° C. Write the date and membrane number on the membrane in biro.
7. Prepare block solution. Leave to cool to room temperature; add Tween-20 to a final concentration of 0.05% just prior to use.
8. Pierce the membrane and agar with a sterile needle to orientate plaques at a later stage and remove membrane, using forceps, into TBS. Brush off any excess agar gently and place membranes, protein side down, into clean petri dishes containing TBS-T. Store plates at 4° C.
9. Wash membrane twice in TBS-T for 5 minutes followed by once in TBS for 5 minutes.
10. Remove TBS and add 20 ml block solution onto membrane using a 25 ml pipette to ensure full coverage. Incubate for 1 hour at room temperature on the shaker.
11. Remove the block solution and wash the membranes four times for 5 minutes in TBS-T, changing the petri dish after the third wash. Finally, wash once with TBS for 5 minutes.
12. Remove TBS and add 20 ml serum to each membrane using a 25 ml pipette as before. Incubate at room temperature on the shaker overnight.
13. Remove membranes from serum and place into clean petri dishes containing TBS-T. Pipette the serum into labelled Falcon tubes and store at 4° C. with necks sealed with parafilm.
14. Wash membranes as in 11.
15. Remove TBS and add 20 ml antibody solution (Rabbit anti-human IgG, Fc fragment specific, alkaline phosphatase conjugated; 1:5000 dilution in 0.5% marvel in TBS-T). Incubate at room temperature on shaker for 1 hour.
16. Remove antibody solution and wash membranes as in 11.
17. Incubate membranes in colour development reagents.
   E. Colour Development
1. Introduce 20 ml AP Colour Development Reagent buffer into a clean 140 mm petri dish. Add 100 μl BCIP (30 mg/ml) and 100 μl NBT (60 mg/ml). Mix thoroughly and cover with a light-protective box (e.g.cardboard).
2. Remove membrane from TBS using forceps, drain excess TBS off onto tissue and place membrane, protein side up, into development solution.
3. Allow colour to develop for up to 30 minutes, keeping reagents in the dark. Strongly positive plaques will appear within 5 minutes, and background will appear within 10 minutes.
4. At end of development reaction, place membranes into dH₂O to stop the reaction.
5. Check membranes when wet, and circle any positive plaques with biro. Dry membranes on filter paper and check again for any positive plaques.
   F. Positive Plaques
1. Put the plate with positive plaques on, upside down, next to the membrane with the positives spots on. Use the needle marks to orientate the plate and membrane.
2. Using a pair of compasses, locate the positive phage from all three needle holes, and number it with a pen. If there is more than one positive plaque, mark the others in the same way.
3. Turn the plate the right way up and place it on a light box. Using a sterile scalpel, cut out the positive plaque and its surrounding 3-4 negative neighbours as one piece, and place into a 1.5 ml eppendorf tube (labelled with the corresponding plaque number) containing 500 μl of SM buffer. Repeat for any other plaques.
4. Incubate tubes on an end-over-end rotator overnight at 4° C. to allow phage to elute into SM buffer.
5. Next morning, add 20 μl chloroform, vortex, centrifuge at 14000 rpm for 1 minute and store at 4° C. until ready to do secondary screening.
   G. Secondary Screening
1. Dry small NZY agar plates (90 mm) at 37° C. for approximately 1 hour, and prepare NZY top agar.
2. Dilute the phage from the primary screen 1:10 in SM buffer.
3. Into a 15 ml Falcon tube, introduce 200 μl of E. coli XL1Blue MRF′ cells (OD₆₀₀=0.5) and 4 μl of diluted phage from step 2. Mix phage and cells by inverting 2-3 times then incubate at 37° C. for 15 minutes.
4. Quickly add 3 ml NZY top agar and 18 μl IPTG (0.5M) to the phage/cells and pour onto the NZY agar plate. Leave to set at room temperature for 10 minutes and then incubate plates, inverted, overnight at 37° C.
5. Perform a 4-hour plaque lift onto nitrocellulose membranes at 37° C.
6. Perform screening as for primary screen with the following exceptions:
   use 10 ml block solution, antibody solution, and serum per membrane.
7. Identify and isolate positive plaques except that in this second round of screening, the positive phage should be isolated alone (with no negatives).
   H. Tertiary Screening
1. Dry large NZY agar plates and prepare NZY top agar.
2. Into a 15 ml Falcon tube introduce 600 μl XL1Blue MRF′ cells (OD₆₀₀=0.5). Quickly add 10 ml NZY top agar and 40 μl IPTG (0.5M) and pour onto NZY agar plates. Leave to set at room temperature for 15 minutes.
3. Spot 1 μl of positive phage onto plate following a template and use 0.8 μl of blue phage as negative control (see FIGS. 1 & 2 for examples of templates).
4. Once the phage has dried into the agar, incubate plates, inverted, overnight at 37° C.
5. Perform a 4-hour plaque lift onto nitrocellulose membranes at 37° C.
6. Perform tertiary screen as for primary screening.
7. Score membranes to identify which plaques show a positive reaction with which serum. This tertiary screen should be performed three times to be confident of results.
   Results

Primary screening of the testis library identified 94 clones that were confirmed as positive after second round screening. Phagemids were in vivo excised from the lambda phage clones according to the manufacturer's instructions to generate cDNA clones in the vector pBK-CMV. The 5′ end of each cDNA clone was then commercially sequenced by MWG-Biotech. In cases where the cDNA clone appeared to be novel (4 clones), the cDNA insert was fully sequenced to publication quality by MWG-Biotech.

These results of sequence analyses indicated that the 94 clones corresponded to 29 different genes, of which 2 are novel and a further 8 are uncharacterised. Two cDNA clones originally thought to encode novel genes are, in fact, longer cDNA sequences for which the additional 5′ sequence had not been deposited in the databases.

Identification of cDNA Sequences Using Publicly-Disclosed Information

DNA (and/or protein) sequences were BLAST searched through the National Center for Biotechnology Information (NCBI) website to obtain any publicly available information concerning the sequences:
1. Against the human genome to identify their chromosome localisation.
2. Against the non-redundant database to see if they corresponded to known genes.
3. Against the EST database to identify any homologous clones.

Additional sequence searches were also performed, where possible using either full length sequences from known genes or as much sequence as possible from those that were novel:
4. Against the patent database.
5. Against the Stanford Lymphochip to identify whether their expression in DLBCL had been investigated in this microarray study at http://llmpp.nih.gov/lymphoma/search.shtml.
6. Against the SEREX database to determine whether these molecules had already been identified as tumour antigens in other tumour types.
7. The TIGR Index to identify theoretical cDNA clones.
8. Unigene/Locus link and Online Mendelian inheritance in Man (OMIM) to identify additional gene information.
9. PubMed to investigate published work on known molecules.
10. Cancer Genome Anatomy Project (CGAP) recurrent chromosomal abberations site to look for disease associations with mapped loci (web site: http://www.ncbi.nlm.nih.gov/ncicgap/).

The majority of these data are summarised in Table 1.

Sequence Data Analysis of OX-TES-1 and Splice Variants

OX-TES-1 is a cDNA of 4109 bp that encodes a predicted protein of 639 amino acids (FIG. 1). Analysis of this protein using databases on the world wide web predicts a molecular weight of 72.7 kDa and a pl of 5.23. This analysis also identified a number of domains that are highlighted in FIG. 2. In addition to those shown, there is a proline-rich region between aa 478 and 639, a lysine-rich region between aa 508 and 548, and a second coiled-coil domain between aa 475 and 557. There are no known homologues of this protein—the closest related protein is the CLOCK protein of the Korean rock fish Sebastes schlegeli (32% similarity) and neuronal PAS domain protein 2 of the mouse Mus masculus (22%). The predicted domains and nuclear localisation signal sequences detected in the OX-TES-1 protein suggest that it may function as a transcription factor.

Analysis of the available human genome sequence shows that OX-TES-1 localises to chromosome X. Searching of the databases revealed a number of ESTs with high similarity to OX-TES-1 at the nucleotide level. This analysis revealed a splice variant of OX-TES-1, also cloned from a testis cDNA library (Accession number BC040301), which is 2850 bp in size. BC040301 maps to chromosome Xq28, which is a locus known to have structural aberrations in DLBCL, follicular lymphoma, and mantle cell lymphoma amongst others. The chromosomal band Xq28 has been a focus of interest in human genetics because more than 20 hereditary disease linked genes have been mapped to this region, as have a number of immunogenic tumour antigens with the characteristic cancer/testis expression pattern including, MAGE-A, NY-ESO-1, LAGE-1, TRAG-3, CSAGE and SAGE. In addition Xq28 has been identified as a genetic region that is altered in lymphomas and which may contain lymphoma associated oncogenes (reviewed in (Goyns et al., 1993; Vineis et al., 1999)); one example being the high level amplification of Xq28 identified in some blastic mantle cell lymphoma patients (Bea et al., 1999). Genetic gain within a common region at Xq28 has also been detected in testicular cancer (Skotheim et al., 2001) and two translocation breakpoints in infertile males have also been reported in this region (Olesen et al., 2001). Thus genes mapped within this region may have additional disease associations.

The difference between the sizes of the cDNAs of BC040301 and OX-TES-1 appears to be the result of intron retention, resulting in a deletion of an additional 1.1 kb of cDNA (between nucleotides 2062 and 3328) in OX-TES-1. The genomic structure of both OX-TES-1 and BC040301 is shown in FIG. 3, whilst FIG. 4 shows an alignment of the cDNA sequences of both variants. Since this additional region contains the translational stop codon in OX-TES-1 (underlined in FIG. 4), BC040301 encodes a protein that is longer than OX-TES-1; the amino acid sequence is identical to OS-TES-1 from aa 1 to 638, but there is an additional 135 amino acids at the C-terminus (shown in FIG. 5). There is only one identifiable domain in this additional region: an ER membrane retention signal.

PAS Domain Protein (OX-TES-1)

Sequence searches using BLAST indicated that the cDNA in TA112 represents a novel gene that has not been deposited in the public databases prior to the present work. Prior to this work, the closest similarity to this sequence was found to be neuronal PAS domain protein 2 and CLOCK. There were two EST sequences corresponding to incomplete regions of our sequence with accession numbers BG723594 and BI458651. Translation of the cDNA sequence in TA112 predicted a 639 amino acid (aa) protein. The poly(A) tail on the cDNA clone and the presence of upstream stop codons in the translated protein sequence indicated that the cDNA encoded a full length protein product. There are two potential methionine start codons for this protein; since the second (aa3) has a slightly better Kozak sequence than the first methionine, it may therefore represent the start of translation.

The protein sequence was analysed using MotifFinder. This programme identified an N-terminal PAS domain (using the Pfam database) between aa 32-96. PAS domains are present in many signalling proteins where they are used as signal sensor domains. Several PAS domain proteins are known to detect their signal using an associated cofactor. Analysis using the PSORT II programme identified an R-2 motif at aa 14 (which is a predicted cleavage site for mitochondrial presequence), a nuclear localisation signal at aa 538, an ER membrane retention signal in the N-terminus, a C-terminal leucine zipper pattern at aa 481-502 and a coiled-coil domain containing the leucine zipper between aa 475-556. These analyses indicate that this molecule is potentially a nuclear DNA binding protein containing a PAS domain.

PAS domains regulate the function of many intracellular signalling pathways in response to both extrinsic and intrinsic stimuli, and regulate circadian rhythmicity in diverse organisms. The PAS domain has been identified as an interface for protein-protein interactions in an evolutionarily related family comprising several hundred proteins.

The PAS domain is found in many archaeal, bacterial, and plant proteins where it is capable of sensing environmental changes in light intensity, oxygen concentration, and redox potentials. The oxygen sensor FixL from Rhizobium species contains a heme-bearing PAS domain and a histidine kinase domain that couples sensing to signalling. The 144-kDa PASKIN protein contains a PAS region similar to the FixL PAS domain and a serine/threonine kinase domain which might be involved in signalling. Thus, PASKIN is likely to function as a mammalian PAS sensor protein (Hofer et al., 2001). PAS domain-regulated histidine kinases are common in prokaryotes; in contrast these kinases are rare in eukaryotes and were, for a long time, thought to be completely absent in mammals. However PAS kinase (PASK) is an evolutionarily conserved gene product present in yeast, flies, and mammals. The amino acid sequence of PASK specifies two PAS domains followed by a canonical serine/threonine kinase domain, indicating that it might represent the first mammalian PAS-regulated protein kinase (Rutter et al., 2001).

The key elements of circadian clockwork and oxygen homeostasis are the PAS protein family members PER and CLOCK and hypoxia-inducible factor lalpha (HIF-1 alpha). Alteration of gene expression is a crucial component of adaptive responses to hypoxia and these responses are mediated by hypoxia-inducible transcription factors (HIFs). Hypoxia is also a potent inducer of tumour angiogenesis, the process of which is mostly mediated by induction of vascular endothelial growth factor (VEGF). Endothelial PAS domain protein 1 (EPAS1) is a basic helix-loop-helix (bHLH)/PAS domain transcription factor that is expressed most abundantly in highly vascularized organs. Studies have shown that endogenous VEGF can be up-regulated transcriptionally by EPAS1, and it has been proposed that EPAS1 may be involved in the angiogenesis of renal cell carcinoma (Xia et al., 2001). The effect of hypoxia on the expression of HIF-1 alpha and EPAS1 has been investigated. These two similar but distinct proteins have been postulated to activate VEGF expression in response to hypoxia. Src family kinases have also been shown to mediate the hypoxia-mediated EPAS1 gene expression, which in turn positively autoregulated its own expression (Sato et al., 2002).

Inhibitory PAS domain protein, IPAS, is a bHLH/PAS protein structurally related to HIFs, IPAS contains no endogenous transactivation function but demonstrates dominant negative regulation of HIF-mediated control of gene expression. Application of an IPAS antisense oligonucleotide to the mouse cornea induced angiogenesis under normal oxygen conditions, and demonstrated hypoxia-dependent induction of VEGF gene expression in hypoxic corneal cells. This indicated a previously unknown mechanism for negative regulation of angiogenesis and maintenance of an avascular phenotype (Makino et al., 2001).

Although other PAS domain proteins have been linked to cancer, the novel gene described here has not to our knowledge, been described previously. The gene is not on the Lymphochip and has not been identified as a SEREX antigen.

Expression of the OX-TES-1 Gene in Normal and Neoplastic Human Tissues

The inventors have characterised the expression of the OX-TES-1 mRNA in normal human tissues and in matched normal and tumour tissues from cancer patients to investigate whether differential expression of the OX-TES-1 mRNA occurs in human cancer.

Multiple Tissue Expression (MTE) and Matched Tumour/Normal (MTN) arrays (BD Biosciences Clontech) were pre-hybridised according to the manufacturer's instructions. A610pb Pvull fragment of OX-TES-1 (nt 3360-3970), which is also present in the splice variant BC040301 and represents a portion of the 3′UTR, was radiolabelled using the High Prime DNA Labelling Kit (Roche Diagnostics GmbH, Germany). The probe was denatured and incubated with C₀t-1 DNA and sheared salmon sperm DNA as described (MTE/MTN Array User Manual, BD Biosciences), before being added to the arrays and incubated overnight with gentle rotation at 60° C. The arrays were then washed for 5×20 min in 2×SSC, 1% SDS at 65° C. followed by 2×20 min in 0.1 SSC, 0.5% SDS at 55° C. The washed membranes were exposed to film at −70° C. Additional information about the tissues and cases on these arrays can be obtained from the BD Biosciences Clontech website (www.clontech.com). Loading of the cDNAs on both arrays is normalised for three housekeeping genes to enable quantitative comparisons between gene expressions in different tissues.

Normal Tissues

The normal tissues on the MTE array come from non-diseased victims of sudden death/trauma and are pooled from a number of individuals. The expression of the OX-TES-1 mRNA in normal human tissues was found to be restricted to testis (FIG. 6). This is supported by the information in the UniGene folder that contains the splice variant (Hs. 160594), where all of the EST clones have been isolated from testis (or from pooled samples containing testis). The other positive signals sen on this array are with cDNA from a colorectal adenocarcinoma cell line and the human genomic DNA (500 ng) control. A positive result with human genomic DNA can indicate the presence of a highly-abundant gene or a gene family. It could also indicate the presence of repetitive sequences in the probe. However, since there was no signal with the C₀t-1 DNA, the former is a more likely explanation.

Neoplastic Tissues

Analysis of OX-TES-1 mRNA expression in human tumour tissues showed differential expression in all tumours, compared to adjacent histologically-normal tissue, included on the MTN array (FIG. 7). It seems likely that this differential expression represents genuine overexpression, as no OX-TES-1 mRNA expression was observed in those normal tissues on the MTE array (FIG. 6). Differential levels of expression were also seen in the human cancer cell lines (FIG. 7 Row P), with the melanoma (P5) and colorectal adenocarcinoma (P8) cell lines showing the highest levels. Incidentally, the colorectal adenocarcinoma cell line on the MTN array is the same as that on the MTE array, where it also gave a positive signal (FIG. 6). Although the data between the two arrays may seem discordant, an important consideration when interpreting this data is that adjacent histologically-normal tissues from a cancer patient are not necessarily genotypically normal, and early genetic changes, which do not affect the appearance of the tissue, may also be present in the matched normal tissue of cancer patients. A positive signal was also seen with E. coli DNA (FIG. 7, G24). Since there is no binding to either the yeast samples or the E. coli rRNA, this is unlikely to be due to non-specific binding. More likely it is due to the presence of a bacterial homologue, or to similarity to bacterial DNA in the region of OX-TES-1 selected as the probe, although BLAST searching of the E. coli genome found no matches.

In conclusion, from both arrays it can be inferred that OX-TES-1 has a very restricted expression in normal tissues—present only in testis—but that it is widely expressed in a number of tumour and adjacent histologically-normal tissues from cancer patients. As such, this points towards OX-TES-1 being a novel Cancer-Testis Antigen (CTA). CTAs are a novel group of SEREX-identified antigens that are expressed only in normal testis (occasionally placenta and/or uterus) and human cancers, and since testis is an immunologically-privileged site, CTAs are ideal targets for immunotherapeutic vaccines (Chen et al., 1998; Jager et al., 2000). CTAs are reported to be expressed more frequently in T-cell lymphomas than B-cell lymphomas (Xie et al., 2003). Therefore the identification of a novel CTA that is expressed in a B-cell lymphoma furthers the possibility of their use in vaccines for B-cell malignancies. The finding that OX-TES-1 is common to a wide range of different tumours raises the possibility that this antigen may have a diagnostic or therapeutic use in a variety of cancers.

Expression of the OX-TES-1 Gene in DLBCL Cell Lines

The inventors have characterised the expression of the OX-TES-1 mRNA in a number of DLBCL cell lines to confirm the expression of OX-TES-1 in lymphomas, and to establish which of the two alternatively-spliced variants might be present and, therefore, relevant to DLBCL.

DLBCL-derived cell lines were maintained in RPMI 1640 medium (Sigma Aldrich, UK) supplemented with 5% foetal calf serum (10% normal human serum for OCILY10) and antibiotics (penicillin (5000 U/ml) and streptomycin (5000 μg/ml); Invitrogen, UK) in an atmosphere of 5% CO₂ at 37° C. Cells were washed in RNase-free PBS prior to mRNA extraction. The cell lines were representative of the activated (poor prognosis) (OCILY3, OCILY10 and HLY-1) and the germinal centre (good prognosis) (SUDHL-6) subtypes, as well as some of unknown subtype (MIEU, LIB and DEAU).

RT-PCR was carried out as follows: Poly(A)+ mRNA from the seven DLBCL-derived cell lines was extracted using μMACS mRNA Isolation kits (Miltenyi Biotech, Germany). cDNA was reverse transcribed at 42° C. for 50 minutes from 20 ng mRNA in a 25 μl reaction containing 200U Superscript II™ RNase H⁻ reverse transcriptase (Invitrogen, UK), 1× First Strand Buffer, 4 mM DTT and 100 ng of either oligo(dT) primer or random hexamers. The integrity of cDNA templates was assessed using gene-specific primers to b-actin. 2 μl cDNA was amplified in a 25 μl PCR containing 200 μM each dNTP, 10 μM each primer, 1× PCR buffer and 1× Advantage 2 Polymerase mix (BD Biosciences Clontech, California, USA). Gene specific primers were designed to amplify fragments from 211 bp to 1500 bp, and their sequences are shown in Table x. Cycling parameters were as follows: 5 mins at 94° C. (initial denaturation) then 45 seconds at 94° C., 45 seconds at appropriate annealing temperature and 2½ or 5 minutes at 72° C. for 30 or 35 cycles (see Table x also). Phagemid DNA containing the appropriate cDNA insert was used as a positive control, whilst the negative control was a PCR mixture with no cDNA template. PCR products were visualised after separation in agarose gels by staining with ethidium bromide. Check table name

Expression of OX-TES-1 in DLBCL Cell Lines

Analysis of OX-TES-1 mRNA with primer set A in DLBCL-derived cell lines successfully amplified fragments, in all cell lines tested, of the same size as the positive control (211 bp) (see FIG. 8B; 8A shows the results from the primary amplification). No product is observed with the negative control. This suggests that OX-TES-1 is transcribed in DLBCL cell lines. Although the PCR is not quantitative, the fact that all products obtained with the cell line cDNAs are of similar intensity suggests that similar levels of expression occur in all types of DLBCL.

Analyses of Expression of OX-TES-1 Splice Variants in DLBCL

Since primer set A is designed to amplify a region of the cDNA that is present in both OX-TES-1 and the splice variant BC040301, RT-PCR was carried out with primers designed to the region that is deleted in the splice variant—i.e. specific to OX-TES-1 (primer set B). FIG. 8D (8C shows the results from the primary amplification) shows that a fragment corresponding to the same size as the positive control (360 bp) is successfully amplified in all cell lines; no product is observed with the negative control. This suggests that the OX-TES-1 variant is transcribed in DLBCL. Interestingly, when the cDNA is synthesised using oligo(dT) primers, an additional fragment of ˜1100 bp is observed (seen to a lesser extent with hexamer-primed cDNA (FIG. 8E) that is much stronger than the expected product. This fragment is too small to be the 3′ RACE product if the transcript in DLBCL is 4109 bp, as the original cDNA is, as this would be expected to be 1929 bp in size. This larger fragment is currently being analysed to determine its sequence and whether it represents another variant of OX-TES-1 that may be present in DLBCL, or just an artefact of the PCR.

In order to determine whether both variants are transcribed in DLBCL, RT-PCR was carried out with primers designed to flank the region that is deleted in splice variant BC040301 (primer set C). With these primers, a 1505bp fragment indicates the presence of OX-TES-1 whilst a 238 bp fragment indicates the presence of the splice variant. FIG. 8G shows that a fragment of ˜1500 bp is amplified in all cell lines, and that there is no ˜200 bp fragment seen in any cell line, suggesting that only the OX-TES-1 variant is transcribed in DLBCL (8F shows the results from the primary amplification where the 1500 bp product is only just visible in some cell lines).

BAC1 (OX-TES-2)

This is a novel cDNA that, when translated, does not encode any large protein product. Further studies will be necessary to identify the protein product of this clone that is recognised by DLBCL patients' sera. The gene is not on the Lymphochip and has not been identified as a SEREX antigen. The sequences of OX-TES-2 are shown in FIGS. 9 and 10

GKAP42 (OX-TES-20)

Searches of the PubMed database for GKAP42 (or FKSG21) identified only a single publication that described this protein as a specific anchoring protein (Yuasa et al., 2000). Such proteins located in various cell compartments are thought to regulate protein kinase specificity through subcellular compartmentalisation. GKAP42 was cloned because of its physical interaction with the cGMP-dependent protein kinase cGK-Ia. Immunocytochemical observations using a GKAP42 polyclonal antibody revealed that it was localized to the Golgi complex while Northern blotting studies suggested that it is predominantly (possibly exclusively) expressed in testis. It has been suggested that GKAP42 functions as an anchoring protein for cGK-Ia, and that cGK-Ia may participate in germ cell development through phosphorylation of Golgi-associated proteins such as GKAP42.

Increased levels of intracellular cAMP inhibit T cell activation and proliferation. One mechanism is via activation of the cAMP-dependent protein kinase (PKA). The specificity of PKA signalling is maintained thorough interactions with A kinase anchoring proteins (AKAPs) that convey spatial and temporal localization to PKA and other signalling molecules. A novel gene myeloid translocation gene 16b has been demonstrated to be a new AKAP that targets PKA to the Golgi of T lymphocytes (Schillace et al., 2002).

To the best of our knowledge, this molecule has no prior association with human cancer and has not been previously identified as a SEREX antigen on the SEREX database. In addition, the gene is not on the Lymphochip used to profile gene expression in DLBCL. Much of the sequence is, however, deposited in the patent database.

Accession numbers: BC014476, AB033132, AF319476, AB033131, NM_{—}025211, AK026487, XM_{—}005807.

Sirtuin (SIRT1/hSir2/Sir2alpha)OX-TES-21

This gene encodes a member of the sirtuin family of proteins that have homology to the yeast Sir2 protein that regulates epigenetic gene silencing and suppresses recombination of rDNA. Little was known about this protein until October 2001, when two papers in Cell linked this protein to the p53 tumour suppressor gene (Luo et al., 2001; Vaziri et al., 2001). Mammalian Sir2alpha has been shown to physically interact with p53 and attenuate p53 mediated functions, including the repression of p53-dependent apoptosis in response to DNA damage and oxidative stress (Luo et al., 2001). The binding of hSir2 to p53 deacetylates this protein with a specificity for its C-terminal Lys382 residue, modification of which has been implicated in activation of p53 as a transcription factor (Vaziri et al., 2001).

The critical role of the p53 protein in tumour development is well established in many different types of cancer. The expression of p53 also has prognostic significance in DLBCL with increased expression being associated with a shorter disease free survival time (Zhang et al., 1999). The above data implicate the hSir2 protein in cancer through its ability to regulate p53 function and therefore apoptosis.

There is no published data indicating that the SIRT1 protein is a tumour antigen (absent from SEREX database) or that this protein has a role in DLBCL (absent from Lymphochip). However much of the sequence is in the patent database.

Accession numbers: NM_{—}012238, AF083106, AL133551, BC012499, AL136741, AF2325040, AK027686.

TZP Transcription Factor: NZF, DKFZp434F0272 (OX-TES-24)

Other names are NZF and DKFZp434F0272. There do not appear to be any publications in PubMed describing this molecule, although the full length sequences are available in GenBank. Analysis of the 1012 aa predicted protein sequence, using the PSORT II programme, identified a large number of potential nuclear localisation signals, a prenylation motif near the C-terminus, a C2H2 zinc finger motif at aa 454, a C-terminal coiled-coil domain (aa 940-968) and predicted that the protein was likely to be nuclear. Analysis using MotifFinder also predicted the C2H2 zinc finger at aa 452-477, and an AT hook motif at 257-269 together with a PHD-finger at aa 654-700.

Information on the sequences submitted to Genbank also reported the presence of a tudor domain.

The C2H2 zinc finger consensus sequence, CX{2,4}CX3(L,I,V,M,F,Y,W,C)X8HX{3,5}H, represents a DNA binding motif that is present within a large number of transcription factors.

The AT-hook is a conserved DNA-binding peptide motif that preferentially binds to the minor groove of stretches of AT-rich sequence. Several copies of this motif are present in high mobility group proteins (HMG). The levels of HMGI(Y) proteins in human cells have been proposed to be sensitive diagnostic indicators of both neoplastic transformation and metastatic progression. Drugs based on the AT-hook motif offer the potential for development of new tumour therapeutic reagents (Reeves, 2000).

PHD (plant homeodomain) fingers are cysteine-rich motifs that were first noted in two closely related plant homeodomain proteins. Subsequently, this motif was identified in a number of transcription factors and was suggested to have a role in chromatin-mediated transcriptional regulation (Aasland et al., 1995). Recent data indicate that these PHD finger proteins are associated with chromatin remodelling complexes (Bochar et al., 2000), contribute to histone acetylation (Loewith et al., 2000), or act, as a general transcriptional activation domain (Halbach et al., 2000).

Tudor domains are conserved protein modules of unknown function that are often present in proteins that associate with RNA (Mushegian et al., 1997) but that have been predicted to mediate protein-protein interactions and may have a role during RNA metabolism and/or transport (Ponting, 1997; Selenko et al., 2001).

The domains present within the TZP protein, together with the presence of a number of single and bipartite nuclear localization signals, suggest that TZP is a nuclear protein with a role in regulation of gene expression.

This gene sequence has not been associated with DLBCL (as it is not present on the Lymphochip). However, this protein has been associated with human cancer, i.e. it has been identified using SEREX as a tumour antigen. There is some association between the location of TZP on chromsome 20q11.1-11.23 and genetic aberrations in DLBCL.

Accession numbers: AL137330, AF348207, AL109965, AY027523, BC006415, L09749.

Serine/Threonine Protein Kinase 11; STK11 (LKB1) (OX-TES-13)

People with Peutz-Jeghers syndrome have an increased risk of various neoplasms because of mutations in a human serine/threonine protein kinase, STK11. There are considerable numbers of papers implicating this molecule in cancer, but none suggesting that it has a role in lymphoma.

This protein has been shown to physically associate with p53, and to regulate p53-dependent apoptosis pathways (Karuman et al., 2001). As described above, p53 is known to be associated with clinical outcome in DLBCL. Knockout mice that lack Lkb1 die at mid-gestation because of deregulation of VEGF expression (Ylikorkala et al., 2001). It is possible, therefore, that this molecule is involved in tumour angiogenesis. STK11 has been identified as a tumour antigen (present in SEREX database) and the mRNA expression is upregulated in a few cases of DLBCL (data on Lymphochip). More importantly recurrent chromsome abberations are reported at the STK11 locus (19p13.3) in both FL and DLBCL. These data are consistent with STK11 having a potentially important role in the pathology of DLBCL.

Accession numbers: NM_{—}000455, AF035625, AF217978, U63333, BC007981.

Hypothetical Protein FLJ10955: PSP1 Paraspeckle Protein 1 (OX-TES-22)

This is an uncharacterised protein that is highly similar to human NonO. NonO is an unusual nucleic acid binding protein not only in that it binds both DNA and RNA but that it does so via functionally separable domains. NonO has been found to have carbonic anhydrase activity and may function to maintain nuclear pH (Karhumaa et al., 2000). This novel gene may thus encode a nuclear pre-mRNA splicing factor containing two RNA recognition motifs. A proteomic study of purified human nucleoli has identified Paraspeckle Protein 1 (PSP1) in a new nucleoplasmic compartment termed paraspeckles (Fox et al., 2002).1

There is no prior published data indicating that this molecule has any association with cancer, although the genomic region to which the gene maps has recurrent aberrations in a wide range of human tumours including DLBCL. Interestingly a large number of ESTs have been cloned from Burkitts lymphoma.

Accession numbers: AK001817, BC014184, NM_{—}018282, XM_{—}007072.

Y-Box Binding Protein 1; NSEP1 (Nuclease Sensitive (OX-TES-23) Element Binding Protein 1); DNA Binding Protein B (DBPB)

Y box-binding protein-1 (YB-1) is a member of the DNA binding protein family that interacts with inverted CCAAT boxes (Y-boxes). Y-boxes are located on the promoter of numerous genes, such as DNA topoisomerase II alpha (Topo II alpha), proliferating cell nuclear antigen (PCNA) and multidrug resistance 1 (MDR1). YB-1 (NSEP1/DBPB) has been reported to be involved in both transcriptional and translational regulation of gene expression with its expression affecting cell proliferation, genomic instability, multidrug resistance, RNA stabilisation and DNA repair.

The expression of HLA class II genes is regulated by a series of cis-acting elements and trans-acting factors. Cis-acting elements include the Y box and studies have suggested that YB-1 is a negative regulator of HLA-DR beta chain mRNA expression (Didier et al., 1988).

Thrombin induces expression of the platelet-derived growth factor B-chain gene in endothelial cells (EC) through activation of the Y-box binding protein YB-1. YB-1 is thrombin activated by a novel mechanism: proteolytic cleavage leads to release from mRNA, nuclear translocation, and induction of thrombin-responsive genes (Stenina et al., 2001).

YB-1 (NSEP1/DBPB) was isolated by screening for proteins that are able to bind to the repressor element in the human granulocyte-macrophage colony-stimulating factor (GM-CSF) promoter. Overexpression of YB-1 led to repression of the GM-CSF promoter (Coles et al., 1996). This study reported the presence of a central cold-shock domain (CSD), which is a highly conserved, approximately 100-amino acid domain with similarity to bacterial cold-shock proteins. The YB-1 gene has been mapped to 1p34 (Makino et al., 1996). A study using transduced YB-1 in eosinophils found that YB-1 stabilized GM-CSF mRNA in an ARE-dependent mechanism, causing increased GM-CSF production and enhanced in vitro survival (Capowski et al., 2001). RNA electrophoretic mobility shift assays (EMSAs) indicated that YB-1 interacted with the GM-CSF mRNA through its 3′ untranslated region ARE. The authors proposed a model whereby activation of eosinophils leads to YB-1 binding to, and stabilization of, GM-CSF mRNA, ultimately resulting in GM-CSF release and prolonged eosinophil survival (Capowski et al., 2001). This is particularly relevant to the field of cancer immunotherapy as GM-CSF is now being used as a therapeutic treatment to generate an anti-tumour immune response in cancer patients.

The effect of YB-1 on stabilizing mRNAs is more general, and it has been demonstrated that YB-1 is the major mRNA-associated protein, also termed p50, which is a potent cap-dependent mRNA stabilizer. YB-1 addition or overexpression dramatically increased mRNA stability in vitro and in vivo, whereas YB-1 depletion resulted in accelerated mRNA decay. The cold shock domain of YB-1 is responsible for the mRNA stabilizing activity, and a blocked mRNA 5′ end is required for YB-1-mediated stabilization (Evdokimova et al., 2001). The finding that YB-1 is necessary for JNK-induced stabilization of the IL-2 mRNA induced by T-cell activation signals (Chen et al., 2000) is particularly interesting, as a subgroup of DLBCLs with an activated phenotype has been linked to poor prognosis.

YB-1 has also been identified as a protein that interacts with a TGF-beta response element in the distal region of the collagen alpha 1(I) gene. YB-1 protein activates the collagen promoter and translocates into the nucleus during TGF-beta addition to fibroblasts, suggesting a role for this protein in TGF-beta signaling (Sun et al., 2001).

A recent study has reported that the Y-box protein YB-1 regulates expression of the P-glycoprotein (P-gp) gene MDR1, which plays a major role in the development of a multidrug-resistant tumour phenotype. They show that in human breast cancer, overexpression and nuclear localization of YB-1 is associated with upregulation of P-gp. YB-1 demonstrated prognostic and predictive significance in breast cancer by identifying high-risk patients in both the presence and absence of postoperative chemotherapy, independent of tumour-biologic factors currently available for clinical decision making (Janz et al., 2002§). A study on lung cancer reported that higher levels of YB-1 expression were associated with T3-4 and Stage III-IV tumours in adenocarcinomas but reported that no relationship was found between YB-1 expression and P-gp expression (Gu et al., 2001). This study did report that YB-1 expression correlates with Topo II alpha and expression in lung cancer (Gu et al., 2001). Interestingly another study reported that NSCL and squamous cell carcinoma patients with nuclear expression of YB-1 in their tumour had a poorer prognosis than did those with a cytoplasmic YB-1 tumour expression. This was not the case in patients with adenocarcinomas (Shibahara et al., 2001).

YB-1 is overexpressed in cell lines that are resistant to the cancer treatment drug cisplatin. Subsequently YB-1 has been shown to bind to PCNA in vivo indicating that YB-1 can function as a recognition protein for cisplatin-damaged DNA and that it may be important in DNA repair or in directing the cellular response to DNA damage (Ise et al., 1999).

A study using an in vitro pull-down assay has demonstrated that YB-1 directly binds to the p53 tumour suppressor protein. This interaction stimulated the binding of p53 to its consensus sequence, and antisense expression of YB-1 inhibited the induction of the p21 promoter by p53 in transient transfection assays (Okamoto et al., 2000).

This is a well characterised molecule that has been identified as a SEREX antigen and is present in the patent database. The cDNA is on the Lymphochip but we were unable to access the data using any of the search terms identified in the BLAST search of the Lymphochip sequences. Interestingly it has been reported that only low amounts of YB-1 mRNA were found in normal lymphoid tissues (Spitkovsky et al., 1992). Abstract number 1284 at the American Society of Hematology meeting in Orlando (9.12.01) presented the results of using oligonucleotide arrays to identify progression-specific genes in T-cell leukaemia/lymphoma (results not in published abstract). This study reported the increased expression of Y-box binding protein 1 (accession J03827) to be associated with disease progression.

Accession numbers: BC002411, J03827, BC015208, BC010430, M85234, M24070, L28809, M83234, NM_{—}004559, X96666.

Similar to Recombining Binding Protein Suppressor of Hairless (Drosophila) (OX-TES-16).

The gene that has been isolated maps to chromosome 4 and shows approximately 97% identity to the suppressor of hairless protein 1 (CBF1) and to the J kappa-recombination signal binding protein. It is therefore likely that this protein is an N-terminal variant of these proteins, and serum from patients with DLBCL may also recognise epitopes on these highly related proteins.

The CBF1 gene, also known as IGKJRB, is well conserved among many species and through a technical artefact, was thought to be involved in the V(D)J recombination process. There is reported to be one functional gene (with 3 isoforms) and 2 types of processed pseudogene (Amakawa et al., 1993). The Drosophila homologue is encoded by the suppressor of the hairless gene and plays a key role in the determination of neuronal cell fate (Schweisguth and Posakony, 1992).

The Epstein-Barr virus EBNA2 protein is a transcriptional activator that interacts with CBF1 to achieve promoter specificity (Ling et al., 1994). EBV immortalizes B lymphocytes and is associated with B-cell malignancies, including lymphomas. The Notch/Lin-12/Glp-1 receptor family participates in cell-cell signaling events that influence cell fate decisions. Studies using a truncated form of Notch which acts as an activated receptor demonstrated that this protein interacted with CBF1 (Hsieh et al., 1996). Thus Epstein-Barr virus-driven immortalisation works by mimicking the Notch signal transduction pathway leading to abolition of CBF1-mediated repression (Hsieh et al., 1996). In addition the recognition sites for CBF1 and NF-kB have been reported to overlap in the atypical NF-kB site in the CYP2B1/2 promoter (Lee et al., 2000).

Recombination signal binding protein itself has been identified as a SEREX antigen and is on the Lymphochip, although again the inventors were unable to access the Lymphochip data. The link between CBF1 and EBV immortalisation suggests that this molecule may be important in DLBCL.

The TA195 cDNA sequence was not a perfect match for anything in the database but it may represent a 5′ variant of the RBP-J kappa gene. The closest matches were to BC020780, an uncharacterised gene, and H. sapiens recombination signal binding protein (L078721). The cDNA sequence in BG614689 (and others) matched some of the 5′ end of TA195 that differed from the BC020780 sequence suggesting that there are a number of 5′ variants of the RBP-J kappa cDNA. All the genes map to chromosome 4 (clone NT_{—}006324.7).

Accession numbers for cDNA clones: BC020780, BG614689, XM029255, L07872

Histone MacroH2A1 (H2AFY) (OX-TES-17)

MacroH2A1 is an unusual variant of the core histone H2A that is enriched in chromatin on the inactive X chromosome of female mammals. MacroH2As have a unique hybrid structure consisting of an amino-terminal domain that is approximately 65% identical to the full-length histone H2A followed by a large large C-terminal nonhistone region containing a small stretch of basic amino acids and a leucine zipper. The human macroH2A1 gene, on chromosome 5, encodes two macroH2A subtypes, macroH2A1.1 and macroH2A1.2, produced by alternate splicing.

Both protein isoforms of histone macroH2Al (mH2A1) are found in mammalian cells. One murine isoform, mH2A1.2 is highly concentrated on the heterochromatinized inactive X chromosome (Xi) of female cells, and mH2A1.2 protein is also present in male cells, but fails to form dense concentrations. The relatively abundant expression of mH2A1 in both sexes suggests that mH2A1 has functions in addition to a possible involvement in X chromosome inactivation (Rasmussen et al., 1999). Centrosomal association of macroH2A1.2 is a widespread phenomenon and is not restricted to undifferentiated and early differentiating ES cells. By indirect immunofluorescence, macroH2A1.2 protein has been detected in a juxtanuclear structure that duplicates once per cell cycle and colocalizes with centrosomal gamma-tubulin in both XX and XY ES cells prior to and throughout their differentiation. MacroH2A1.2 localization to the centrosome is also observed in female and male somatic cells, both in interphase and in mitosis (Mermoud et al., 2001).

Genetic instability is a common feature of almost all human cancers including non-Hodgkin's lymphoma (NHL). In NHL, numerical chromosome aberrations have been shown to provide information about the clinical course and the risk of transformation to a high grade malignancy. Recently centrosome aberrations have been described as a possible cause of aneuploidy in many human tumours (Marx, 2001). A poster (No. 1407) presented by Schwiezer et. al. at the American Society of Hematology Meeting in Orlando (December 2001) reported that centrosome defects are a common feature of NHL, including DLBCL, and that they contribute to the karyotypic instability commonly seen in high grade NHL.

Recombinant macroH2A1.2 was able to efficiently replace both of the conventional H2As in reconstituted nucleosomes. The substitution of macroH2A1.2 for H2A did not appear to grossly perturb the basic structure of the nucleosome core. However, minor differences have suggested that macroH2A1.2 may promote interactions between nucleosomes (Changolkar and Pehrson, 2002).

Interestingly a novel gene, BAL1, encodes a previously uncharacterized 88-kDa nuclear protein with a duplicated N-terminal domain similar to the nonhistone portion of histone-macroH2A and a C-terminal alpha-helical region with 2 short coiled-coil domains. BAL is expressed at increased levels in fatal high-risk DLBCLs and DLBCL cell lines with an activated peripheral B cell, rather than those low risk lymphomas with a germinal centre B cell, phenotype. Studies showed that the BAL gene promoted malignant B-cell migration which may cause the characteristic dissemination of high risk DLBCLs (Aguiar et al., 2000).

MacroH2A1 has been identified as a SEREX antigen but is not present on the Lymphochip. The related H2AX gene is on the Lymphochip and shows increased expression in DLBCL.

The first 86 nucleotides of this cDNA sequence do not correspond to that of publicly disclosed sequences, and this clone therefore has a longer 5′ untranslated region than other cDNA clones encoding this molecule.

Accession numbers: XM_{—}047728, AK023409, XM_{—}047729, AF041483, AF054174, BC013331, NM_{—}004893, AF044286.

Histone H3.3B (H3F3B) (OX-TES-5)

In contrast to the cell-cycle-dependent histone genes, replacement histone genes are transcribed independently of DNA replication and their expression is upregulated during differentiation. Studies on expression of the mouse H3.3 replacement variant mRNA indicated that steady state levels increased during the G_{O} to S-phase transition, apparently as the result of two mechanisms: one was related to cell growth, whereas the other was linked to cellular DNA synthesis (Hraba-Renevey and Kress, 1989).

Differential hybridization to a cDNA library made from the mRNA of differentiating mouse erythroleukaemia (MEL) cells has been used to identify sequences that are induced during the early stages of MEL cell differentiation. Histone H3.3B was differentially expressed being coordinately induced during the first few hours of MEL cell differentiation and subsequently down regulated as cells underwent terminal differentiation. Nuclear run-on transcription experiments indicated that the accumulation and decay of these mRNAs was controlled at the post-transcriptional level. Unlike the polyadenylated mRNAs of two H1 histone genes which exhibit similar kinetics of induction and decay controlled by c-myc, induction of the H3.3 mRNAs was unaffected by deregulated expression of c-myc (Krimer et al., 1993), which is a common feature of lymphomas including DLBCL. During rat brain development the concentration of both H1(0) and H3.3B mRNAs decreased from the embryonal day 18 (E18) to the postnatal day 10 (P10), with an inverse correlation to protein accumulation (Castiglia et al., 1994). The effects of transcription inhibition by actinomycin D and the results of nuclear run-on experiments, suggest that H3.3 expression is regulated mainly at the post-transcriptional level (Scaturro et al., 1995). This finding is particularly significant as it suggests that studying the mRNA expression of this gene (for example using gene-expression profiling studies) is unlikely to represent the levels of protein expression.

Investigation of the transcriptional regulation of the replacement histone gene H3.3B showed that promoter activity largely depended on an intact Oct and CRE/TRE element within the proximal 145 bp of the promoter. DNase I footprinting revealed binding of proteins to a 40-bp region covering these two elements. Band shift experiments identified these binding proteins as Oct-1 and factors of the CREB/ATF and AP-1 family, respectively (Witt et al., 1997). The Oct-1 transcription factor regulates a variety of tissue-specific and general housekeeping genes by recruiting specialized co-activators of transcription. Of particular interest is that Oct-1 synergistically acts with the B-cell-specific coactivator Bob1 (OCA-B, OBF-1) to stimulate the B-cell specific transcription of immunoglobulin genes. Activation of the protein kinase C pathway by phorbol 12-myristate 13-acetate (PMA) has been shown to result in an early up-regulation of H3.3B gene expression via the CRE/TRE element. Furthermore, treatment with PMA leads to differential induction of H3 histone subtype genes, and can result in a remodelling of chromatin structure of cells before or during differentiation processes (Witt et al., 1998).

This gene has not been cloned as a SEREX antigen and the cDNA is not on the Lymphochip. There does not appear to be any prior data implicating this gene in cancer.

Accession numbers: Z48950, NM 005324, BC012813, BC006497, BC001124, AK054591, BC017558, AF218029.

SFRS Protein Kinase 1 (SRPK1) (OX-TES-10)

Serine/arginine-rich (SR) proteins are essential for pre-mRNA splicing. These proteins modify the choice of splice site during alternative splicing in a process apparently regulated by protein phosphorylation. Serine/arginine protein kinases (SRPKs) have been conserved throughout evolution and are now thought to play important roles in the regulation of mRNA processing, nuclear import, germline development, polyamine transport, and ion homeostasis.

The SRPK1 kinase has been shown to be regulated by the cell cycle and is specific for SR proteins. SRPK1 is related to a Caenorhabditis elegans kinase and to the fission yeast kinase Dsk1. SRPK1 specifically induces the disassembly of nuclear speckles, and a high level of SRPK1 inhibits splicing in vitro. Small nuclear ribonucleoprotein particles (snRNPs) and non-snRNP splicing factors containing a serine/arginine-rich domain (SR proteins) concentrate in ‘speckles’ in the nucleus of interphase cells. It is believed that nuclear speckles act as storage sites for splicing factors while splicing occurs on nascent transcripts. Splicing factors redistribute in response to transcription inhibition or viral infection, and nuclear speckles break down and reform as cells progress through mitosis. This study suggested that SRPK1 may have a central role in the regulatory network for splicing, controlling the intranuclear distribution of splicing factors in interphase cells, and the reorganization of nuclear speckles during mitosis (Gui et al., 1994).

Cloning of murine mSRPK1 and mSRPK2, and their expression analyses, revealed the ubiquitous expression of mSRPK1 in all tissues examined, and the tissue-specific expression of mSRPK2 in testis, lung, and brain. Both kinases phosphorylated SF2/ASF, a member of SR proteins in vitro and the phosphopeptide maps were identical, indicating that these kinases phosphorylated the same site of SF2/ASF. The results indicated that SRPK family members may regulate the disassembly of the SR proteins in a tissue-specific manner (Kuroyanagi et al., 1998).

Studies on expression of the human genes and the proteins they encode indicated that the biochemical and functional similarities between SRPK1 and 2 contrasted with their differences in expression patterns. SRPK1 was highly expressed in pancreas, whereas SRPK2 was highly expressed in brain, although both were co-expressed in other human tissues and in many experimental cell lines. Interestingly, SRPK2 also contained a proline-rich sequence at its NH2 terminus, and a recent study showed that this NH2-terminal sequence has the capacity to interact with a WW domain protein in vitro. Different SRPK family members may be uniquely regulated and targeted, thereby contributing to splicing regulation in different tissues, during development, or in response to signalling (Wang et al., 1998).

The therapeutic potential of cisplatin, one of the most active and widely used anti-cancer drugs, is severely limited by the occurrence of cellular resistance. Recent studies have strongly suggested that SRPK1 is involved in cisplatin-induced cell killing, and indicated that SRPK1 might be of potential importance for studying clinical drug resistance (Schenk et al., 2001).

This is a well-characterised molecule that has been identified as a SEREX antigen and shows heterogeneous expression in the Lymphochip data.

Accession numbers: Z99128, AJ318054, NM_{—}003137, U09564, AL117648, XM_{—}043350.

Heterogeneous Nuclear Ribonucleoprotein A2/B1: HNRPA2/B1 (OX-TES-12)

The splicing of pre-mRNA into the mature product occurs in a multicomponent complex constituted by small nuclear ribonucleoprotein particles (snRNPs) and proteins such as splicing factors and heterogeneous nuclear ribonucleoproteins (hnRNPs). The hnRNP proteins are a family of at least 20 polypeptides whose precise role in RNA processing remains to be fully defined. The hnRNP A1, A2, B1, and B2, belong to the basic protein subset of the hnRNP complex. These proteins share a modular structure consisting of 2 N-terminal conserved RNA binding domains linked to less conserved C-terminal glycine-rich domains.

HnRNPs are predominantly nuclear RNA-binding proteins that form complexes with RNA polymerase II transcripts. HnRNPs have been implicated in most stages of cellular mRNA metabolism including processing, nucleocytoplasmic transport, stability, and localization. Several hnRNPs are also known to participate in key early developmental decisions. The hnRPA2/B1 gene produces both the B1 and A2/B1 mRNAs as a consequence of alternative splicing. The transcripts were found in all cells, with the B1 isoform occurring at about 2-5% of the A2/B1 level (Biamonti et al., 1994; Kozu et al., 1995).

The overexpression of hnRNPA2/B1 has been reported to be informative as a marker for the early detection of lung cancer, although the biological reason is not clear. The expression of hnRNP A2/B1 is, however, not specific for lung cancer, and quantitative determination of A2/B1 is required to elucidate its significance in carcinogenesis (Satoh et al., 2000). It has been shown that microsatellite instability correlates with overexpression of hnRNPA2/B1 mRNA in lung tumours, leading to a conclusion that lung tumour cells undergoing clonal expansion frequently upregulate hnRNPA2/B1 (Zhou et al., 2001). Early results also suggest that the further evaluation of hnRNPA2/B1 as a marker of breast carcinogenesis is worthwhile (Zhou et al., 2001).

A2/B1 proteins have been reported to bind telomeric DNA repeats, and functional assays have demonstrated that B1 and B0b bind telomeric repeats in a tandem fashion, protecting them from a nuclease and promote telomerase activity. A2/B1 proteins, especially B1 and B0b, may therefore function as telomeric ssDNA-binding proteins in cancer and reproductive cells (Kamma et al., 2001).

Three anti-A2/B1 monoclonal antibodies have been developed using recombinant A2 protein and synthesized peptides, around the second splicing site. These antibodies separately recognize A2 and B1, and this specificity makes them useful in the study of the biochemical and functional properties of A2 and B1, demonstrating the differences between A2 and B1 in their intracellular distribution and in their metabolism through cell cycle. They may be valuable reagents to clarify the clinical significance of A2/B1 in autoimmune diseases and cancers (Kamma et al., 2001).

Recently, in addition to the detection of circulating tumour cells in peripheral blood of patients with solid tumours, the presence of free circulating nucleic acids in the plasma and serum has also been described. Using RT-PCR, the hnRNPB1 mRNA was detectable in 14/18 sera from lung cancer patients. Combining amplification of Her2/neu-specific mRNA with hnRNPB1 mRNA; all patients with a malignant lung tumour were identified (Fleischhacker et al., 2001).

The hnRNPA/B proteins represent a group of novel autoantigens that are targeted by autoantibodies from patients with rheumatoid arthritis (RA), systemic lupus erythematosus (SLE), and mixed connective tissue disease (MCTD). Anti-A2/RA33 autoantibodies target the hnRNP proteins A2, B1, B2 (the ‘RA33 complex’), and anti-A1 autoantibodies are directed to the hnRNP proteins A1 and A1b. However, compared with MCTD and SLE patients, RA patients have a more restricted immune response to the spliceosome: they react to hnRNP proteins, particularly to hnRNPA2/RA33, but not to snRNPs (Hassfeld et al., 1995). This finding is of particular interest as we have identified antibodies against the heterogeneous nuclear ribonucleoprotein (hnRNP) A2/B1 protein in patients with DLBCL

It is possible that the detection of hnRNPA2/B1 mRNA in serum from DLBCL patients, or measuring the titre of anti-hnRNPA2B1 antibodies in their serum, could be used as a marker of tumour burden.

Abstract number 1284 at the American Society of Hematology meeting in Orlando (9.12.01) presented the results of using oligonucleotide arrays to identify progression-specific genes in T-cell leukaemia/lymphoma (results not in published abstract). This study reported the increased expression of a related gene, hnRNPA1 (accession X04347), to be associated with disease progression.

This molecule has been identified as a SEREX antigen but was not included on the Lymphochip and does not appear to have been investigated in DLBCL.

Accession numbers: M29065, M29064, AK026373, NM_{—}002137, NM_{—}031243, D28877, BC000506.

KIAA0643 (OX-TES-18)

This molecule has been fully sequenced but as yet there are no publications describing its potential function. Analysis of the protein coding sequence using the PSORT II programme identified the presence of a very large C-terminal coiled-coil domain and predicted a nuclear localisation for the protein.

The KIAA0643 protein is weakly similar to nucleolar transcription factor 1 also known as UBF1. The expression of UBF, also known as the autoantigen NOR-90, is an important regulatory mechanism involved in the acceleration, and possibly deceleration, of rDNA transcription observed during mitogenic stimulation and inhibition of blood cells. In this process, the upstream binding factor (UBF) is involved in regulating rDNA transcription at the nucleolus, together with RNA polymerase I. Ribosomal RNA synthesis is a key molecular process for understanding the mechanisms that drive cell proliferation.

The nucleolar organizer regions (NORs) of human chromosomes can be identified in interphase and mitotic cells by localization of some intrinsic components, such as the associated enzyme RNA polymerase I. A new sensitive staining method for NORs has been described using a specific antibody to the ribosomal transcription factor UBF. NORs stained in benign and malignant cells from a variety of tissues with anti-UBF serum showed significant morphological differences that correlated well with histopathological evaluation, and the number of NORs per cell in malignant preparations increased. UBF has been demonstrated to be a substrate for selective cleavage by specific proteases during apoptosis and the disappearance of UBF and preservation of other NOR proteins is consistent with the pattern of selective proteolysis by caspases described in early stages of apoptosis. Interestingly the protein encoded by the retinoblastoma susceptibility gene (Rb) functions as a regulator of transcription by RNA polymerase I (rDNA transcription) by inhibiting UBF-mediated transcription. The immunohistochemical staining pattern for caspase-3 has been linked to clinical outcome in DLBCL (Donoghue et al., 1999).

This gene is not on the Lymphochip but it has been identified as an antigen in prior SEREX studies.

AK023319, AB014543, AK023359, NM_{—}024793, BC017070

Human DNA Repair Helicase: ERCC3 (OX-TES-19)

DNA repair is central to the integrity of the human genome. Reduced DNA repair capacity has been linked to genetic susceptibility to cancer, and an adequate expression level of DNA repair genes is essential for normal DNA repair activities. Nucleotide excision repair (NER) is the DNA repair pathway through which cisplatin-DNA intrastrand adduct is repaired. Clinical studies have shown that increased mRNA expression of selected genes involved in the rate-limiting step of NER appears to be closely associated with clinical resistance to platinum agents.

Human DNA repair genes have been identified using UV-sensitive Chinese hamster cells as recipients for DNA-mediated gene transfer. The human genes correcting the rodent repair defects are termed excision-repair cross-complementing or ERCC genes. A number after the symbol refers to the rodent complementary group that is corrected by the human gene. ERCC3 has been shown to be the same as the gene for xeroderma pigmentosum complementation group B (XPB). Mutations in several DExH-containing DNA helicases, including XPB, are associated with rare familial cancer syndromes characterized by genomic instability and cancer susceptibility. Known cellular activities of these helicases include DNA replication, repair, recombination, and/or transcription. Physical and functional interactions of the p53 tumour suppressor with DExH-containing DNA helicases have been described. Such interactions could be compromised in inherited disorders and contribute to their cancer susceptibility (Robles and Harris, 2001).

ERCC3 is one of the components of the human transcription factor BTF2/TFIIH required for a late step in the initiation of transcription of genes with the class II promoter indicating that transcription and nucleotide excision repair share common factors (Schaeffer et al., 1993). Through TFIIH, FUSE-binding protein (FBP) facilitates transcription until promoter escape, whereas after initiation, FBP-interacting repressor (FIR) uses TFIIH to delay promoter escape. Mutations in TFIIH that impair regulation by FBP and FIR affect proper regulation of MYC expression and have implications in the development of malignancy (Liu et al., 2001).

In patients with H. pylori infection a significant reduction has been reported for ERCC3 mRNA levels in infected compared with uninfected gastric mucosa in patients without peptic diseases (Chiou et al., 2001). This is interesting because of the association between H. pylori infection and the development of MALT lymphoma.

The previously uncharacterized CDC24 homology domain of BCR, which is missing in the P185 BCR-ABL oncogene of Philadelphia chromosome (Ph1)-positive acute lymphocytic leukaemia but is retained in P210 BCR-ABL of chronic myelogeneous leukaemia, was found to bind to XPB. The binding appeared to be required for XPB to be tyrosine-phosphorylated by BCR-ABL. The interaction not only reduced the ATPase and the helicase activities of XPB purified in the baculovirus system, but also impaired XPB-mediated cross-complementation of the repair deficiency in rodent UV-sensitive mutants of group 3. The persistent dysfunction of XPB may in part underlie genomic instability in blastic crisis (Takeda et al., 1999).

ERCC3 has not been identified as a SEREX antigen and, although the cDNA was included on the Lymphochip, there was no change in expression levels in DLBCL.

Accession numbers: NM_{—}000122, BC008820, M31899

FLJ13942 (OX-TES-25)

This is a hypothetical protein that has some similarity to myosin. Analysis of the 519 aa protein sequence using the PSORT II server identified a large coiled-coil region (aa 32-360) and a potential nuclear localisation signal at aa 422. The programme predicted a nuclear localisation for this protein and searches against the Pfam database found some similarity to the basic and leucine zipper regions of the bZIP transcription factors.

This molecule has been identified as a SEREX antigen but was not included on the Lymphochip.

Accession numbers: AK024004, BC009055, NM_{—}024581, AL136767, XM_{—}044922

HISI (HEXIMI): HMBA-Inducible mRNA (OX-TES 14)

HIS1 is a gene of unknown function whose mRNA expression is induced by hexamethylene-bis-acetamide (HMBA) in vascular smooth muscle cells. PSORT II analysis of the 266 aa protein sequence identified several potential N-terminal nuclear localisation sites, two ER membrane retention signals, and a peroxisomal targeting signal at aa 238. The protein is predicted to have a nuclear localisation and contains a C-terminal coiled-coil region (aa 185-258).

HMBA, a differentiation inducer belonging to the class of hybrid polar compounds, is known to induce terminal differentiation of a number of leukaemic and solid tumour cell lines. Therefore, HMBA has been used in the differentiation therapy of cancer for patients with both haematological and solid malignancies. The HIS1 protein may therefore be one of the factors responsible for HMBA induction of differentiation.

During HMBA-induced differentiation of murine erythroleukaemia (MEL) cells, erythroid genes are transcriptionally activated while c-myb and several other nuclear proto-oncogenes are down-regulated. High levels of c-myb expression are necessary for the proliferation of haematopoietic precursor cells. Transcriptionally active NF-kB (p50/RelB) complexes, but not p50 or RelB alone, prevented the early and late down-regulation of c-myb mRNA and increased c-myb transcriptional elongation in HMBA-induced MEL cells. The increase in c-myb expression was sufficient to block erythroid differentiation and allow continuous proliferation of cells in the presence of HMBA. Steady-state c-myb mRNA levels in untreated cells were not affected by overexpression of NF-kB, suggesting that p50/RelB specifically modulated the efficiency of transcriptional attenuation during MEL cell differentiation (Suhasini and Pilz, 1999). During an oral presentation at the American Society of Hematology meeting in Orlando (Sep. 12, 2001), Dr Louis Staudt presented data indicating that the NF-kB pathway was active in the poor prognosis activated-B like group of DLBCLs. He suggested that the NF-kB pathway was a new therapeutic target for this type of DLBCL.

The development of chemoresistance is common for patients with multiple myeloma. HMBA has been shown to inhibit the growth of several human myeloma cell lines, including doxorubicin-resistant RPMI 8226 variants that overexpress the multidrug-resistance gene, MDR-1, and its product, P-gp. In addition to growth arrest and suppression of clonogenicity, HMBA induced apoptosis and decreased BCL-2 protein expression in myeloma cells. Overexpression of BCL-2 protein in ARP-1 cells conferred resistance to HMBA-induced apoptosis. HMBA appears to be a potent inducer of apoptosis in human myeloma cells, which may act through suppressing the anti-apoptotic function of the bcl-2 gene (Siegel et al., 1998).

HIS1 has been identified as a SEREX antigen but was not on the Lymphochip. This gene maps to 17q21, a region associated with recurrent aberrations in DLBCL and FL.

Accession numbers: AB021179, BC006460, NM_{—}006460, AK023624, XM_{—}008348

Adducin 1 (alpha): ADD1 (OX-TES-26)

Adducin is a ubiquitously expressed membrane-skeletal protein localised at spectrin-actin junctions. It binds calmodulin and is an in vivo substrate for Protein Kinase C (PKC) and Rho-associated kinase. Adducin is a tetramer comprised of either alpha/beta or alpha/gamma heterodimers. Adducin subunits are related in sequence and all contain an N-terminal globular head domain, a neck domain and a C-terminal protease-sensitive tail domain. The tail domains of all adducin subunits end with a highly conserved 22-residue myristoylated alanine-rich C kinase substrate (MARCKS)-related domain that has homology to MARCKS protein. Adducin caps the fast-growing ends of actin filaments and also preferentially recruits spectrin to the ends of filaments. Both the neck and the MARCKS-related domains are required for these activities. The neck domain self-associates to form oligomers. The MARCKS-related domain binds calmodulin and contains the major phosphorylation site for PKC. Calmodulin, gelsolin and phosphorylation by the kinase inhibit in vitro activities of adducin involving actin and spectrin. Recent observations suggest a role for adducin in cell motility, and as a target for regulation by Rho-dependent and Ca²⁺ dependent pathways. Prominent physiological sites of regulation of adducin include dendritic spines of hippocampal neurons, platelets and growth cones of axons (abstract from (Matsuoka et al., 2000)). Genetic variation in adducin, a protein associated with the inner leaflet of the plasma membrane, may be in part responsible for salt-sensitive hypertension.

PKC isoforms are serine/threonine kinases involved in signal transduction pathways that govern a wide range of physiological processes including differentiation, proliferation, gene expression, brain function, membrane transport and the organization of cytoskeletal and extracellular matrix proteins. PKC isoforms are often overexpressed in disease states such as cancer. Adducin is a significant in vivo substrate for PKC or other PMA-activated kinases in a variety of cells, and phosphorylation of adducin occurs in dendritic spines that are believed to respond to external signals by changes in morphology and reorganization of cytoskeletal structures (Matsuoka et al., 1998). Therefore PKC-dependent phosphorylation of cytoskeletal substrate proteins, such as adducin, provides a mechanistic link between increased PKC activity and phenotypic changes in cytoskeletal-dependent processes such as migration and attachment, two processes that are relevant to metastatic potential. The reciprocal growth effects of expressing PKC delta or its regulatory domain as gain and loss of function constructs, respectively, have provided strong evidence that PKC delta regulates processes important for anchorage-independent growth in mammary tumour cell lines (Kiley et al., 1999).

Studies investigating the role of increased PKC activity in the promotion and progression of renal cancer were undertaken using normal and progressively transformed renal neoplasias from Eker rats. In normal proximal tubules, total adducin levels (measured with a phosphorylation state-insensitive antibody) were relatively high, whereas pSer660-adducin (measured with a phosphorylation state-sensitive antibody) levels were very low. In comparison, in renal carcinomas, total adducin levels were decreased, and pSer-660-adducin levels were increased. Changes in adducin expression levels, phosphorylation state, and localization parallelled the increased growth potential and loss of differentiation of the progressive tumour phenotypes (Fowler et al., 1998).

Rho-associated kinase (Rho-kinase), which is activated by the small guanosine triphosphatase Rho, phosphorylates alpha-adducin and thereby enhances the F-actin-binding activity of alpha-adducin in vitro. Studies have indicated that Rho-kinase phosphorylates alpha-adducin downstream of Rho in vivo, and that the phosphorylation of adducin by Rho-kinase plays a crucial role in the regulation of membrane ruffling and cell motility (Fukata et al., 1999). A model has also been proposed in which increased phosphorylation of alpha-adducin due to cisplatin leads to dissociation from the cytoskeleton, a situation rendered irreversible by caspase-3-mediated cleavage of alpha-adducin (van de Water et al., 2000).

ADD1 has not been identified as a SEREX antigen. The ADD1 gene is not on the Lymphochip, however gamma adducin is, and its mRNA expression is down-regulated in approximately half of the DLBCL cases.

Accession numbers: BC013393, AK025413, Z68280, L07261, S70314, XM_{—}055972

Ring Finger Protein 20: RNF20 (OX-TES-11)

RNF20 is a 975 aa uncharacterised protein belonging to the RING finger family on the basis of its C-terminal C3H4 type zinc finger motif (aa 937-946). Analysis of the RNF20 protein sequence using the PSORT II programme identified an R-2 mitochondrial presequence cleaveage site at aa 18, bipartite nuclear localisation signals at aa 498 and aa 689, a peroxisomal targeting signal at aa 433, a number of extensive coiled-coil regions, and predicted the likely localisation of this protein to be the nucleus.

The RING finger is a small zinc-binding domain found in many functionally distinct proteins. RING fingers are generally found close to an amino or carboxyl terminus and can also be associated with other domains to form larger conserved motifs, such as the RING finger-B box-alpha-helical coiled-coil (RBCC) motif. New studies have shown that the RING finger can specifically interact with E2 ubiquitin conjugating enzymes, thereby promoting ubiquitination, and that proteins which contain a ring finger may act as E3 ubiquitin protein ligases.

The RING finger domain has been shown to be essential for the function of the proteins in which they are found. There are a number of important members of this family including the TNF receptor-associated factor (TRAF) proteins which transduce signals from the tumour necrosis factor (TNF) receptor superfamily to the transcription factor NF-kB. BRCA1 is a breast cancer-susceptibility gene required for chromosomal stability and the biological response to DNA damage. The c-cbl proto-oncogene is a negative regulator of a number of protein-tyrosine kinases, including the ZAP-70/Syk tyrosine kinases that are critical for signalling in haematopoietic cells, and induces pre-B lymphomas and myeloid leukaemias in mice. Cbl becomes oncogenic in vivo as a result of mutation or deletion of the RING finger, and works normally by promoting polyubiquitination of receptor protein tyrosine kinase. Bmi1 is a protein that has been implicated as a collaborator of c-Myc in lymphomagenesis. The RING-finger promyelocytic leukaemia (PML) protein is the product of the PML gene that fuses with the retinoic acid receptor-alpha gene in the t(15;17) translocation of acute promyelocytic leukaemia. The proto-oncogene product MDM2 has a variant RING finger and is a critical regulator of p53.

There is now significant evidence supporting the involvement of a number of different, and functionally distinct, RING finger proteins in ubiquitin or ubiquitin-like pathways and events (reviewed in (Freemont, 2000)).

RNF20 maps to a chromosomal locus showing recurrent aberrations in DLBCL. It has been identified as a SEREX antigen but was not present on the Lymphochip.

Accession numbers: AK022300, AK022532, AK002051, NM_{—}019592, AF265230

KIAA0352 (OX-TES-7)

The 712 aa KIAA0352 gene product is uncharacterised. Analysis of the protein sequence using the PSORT II programme identified a potential cleavage site for mitochondrial pre-sequences at aa 14, a nuclear localisation signal at aa 577, both C- and N-terminal ER membrane retention signals, and five C-terminal zinc finger motifs at aa 374, 510, 540, 607, and 635. The protein is predicted to have a nuclear localisation. Analysis using MotifFinder found seven potential C2H2 zinc finger motifs and an N-terminal Broad-Complex, Tramtrack, and Bric a brac/poxvirus and zinc-finger (BTB/POZ) domain found in actin binding proteins or transcriptional regulators involved in chromatin modelling between aa 14-126.

The BTB domain is found primarily in Zinc finger proteins and defines an evolutionarily conserved family. The POZ domain is a conserved protein-protein interaction motif which mediates homo- or heterodimerisation. The BCL-6 gene product contains a BTB/POZ domain that has been identified as an autonomous repressing domain through interaction with N-COR and SMRT which are components of the histone deacetylase co-repressor complex. The POZ or BTB domain is also known as BR-C/Ttk or Zin.

The human B cell lymphoma proto-oncogene (BCL-6) and its family gene, BAZF, encode sequence-specific transcriptional repressors that contain the BTB/POZ domain in NH2-terminal region, and zinc finger motifs in COOH-terminal region. Other BTB/POZ domain proteins that have C-terminal zinc fingers include the DPZF protein which shares close similarity to BCL-6, with the highest similarity present in the BTB/POZ and zinc finger domains (Zhang et al., 2001), and Kaiso, which was cloned as a binding partner of p120(ctn) (Daniel and Reynolds, 1999). KIAA0352 may be an additional member of this gene family.

Deregulated BCL-6 expression caused by chromosomal rearrangements and point mutations of the BCL-6 promoter region are implicated in the pathogenesis of B-cell lymphoma (reviewed by (Staudt et al., 1999)). BAZF (encoding Bcl6-associated zinc finger protein) was found to be associated with BCL-6 at the BTB/POZ domain and localized in the nucleus. The zinc finger motifs of BAZF were 94% identical to those of BCL-6 at the amino acid level, and BAZF bound specifically to the DNA-binding sequence of BCL-6 and functioned as a transcriptional repressor. Expression of BAZF mRNA, like that of BCL-6 mRNA, was induced in activated lymphocytes as an immediate-early gene, although its tissue expression pattern was different (Okabe et al., 1998). Like BCL-6, the DPZF gene is localized on chromosome 3. It is widely expressed in haematopoietic tissues, including dendritic cells, monocytes, B cells, and T cells. DPZF protein expression is detectable in lymphoid neoplasms, especially B lymphoma. Therefore DPZF may also be involved in haematopoiesis, oncogenesis, and immune responses (Zhang et al., 2001).

Hypermethylated in cancer (HIC-1) is a new candidate tumour suppressor gene located in 17p13.3 that encodes a protein with five C2H2 zinc fingers and an N-terminal BTB/POZ domain. In the BCL-6 and promyelocityc leukaemia (PLZF) oncoproteins, this domain mediates transcriptional repression through its ability to recruit an HDAC complex. In contrast with BCL-6 and PLZF, HIC-1 did not interact with members of the HDAC complexes (SMRT/N-CoR, mSin3A or HDAC-1) in vivo or in vitro. In addition, a general and specific inhibitor of HDACs, trichostatin A, did not alleviate the HIC-1 mediated transcriptional repression, as previously shown for BCL-6. Therefore the recruitment onto target promoters of an HDAC complex is not a general property of transcriptional repressors containing a conserved BTB/POZ domain (Deltour et al., 1999).

KIAA0352 is uncharacterised and has not been identified as a SEREX antigen and was not on the Lymphochip. This data is the first to implicate this molecule as having a role either in DLBCL, or any other cancer.

Accession numbers: NM_{—}014830, AB002350, XM_{—}006763

Androgen Receptor Associated Coregulator 267: ARA267 (OX-TES-27)

The androgen receptor (AR) is a member of the steroid receptor superfamily that binds to the androgen response element to regulate target gene transcription. In addition to contacting the basal transcriptional machinery directly steroid receptors may inhibit or enhance transcription by recruiting an array of co-regulators. Numerous nuclear receptor (NR) coregulators have been identified, with diverse structures and potential mechanisms of coregulation, creating an increasingly complicated picture of NR action (reviewed in (Sampson et al., 2001)).

ARA267-alpha is a new AR coregulator containing 2427 aa, including one Su(var)3-9, Enhancer-of-zeste, and Trithorax (SET) domain (possible conserved function in relation to chromatin organization and function), two LXXLL motifs, three nuclear translocation signal (NLS) sequences, and four plant homeodomain (PHD) finger domains. PHDs with C4H3 motifs have been described in a number of proteins, some of which are known to be implicated in chromatin-mediated transcriptional regulation. ARA267-alpha mRNA is expressed predominantly in the lymph node as 13- and 10-kilobase transcripts and can enhance AR transactivation in prostate cancer cells (Wang et al., 2001). Interruption of the interaction between AR and these proteins may serve as a new therapeutic target in the treatment of prostate cancer.

The C-terminal half of the ARA267 protein is identical to that of the NSD1 protein. These genes are grouped in the same Unigene folder and map to the same human genome clone. In all likelihood these together with ARA267 a and b represent variants of the same gene.

The mouse NSD1 protein was originally identified through its interaction with the ligand binding domain of RARA (Huang et al., 1998). Recently, two publications have reported findings on the human NSD1 gene. The first report, of the novel gene NSD1 being implicated in human malignancy, was a result of the finding that this gene was a partner in the recurrent translocation t(5;11)(q35;p15.5) reported in childhood acute myeloid leukaemia, fusing NSD1 to the nucleoporin gene (NUP98) at 11p15.5 (Jaju et al., 2001). Another study reported that human NSD1 shows 86% identity with the mouse Nsdl at the nucleotide level, and 83% at the amino acid level. NSD1 is expressed in the fetal/adult brain, kidney, skeletal muscle, spleen, and the thymus, and faintly in the lung. Two different transcripts (9.0 and 10.0 kb) were consistently observed in various foetal and adult tissues examined. These findings favor the character of NSD1 as a nucleus-localized, basic transcriptional factor and also a bifunctional transcriptional regulator, such as that of the mouse Nsd1 (Kurotaki et al., 2001). Interestingly, the NSD3 gene, like NSD1 and NSD2 contains conserved SET and PHD domains. NSD3 has been found to be amplified in several tumour cell lines and primary breast carcinomas (Angrand et al., 2001).

The ARA267 gene has not been cloned as a SEREX antigen and was not present on the Lymphochip. NSD1 has been mapped to 5q35 a region that is associated with recurrent aberrations in DLBCL.

Accession numbers: AF322907, AF380302, AY049721, AK026066, AK025916, NM_{—}022455

MAPK6: ERK3, PRKM6 (OX-TES-15)

Mitogen-activated protein kinases (MAPKs) are serine-threonine protein kinases that are part of complex protein kinase cascades activated in response to a wide variety of extracellular stimuli, and they are encoded by a multigene family. There are several distinct classes of MAPKs, one of which includes the extracellular signal-regulated kinases ERK1, ERK2 and ERK3. Despite marked similarities to ERK1 and ERK2, ERK3 does not phosphorylate typical MAPK substrates, indicating that it has distinct functions (Cheng et al., 1996).

Tyrosine kinase growth factor receptors activate MAP kinase by a complex mechanism involving the SH2/3 protein Grb2, the exchange protein Sos, and Ras. The GTP-bound Ras protein binds to the Raf kinase and initiates a protein kinase cascade that leads to MAPK activation. Subsequently, the activated MAPK translocates into the nucleus where many of the physiological targets of the MAPK signal transduction pathway are located. These substrates include transcription factors that are regulated by MAPK phosphorylation (e.g., Elk-1, c-Myc, c-Jun, c-Fos, and C/EBP beta). Thus, the MAPK pathway represents a significant mechanism of signal transduction by growth factor receptors from the cell surface to the nucleus that results in the regulation of gene expression (Davis, 1995).

MAPK6 has not been identified as a SEREX antigen, and although the gene is present on the Lymphochip, its mRNA expression is only reduced in two cases of DLBCL; otherwise there is no real expression difference in the cases of DLBCL studied. A recent study on DLBCL gene expression profiling reported that genes involved in serine/threonine phosphorylation pathways were implicated in DLBCL outcome (Shipp et al., 2002) making MAPK6 a prime candidate for involvement in DLBCL.

Accession numbers: AF420474, NM_{—}002748, L77964, X80692, XM_{—}035575

ZHX1 (OX-TES-8)

NF—YA, B, and C comprise the heterotrimeric transcription factor known as nuclear factor Y (NF—Y) or CCAAT-binding protein (CBF). NF—Y binds many CCAAT and Y box (an inverted CCAAT box, ATTGG) elements. Mutations of these elements that disrupt the binding of NF—Y result in decreased transcription from various tissue-specific and inducible promoters. A yeast two-hybrid system was employed to isolate proteins that interacted with NF—Y and thus might play a role in tissue-specific or hormone-inducible promoter activity. One such interacting protein was encoded by a novel gene, ZHX1, and contains two zinc fingers and five homeodomain motifs. Northern blot analysis of poly(A)⁺ RNA isolated from various tissues revealed two major ZHX1 transcripts. Both transcripts were expressed ubiquitously, although the 5-kilobase transcript was of greater abundance in most tissues examined. The human ZHX1 gene is located on chromosome 8q, between markers CHCL.GATA50B06 and CHLC. GATA7G07 (Yamada et al., 1999).

As a result of differential splicing, one subunit of NF—Y consists of two major isoforms designated short (NF—YaS) and long (NF—YaL). In proliferating normal human fibroblasts, NF—YaL is by far the more abundant isoform. Surprisingly, NF—YaS was found by immunoblotting to be as prominent as NF—YaL in simian virus 40 (SV40)-transformed cell derivatives. As a consequence, two NF—Y/DNA complexes, one containing the long and the other the short isoform, were formed with extracts from transformed cells and a target promoter element in electrophoretic mobility-shift assays. Only the complex containing NF—YaL was detected with extracts from normal fibroblasts. Furthermore, the NF—Y recognition motif contributed to promoter activation in SV40-transformed cells but not in normal cells. This finding links transcription stimulation in transformed cells to quantitative changes in the expression of an NF—Ya subunit (Gu et al., 1999).

Abnormally low levels of the cyclin-dependent kinase inhibitor p27Kip1 are found frequently in human carcinomas, and these levels correlate directly with both histological aggressiveness and patient mortality. p27Kip1 is haplo-insufficient for tumour suppression and thus may be a useful molecule for the development of cancer therapies. p27Kip1 promoter analysis using 5′-deletion mutants revealed that a 39-bp region between −549 and −511 was required for maximal promoter activity. Point mutation analysis revealed that a CCAAT box within this region was essential for promoter activity. Gel shift assays and cotransfection experiments using a dominant negative form of the NF—Y transcription factor showed that NF—Y directly regulates p27Kip1 transcription through this CCAAT box (Kamiyama et al., 1999).

The p53 tumour suppressor protein regulates the transcription of regulatory genes involved in cell cycle arrest and apoptosis. Transient transfection analysis showed that wild type p53 represses cdc2 whereas various dominant negative mutants of p53 increase its transcription. Since the cdc2 promoter did not contain a TATA sequence, deletion and point mutation analyses were performed and these identified the inverted CCAAT sequence located at -76 as a cis-acting element for the p53-mediated regulation. A specific DNA-protein complex was formed at the CCAAT sequence and this complex contained the NF—Y transcription factor. Consistently, a dominant negative mutant of the NF—YA subunit, NF—YAm29, decreased the cdc2 promoter, and p53 did not further decrease the promoter activity in the presence of NF—YAm29. These results suggest that p53 negatively regulates cdc2 transcription and that the NF—Y transcription factor is required for the p53-mediated regulation (Yun et al., 1999). Recent studies have identified two p53 homologues, p63 and p73. They activate p53-responsive promoters and induce apoptosis when overexpressed in certain human tumours. The DNA binding activity of the NF—Y transcription factor, which is essential for transcription of the cdk1 and cyclin B genes and inactivated in senescent fibroblasts, is significantly decreased by expression of either p53, p63, or p73. Since NF—Y binds to many promoters besides the cdk1 and cyclin B promoters, inactivation of NF—Y by p53 family genes may be a general mechanism for transcription repression in replicative senescence (Jung et al., 2001).

The multidrug resistance gene (MDR1) promoter is subject to control by various internal and external stimuli. The CCAAT box-binding protein, NF—Y, mediates MDR1 activation by the histone deacetylase inhibitors, trichostatin A and sodium butyrate, through the recruitment of the co-activator, P/CAF. Activation of the MDR1 promoter by UV irradiation is also dependent on the CCAAT box (−82 to −73) as well as a proximal GC element (−56 to −42). Gel shift and supershift analyses with nuclear extracts prepared from human KB-3-1 cells identified NF—Y as the transcription factor interacting with the CCAAT box, while Sp1 was the predominant factor binding to the GC element. Mutations that prevented binding of either of these factors reduced or abolished activation by ultraviolet irradiation; moreover, co-expression of a dominant-negative NF—Y protein (NF—YA29) reduced UV-activated transcription. Interestingly, YB-1, a transcription factor that also recognizes the CCAAT motif and had been reported to mediate induction of the MDR1 promoter by ultraviolet light, was incapable of interacting with the double-stranded MDR1 CCAAT box oligonucleotide in nuclear extracts, although it dfd interact with a single-stranded oligonucleotide. Furthermore, a mutation that abolished activation of MDR1 by UV-irradiation had no effect on YB-1 binding, and co-transfection of a YB-1 expression plasmid had a repressive effect on UV-inducible transcription. Taken together, these results indicate a role for both NF—Y and Sp1 in the transcriptional activation of the MDR1 gene by genotoxic stress, and indicate that YB-1, if involved, is not sufficient to mediate this activation (Hu et al., 2000).

Thus because of its interaction with NF—Y, ZHX1 is implicated in regulation of cell cycle and multidrug resistance although there is little published data on this protein. ZHX1 has been identified as a SEREX antigen but is not on the Lymphochip. The gene maps to 8p22 a chromosome region with an exceptionally high number of reported aberrations in DLBCL and FL.

Accession numbers: NM_{—}007222, AF195766, AF106862, AK025236

Ran Binding Protein 2, RanBP2: Nup358 (OX-TES-6)

The partial protein encoded by TA204 does not appear to have been previously described. This protein is extremely similar to RanBP2 and both genes map to chromosome 2. Given the similarity between the proteins it is possible that serum from patients with DLBCL will also recognise RanBP2 and that both proteins will have a similar function.

Ran is a small Ras-related GTPase that has been implicated in a variety of nuclear processes, including the maintainance of nuclear structure, protein import, mRNA processing and export, and cell cycle regulation. There has been significant progress in determining the role of Ran in nuclear protein import. However, it has been unclear whether this role is sufficient to account for the diverse effects of disrupting Ran functions. Recently, several proteins have been identified that bind specifically to Ran and are, therefore, possible effectors. Proteins that regulate the GTPase cycle and subcellular distribution of Ran include the cytoplasmic GTPase-activating protein (RanGAP) and its co-factors (RanBP1, RanBP2), the nuclear guanine nucleotide exchange factor (RanGEF), and the Ran import receptor (NTF2).

Ran has also been implicated in nuclear transport. By screening a HeLa cell lambda expression library with Ran-GTP, a novel protein termed Nup358 (for nucleoporin of 358 kDa) was identified. Nup358 contained a leucine-rich region, four potential Ran binding sites (i.e. Ran binding protein 1 homologous domains) flanked by nucleoporin-characteristic FXFG or FG repeats, eight zinc finger motifs, and a C-terminal cyclophilin A homologous domain. Consistent with the location of Nup358 at the cytoplasmic fibers of the nuclear pore complex (NPC), decoration with Ran-gold was found only at the cytoplasmic side of the NPC. Thus, Nup358 was the first nucleoporin shown to contain binding sites for two of three soluble nuclear transport factors isolated at that time, namely karyopherin and Ran-GTP (Wu et al., 1995).

There are no orthologues of the RanBP2 gene in yeast and Drosophila genomes. In humans, this bona fide gene is partially duplicated in a RanBP2 gene cluster and lies in a hot spot for recombination on chromosome 2q. This genetic heterogeneity renders further significance of this genomic region in human disease due to its possible involvement in genetically linked disorders such as juvenile nephronophthisis, congenital hepatic fibrosis, and chorioretinal dysplasia.

During mitosis in higher eukaryotic cells, the nuclear envelope membranes break down into distinct populations of vesicles, and the proteins of the nuclear lamina and the NPCs disperse in the cytoplasm. Phosphorylation can alter protein-protein interactions and membrane traffic, and Nup358 was phosphorylated throughout the cell cycle and hyperphosphorylated during M phase. Several nuclear pore complex proteins are differentially phosphorylated during mitosis when pore complexes disassemble and reassemble (Favreau et al., 1996).

The combination of the Ran-binding domain 4 and cyclophilin domains of Ran-binding protein 2 selectively associate with a subset of G protein-coupled receptors. Another domain of Ran-binding protein 2, the cyclophilin-like domain, specifically associates with the 112-kDa subunit, P112, and other subunits of the 19 S regulatory complex of the 26 S proteasome in the neuroretina. Also, the interaction of Ran-binding protein 2 with P112 regulatory subunit of the 26 S proteasome involves yet another protein, a putative kinesin-like protein. Ran-binding protein 2 is a key component of a macro-assembly complex, selectively linking protein biogenesis with the proteasome pathway and, thus, with potential implications for the presentation of misfolded and ubiquitin-like modified proteins to this proteolytic machinery (Ferreira et al., 1998).

In eukaryotic cells, both soluble transport factors and components of the NPC mediate protein and RNA trafficking between the nucleus and the cytoplasm. Four nucleoporins, Nup153, RanBP2, Nup214 and Tpr are cleaved by caspases during apoptosis. Thus the apoptotic programme includes modifications in the machinery responsible for nucleocytoplasmic transport, which are independent from caspase-mediated degradation of nuclear proteins (Ferrando-May et al., 2001).

Small ubiquitin-like modifier (SUMO) belongs to a growing number of ubiquitin-like proteins that covalently modify their target proteins. Although SUMO modification may have a role in regulating protein stability, it is most likely that SUMO alters the interaction properties of its targets, often affecting their subcellular localization behaviour. Examination of the PML nuclear bodies, whose principal components are SUMO-modified, has revealed this modification to be essential for their structural and functional integrity. Thus SUMO is thought to regulate the stability not of individual proteins, but rather that of entire multiprotein complexes. SUMOylation requires the E1 enzyme, Aos1/Uba2, and the E2 enzyme, Ubc9. Ubc9, a conjugation enzyme for the ubiquitin-related modifier SUMO, is present predominantly in the nucleus and at the NPC. A PEST element, containing129 amino acid residues, designated IR1+2, on the human nucleoporin RanBP2/Nup358 binds directly to Ubc9 with high affinity both in vitro and in vivo. When IR1+2 tagged with green fluorescent protein was transfected into COS-7 cells, ˜90% of the nuclear Ubc9 was sequestered in the cytoplasm. Both SUMO-1 and SUMO-2/3 were mislocalised, and PML protein formed an enlarged aggregate in the nucleus. Moreover, the homologous recombination protein, Rad51, mislocalized to the cytoplasm and Rad51-foci, a hallmark of functional association of Rad51 with damaged DNA, did not form efficiently, even in the presence of a DNA strand-breaker (Saitoh et al., 2001).

RanBP2/Nup358 also has SUMO-1 E3-like activity. RanBP2 directly interacts with the E2 enzyme Ubc9 and strongly enhances SUMO-1-transfer from Ubc9 to the SUMO-1 target Sp100. The E3-like activity is contained within a 33 kDa domain of RanBP2 that lacks RING finger motifs and does not resemble PIAS family (E3-like factors) proteins. These findings place SUMOylation at the cytoplasmic filaments of the NPC, and suggest that, at least for some substrates, modification and nuclear import are linked events (Pichler et al., 2002).

These data implicate RanBP2 in a wide range of biologically important functions. Its association with Rad51 raises the possibility that expression of RanBP2 is associated with DNA repair and possibly the somatic hypermutation in DLBCL. The related gene that we have identified has not been previously characterised. The RanBP2 protein itself, however, has been previously identified as a SEREX antigen and the Lymphochip data indicates that there is some low level differential mRNA expression of this gene in cases of DLBCL.

The sequence encoded by TA204 is most similar to a hypothetical gene (XM_{—}016176) both of which are highly related to RanBP2. Both our sequence and the XM_{—}016176 sequence map (and are identical to) to human genome sequence on chromsome 2 (NT_{—}029235). Human RanBP2 also maps to chromsome 2 but is encoded by the sequences in NT_{—}005224 and NT_{—}029895. RanBP2 and the protein product from TA204 are slightly different, and represent highly-related proteins, raising the possibility that either our protein or RanBP2 may be recognised by the serum from patients with DLBCL.

Accession numbers: XM_{—}016176.1,

Accession numbers: RANBP2; NM_{—}006267, D42063, L41840, AK025711, AK025462, AK026993.

FLJ31673 (OX-TES-4)

This antigen is encoded by a previously uncharacterised gene, FLJ31673. One cDNA clone represented a new 5′ splice variant of this molecule which encoded an N-terminally truncated protein. Analysis of the predicted 760 aa FLJ31673 protein sequence using the PSORT II program identified potential nuclear localisation sites at aa 395/398 and aa 282, a peroxisomal targeting signal at aa 682, a C-terminal coiled-coil at aa 584-701, and predicted that the protein was likely to have a nuclear localisation. No significant domains were identified using MotifFinder.

This molecule has been identified as a SEREX antigen but was not on the Lymphochip.

The cDNA sequence from TA68 showed no similarity to FLJ31673 using BLAST searches but mapped to the same genomic clone (AL158821). Subsequently, a 25bp overlap was identified between TA68 and TA171 (which did correspond bp of the FLJ31673 cDNA sequence and differed from this sequence at nt 219 (AK056235), where this sequence matches the human genome sequence and has an A, while the FLJ31673 (AK056235) sequence has a T. Alignment to the human genome sequence indicated that nt 715 of our sequence represented an intron-exon boundary. Our cDNA sequence therefore identified four additional 5′ exons of the FLJ31673 gene.

The sequence of the predicted protein product of this extended cDNA sequence lacked the first 50 aa predicted for the FLJ31673 protein due to the presence of an upstream stop codon. The first methionine codon after this stop corresponds to aa 76 of the FLJ31673 protein sequence. An additional change in the aa Y in place of the H found at aa 130 of the FLJ31673 protein sequence was observed.

Accession number: AK056235 (FLJ31673)

OX-TES-4 is a cDNA of ˜4.2kb that encodes a predicted protein (FIG. 15). OX-TES-4 has not been sequenced fully, but the data obtained shows a very high similarity to FLJ31673, an uncharacterised cDNA that encodes a protein of 760aa (shown in FIG. 16). Analysis of FLJ31673 predicts a molecular weight of 87.5kDa and a pI of 6.19. This analysis also identified a number of domains highlighted in FIG. 15—a signal peptide, a putative peptidase Ml family domain, nuclear localisation signals, a peroxisomal targeting signal and a coiled-coil domain. There is a known murine homologue of FLJ31673 that shows 77% similarity over 151 aa, and there are predicted homologues in cow (Bos taurus), pig (Sus scrofa) and Norway rat (Rattus norvegicus) based upon EST data. FLJ31673 and OX-TES-4 map to chromosome Xq22.3-23, a locus where there are few known aberrations in DLBCL. FIG. 17 shows an alignment of the DNA sequences of OX-TES-4 and FLJ31673, illustrating that OX-TES-4 only matches FLJ31673 from nt562 onwards. The upstream DNA sequence of OX-TES-4 maps to a different region of the chromosome, which suggests that OX-TES-4 might be a splice variant of FLJ31673. Although there is additional 5′ sequence in OX-TES-4, it appears to encode a truncated version of the predicted FLJ31673 (FIG. 18). There is no known or suggested function for FLJ31673.

Expression of the OX-TES-4 Gene in Normal and Neoplastic Human Tissues.

The inventors have characterised the expression of the OX-TES-4 mRNA in both normal human tissues and in matched normal and tumour tissues from cancer patients to investigate whether differential expression of the OX-TES-4 mRNA occurs in human cancer.

An 830bp EcoRI fragment of OX-TES-4 (nt1684 to 2473) encoding aa 353-630 of FLJ31673, was used to probe the Multiple Tissue Expression (MTE) and Matched Tumour/Normal (MTN) arrays (BD Biosciences), as detailed for OX-TES-1.

Normal Tissues

The normal tissues on the MTE array come from non-diseased victims of sudden death/trauma and are pooled from a number of individuals. OX-TES-4 mRNA in normal human tissues showed a widespread expression (FIG. 19). The strongest expression was observed in placenta, testis, adrenal glands, uterus and a lung carcinoma cell line (in order of decreasing expression). There are lower levels of expression of OX-TES-4 mRNA in the brain in comparison with gastrointestinal, heart and foetal tissues. OX-TES-4 mRNA is widely expressed, although at even more reduced levels, in lymphoid tissues, whilst the leukaemia cDNAs show the lowest (almost negative) expression levels. No positive signals were observed with any of the control samples, indicating that all binding is specific and genuine.

Neoplastic Tissues

Analysis of OX-TES-4 mRNA expression in human tumour tissues showed there to be almost no expression in any tumour or adjacent histologically-normal tissues from any source with the exception of the lung adenocarcinoma cell line (FIG. 20, P6), also seen on the MTE array (FIG. 19), a prostate tumour and a stomach tumour (FIG. 20).

In conclusion, it can be inferred that OX-TES-4 is widely expressed in normal human tissues, with the strongest expression in placenta, testis, adrenal glands and uterus. Although the definition of a CTA is based on its expression in normal testis and human malignancies, some CTAs are occasionally expressed in placenta and the female reproductive organs whilst others show ubiquitous expression in normal tissues (Zendman et al., 2003). The majority of CTAs map to chromosome X, as OX-TES-4 does, therefore OX-TES-4 may also be a CTA that is expressed in DLBCL.

Expression of the OX-TES-4 Gene in DLBCL Cell Lines

The inventors have characterised the expression of the OX-TES-4 mRNA in a number of DLBCL cell lines to investigate whether OX-TES-4 expression occurs.

The protocol used was as described for OX-TES-1. Gene specific primers were designed to amplify a fragment of 685 bp, and the sequences are shown in Table 2.

Expression of OX-TES-4 in DLBCL Cell Lines

Analysis of OX-TES-4 mRNA in DLBCL-derived cell lines successfully amplified a fragment, in all cell lines tested, of the same size as the positive control (685 bp; see FIG. 21a). No product is observed with the negative control. This suggests that OX-TES-4 is transcribed in DLBCL cell lines. Interestingly, the primary PCR shows a fragment of fainter intensity for SUDHL6 compared to the remaining cell lines (FIG. 21A). Although the PCR is not quantitative, this result suggests that there may be lower levels of expression of OX-TES-4 in germinal centre-compared to activated-DLBCL. After a second round of PCR, the difference in intensities is no longer observed (FIG. 21b). This result is reproducible and is independent of whether the cDNA is oligo(dT)- or hexamer-primed. FIG. 21C shows the products obtained when primers for β-actin are used on these cDNAs. Strong bands are obtained with all cell lines, which implies that the cDNAs are all suitable for PCR and will give comparable products where expression is equal, adding further support to the suggestion of genuine differential expression of OX-TES-4 mRNA. This result suggests that OX-TES-4 may be a suitable reagent for subtyping DLBCL.

Additional germinal center DLBCL cell lines (SUDHL10 and DB) were analysed for OX-TES-4 mRNA expression using RT-PCR as described above. No PCR product was observed in these two cell lines providing additional support for the utility of OX-TES-1 expression levels in subtyping DLBCL. Further work is underway to quantitate this differential expression.

Protein Phosphatase 1A (formerly 2C) Alpha Isoform: PPM1A, PP2CA, PP2C-Alpha (OX-TES-3)

Protein phosphorylation and dephosphorylation are key regulatory mechanisms in many cellular processes. Four major classes of serine/threonine protein phosphatases have been described: PP1, PP2A, PP2B, and PP2C. These proteins differ in their substrate specificity, divalent cation requirements, and sensitivity to inhibitors. PP1, PP2A, and PP2B phosphatases have catalytic subunits with significant sequence similarity, and regulatory subunits that are believed to regulate activity or cellular localization. In contrast, PP2C phosphatases are monomeric and have amino acid sequences that are distinct from those of the other phosphatases.

MAPK (mitogen-activated protein kinase) cascades are common eukaryotic signalling modules that consist of a MAPK, a MAPK kinase (MAPKK) and a MAPKK kinase (MAPKKK). Because phosphorylation is essential for the activation of both MAPKKs and MAPKs, protein phosphatases are likely to be important regulators of signalling through MAPK cascades. The prototypic MAPKs, ERKs, are activated by mitogenic stimulation while two other types of MAPKs, namely JNK (SAPK) and p38 are activated by environmental stresses such as osmotic shock, UV irradiation, heat shock, wound stress and inflammatory factors. JNK is activated by SEK1 or MKK7 while p38 is activated by either MKK3 or MKK6.

Human PPM1A was cloned using a rat PP2C-alpha gene as the probe (Mann et al., 1992). PP2C-alpha was also later identified in a genetic screen to identify protein phosphatases that negatively regulate the p38 and JNK stress-activated MAPK cascades (Takekawa et al., 1998). There were 2 forms of the protein, which were termed PP2C-alpha-1 (382 amino acids) and PP2C-alpha-2 (324 amino acids), and they differed at the C terminus. Using immunohistochemical analysis PPM1A was detected in both the cytoplasm and nucleus of mammalian cells, consistent with a role in dephosphorylating components of stress-activated pathways (Das et al., 1996). Using monoclonal antibodies against the recombinant PP2Calpha the immunoreactivity of normal human tissues was evaluated. The reactivity was strong in normal skin, the digestive tract, lung, kidney, breast, prostate, endocrine glands, and brain, while it was moderate in the ovary, testis, and liver. Epithelial cells revealed high levels of PP2Calpha expression, but stromal cells, including fibroblasts and endothelial cells, showed little or no PP2Calpha expression. Given the broad reactivity in endocrine and secreting epithelial cells, PP2Calpha expression might contribute to secretory cell function (Lifschitz-Mercer et al., 2001).

Cystic fibrosis is an important genetic disease which results from mutations in the cystic fibrosis transmembrane conductance regulator (CFTR). cAMP-dependent phosphorylation activates CFTR in epithelia. In airway and colonic epithelia, neither okadaic acid nor FK506 prevented inactivation of CFTR when cAMP was removed. These results suggested that a phosphatase, distinct from PP1, PP2A, and PP2B, was responsible. The results of this study suggested that PP2C-alpha may be the okadaic acid-insensitive phosphatase that regulates CFTR in human airway and T84 colonic epithelia (Travis et al., 1997).

It has been demonstrated that PPM1A inhibits the activation of the stress-responsive p38 and JNK MAPK cascades. In vivo and in vitro observations indicated that PPM1A dephosphorylates and inactivates MAPKKs (MKK6), SEK1 and a MAPK (p38) in the stress-responsive MAPK cascades. Using coimmunoprecipitation assays, PPM1A was shown to directly interact with p38 (Takekawa et al., 1998). PP2C selectively inhibits the SAPK pathways through suppression of MKK3b, MKK4, MKK6b and MKK7 activities in mammalian cells (Hanada et al., 1998).

The activating phosphorylation on cyclin-dependent kinases in yeast (Cdc28p) and in humans (Cdk2) is removed by type 2C protein phosphatases. This activity is due to PP2C beta 2, a novel PP2C beta isoform, and to PP2C alpha, and purified recombinant PP2C alpha and PP2C beta 2 proteins efficiently dephosphorylated monomeric Cdk2/Cdk6 in vitro. The dephosphorylation of Cdk2 and Cdk6 by PP2C isoforms was inhibited by the binding of cyclins. The PP2C-like activity in HeLa cell extracts, partially purified HeLa PP2C alpha and PP2C beta 2 isoforms, and the recombinant PP2Cs exhibited a comparable substrate preference for a phosphothreonine containing substrate, consistent with the conservation of threonine residues at the site of activating phosphorylation in CDKs (Cheng et al., 2000).

PPM1A alpha has not been identified as a SEREX antigen and although one EST is present on the Lymphochip, we were unable to access this profile. A recent study on DLBCL gene expression profiling reported that genes involved in serine/threonine phosphorylation pathways were implicated in DLBCL outcome (Shipp et al., 2002), making PPM1A a prime candidate for involvement in DLBCL.

This cDNA clone is a 5′ splice variant of the other PPM1A cDNA clones in the public databases. The first 278 bp do not match the PPM1A sequences although the PPM1A sequences do have an additional 337 bp before the homologous region. Searches against the human genome sequence confirm that our 5′ sequence comes from an adjacent region on chromosome 14, and that nt 279 is the beginning of the 4^{th} exon. These changes are not predicted to change the protein sequence but may affect gene regulation.

Accession numbers: NM_{—}021003, AF070670, S87759

VAT1 Membrane Protein of Cholinergic Synaptic Vesicles (OX-TES-9)

Synaptic vesicles are responsible for regulating the storage and release of neurotransmitters in the nerve terminal. Expression screening of a Pacific electric ray (Torpedo californica) lobe library identified a cDNA encoding a 379-amino acid synaptic vesicle integral membrane protein, which was termed VAT1. Northern blot analysis revealed that a 5.8-kb transcript was expressed in electromotor neurons. Western blot analysis determined expression of a 42-kD protein in the electric organ that copurified with synaptic vesicles (Linial et al., 1989). VAT-1 was expressed in E. coli and the product was purified and analyzed. The protein binds specifically to an ATP column and displays an ATPase activity as measured by the kinetics of [³²P] phosphate release. The activity is dependent on divalent ions, with both Mg²⁺ and Ca²⁺ supporting the reaction. This ATPase activity is not affected by known inhibitors of the vesicular V- and P-type ATPases such as vanadate and N-ethylmaleimide. It has been suggested that VAT-1 activity may affect ATP-dependent reactions in Torpedo nerve terminals, such as phosphorylation and dephosphorylation of proteins (Linial and Levius, 1993).

While attempting to identify the BRCA1 gene, cDNAs for a number of genes on chromosome 17q21 were cloned, including one with significant homology to Torpedo VAT1 (Friedman et al., 1995). By random sequencing of 4 cosmids from a human chromosome 17-specific library, VAT1, was sequenced. The coding sequence of VAT1 predicts a 301-amino acid peptide and a CpG island precedes the VAT1 gene, which contains 6 exons spanning 8.1 kb (Smith et al., 1996).

A VAT-1 homologue has also been isolated from a murine breast cancer cell line (Ehrlich ascites tumour) and identified by sequencing of cleavage peptides. The isolated VAT-1 homologous protein displayed an ATPase activity and exists in two isoforms, with isoelectric points of 5.7 and 5.8. The known part of the murine and the human translated sequences share 97% identity. The size of the VAT-1 homologue mRNA in both murine and human (T47D) breast cancer cells was determined to be 2.8 kb. A modified gene structure of the human VAT-1 homologue with an extended exon 1 has been proposed. VAT-1 and the mammalian VAT-1 homologue form a subgroup within the protein superfamily of medium-chain dehydrogenases/reductases (Hayess et al., 1998). The VAT-1 gene was not included on the Lymphochip and has not previously been reported as a SEREX antigen in the SEREX database.

Accession numbers: AK000237, BC001913, BC008725, BC015041, U25779, BC014279, U18009, NM_{—}006373, L78833

BRCAL Associated Protein 1, BAP1 (OX-TES-28)

Inheritance of one defective copy of either of the two breast-cancer-susceptibility genes, BRCA1 and BRCA2, predisposes individuals to breast, ovarian and other cancers. Both genes encode very large protein products; these bear little resemblance to one another or to other known proteins, and their precise biological functions remain uncertain. Recent studies reveal that the BRCA proteins are required for maintenance of chromosomal stability in mammalian cells, and function in the biological response to DNA damage. The new work suggests that, although the phenotypic consequences of their disruption are similar, BRCA1 and BRCA2 play distinct roles in the mechanisms that lead to the repair of DNA double-strand breaks (reviewed by (Venkitaraman, 2001)). BAP1 is a protein that was identified through its binding to the RING finger domain of BRCA1. BAP1 is a nuclear-localized, ubiquitin carboxy-terminal hydrolase, suggesting that deubiquitinating enzymes may play a role in BRCA1 function. BAP1 binds to the wild-type BRCA1-RING finger, but not to germline mutants of the BRCA1 RING finger found in breast cancer kindreds. BAP1 and BRCA1 are temporally and spatially co-expressed during murine breast development and remodelling, and show overlapping patterns of subnuclear distribution. BAP1 resides on human chromosome 3p21.3; intragenic homozygous rearrangements and deletions of BAP1 have been found in lung carcinoma cell lines. BAP1 enhances BRCA1-mediated inhibition of breast cancer cell growth and is the first nuclear-localized ubiquitin carboxy-terminal hydrolase to be identified. BAP1 may be a new tumour suppressor gene which functions in the BRCA1 growth control pathway (Jensen et al., 1998).

Dr F. J. Rauscher 3^{rd} presented additional work on BAP1 as an oral communication at the Cancer Prevention and Detection meeting in Geneva (28.10.00). His further studies found that a loss of BAP1 led to loss of DNA repair facilities. BRCA1 may recruit proteins to damage in active genes (transcription coupled repair) and this group believe that BAP1 turns this system off. BAP1 null cells were reported to be deficient in DNA repair, and tumour-derived mutations in BAP1 abolished both ubiqutin hydrolase and transcription coupled repair functions. There was no change in BRCA1 levels in BAP1 transfectants, therefore its substrate was probably not BRCA1.

These data, together with the the reported somatic hypermutation in DLBCL which occurs in transcriptionally active genes, makes BAP1 an important candidate gene for disrupting the repair of damaged genes in DLBCL. This gene is not in the SEREX database or on the Lymphochip.

Accession numbers: D87462, NM_{—}004656, AF045581, BC001596, D88812, AB002534, XM_{—}037408

Accession numbers: D87462, NM_{—}004656, AF045581, BC001596, D88812, AB002534, XM_{—}037408

Selection of Patients' Serum for Testis Library Screening Using the SEREX Technique.

SEREX screening of a cDNA library requires approximately 20 ml of serum from a DLBCL patient. Because this volume of serum required a blood sample to be taken, that was not part of routine monitoring or treatment, ethical permission was required to obtain the serum sample (ethical permission was obtained for this project), as was the informed consent of the subject. Sufficient volumes of serum were obtained from three DLBCL patients attending Dr Chris Hatton's Lymphoma Clinic at the John Radcliffe Hospital in Oxford. All three sera were cleaned using the serum cleaning protocol described in the SEREX methodology.

Patients' serum samples, both before and after cleaning, were used to immunostain DLBCL derived cell lines (HLY-1, Mieu, Lib, SUDHL6). Cytocentrifuge preparations of the DLBCL cell lines were stained using an immunoperoxidase technique, as described previously to detect antibodies to NPM-ALK in plasma samples from patients with ALCL (Pulford et al, 2000). The serum sample selected showed immunoreactivity with the DLBCL cell lines, both before and after cleaning, confirming that antibodies to proteins expressed by this lymphoma were present in the serum sample used to screen the testis cDNA library (FIG. 25). Each panel in the figure is labelled with the cell line that was immunostained, and the dilution of human serum used (“cleaned” represents staining using cleaned serum). FIG. 26 illustrates the immunostaining data of one of the additional DLBCL sera that was used in the tertiary screening process. This demonstrates that this serum also has reactivity on DLBCL cell lines, even after being cleaned.

Results from Screening DLBCL Antigens with Sera from Additional Patients.

The 28 testis antigens identified by the inventors were those proteins that were immunologically recognised by the serum from a single patient with DLBCL. To assess the relevance of these antigens in DLBCL, screening with serum from increased numbers of patients and from control subjects was required. To assess the frequency with which these proteins were recognised by DLBCL patients' all 94 clones were screened with serum from an additional 4 DLBCL patients, and with serum from 5 age- and sex-matched control subjects (provided by the Blood Transfusion Service). The age- and sex-matching is important as the immune response varies with age and sex, for example women over 40-50 naturally have a higher titre of autoantibodies in their serum. This additional information has provided an indication of the tumour specificity of the immune response to these antigens, and a summary of the data is presented below.

1. Antigens Specifically Recognised by Multiple DLBCL Patients' Sera

| | |
|---|---|
| FLJ31673 | 4 patients and no normals |
| PPlAa | 3 patients and no normals. The mRNA |
| | encoding this gene is |
| | differentially expressed in DLBCL |
| | (Lymphochip data). |
| Histone H3.3B | 3 patients and no normals |
| Novel PAS protein | 3 patients and no normals |
| RANBP2 | 5 patients and no normals. Some low |
| | level differential expression of |
| | this mRNA was observed in both FL |
| | and DLBCL (Lymphochip data). |

2. Patient Specific Antigens Recognised Only by Serum Used for Library Screening

| | |
|---|---|
| Bacl | 1 patient and no normals |
| KIAA0352 | 1 patient and no normals |
| ZHX1 | 1 patient and no normals |
| VAT1 | 1 patient and no normals |

3. Antigens Recognised with a Higher Frequency by Patients' Sera

| | |
|---|---|
| SRPK1 | 4 patients and 1 normal. The mRNA |
| | encoding this gene is differentially |
| | expressed in DLBCL (Lymphochip data). |
| RNF2O | 3 patients and 1 normal |
| HnRNP A2/B1 | 4 patients and 1 normal |
| STK11 | 5 patients and 2 normals. The mRNA |
| | encoding this gene shows increased |
| | expression in DLBCL compared to FL and |
| | there was some differential expression |
| | within DLBCL (Lymphochip data). |
| HIS1 | 5 patients and 2 normals |
| MAPK6 | 3 patients and 1 normal. The mRNA |
| | encoding this gene shows higher |
| | expression in DLBCL compared to FL. There |
| | is some differential expression within |
| | DLBCL (Lymphochip data). |

4. Antigens Recognised by Both Patients' and Normal Sera

| | |
|---|---|
| CBF1 | 4 patients and 3 normals. The mRNA |
| | expression of this gene is significantly |
| | higher in the majority of DLBCL patients |
| | compared to FL patients. Within the DLBCL |
| | cases on the Lymphochip there is |
| | differential expression of this gene. |
| MacroHistone H2A | 1 patient and 2. normal |
| KIAA0643 | 4 patients and 5 normals |
| ERCC3 | 2 patients and 1 normal. The mRNA |
| | encoding this gene is expressed in |
| | DLBCL but no differential expression |
| | was observed (Lymphochip data). |
| GKAP42 | 4 patients and 2 normals |
| Sirtuin | 2 patients and 1 normal |
| PSP1 | 2 patients and 1 normal |
| YB1 | 5 patients and 4 normals |
| TZP | 5 patients and 4 normals |
| FLJ13942 | 5 patients and 3 normals |
| Adducin 1a | 4 patients and 3 normals |
| ARA267b | 2 patients and 2 normals |
| BAP1 | 2 patients and 2 normals |

The antigens are divided into four groups based on the frequency at which antibodies against the proteins were detected in either DLBCL patients' or control sera. Group one contains 5 antigens that are recognised by serum from more than one patient (in all cases the majority of DLBCL patients contain antibodies to these proteins) and none of the control sera. This is the most significant group at this stage of the analysis. Two of these genes are already known from the Lymphochip data to be expressed in DLBCL, confirming that genes cloned from the testis library are expressed in DLBCL patients. These antigens are potentially immunologically tumour-specific, and may represent good candidates for immunotherapy.

Group 2 contains 4 antigens that are specifically recognised only by the serum that was used to screen the testis library. Further studies are required with larger numbers of patients to assess the overall frequency and relevance of these data. If the clinical characteristics of this patient are significantly different to the additional patients studied, then screening with increased numbers of sera may identify more patients that recognise these antigens. These are possibly of less general interest in DLBCL, but may be valuable for identifying and possibly treating a subset(s) of patients with different clinical characteristics.

Group 3 contains 6 antigens that are recognised by the majority of DLBCL patients' sera and one or two normal sera. Of these, 4 are only recognised by serum from a single normal individual. It is always possible that one of the control individuals has undiagnosed cancer (although no one normal serum is reactive with all these clones) thus larger numbers of patients and control individuals will determine whether this is an infrequent event. The expression of these antigens is still likely to be of interest in DLBCL. Three of these genes have differential mRNA expression within DLBCL patients and 2 of these 3 show increased expression in DLBCL patients when compared to patients with another germinal centre derived B-cell lymphoma, FL (that can transform into DLBCL). Not all clinically relevant lymphoma antigens show tumour specific expression; for example BCL-2 and BCL-6 are both expressed in a subset of normal B cells in addition to B cell lymphomas.

Group 4 contains 13 antigens that are recognised by serum from both DLBCL patients and controls. Immunologically these antigens are the least interesting group. However the Lymphochip mRNA expression data for CBF1 indicates that the mRNA encoding this antigen is expressed at higher levels in the majority of DLBCL patients when compared to those with FL. The expression levels of CBF1 and other members of this group may therefore still have clinical significance in DLBCL that could influence diagnosis or prognosis.

Since a testis library was screened with DLBCL serum, the relevance of the identified antigens to DLBCL needed to be established. For a number of antigens, previous microarray studies had demonstrated that these genes are expressed in DLBCL (Alizadeh et al., 2000). These are OX-TES-6, 10, 13, 15, 16 and 19. The inventors have characterised the expression of a number of the remaining OX-TES antigen mRNAs in a number of DLBCL cell lines to investigate whether expression occurs, alongside some of those already known to be expressed in DLBCL acting as controls. The protocol used was as described for OX-TES-1. Gene specific primers were designed to amplify fragments of 100-800 bp, and sequences are shown in Table 2.

Expression of OX-TES Antigens in DLBCL Cell Lines

All of the cell lines tested, which included both ABC-DLBCLs and GC-DLBCLs, gave a product of the expected size for the eighteen antigens tested (see FIG. 16) with the exception of OX-TES-12. Here, the product was slightly larger than the positive control but sequencing data revealed that the correct fragment had been amplified. These results indicate that transcripts of these antigens are certainly present in DLBCL. Most of the antigens required two rounds of PCR to amplify a product of similar intensity of the positive control in each case (after staining with ethidium bromide) due to the small amount of starting material. However, OX-TES-11, 12 and 21 required only one round, which, although the PCR was not quantitative, might suggest a high expression level of these antigens in DLBCL. Since overexpressed antigens are a common group of SEREX-defined antigens, this result was not surprising, although further work is required to determine whether there is genuine overexpression in comparison with normal tissues. Interestingly, only six of the twenty-eight antigens (21%) identified in this study are included on the original microarray study (Alizadeh et al., 2000), which suggests that there are possibly a wide variety of DLBCL-associated antigens as yet unidentified, and that SEREX is a successful approach to use in uncovering them.

Recognition of SEREX-Defined Antigens by Sera o Lymphoma/Leukaemia Patients and Healthy Individuals

To determine whether the antigens identified in this study were DLBCL-related, and to assess the frequency and specificity of antibody responses to these antigens in DLBCL, AML and CML patients, the antigens were screened with sera from additional patients and control individuals, resulting in data from ten DLBCL, ten AML and ten CML patients, as well as twenty control individuals in a tertiary screen. The screening data is shown in Tables 3 and 4. With respect to DLBCL only, eight antigens (OX-TES-2 to 9) appear to be disease-specific, whilst twenty-three antigens (all except OX-TES-17, 18, 19, 25 and 28) show a higher reactivity with patient sera over healthy controls. Taking AML and CML into account, of the fifteen antigens tested, three, OX-TES-3, 4 and 6, are now disease-specific, but only one, OX-TES-4, is specific to DLBCL, showing no reactivity with CML, AML or normal control sera, and reacting with 50% of sera from DLBCL patients. Twelve of the antigens (OX-TES-1, 3, 4, 5, 11, 12, 13, 15, 20, 22, 24 and 28) now show a higher frequency of reactivity with sera from patients with disease over normal individuals, but this higher frequency is restricted to DLBCL for OX-TES-1, 4, 12, 15 and 28, i.e. there is no reactivity detected with CML and AML sera for these antigens. None of the antigens have a greater reactivity with any of the leukaemia sera over DLBCL sera, suggesting that these might be more specific to DLBCL, although OX-TES-6, 10 and 21 elicit responses in a similar number of DLBCL and CML patients. Overall, the antigens seem to have a patient reactivity of DLBCL>CML>AML. Only OX-TES-18 and 24 have a greater reactivity with normal control sera over DLBCL, CML or AML sera, whilst OX-TES-19, 25 and 28 show an identical level of reactivity. Four of the antigens (OX-TES-2, 7, 8, and 9) reacted only with the serum used to screen the library. These antigens may therefore be patient- rather than disease-related.

Cancer-related immunogenicity implies a possible biological relevance of the antigens to tumourigenesis and/or disease progression, whilst antigens detected at a higher frequency in patients may also serve as diagnostic markers for disease diagnosis and monitoring (Chen 2000). For antigens to be diagnostically useful, antibodies against the antigen must be found in a considerable proportion of patients, and the antibody response in normal sera must be at a much lower frequency; the diagnostic/marker potential of the SEREX-defined DLBCL antigens is exemplified by the fact that serum samples from 70% of DLBCL patients detect three or more of these antigens. The lower cross-reactivity with AML and CML sera suggests that these antigens may only be lymphoma specific and not applicable to all haematological malignancies.

On the whole it is unclear as to why some antigens are immunogenic in apparently healthy controls; one suggestion is that the antigens are expressed in normal cells under non-malignant conditions such as viral infection or inflammatory processes (Sahin et al., 1995; Tureci et al., 1997). The fact that some antigens may be ubiquitously expressed but become immunogenic in a cancer patient suggests that the context in which the antigen is presented in is relevant i.e. the presentation of a protein to the immune system in a context of danger is more decisive for its immunogenicity (and breaking of tolerance) rather than its restricted expression in a given tissue (the idea of “contextual immunogenicity” (Ono et al., 2000; Preuss et al., 2002; Schmits et al., 2002) Although it is possible that the antibodies detected in healthy donors may not be directed at the same B-cell epitopes on these proteins, it cannot be ruled out that the normal donor may have the disease unknowingly, or that the epitope is not common to some other protein, or that the antibody in the control sera is against a wild-type protein and it is cross-reacting with the similar epitope on the mutated cancer-related antigen. Significantly, it remains to be determined if any of the antibody responses identified here arose as a result of treatment; OX-TES-10 (SRPK1), OX-TES-14 (HIS1) and OX-TES-23 (NSEP1) have a known relationship with cisplatin resistance (Ohga et al., 1996; Rifkind et al., 1996; Ise et al., 1999; Schenk et al., 2001; Yahata et al., 2002). In this study we used serum from a patient who was very young to have DLBCL, and had a very aggressive form that was never in remission. The serum was taken post-treatment and therefore the detection of antigens identified in this study may be related to the failure of the treatment, or indicative of a form of DLBCL that will be resistant to current treatment.

References

Aasland, R., T. J. Gibson and A. F. Stewart (1995). The PHD finger: implications for chromatin-mediated transcriptional regulation. Trends Biochem. Sci. 20: 56-59.

Aguiar, R. C., Y. Yakushijin, S. Kharbanda, et al. (2000). BAL is a novel risk-related gene in diffuse large B-cell lymphomas that enhances cellular migration. Blood 96: 4328-4334.

Alizadeh, A. A., M. B. Eisen, R. E. Davis, et al. (2000). Distinct types of diffuse large B-cell lymphoma identified by gene expression profiling. Nature 403: 503-511.

Amakawa, R., W. Jing, K. Ozawa, et al. (1993). Human Jk recombination signal binding protein (IGKJRB): comparison with its mouse homologue. Genomics 17: 306-315.

Angrand, P. O., F. Apiou, A. F. Stewart, et al. (2001). NSD3, a new SET domain-containing gene, maps to 8p12 and is amplified in human breast cancer cell lines. Genomics 74: 79-88.

Baars, J. W., D. de Jong, E. M. Willemse, et al. (1999). Diffuse large B-cell non-Hodgkin lymphomas: the clinical relevance of histological subclassification. Br. J. Cancer 79: 1770-1776.

Banerjee, D. (1999). Technology evaluation: G-3139. Curr Opin Mol Ther 1: 404-8.

Bea, S., M. Ribas, J. M. Hernandez, et al. (1999). Increased number of chromosomal imbalances and high-level DNA amplifications in mantle cell lymphoma are associated with blastoid variants. Blood 93: 4365-4374.

Bei, R., L. Masuelli, E. Moriconi, et al. (1999). Immune responses to all ErbB family receptors detectable in serum of cancer patients. 18: 1267-1275.

Benharroch, D., Z. Meguerian-Bedoyan, L. Lamant, et al. (1998). ALK-positive lymphoma: a single disease with a broad spectrum of morphology. Blood 91: 2076-2084.

Biamonti, G., M. Ruggiu, S. Saccone, et al. (1994). Two homologous genes, originated by duplication, encode the human hnRNP proteins A2 and A1. Nucleic Acids Res. 22: 1996-2002.

Bochar, D. A., J. Savard, W. Wang, et al. (2000). A family of chromatin remodeling factors related to Williams syndrome transcription factor. Proc. Natl. Acad. Sci. U.S.A. 97: 1038-1043.

Boon, T. and L. J. Old (1997). Cancer Tumor Antigens. Curr Opin Immunol 9: 681-3.

Brass, N. and e. al. (1997). Translation initiation factor eIF-4g is encoded by an amplified gene and induces an immune response in squamous lung carcinoma. Hum. Mol. Genet. 6: 33-39.

Brichard, V., A. Van Pel, T. Wölfel, et al. (1993). The tyrosinase gene codes for an antigen recognized by autologous cytolytic T lymphocytes on HLA-A2 melanomas. J. Exp. Med. 178: 489-495.

Capowski, E. E., S. Esnault, S. Bharracharya, et al. (2001). Y box-binding factor promotes eosinophil survival by stabilizing granulocyte-macrophage colony-stimulating factor mRNA. J. Immunol. 167: 5970-5976.

Caron de Fromental, C., F. May-Levin, H. Mouriesse, et al. (1987). Presence of circulating antibodies against cellular protein p53 in a notable proportion of children with B cell lymphoma. Int. J. Cancer 39: 185-189.

Castiglia, D., A. Cestelli, M. Scaturro, et al. (1994). H1(0) and H3.3B mRNA levels in developing rat brain. Neurochem. Res. 19: 1531-1537.

Changolkar, L. N. and J. R. Pehrson (2002). Reconstitution of nucleosomes with Histone MacroH2A1.2. Biochem. 41: 179-184.

Chen, C. Y., R. Gherzi, J. S. Anderson, et al. (2000). Nucleolin and YB-1 are required for JNK-mediated interleukin-2 mRNA stabilization during T-cell activation. Genes Dev. 14: 1236-1248.

Chen, Y. T. and L. J. Old (1999). Cancer-testis antigens: targets for cancer immunotherapy. Cancer J. Sci. Am. 5: 16-17.

Chen, Y. T. (2000). “Cancer vaccine: identification of human tumor antigens by SEREX.” Cancer J Sci Am 6 Suppl 3: S208-17.

Chen, Y. T., Gure, A. O., Tsang, S., Stockert, E., Jager, E., Knuth, A. and Old, L. J. (1998). “Identification of multiple cancer/testis antigens by allogeneic antibody screening of a melanoma cell line library.” Proc Natl Acad Sci USA 95(12): 6919-23.

Cheng, A., P. Kaldis and M. J. Solomon (2000). Dephosphorylation of human cyclin-dependent kinases by protein phosphatase type 2C alpha and beta 2 isoforms. J. Biol. Chem. 275: 34744-34749.

Cheng, M., T. G. Bouton and M. H. Cobb (1996). ERK3 is a constitutively nuclear protein kinase. J. Biol. Chem. 271: 8951-8958.

Chiou, C. C., C. C. Chan, D. L. Sheu, et al. (2001). Helicobacter pylori infection induced alteration of gene expression in human gastric cells. Gut 48: 598-604.

Cigudosa, J. C., N. Z. Parsa, D. C. Louie, et al. (1999). Cytogenetic analysis of 363 consecutively ascertained diffuse large B-cell lymphomas. Genes Chrm. Cancer 25: 123-133.

Coles, L. S., P. Diamond, F. Occhiodoro, et al. (1996). Cold shock domain proteins repress transcription from the GM-CSF promoter. Nucl. Acids Res. 24: 2311-2317.

Cordell, J. L., K. A. F. Pulford, B. Bigerna, et al. (1999). Detection of normal and chimeric nucleophosmin in human cells. Blood 93: 632-642.

Crawford, L. V., D. C. Pim and R. D. Bulbrook (1982). Detection of antibodies against the cellular protein p53 in sera from patients with breast cancer. Int. J. Cancer 30: 403-408.

Dalla-Favera, R., B. H. Ye, F. Le Coco, et al. (1994). Identification of genetic lesions associated with diffuse large-cell lymphoma. Ann. Oncol. 5 Suppl: S55-S60.

Daniel, J. M. and A. B. Reynolds (1999). The catenin p120 (ctn) interacts with Kaiso, a novel BTB/POZ domain zinc finger transcription factor. Mol. Cell Biol. 19: 3614-3623.

Das, A. K., N. R. Helps, P. T. W. Cohen, et al. (1996). Crystal structure of the protein serine/threonine phosphatase 2C at 2.0 angstrom resolution. EMBO J. 15: 6798-6809.

Davis, R. J. (1995). Transcriptional regulation by MAP kinases. Mol. Reprod. Dev. 42: 459-467.

Deltour, S., C. Guerardel and D. Leprince (1999). Recruitment of SMRT/N-CoR-mSin3A-HDAC-repressing complexes is not a general mechanism for BTB/POZ transcriptional repressors: the case of HIC-1 and gammaFBP-B. Proc. Natl. Acad. Sci. U.S.A. 96: 14831-14836.

Didier, D. K., J. Schiffenbauer, S. L. Woulfe, et al. (1988). Characterization of the cDNA encoding a protein binding to the major histocompatibility complex class II Y box. Proc. Natl. Acad. Sci. U.S.A. 85: 7322-7326.

Disis, M. L. and M. A. Cheever (1996). Oncogenic proteins as tumour antigens. Curr. Opin. Immunol. 8: 637-642.

Disis, M. L., Calenoff, E., McLaughlin, G., Murphy, A. E., Chen, W., Groner, B., Jeschke, M., Lydon, N., McGlynn, E., Livingston, R. B. and et al. (1994). “Existent T-cell and antibody immunity to HER-2/neu protein in patients with breast cancer.” Cancer Res 54(1): 16-20.

Donoghue, S., H. S. Baden, I. Lauder, et al. (1999). Immunohistochemical localization of caspase-3 correlates with clinical outcome in B-cell diffuse large-cell lymphoma. Cancer Res. 59: 5386-5391.

Dropcho, E. J., Y.-T. Chen, J. B. Posner, et al. (1987). Cloning of a brain protein identified by autoantibodies from a patient with paraneoplastic cerebellar degeneration. Proc. Natl. Acad. Sci. USA 84: 4552-4556.

Dunbar, P., G. Ogg, J. Chen, et al. (1998). Direct isolation, phenotyping and cloning of low-frequency antigen specific cytotoxic T lymphocytes from peripheral blood. Curr. Biol. 8: 413-6.

Evdokimova, V., P. Ruzanov, H. Imataka, et al. (2001). The major mRNA-associated protein YB-1 is a potent 5′ cap-dependent mRNA stabilizer. EMBO J. 20: 2491-5502.

Falini, B., Pileri, S., Zinzani, P. L., Carbone, A., Zagonel, V., Wolf-Peeters, C., Verhoef, G., Menestrina, F., Todeschini, G., Paulli, M., Lazzarino, M., Giardini, R., Aiello, A., Foss, H D., Araujo, I., Fizzotti, M., Pelicci, P G., Flenghi, L., Martelli, M F., Santucci, A., 1999. ALK+ lymphoma: clinico-pathological findings and outcome. Blood 93: p2697-706.

Favreau, C., H. J. Worman, R. W. Wozniak, et al. (1996). Cell cycle-dependent phosphorylation of nucleoporins and nuclear pore membrane protein Gp210. Biochem. 35: 8035-8044.

Ferrando-May, E., V. Cordes, I. Biller-Ckovric, et al. (2001). Caspases mediate nucleoporin cleavage, but not early redistribution of nuclear transport factors and modulation of nuclear permeability in apoptosis. Cell Death Differ. 8: 495-505.

Ferreira, P. A., C. Yunfei, D. Schick, et al. (1998). The cyclophilin-like domain mediates the association of Ran-binding protein 2 with subunits of the 19 S regulatory complex of the proteasome. J. Biol. Chem. 273: 24676-24682.

Fleischhacker, M., T. Beinert, M. Ermitsch, et al. (2001). Detection of ampifiable messenger RNA in the serum of patients with lung cancer. Ann. N.Y. Acad. Sci. 945: 179-188.

Fox, A. H., Lam, Y. W., Leung, A. K., Lyon, C. E., Andersen, J., Mann, M., Lamond, A. I., 2002. Paraspeckles. A novel nuclear domain. Curr Biol. 12: p13-25.

Fowler, L., J. Everitt, J. L. Stevens, et al. (1998). Redistribution and enhanced protein kinase C-mediated phosphorylation of alpha- and gamma-adducin during renal tumor progression. Cell Growth Differ. 9: 405-413.

Freemont, P. S. (2000). RING for destruction? Curr. Biol. 10: R84-87.

Friedman, L. S., E. A. Ostermeyer, E. D. Lynch, et al. (1995). 22 genes from chromosome 17q21: cloning, sequencing, and characterization of mutations in breast cancer families and tumors. Genomics 25: 256-263.

Fukata, Y., N. Oshiro, N. Kinoshita, et al. (1999). Phosphorylation of adducin by Rho-kinase plays a crucial role in cell motility. J. Cell Biol. 145: 347-361.

Gascoyne, R. D., Aoun, P., Wu, D., Chhanabhai, M., Skinnider, B. F., Greiner, T. C., Morris, S. W., Connors, J. M., Vose, J. M., Viswanatha, D. S., Coldman, A., Weisenburger, D. D., 1999. Prognostic significance of anaplastic lymphoma kinase (ALK) protein expression in adults with anaplastic large cell lymphoma. Blood 93: p3913-21.

Gilboa, E. (2001). The risk of autoimmunity associated with tumor immunotherapy. Nature Immunol. 2: 789-792.

Goyns, M. H., D. W. Hammond, C. J. Harrison, et al. (1993). Structural abnormalities of the X-chromosome in non-Hodgkins-lymphoma. Leukemia 7: 848-852.

Grillo-Lopez, A. J., B. K. Dallaire, A. McClure, et al. (2001). Monoclonal antibodies: a new era in treatment of Non-Hodgkin's lymphoma. Curr Pharm Biotechnol 2: 301-311.

Gu, C., T. Oyama, T. Osaki, et al. (2001). Expression of Y box-binding protein-1 correlates with DNA topoisomerase IIalpha and proliferating cell nuclear antigen expression in lung cancer. Anticancer Res. 21: 2357-2362.

Gu, Z., G. Kuntz-Simon, J. Rommelaere, et al. (1999). Oncogenic transformation-dependent expression of a transcription factor NF-Y subunit. Mol. Carcinog. 24: 294-299.

Gui, J. F., W. S. Lane and X. D. Fu (1994). A serine kinase regulates intracellular localization of spicing factors in the cell cycle. Nature 369: 678-682.

Halbach, T., N. Scheer and W. Werr (2000). Transcriptional activation by the PHD finger is inhibited through an adjacent leucine zipper that binds 14-3-3 proteins. Nuc. Acids Res. 28: 3542-3550.

Hanada, M., T. Kobayashi, M. Ohnishi, et al. (1998). Selective suppression of stress-activated protein kinase pathway by protein phosphatase 2C in mammalian cells. FEBS Lett. 437: 172-176.

Harris, N. L., E. S. Jaffe, H. Stein, et al. (1994). A revised European-American classification of lymphoid neoplasms—a proposal from the International Lymphoma Study-Group. Blood 84: 1361-1392.

Harris, N. L., E. S. Jaffe, J. Diebold, et al. (1999). The World Health Organization classification of neoplastic diseases of the hematopoietic and lymphoid tissues. Report of the Clinical Advisory Committee meeting, Airlie House, Va., November, 1997. Ann Oncol. 10: 1419-1432.

Hassfeld, W., G. Steiner, A. Studnicka-Benke, et al. (1995). Autoimmune response to the spliceosome. An immunologic link between rheumatoid arthritis, mixed connective tissue disease, and systemic lupus erythematosus. Arthritis Rheum. 38: 777-785.

Hayess, K., R. Kraft, J. Sachsinger, et al. (1998). Mammalian protein homologous to VAT-1 of Torpedo californica: isolation from Ehrlich ascites tumor cells, biochemical characterization, and organization of its gene. J. Cell Biochem. 69: 304-315.

Hofer, T., P. Spielmann, P. Stengel, et al. (2001). Mammalian PASKIN, a PAS-serine/threonine kinase related to bacterial oxygen sensors. Biochem. Biophys. Res. Commun. 288: 757-764.

Hraba-Renevey, S. and M. Kress (1989). Expression of a mouse replacement histone H3.3 gene with a highly conserved 3′ noncoding region during SV40- and polyoma-induced Go to S-phase transition. Nucleic Acids Res. 17: 2449-2461.

Hsieh, J. J.-D., T. Henkel, P. Salmon, et al. (1996). Truncated mammalian Notch1 activates CBF1/RBPJk-repressed genes by a mechanism resembling that of Epstein=Barr virus EBNA2. Mol. Cell. Biol. 16: 952-959.

Hsu, F. J., C. B. Caspar, C. Czerwinski, et al. (1997). Tumor-specific idiotype vaccines in the treatment of patients with B-cell lymphoma: long term results of a clinical trial. Blood 89: 3129-3135.

Hu, Z., S. Jin and K. W. Scott (2000). Transcriptional activation of the MDR1 gene by UV irradiation. Role of NF-Y and Sp1. J. Biol. Chem. 275: 2979-2985.

Huang, N., E. vom Baur, J. M. Garnier, et al. (1998). Two distinct nuclear receptor interaction domains in NSD1, a novel SET protein that exhibits characteristics of both corepressors and coactivators. EMBO J. 17: 3398-3412.

Ise, T., G. Nagatani, T. Imamura, et al. (1999). Transcription factor Y-box binding protein 1 binds preferentially to cisplatin-modified DNA and interacts with proliferating cell nuclear antigen. Cancer Res. 59: 342-346.

Ise, T., Nagatani, G., Imamura, T., Kato, K., Takano, H., Nomoto, M., Izumi, H., Ohmori, H., Okamoto, T., Ohga, T., Uchiumi, T., Kuwano, M. and Kohno, K. (1999). “Transcription Factor Y-Box Binding Protein 1 Binds Preferentially to Cisplatin-modified DNA and Interacts with Proliferating Cell Nuclear Antigen.” Cancer Res 59(2): 342-346.

Itoh, M., M. Watanabe, Y. Yamada, et al. (1999). HUB1 is an autoantigen frequently eliciting humoral immune response in patients with adult T cell leukemia. Int. J. Oncol. 14: 703-708.

Jager, E., Jager, D. and Knuth, A. (2000). “Peptide Vaccination in Clinical Oncology.” Onkologie 23(5): 410-415.

Jäger, E., D. Jäger and A. Knuth (2000). Peptide vaccination in clinical oncology. Onkologie 23: 410-415.

Jager, E., Chen, Y. T., Drijfhout, J. W., Karbach, J., Ringhoffer, M., Jager, D., Arand, M., Wada, H., Noguchi, Y., Stockert, E., Old, L. J. and Knuth, A. (1998). “Simultaneous humoral and cellular immune response against cancer-testis antigen NY-ESO-1: definition of human histocompatibility leukocyte antigen (HLA)-A2-binding peptide epitopes.” J. Exp. Med. 187(2): 265-70.

Jäger, E., Y.-T. Chen, J. Drijfhout, et al. (1998). Simultaneous humoral and cellular immune response against cancer-testis antigen NY-ESO-1: Definition of human histocompatibility leucocyte antigen (HLA)-A2-binding peptide epitopes. J. Exp. Med. 187: 265-270.

Jäger, E., Y. Nagata, S. Gnjatic, et al. (2000). Monitoring CD8 T cell responses to NY-ESO-1: Correlation of humoral and cellular immune responses. Proc. Natl. Acad. Sci. U.S.A. 97: 4760-4765.

Jaju, R. J., C. Fidler, O. A. Haas, et al. (2001). A novel gene, NSD1, is fused to NUP98 in the t(5;11)(q35;p15.5) in de novo childhood acute myeloid leukemia. Blood 98: 1264-1267.

Janz, M., N. Harbeck, P. Dettmar, et al. (2002§). Y-box factor YB-1 predicts drug resistance and patient outcome in breast cancer independently of clinically relevant tumor biologic factors HER2, uPA and PAI-1. Int. J. Cancer 97: 278-282.

Jarrett, R. F. and J. MacKenzie (1999). Epstein-Barr virus and other candidate viruses in the pathogenesis of Hodgkin's disease. Sem. in Haematol. 36: 260-269.

Jensen, D. E., M. Proctor, S. T. Marquis, et al. (1998). BAP1: a novel ubiquitin hydrolase which binds to the BRCA1 RING finger and enhances BRCA1-mediated cell growth suppression. Oncogene 16: 1097-1112.

Jung, M. S., J. Yun, H. D. Chae, et al. (2001). p53 and its homologues, p63 and p73, induce a replicative senescence through inactivation of NF-Y transcription factor. Oncogene 20: 5818-5825.

Kamihira, S., K. Toriya, T. Amagasaki, et al. (1989). Antibodies against p40tax gene product of human T-lymphotropic virus type-1 (HTLV-1) under various conditions of HTLV-1 infection. Jpn. J. Cancer Res. 80: 1066-1071.

Kamiyama, J., T. Inoue, N. Ohtani-Fujita, et al. (1999). The ubiquitous transcription factor NF-Y positively regulates the transcription of human p27Kip1 through a CCAAT box located in the 5-upstream region of the p27Kip1 gene. FEBS Lett. 455: 281-285.

Kamma, H., M. Fujimoto, M. Fujiwara, et al. (2001). Interaction of hnRNP A2/B1 isoforms with telomeric ssDNA and the in vitro function. Biochem. Biophys. Res. Commun. 280: 625-630.

Kamma, H., H. Satoh, M. Matusi, et al. (2001). Characterization of hnRNP A2 and B1 using monoclonal antibodies: intracellular distribution and metabolism through cell cycle. Immmunol. Lett. 76: 49-54.

Karhumaa, P., S. Parkkila, A. Waheed, et al. (2000). Nuclear NonO/p54(nrb) protein is a nonclassical carbonic anhydrase. J. Biol. Chem. 275: 16044-16049.

Karuman, P., O. Gozani, R. D. Odze, et al. (2001). The Peutz-Jegher gene product LKB1 is a mediator of p53-dependent cell death. Mol. Cell. 7: 1307-1319.

Kiley, S. C., K. J. Clark, S. K. Duddy, et al. (1999). Increased protein kinase C delta in mammary tumor cells: relationship to transformtion and metastatic progression. Oncogene 18: 6748-6757.

King, C. A., M. B. Spellerberg, D. Zhu, et al. (1998). DNA vaccines with single-chain Fv fused to fragment C of tetanus toxin induce induce protective immunity against lymphoma and myeloma. Nature Med. 4: 1281-1286.

Kozu, T., B. Henrich and K. P. Schafer (1995). Structure and expression of the gene (HNRPA2B1) encoding the human hnRNP protein A2/B1. Genomics 25: 365-371.

Krackhardt, A. M., Witzens, M., Harig, S., Hodi, F. S., Zauls, A. J., Chessia, M., Barrett, P. and Gribben, J. G. (2002). “Identification of tumor-associated antigens in chronic lymphocytic leukemia by SEREX.” Blood 100(6): 2123-31.

Krimer, D. B., G. Cheng and A. I. Skoultchi (1993). Induction of H3.3 replacement histone mRNAs during the precommitment period of murine erythroleukemia cell differentiation. Nucleic Acids Res. 21: 2873-2879.

Kurotaki, N., N. Harada, K. Yoshiura, et al. (2001). Molecular characterization of NSD1, a human homologue of the mouse Nsd1 gene. Gene 279: 197-204.

Kuroyanagi, N., H. Onogi, T. Wakabayashi, et al. (1998). Novel SR-protein-specific kinase, SRPK2, disassembles nuclear speckles. Biochem. Biophys. Res. Commun. 242: 357-364.

Kwak, L., M. Wilson, L. M. Weiss, et al. (1991). Clinical significance of morphological subdivision in diffuse large B cell lymphoma. Cancer 68: 1988-1993.

Kwak, L. W., D. D. Taub, P. L. Duffey, et al. (1995). Transfer of myeloma idiotype-specific immunity from an actively immunized marrow donor. Lancet 345: 1016-1020.

Kwak, L. W., M. J. Campbell, D. K. Czerwinski, et al. (1993). Induction of immune responses in patients with B-cell lymphoma against the surface-immunoglobulin idiotype expressed by their tumors. New Engl. J. Med. 327: 1209-1215.

LaFond, R. E., R. B. Eaton, R. A. Watt, et al. (1992). Autoantibodies to c-myc protein: elevated levels in patients with African Burkitt's lymphoma and normal Ghanians. Autoimmunity 13: 215-224.

Lee, S. H., X. Wang and J. DeJpng (2000). Functional interactions between an atypical NF-kappaB site from the rat CYP2B1 promoter and the transcriptional repressor RBP-Jkappa/CBF1. Nucleic. Acids Res. 28: 2091-2098.

Lennert, K., N. Mohri, H. Stein, et al. (1975). The histopathology of malignant lymphoma. Br. J. Haematol. 31(Suppl): 193-203.

Lifschitz-Mercer, B., Y. Sheinin, D. Ben-Meir, et al. (2001). Protein phosphatase 2Calpha expression in normal human tissues: an immunohistochemical study. Histochem. Cel Biol. 116: 31-39.

Ling, M., Y.-J. Wen and S. H. Lim (1998). Prevalence of antibodies against proteins derived from leukemia cells in patients with chronic myeloid leukemia. Blood 92: 4764-4770.

Ling, P. D., J. J.-D. Hsieh, I. K. Ruf, et al. (1994). EBNA-2 upregulation of Epstein-Barr virus latency promoters and the cellular CD23 promoter utilizes a common targeting intermediate, CBF1. J. Virol. 68: 5375-5383.

Linial, M. and O. Levius (1993). The protein VAT-1 from Torpedo electric organ exhibits an ATPase activity. Neirosci Lett. 152: 155-157.

Linial, M., K. Miller and R. H. Scheller (1989). VAT-1: an abundant membrane protein from Torpedo cholinergic synaptic vesicles. Neuron 2: 1265-1273.

Liu, J., S. Akoulitchev, A. Weber, et al. (2001). Defective interplay of activators and repressors with TFHIIH in xeroderma pigmentosum. Cell 104: 353-363.

Loewith, R., M. Meijer, S. P. Lees-Miller, et al. (2000). Three yeast proteins related to the human candidate tumor suppressor p33(ING1) are associated with histone acetyltransferase activities. Mol. Cell. Biol. 20: 3807-3816.

Lubin, R., B. Schlichtholz, J. L. Teillaud, et al. (1995). p53 antibodies in patients with various types of cancer: Assay, identification, and characterization. Clin. Can. Res. 1: 1463-1469.

Luo, J., A. Y. Nikolaev, S. Imai, et al. (2001). Negative control of p53 by SIR2alpha promotes cell survival under stress. Cell 107: 137-148.

Makino, Y., R. Cao, K. Svensson, et al. (2001). Inhibitory PAS domain protein is a negative regulator of hypoxia-inducible gene expression. Nature 414: 550-554.

Makino, Y., T. Ohga, S. Toh, et al. (1996). Structural and functional analysis of the human Y-box binding protein (YB-1) gene promoter. Nucleic Acids Res. 24: 1873-1878.

Mann, D. J., D. G. Campbell, C. H. McGown, et al. (1992). Mammalian protein serine/threonine phosphatase 2C: cDNA cloning and comparative analysis of amino acid sequences. Biochim. Biophys. Acta 1130: 100-104.

Marchand, M., N. van Baren, P. Weynants, et al. (1999). Tumor regressions observed in patients with metastatic melanoma treated with an antigenic peptide encoded by gene MAGE-3 and presented by HLA-A1. Int. J. Cancer 80: 912.

Marx, J. (2001). Science 292: 426-429.

Matsuoka, Y., X. Li and V. Bennett (2000). Adducin: structure, function and regulation. Cell. Mol. Life Sci. 57: 884-895.

Matsuoka, Y., X. Li and V. Bennett (1998). Adducin is an in vivo substrate for protein kinase C: phosphorylation in the MARCKS-related domain inhibits activity in promoting spectrin-actin complexes and occurs in many cells, including dendritic spines of neurons. J. Cell. Biol. 142: 485-497.

Mermoud, J. E., A. M. Tassin, J. R. Pehrson, et al. (2001). Centrosomal association of histone macroH2A1.2 in embryonic stem cells and somatic cells. Exp. Cell. Res. 268: 245-251.

Monni, O., H. Joensuu, K. Franssila, et al. (1996). DNA copy number changes in diffuse large B-cell lymphoma comparative genomic hybridization study. Blood 87: 5269-5278.

Mosolits, S., U. Harmenberg, U. Ruden, et al. (1999). Autoantibodies against the tumour-associated antigen GA733-2 in patients with colorectal carcinoma. Cancer Immunol. Immunother. 47: 315-320.

Mushegian, A. R., D. E. J. Bassett, M. S. Boguski, et al. (1997). Positionally cloned human disease genes: patterns of evolutionary conservation and functional motifs. Proc. Natl. Acad. Sci. U.S.A. 20: 5831-5836.

Nestle, F. O., S. Alijagic, M. Gilliet, et al. (1998). Vaccination of melanoma patients with peptide- or tumor lysate pulsed dendritic cells. Nat. Med. 4: 328-.

Nishikawa, H., Tanida, K., Ikeda, H., Sakakura, M., Miyahara, Y., Aota, T., Mukai, K., Watanabe, M., Kuribayashi, K., Old, L. J. and Shiku, H. (2001). “Role of SEREX-defined immunogenic wild-type cellular molecules in the development of tumor-specific immunity.” Proc Natl Acad Sci USA 98(25): 14571-6.

Ohga, T., Koike, K., Ono, M., Makino, Y., Itagaki, Y., Tanimoto, M., Kuwano, M. and Kohno, K. (1996). “Role of the human Y box-binding protein YB-1 in cellular sensitivity to the DNA-damaging agents cisplatin, mitomycin C, and ultraviolet light.” Cancer Res 56(18): 4224-8.

Offit, K., S. C. Jhanwar, M. Ladanyi, et al. (1991). Cytogenetic analysis of 434 consecutively ascertained specimens of non-Hodgkin's lymphoma: correlations between recurrent aberations, histology and exposure of cytotoxic treatment. Genes Chrom. Cancer 3: 189-201.

Okabe, S., T. Fukuda, K. Ishibashi, et al. (1998). BAZF, a novel Bcl6 homolog, functions as a transcriptional repressor. Mol. Cell Biol. 18: 4235-4244.

Okamoto, T., H. Izumi, T. Imamura, et al. (2000). Direct interaction of p53 with the Y-box binding protein, YB-1: a mechanism for regulation of human gene expression. Oncogene 19: 6194-6202.

Old, L. J. and Chen, Y.-T. (1998). “New paths in human cancer serology.” Journal of Experimental Medicine 187(8): 1163-1167.

Olesen, C., C. Hansen, E. Bendsen, et al. (2001). Identification of human candidate genes for male infertility by digital differential display. Mol. Hum. Reprod. 7: 11-20.

Ono, T., Sato, S., Kimura, N., Tanaka, M., Shibuya, A., Old, L. J. and Nakayama, E. (2000). “Serological analysis of BALB/C methylcholanthrene sarcoma Meth A by SEREX: identification of a cancer/testis antigen.” Int J Cancer 88(6): 845-51.

Pasqualucci, L., P. Neumeister, T. Goossens, et al. (2001). Hypermutation of multiple proto-oncogenes in B-cell diffuse large-cell lymphomas. Nature 412: 341-346.

Pichler, A., A. gast, J. S. Seeler, et al. (2002). The nucleoporin RanBP2 has SUMO1 E3 ligase activity. Cell 108: 109-120.

Ponting, C. P. (1997). Tudor domains in proteins that interact with RNA. Trends Biochem. Sci. 22: 51-52.

Preuss, K. D., Zwick, C., Bormann, C., Neumann, F. and Pfreundschuh, M. (2002). “Analysis of the B-cell repertoire against antigens expressed by human neoplasms.” Immunol Rev 188(1): 43-50.

Project., T. N.-H. L. C. (1997). A clinical evaluation of the International Lymphoma Study Group classification of non-Hodgkin's lymphoma. Blood 89: 3909-3918.

Pulford, K., B. Falini, A. H. Banham, et al. (2000). Immune response to the ALK oncogenic tyrosine kinase in patients with anaplastic large-cell lymphoma. Blood 96: 1605-1607.

Pulford, K., H. Roberton, A. H. Banham, et al. (2001). Immunochemical studies of antigenic lymphoma-associated proteins. Brit. J. Haematol. In Press:

Pulford, K., L. Lamant, S. W. Morris, et al. (1997). Detection of anaplastic lymphoma kinase (ALK) and nucleolar protein nucleophosmin (NPM)-ALK proteins in normal and neoplastic cells with the monoclonal antibody ALK1. Blood 89: 1394-1404.

Rao, P. H., J. Houldsworth, K. Dyomina, et al. (1998). Chromosomal and gene amplification in diffuse large B-cell lymphoma. Blood 92: 234.

Rasmussen, T. P., T. Huang, M. A. Mastrangelo, et al. (1999). Messenger RNAs encoding mouse histone macroH2Al isoforms are expressed at similar levels in male and female cells and result from alternative splicing. Nucleic Acids Res. 27: 3685-3689.

Reeves, R. (2000). Structure and function of the HMGI (Y) family of architectural transcription factors. Environ. Health Perspect. 108: 803-809.

Rifkind, R. A., Richon, V. M. and Marks, P. A. (1996). “Induced differentiation, the cell cycle, and the treatment of cancer.” Pharmacol Ther 69(2): 97-102.

Robles, A. I. and C. C. Harris (2001). p53-mediated apoptosis and genomic instability diseases. Acta Oncol. 40: 696-701.

Romero, P., P. Dunbar, D. Valmori, et al. (1998). Ex vivo staining of metastatic lymph nodes by class I major histocompatibility complex tetramer labelling reveals high numbers of antigen-experienced tumour-specific cytolytic T lymphocytes. J. Exp. Med. 188: 1641-50.

Rosenberg, S. A. (1996). The immunotherapy of solid cancers based on cloning the genes encoding tumour-rejection antigens. Annu. Rev. Med. J. Natl. Can. Inst. 88: 1635-1644: 481-91.

Rutter, J., C. H. Michnoff, S. M. Harper, et al. (2001). PAS kinase: an evolutionarily conserved PAS domain-regulated serine/threonine kinase. Proc. Natl. Acad. Sci. U.S.A. 98: 8991-8996.

Sahin, U., Ö. Türeci, H. Schmitt, et al. (1995). Human neoplasms elicit multiple specific immune responses in the autologous host. Proc. Natl. Acad. Sci. USA 92: 11810-11813.

Sahin, U., Tureci, O., Schmitt, H., Cochlovius, B., Johannes, T., Schmits, R., Stenner, F., Luo, G., Schobert, I. and Pfreundschuh, M. (1995). “Human neoplasms elicit multiple specific immune responses in the autologous host.” PNAS USA 92: 11810-11813.

Saitoh, H., M. Delli Pizzi and J. Wang (2001). Perturbation of SUMOlation enzyme Ubc9 by distinct domain within nucleoporin RanBP2/Nup358. J. Biol. Chem. epub ahead of print:

Sampson, E. R., S. Y. Yeh, H. C. Chang, et al. (2001). Identification and characterization of androgen receptor associated coregulators in prostate cancer cells. J. Biol. Regul. Homeost. Agents 15: 123-129.

Sato, M., T. Tanaka, T. Maeno, et al. (2002). Inducible Expression of Endothelial PAS Domain Protein-1 by Hypoxia in Human Lung Adenocarcinoma A549 Cells. Role of src family kinases-dependent pathway. Am. J. Respir. Cell. Mol. Biol. 26: 127-134.

Satoh, H., H. Kamma, H. Ishikawa, et al. (2000). Expression of hnRNP A2/B1 proteins in human cancer cell lines. Int. J. Oncol. 16: 763-767.

Scanlan, M. J. and Jager, D. (2001). “Challenges to the development of antigen-specific breast cancer vaccines.” Breast Cancer Res 3(2): 95-8.

Scanlan, M. J., Y.-T. Chen, B. Williamson, et al. (1998). Charaterization of human colon cancer antigens recognized by autologous antibodies. Int. J. Cancer 76: 652-658.

Scaturro, M., A. Cestelli, D. Castiglia, et al. (1995). Posttranscriptional regulation of H1 zero and H3.3B histone genes in differentiating rat cortical neurons. Neurochem. Res. 20: 969-976.

Schaeffer, L., R. Roy, S. Humbert, et al. (1993). DNA repair helicase: a component of BTF2 (THFIIH) basic transcription factor. Science 260: 58-63.

Schenk, P. W., A. W. Boersma, J. A. Brandsma, et al. (2001). SKY1 is involved in cisplatin-induced cell kill in Saccharomyces cerevisiae, and inactivation of its human homologue, SRPK1, induces cisplatin resistance in a human ovarian carcinoma cell line. Cancer Res. 61: 6982-6986.

Schenk, P. W., Boersma, A. W., Brandsma, J. A., den Dulk, H., Burger, H., Stoter, G., Brouwer, J. and Nooter, K. (2001). “SKY1 is involved in cisplatin-induced cell kill in Saccharomyces cerevisiae, and inactivation of its human homologue, SRPK1, induces cisplatin resistance in a human ovarian carcinoma cell line.” Cancer Res 61(19): 6982-6.

Schmits, R., Cochlovius, B., Treitz, G., Regitz, E., Ketter, R., Preuss, K. D., Romeike, B. F. and Pfreundschuh, M. (2002). “Analysis of the antibody repertoire of astrocytoma patients against antigens expressed by gliomas.” Int J Cancer 98(1): 73-7.

Schillace, R. V., S. F. Andrews, G. A. Liberty, et al. (2002). Identification and characterization of myeloid translocation gene 16b as a novel kinase anchoring protein in T lymphocytes. J. Immunol. 168: 1590-1599.

Schultze, J. L. (1997). Vaccination as immunotherapy for B cell lymphoma. Hematol. Oncol. 15: 129-139.

Schwab, M. and L. C. Amler (1990). Amplification of cellular oncogenes: A predictor of clinical outcome in human cancer. Genes Chrm. Cancer 1: 81-.

Schweisguth, F. and J. W. Posakony (1992). Suppressor of hairless, the drosophila homolog of the mouse recombination signal-binding protein gene, controls sensory organ cell fates. Cell 69: 1199-1212.

Selenko, P., R. Sprangers, G. Stier, et al. (2001). SMN tudor domain structure and its interaction with the Sm proteins. Nat. Struct. Biol. 8: 27-31.

Shibahara, K., K. Sugio, T. Osaki, et al. (2001). Nuclear expression of the Y-box binding protein, YB-1, as a novel marker of disease progression in non-small cell lung cancer. Clin. Cancer Res. 7: 3151-3155.

Shipp, M. A., K. N. Ross, P. Tamayo, et al. (2002). Diffuse large B-cell lymphoma outcome prediction by gene-expression profiling and supervised machine learning. Nature Med. 8: 68-74.

Siegel, D. S., X. Zhang, R. Feinman, et al. (1998). Hexamethylene bisacetamide induces programmed cell death (apoptosis) and down-regulates BCL-2 expression in human myeloma cells. Proc. Natl. Acad. Sci. U.S.A. 95: 162-166.

Skotheim, R. I., S. M. Kraggerud, S. D. Fossa, et al. (2001). Familial/bilateral and sporadic testicular germ line tumors show frequent genetic changes at loci with suggestive linkage evidence. Neoplasia 3: 196-203.

Smith, C. and V. Cerundolo (2001). Immunotherapy of melanoma. Immunology 104: 1-7.

Smith, T. M., M. K. Lee, C. I. Szabo, et al. (1996). Complete genomic sequence and analysis of 117 kb of human DNA containing the gene BRCA1. Genome Res. 6: 1029-1049.

Spitkovsky, D. D., B. Royer-Pokora, H. Delius, et al. (1992). Tissue restricted expression and chromosomal localzation of the YB-1 gene encoding a 42 kD nuclear CCAAT binding protein. Nucl. Acids Res. 20: 797-803.

Staudt, L. M., A. L. Dent, A. L. Shaffer, et al. (1999). Regulation of lymphocyte cell fate decisions and lymphomagenesis by BCL-6. Int. Rev. Immunol. 18: 381-403.

Stein, H. and F. Dallenbach (1992). Diffuse large cell lymphomas of B and T cell type. Neoplastic Hematopathology. Baltimore, Williams & Wilkins. 675-714.

Stenina, O. I., K. M. Shaneyfelt and P. E. DiCorleto (2001). Thrombin induces the release of the Y-box protein dbpB from mRNA: a mechnism of trancriptional activation. Proc. Natl. Acad. Sci. U.S.A. 98: 7277-7282.

Stevenson, F. K. (1999). DNA vaccines against cancer: from genes to therapy. Ann Oncol 10: 1413-8.

Stevenson, F. K., D. Zhu and J. Rice (2001). New strategies for vaccination and immunomodulation in NHL. Ann Hematol 80, Suppl 3: B132-4.

Suhasini, M. and R. B. Pilz (1999). Transcriptional elongation of c-myb is regulated by NF-kappaB (p50/RelB). Oncogene 18: 7360-7369.

Sun, W., F. Hou, M. P. Panchenko, et al. (2001). A member of the Y-box family interacts with an upstream element in the alpha1 (I) collagen gene. Matrix Biol. 20: 527-541.

Syrengelas, A. D., T. T. Chen and R. Levy (1996). DNA immunization induces protective immunity against B-cell lymphoma. Nat. Med. 2: 1038-1041.

Takahashi, K., S. Shichijo, M. Noguchi, et al. (1995). Identification of MAGE-1 and MAGE-4 proteins in spermatogonia and primary spermatocytes of testis. Cancer Res. 55: 3478-3482.

Takayama, T., A. E. Morelli, P. D. Robbins, et al. (2000). Feasibility of CTL4AIg gene delivery and expression in vivo usibg retrovirally transduced myeloid dendritic cells that induce alloantigen-specific T cell anergy in vitro. Gene Ther 7: 1265-73.

Takeda, N., M. Shibuya and Y. Maru (1999). The BCR-ABL oncoprotein potentially interacts with the xeroderma pigmentosum group B protein. Proc. Natl. Acad. Sci. U.S.A 96: 203-207.

Takekawa, M., T. Maeda and H. Saito (1998). Protein phosphatase 2C-alpha inhibits the human stress-responsive p38 and JNK MAPK pathways. EMBO J. 17: 4744-4752.

Tarte, K., Z. G. Zhang, E. Legouffe, et al. (1999). Induced expresion of B7-1 on myeloma cells following retroviral gene transfer results in tumor-specific recognition by cytotoxic T cells. J Immunol 163: 514-24.

Travis, S. M., H. A. Berger and M. J. Welsh (1997). Protein phosphatase 2C dephosphorylates and inactivates cystic fibrosis transmembrane conductance regulator. Proc. Natl. Acad. Sci. U.S.A. 94: 11055-11060.

Tureci, O., Sahin, U. and Pfreundschuh, M. (1997). “Serological analysis of human tumor antigens: molecular definition and implications.” Mol Med Today 3(8): 342-9.

Türeci, O., U. Sahin and M. Pfreundschuh (1997). Serological analysis of human tumor antigens: molecular definition and implications. Mol. Med. Today 3: 342-349.

Türeci, Ö., U. Sahin, C. Zwick, et al. (1998). Identification of a meiosis-specific protein as a member of the class of cancer/testis antigens. Proc. Natl. Acad. Sci. U.S.A. 95: 5211-5216.

Türeci, Ö., U. Sahin, C. Zwick, et al. (1999). Exploitation of the antibody repertoire of cancer patients for the identification of human tumor antigens. Hybridoma 18: 23-28.

van der Bruggen, P., C. Traversari, P. Chomez, et al. (1991). A gene encoding an antigen recognized by cytolytic T lymphocytes on a human melanoma. Science 254: 1643-1647.

van der Bruggen, P., Szikora, J. P., Boel, P., Wildmann, C., Somville, M., Sensi, M. and Boon, T. (1994). “Autologous cytolytic T lymphocytes recognize a MAGE-1 nonapeptide on melanomas expressing HLA-Cw*1601.” Eur J Immunol 24(9): 2134-40.

van de Water, B., I. B. Tijens, A. Verbrugge, et al. (2000). Cleavage of the actin-capping protein alpha -adducin at Asp-Asp-Ser-Asp633-Ala by caspase-3 is preceded by its phosphorylation on serine 726 in cisplatin-induced apoptosis of renal epithelial cells. J. Biol. Chem. 275: 25805-25813.

Vaziri, H., S. K. Dessain, E. N. Eaton, et al. (2001). hSIR2 (SIRT1) functions as an NAD-dependent p53 deacetylase. Cell 107: 149-159.

Venkitaraman, A. R. (2001). Functions of BRCA1 and BRCA2 in the biological response to DNA damage. J. Cell Sci. 114: 3591-3598.

Vineis, P., G. Masala and A. S. Costantini (1999). Does a gene in the Xq28 region increase the risk of non-Hodgkin's lymphomas? Working group for the Epidemiology of Hematolymphopoietic Malignancies in Italy. Ann. Oncol. 10: 471-473.

Wang, H. Y., W. Lin, J. A. Dyck, et al. (1998). SRPK2: a differentially expressed SR protein-specific kinase involved in mediating the interaction and localization of pre-mRNA splicing factors in mammalian cells. J. Cell Biol. 140: 737-750.

Wang, X., S. Yeh, G. Wu, et al. (2001). Identification and characterization of a novel androgen receptor coregulator ARA267-alpha in prostate cancer cells. J. Biol. Chem. 276: 40417-40423.

Witt, O., W. Albig and D. Doenecke (1997). Transcriptional regulation of the human replacement hitsone gene H3.3B. FEBS Lett. 408: 255-260.

Witt, O., W. Albig and D. Doenecke (1998). cAMP/phorbol ester response element is involved in transcriptional regulation of the human replacement histone gene H3.3B. Biochem J. 329: 609-613.

Wu, J., M. J. Matunis, D. Kraemer, et al. (1995). Nup358, a cytoplasmically exposed nucleoporin with peptide repeats, Ran-GTP binding sites, zinc fingers, a cyclophilin A homologous domain, and a leucine-rich region. J. Biol. Chem. 270: 14209-14213.

Xia, G., Y. Kageyama, T. Hayashi, et al. (2001). Regulation of vascular endothelial growth factor transcription by endothelial PAS domain protein 1 (EPAS1) and possible involvement of EPAS1 in the angiogenesis of renal cell carcinoma. Cancer 91: 1429-1436.

Xie, X., Wacker, H.-H., Huang, S., Regitz, E., Preuss, K.-D., Romeike, B., Parwaresch, R., Tiemann, M. and Pfreundschuh, M. (2003). “Differential Expression of Cancer Testis Genes in Histological Subtypes of Non-Hodgkin's Lymphomas.” Clin Cancer Res 9(1): 167-173.

Yahata, H., Kobayashi, H., Kamura, T., Amada, S., Hirakawa, T., Kohno, K., Kuwano, M. and Nakano, H. (2002). “Increased nuclear localization of transcription factor YB-1 in acquired cisplatin-resistant ovarian cancer.” J Cancer Res Clin Oncol 128(11): 621-6.

Yamada, K., R. L. Printz, H. Osawa, et al. (1999). Human ZHX1: cloning, chromosomal location, and interaction with transcription factor NF-Y. Biochem. Biophys. Res. Commun. 261: 614-621.

Ylikorkala, A., D. J. Rossi, N. Korsisaari, et al. (2001). Vascular abnormalities and deregulation of VEGF in Lkb1-deficient mice. Science 293: 1323-1326.

Yuasa, K., K. Omori and N. Yanaka (2000). Binding and phosphorylation of a novel male germ cell-specific cGMP-dependent protein kinse-anchoring protein by cGMP-dependent protein kinase Ia. J. Biol. Chem. 275: 4897-4905.

Yun, J., H. D. Chae, H. E. Choy, et al. (1999). p53 negatively regulates cdc2 transcription via the CCAAT-binding NF-Y transcription factor. J. Biol. Chem. 274: 29677-29682.

Zendman, A. J., Ruiter, D. J. and Van Muijen, G. N. (2003). “Cancer/testis-associated genes: Identification, expression profile, and putative function.” J Cell Physiol 194(3): 272-88.

Zhang, A., K. Ohshima, K. Sato, et al. (1999). Prognostic clinicopathologic factors, including immunologic expression in diffuse large B-cell lymphomas. Pathol. Int. 49: 1043-1052.

Zhang, W., J. Mi, N. Li, et al. (2001). Identification and characterisation of DPZF, a novel human BTB/POZ zinc finger protein sharing homology to BCL-6. Biochem. Biophys. Res. Commun. 282: 1067-1073.

Zhou, J., D. C. Allred, I. Avis, et al. (2001). Differential expression of the early lung cancer detection marker, heterogeneous nuclear ribonucleoprotein-A2/B1 (hnRNP-A2/B1) in normal breast and neoplastic breast cancer. Breast Cancer Res. Treat. 66: 217-224.

Zhou, J., L. Nong, M. Wloch, et al. (2001). Expression of early lung cancer detection marker: hnRNP-A2/B1 and its relation to microsatellite alteration in non-small cell lung cancer. Lung Cancer 34: 341-350.

| | | | | Recurrent | | |
|---|---|---|---|---|---|---|
| | Frequency | | | chromosomal | Previously- | |
| | of | Identity/ | | aberrations in | identified | mRNA expression |
| | identification | Unigene | Chromosomal | DLBCL | SEREX | data from LLMPP |
| Antigen | (/94) | name | localisation | (CGAP) | antigen? (Y/N) | Lymphochip |
| OX-TES-1 | 1 | Novel PAS | X | Specific locus | N | No data |
| | | domain | | unknown | | |
| | | protein | | | | |
| OX-TES-2 | 1 | Novel protein | 2 | Specific locus | N | No data |
| | | | | unknown | | |
| OX-TES-3 | 1 | PPM1A | 14q22.1-q22.3 | Infrequent | N | No data |
| OX-TES-4 | 3 | FLJ11565 | Xq22.3-23 | Infrequent | Y (breast | No data |
| | | | | | cancer) | |
| OX-TES-5 | 1 | H3F3B | 17q25 | Recurrent | N | No data |
| OX-TES-6 | 1 | RANBP2/ | 2q11-q13 | None | Y (testis; glioma | No differential |
| | | RANBP2L1 | | | and breast | expression in DLBCL |
| | | | | | cancer; giant | |
| | | | | | cell arteritis) | |
| OX-TES-7 | 1 | KIAA0352 | 12q13.2 | Infrequent | N | No data |
| OX-TES-8 | 1 | ZHX1 | 8p22 | Recurrent | Y (stomach | No data |
| | | | | | cancer) | |
| OX-TES-9 | 1 | VAT1 | 17q21 | Infrequent | N | No data |
| OX-TES-10 | 4 | SRPK1 | 6p21.3-p21.2 | Recurrent | Y (breast | Some differential |
| | | | | | cancer) | expression |
| OX-TES-11 | 2 | RNF20 | 9q22 | Recurrent | Y (stomach and | No data |
| | | | | | breast cancers) | |
| OX-TES-12 | 8 | HNRPA2B1 | 7p15 | Recurrent | Y (testis only) | No data |
| OX-TES-13 | 5 | STK11 | 19p13.3 | Recurrent | Y (renal cancer) | Some differential |
| | | | | | | expression in DLBCL |
| OX-TES-14 | 3 | HIS1 | 17q21.32 | Recurrent | Y (testis; | No data |
| | | | | | ovarian cancer) | |
| OX-TES-15 | 1 | MAPK6 | 15q21 | Recurrent | N | No differential |
| | | | | | | expression in DLBCL |
| OX-TES-16 | 1 | RBPSUH | 4 | Specific locus | Y (breast, renal, | Differential expression |
| | | | | unknown | stomach & lung | |
| | | | | | cancers; | |
| | | | | | melanoma | |
| OX-TES-17 | 1 | H2AFY | 5q31.3-q32 | Infrequent | Y (Systemic | No data |
| | | | | | lupus | |
| | | | | | erythematosus) | |
| OX-TES-18 | 14 | KIAA0643 | 16 | Specific locus | Y (ovarian and | No data |
| | | | | unknown | colon cancers) | |
| OX-TES-19 | 1 | ERCC3 | 2q21 | Infrequent | N | No differential |
| | | | | | | expression |
| OX-TES-20 | 7 | GKAP42 | 9 | Specific locus | N | No data |
| | | | | unknown | | |
| OX-TES-21 | 3 | SIRT1 | 10q21.3 | None | N | No data |
| OX-TES-22 | 2 | PSP1 | 13q11 | None | N | No data |
| OX-TES-23 | 3 | NSEP1 | 1p34 | Infrequent | Y (testis; | No data |
| | | | | | ovarian cancer; | |
| | | | | | giant cell | |
| | | | | | arteritis) | |
| OX-TES-24 | 9 | C20orf104 | 20q11.1-q11.23 | Infrequent | Y (testis; renal | No data |
| | | | | | and ovarian | |
| | | | | | cancers; glioma; | |
| | | | | | hepatocellular | |
| | | | | | carcinoma) | |
| OX-TES-25 | 7 | FLJ13942 | 6 | Specific locus | Y (stomach | No data |
| | | | | unknown | cancer) | |
| OX-TES-26 | 2 | ADD1 | 4p16.3 | Infrequent | N | No data |
| OX-TES-27 | 1 | NSD1 | 5q35 | Infrequent | N | No data |
| OX-TES-28 | 1 | BAP1 | 3p21.31-p21.2 | Infrequent | N | No data |

| | | Annealing | Extension | |
|---|---|---|---|---|
| | | temperature | time | Cycle |
| Primer sets | Primer sequence 5′-3′ | (° C.) | (mins) | number |
| OX-TES-1A | F:TACAGGAGCGGAAGAAGTGG | 60 | 2½ | 30 |
| | R:ACAGGAACAATGGGTTGGG | | | |
| OX-TES-1B | F:TCTCATCAATAGCAACTTGCTC | 55 | 2½ | 30 |
| | R:TCACACTCACTTCCCTCTTAC | | | |
| OX-TES-1C | F:TCCAGAGAGCAGGCTGAACAA | 60 | 5 | 35 |
| | R:AAGCCGGATGTAATCCTGTG | | | |
| OX-TES-3 | F:GGTAATGGGTTGCGATATGG | 55 | 2½ | 30 |
| | R:GCGGATTACTTGGTTTGTGA | | | |
| OX-TES-4 | F:ACACCACCATACCTTTTCCC | 55 | 2½ | 30 |
| | R:TTCTTTCCAGCTCAGCTTTC | | | |
| OX-TES-5 | F:AGGATTTCAAAACCGACCTG | 55 | 2½ | 30 |
| | R:CAACTGGATGTCTTTGGGC | | | |
| OX-TES-6 | F:ACATACCACTTCAAACTCCCC | 55 | 2½ | 30 |
| | R:ATCTTTATATCACCAATGCCCC | | | |
| OX-TES-8 | F:ACCAGGAAGAGGATGAAGAAG | 55 | 2½ | 30 |
| | R:ACGTTTTAGGCAGATTTCAGTC | | | |
| OX-TES-9 | F:TCCCAATCTCGTCCTCTGTC | 55 | 2½ | 30 |
| | R:ACCCTCCCTAATCCTAGCTC | | | |
| OX-TES-10 | F:AAGAGCAAGAACATAACGGAC | 55 | 2½ | 30 |
| | R:CGACCACTCATACTTCTCCAC | | | |
| OX-TES-11 | F:GAATAAACGCAAGGCAATGG | 55 | 2½ | 30 |
| | R:CAAGGAGAGAAGAGGTAGAGG | | | |
| OX-TES-12 | F:GGCTTTGGCTTTGTTACTTTTG | 55 | 2½ | 30 |
| | R:TATCCTCCTCTTCCTCCTCC | | | |
| OX-TES-13 | F:GGAAATTCAACTACTGAGGAGG | 55 | 2½ | 30 |
| | R:AGGTCGGAGATTTTGAGGG | | | |
| OX-TES-14 | F:GCTGGGGAAGAAAAAACATAGG | 55 | 2½ | 30 |
| | R:GATCATCCATGAGGAACTGC | | | |
| OX-TES-15 | F:ATGGTTTGTTGCCTACCTAGC | 55 | 2½ | 30 |
| | R:ATTCATGCTTGCCTTGGGATG | | | |
| OX-TES-19 | F:CACAACCCCAAAATTAACACC | 55 | 2½ | 30 |
| | R:ACAGAAATGACCCCACTCC | | | |
| OX-TES-20 | F:TTAGTTCTGTTCCCACCACC | 60 | 2½ | 30 |
| | R:GCTCATCTCTTTGTCTCCACTC | | | |
| OX-TES-21 | F:AAACTACTTCGCAACTATACCC | 55 | 2½ | 30 |
| | R:TCACCACCTAACCTATGACAC | | | |
| OX-TES-22 | F:AAATCTGCCCACCGACATC | 55 | 2½ | 30 |
| | R:CCCCATCACCACATCTTTCC | | | |
| OX-TES-24 | F:AGAAAAGCGACCCAAGCAG | 55 | 2½ | 30 |
| | R:TGAATCCACAGCAGTAGGAG | | | |
| OX-TES-26 | F:ACAAAGCCAAGTCCCGTTC | 55 | 2½ | 30 |
| | R:ATCACTACACCACACAAAACC | | | |
| OX-TES-28 | F:ATCAGACCAATCCAAGGCAG | 60 | 2½ | 30 |
| | R:AGCATGGAGATAAAGGTGCAG | | | |
| β-actin | F:ATCTGGCACCACACCTTCTACAATGAGCTGCG | 60 | 2½ | 30 |
| | R:CGTCATACTCCTGCTTGCTGATCCACATCTGC | | | |

Immunoreactivity

| Antigen | DLBCL (n = 10) | Healthy (n = 10) |
|---|---|---|
| OX-TES-1 | 3 | 1 |
| OX-TES-2 | 1 | 0 |
| OX-TES-3 | 4 | 0 |
| OX-TES-4 | 5 | 0 |
| OX-TES-5 | 4 | 0 |
| OX-TES-6 | 5 | 0 |
| OX-TES-7 | 1 | 0 |
| OX-TES-8 | 1 | 0 |
| OX-TES-9 | 1 | 0 |
| OX-TES-10 | 4 | 1 |
| OX-TES-11 | 3 | 1 |
| OX-TES-12 | 4 | 3 |
| OX-TES-13 | 5 | 3 |
| OX-TES-14 | 5 | 4 |
| OX-TES-15 | 3 | 1 |
| OX-TES-16 | 5 | 4 |
| OX-TES-17 | 1 | 1 |
| OX-TES-18 | 5 | 6 |
| OX-TES-19 | 2 | 2 |
| OX-TES-20 | 7 | 4 |
| OX-TES-21 | 3 | 1 |
| OX-TES-22 | 3 | 1 |
| OX-TES-23 | 7 | 6 |
| OX-TES-24 | 7 | 5 |
| OX-TES-25 | 6 | 6 |
| OX-TES-26 | 4 | 3 |
| OX-TES-27 | 3 | 2 |
| OX-TES-28 | 2 | 2 |

| | Immunoreactivity | | |
|---|---|---|---|
| | AML | CML | Healthy |
| Antigen | (n = 10) | (n = 10) | (n = 10) |
| OX-TES-1 | 0 | 0 | 0 |
| OX-TES-3 | 1 | 0 | 0 |
| OX-TES-4 | 0 | 0 | 0 |
| OX-TES-5 | 1 | 0 | 1 |
| OX-TES-6 | 4 | 2 | 0 |
| OX-TES-10 | 4 | 0 | 0 |
| OX-TES-11 | 1 | 0 | 0 |
| OX-TES-12 | 0 | 0 | 0 |
| OX-TES-13 | 1 | 2 | 1 |
| OX-TES-15 | 0 | 0 | 0 |
| OX-TES-20 | 1 | 0 | 0 |
| OX-TES-21 | 3 | 0 | 0 |
| OX-TES-22 | 0 | 1 | 0 |
| OX-TES-24 | 3 | 1 | 4 |
| OX-TES-28 | 0 | 0 | 0 |

1. A nucleic acid molecule encoding a tumour-associated antigen having the amino acid sequence illustrated in any of FIGS. 2 or 10, or a functional equivalent thereof.

2. A molecule according to claim 1 which is a DNA or RNA molecule.

3. A molecule according to claim 1 which is a cDNA molecule.

4. A molecule according to claim 1, wherein said nucleic acid molecule encoding said tumour-associated antigens comprises the sequence in any of FIGS. 1 or 9 respectively.

5. A nucleic acid molecule capable of hybridising to a molecule according to claim 1 under conditions of high stringency.

6. A nucleic acid molecule according to claim 1, wherein said tumour antigen is one associated with lymphomas.

7. A nucleic acid molecule according to claim 6, wherein said lymphoma is a B cell lymphoma.

8. A nucleic acid molecule according to claim 1, wherein said tumour antigen is a solid tumour.

9. A nucleic acid molecule according to claim 8 wherein said solid tumour is any of, kidney, breast, uterus, cervix, colon, lung, stomach, rectum or small intestine.

10. A nucleic acid molecule encoding a tumour associated antigen having the amino acid sequence illustrated in any of FIGS. 12, 14, 18 or 23.

11. A nucleic acid molecule according to claim 10 which is a DNA molecule.

12. A nucleic acid molecule according to claim 10 having the sequence of nucleotides in any of FIGS. 11, 13, 17 or 22

13. A nucleic acid molecule which is capable of hybridising to the nucleic acid molecule of claim 10 under conditions of high stringency.

14. A tumour associated antigen encoded by a nucleic acid molecule according to claim 1.

15. A tumour-associated antigen encoded by a nucleic acid molecule having a nucleotide sequence illustrated in any of FIGS. 1 or 9.

16. A tumour associated antigen having the amino acid sequence according to any of FIGS. 2 or 10.

17. A tumour associated antigen encoded by a nucleic acid molecule having the nucleotide sequence according to any of FIGS. 11, 13, 17 or 22.

18. A tumour associated antigen having the amino acid sequence according to any of FIGS. 12, 14, 18 or 23.

19. An expression vector comprising a nucleic acid molecule according to claim 1.

20. A host cell or organism transformed or transfected with an expression vector according to claim 19.

21. A transgenic non-human organism comprising a transgene capable of expressing a tumour antigen according to claim 14.

22. An antibody or fragment thereof capable of binding to a tumour-associated antigen according to claim 14.

23. An antibody according to claim 22 which is any of a polyclonal or monoclonal antibody.

24. An antibody according to claim 22 which is a humanised antibody.

25. An antibody according to claims 22 which is an autoantibody.

26. An antibody according to claim 22 further comprising a toxic or reporter molecule.

27. An antibody according to claim 26 wherein said reporter molecule is a radioisotope.

28. A method of identifying malignant cells or tumours in a mammal which method comprises identifying in a sample of bodily fluid from said mammal antibodies from said mammal capable of forming complexes with a tumour-associated antigen according to claim 14, or an epitope thereof.

29. A method of identifying malignant cells or tumours in a mammal which method comprises identifying in a sample of bodily fluid from said mammal antibodies from said mammal capable of forming complexes with any of the tumour-associated antigens designated as OX-TES-1, 2, 3, 4, 5, 7, 9, 15, 19, 20, 21, 22, 26, 27 or 28 in Table 1, or an epitope thereof.

30. A method according to claim 28 wherein said mammal is a human and the antibodies are human autoantibodies.

31. A method according to claim 28 wherein said malignancy is any of a solid tumour, a leukaemia or a lymphoma.

32. A method according to claim 31 wherein said solid tumour is from any of kidney, breast, uterus, cervix, colon, lung, stomach, rectum or small intestine.

33. A method according to claim 31 wherein said lymphoma is a B cell lymphoma such as any of diffuse large B-cell lymphoma (DLBCL), follicular lymphoma, Burkitt's lymphoma, but preferably DLBCL.

34. A method according to claim 28 wherein said bodily fluid comprises human serum or plasma.

35. A method of detecting lymphoma in a patient which method comprises identifying autoantibodies from a bodily fluid of said patient capable of forming complexes with any of the antigens designated OX-TES-1-28 as set forth in Table 1, or an epitope thereof.

36. A method according to claim 35 wherein said lymphoma is a B cell lymphoma including any of diffuse large B-cell lymphoma, follicular lymphoma or Burkitt's lymphoma.

37. A method according to claim 35, wherein said bodily fluid comprises human serum or plasma.

38. A method of treating malignant cells or tumours in a patient which method comprises administering to said patient an antibody according to claim 22 or a epitope thereof.

39. A method of treating malignant cells or tumours in a patient which method comprises administering to said patient an antibody capable of forming complexes with any of the tumour-associated antigens designated as OX-TES-1, 2, 3, 4, 5, 7, 9, 15, 19, 20, 21, 22, 26, 27 or 28 in Table 1 or a epitope thereof.

40. A method according to claim 38 wherein said tumour is any of a solid tumour, a leukaemia or a lymphoma.

41. A method according to claim 40 wherein solid tumour is from any of kidney, breast, uterus, cervix, colon, lung, stomach, rectum or small intestine.

42. A method according to claim 40 wherein said lymphoma is a B cell lymphoma such as any of diffuse large B-cell lymphoma (DLBCL), follicular lymphoma, Burkitt's lymphoma, but preferably DLBCL.

43. A method of treating lymphoma in a patient which method comprises administering to said patient an antibody capable of binding to or forming complexes with any of the antigens identified as OX-TES-1-28 in Table 1, or an epitope thereof.

44. A method according to claim 42 wherein said lymphoma is a B cell lymphoma such as any of diffuse large B-cell lymphoma (DLBCL), follicular lymphoma, Burkitts lymphoma, but preferably DLBCL.

45. A method of treating malignant cells or tumours in a patient, which method comprises administering to said patient a nucleic acid molecule according to claim 5.

46. A method according to claim 45 wherein said nucleic acid molecule is an antisense molecule.

47. A method according to claim 45 wherein said nucleic acid molecule is double stranded RNA.

48. A method of treating lymphoma in a patient, which method comprises administering to said patient a nucleic acid molecule capable of hybridising to the nucleic acid molecules encoding any of the antigens designated as OX-TES-1 to 28 in Table 1, under conditions of high stringency.

49. An assay kit for detecting malignant cells or tumours in a patient said kit comprising one or more tumour-associated antigens according to claim 14, and means for contacting said antigen with a sample of bodily fluid from said patient

50. An assay kit for detecting lymphomas in a patient said kit comprising one or more of any of the antigens designated OX-TES-1 to 28 as set forth in Table 1, and means for contacting said antigen with a sample of bodily fluid from a patient.

51. Use of the method according to claim 27 in determining a malignant cell/tumour-associated antigen profile of an individual suffering from a malignancy or tumour.

52. Use of the method according to claim 35 in determining a lymphoma-associated antigen profile of an individual suffering from a lymphoma.

53. A malignancy or tumour diagnostic reagent comprising mammalian autoantibodies having a specificity for at least one of the tumour-associated antigens according to claim 14, or an epitope thereof.

54. Use of a method according to claim 27 to screen for minimal residual disease,recurrence of malignancy or tumour after treatment, or to monitor the progress of the treatment of an individual for a malignancy or tumour.

55. Use according to claim 54 wherein said malignancy or tumour is a lymphoma, leukaemia or a solid tumour.

56. Use of a method according to claim 35 to screen for reccurrence of a lymphoma after treatment, or to monitor the progress of the treatment of an individual for said lymphoma.

57. An immortalised cell population capable of producing antibodies against an epitope of tumour-associated antigens in an individual according to claim 14.

58. An immortalised cell population capable of producing antibodies against an epitope of lymphoma-associated antigens in an individual according to claim 14, or the antigens designated as OX-TES-1 to 28 of Table 1.

59. An immortalised cell population according to claim 57 wherein said antibodies are directed against an epitope of DLBCL-associated tumour antigens.

60. A vaccine composition comprising an effective amount of a polypeptide according to claim 14, or any isolated antigen as indicated in Table 1, or an epitope or immunogenic fragment thereof, together with a pharmaceutically acceptable carrier.

61. A vaccine composition comprising an effective amount of a nucleic acid molecule according to claim 1, or a nucleotide sequence encoding any of the antigens set forth in Table 1, or an epitope or immunogenic fragment thereof, together with a pharmaceutically acceptable carrier.

62. A vaccine composition comprising an effective amount of antigen presenting cells modified to express any of the antigens according to claim 14 or the antigens illustrated in Table 1 or an epitope or immunogenic fragment thereof, together with a pharmaceutically acceptable carrier.

63. A vaccine composition according to claim 60 which further comprises an appropriate adjuvant.

64. A nucleic acid molecule according to claim 1, or encoding an antigen as set out in Table 1 for use in treating a human or animal body.

65. A tumour or lymphoma associated antigen according to claim 14, or an antigen as set out in Table 1, or an epitope thereof for use in the treatment of a human or animal body.

66. An antibody according to claim 22, or an antibody specific for an antigen as set out in Table 1 or an epitope thereof, for use in treatment of a human or animal body.

67. A method of identifying lymphoma patients having a poor prognosis or that do not respond to treatment, comprising identifying those patients expressing OX-TES-4.

68. A method of monitoring CTL responses in a patient to antigens OX-TES-1 to 28 in Table 1, which comprises contacting a bodily fluid from said patient with a tetramer complex of an antigenic fragment any of said antigens OX-TES-1 to 28 and MHC Class I molecules and monitoring the level of CTL responses for said antigens.

69. A method of purifying CTL cells from a patient, which comprises contacting a bodily fluid from said patient with a tetramer complex of an antigenic fragment any of said antigens OX-TES-1 to 28 and MHC Class I molecules and removing any bound cells that recognise the antigenic peptide(s).
